(19) United States
(12) Patent Application Publication
(10) Pub. No.: US 2021/0380994 A1
(43) Pub. Date: Dec. 9, 2021

(54) LACTOCOCCUS LACTIS EXPRESSION SYSTEM FOR DELIVERING PROTEINS EFFICACIOUS FOR THE TREATMENT OF EPITHELIAL BARRIER FUNCTION DISORDERS

(71) Applicants: Daniela KEILBERG, San Francisco, CA (US); Andrew Wonhee HAN, San Francisco, CA (US); Andrew W. GOODYEAR, San Francisco, CA (US); Second Genome, Inc., Brisbane, CA (US)

(72) Inventors: Michi Izumi Willcoxon, Brisbane, CA (US); Daniela Keilberg, Brisbane, CA (US); Andrew Wonhee Han, Brisbane, CA (US); Andrew W. Goodyear, Brisbane, CA (US)

(21) Appl. No.: 17/282,857

(22) PCT Filed: Oct. 9, 2019
(86) PCT No.: PCT/US2019/055476

§ 371 Date: Apr. 5, 2021

Related U.S. Application Data
(60) Provisional application No. 62/743,372, filed on Oct. 9, 2018.

(51) Int. Cl. C12N 15/74; A61K 35/744; C07K 14/195; C12N 15/70; A61P 1/00; A61P 29/00

(52) U.S. Cl. CPC C12N 15/746; A61K 35/744; C07K 14/195; C12N 2800/101; A61P 1/00; A61P 29/00; C12N 15/70

(57) ABSTRACT

The disclosure relates to live biotherapeutic products, probiotics, and therapeutic composition comprising said probiotics having therapeutic proteins, and methods of using them to treat various human diseases. In particular aspects, the disclosure provides such compositions comprising strains of the Lactococcus lacus bacterium within which said therapeutic protein are present. The disclosed pharmaceutical compositions are useful for treating gastrointestinal inflammatory diseases and gastrointestinal conditions associated with decreased epithelial cell barrier function or integrity, especially, for treating or preventing various types of mucositis.

### CROSS REFERENCE TO RELATED APPLICATIONS

This application claims priority to U.S. Provisional Application Ser. No. 62/743,372, filed on Oct. 9, 2018, which is incorporated by reference in its entirety herein.

### DESCRIPTION OF THE TEXT FILE SUBMITTED ELECTRONICALLY

The contents of the text file submitted electronically herewith arm incorporated herein by reference in their entirety: A computer readable format copy of the Sequence Listing filename: 47192_0028WO1_S725.txt, date recorded, Oct. 9, 2019, file size=164 kilobytes.

### Field

In some aspects, the present disclosure relates to live biotherapeutic products, probiotics, and therapeutic compositions comprising live bacteria expressing therapeutic proteins, and methods of using them to treat various human diseases. The microbial compositions have application, inter alia, in treatment of gastrointestinal inflammatory diseases and epithelial barrier function disorders. In some embodiments, compositions provided herein can be used in the treatment, or prevention of treatment or prevention of disease states associated with abnormally permeable epithelial barriers as well as various types of mucositis.

### BACKGROUND

Mucositis a pathological condition characterized by mucosal damage, ranging from mild inflammation to deep ulcerations of the mucous membranes lining the digestive tract. It affects one or more parts of the alimentary tract from the mouth to the anus. Mucositis usually occurs as an adverse effect of chemotherapy and radiotherapy treatment of diseases such as cancer. Cell death resulting from chemotherapy or radiotherapy, makes the mucosal lining of the alimentary track to become thin, then inflamed and/or ulcerated.

Oral and gastrointestinal (GI) mucositis occurs in association with many diseases and by many different mechanisms. For example, recurrent oral ulceration is a condition in which a break or an erosion in the mucous membrane occurs recurrently in the mouth. While specific triggers of recurrent oral ulceration remain poorly defined, family tendency, trauma, hormonal factors, food or drug hypersensitivity, emotional stress, chemotherapy, irradiation therapy, neutropenic conditions and autoimmune diseases are known to be predisposing conditions for recurrent oral ulceration.

While many current therapies target inflammation and ulceration of the mucous membranes lining the digestive tract, the lack of therapies promoting mucosal healing provides an opportunity for novel therapies promoting epithelial repair and intestinal barrier integrity.

Therapeutics available in the market typically merely aim to aid increasing oral hygiene so as to prevent the mucositis from becoming worse. While this treatment can be helpful, this narrow and indirect therapeutic mode of action generally disregards the important contribution that epithelial barrier integrity plays in the cause of mucositis and associated complications thereof. Also, current therapy for mucositis is predominantly palliative and focused on pain control; however, it is often insufficient to control mucositis pain.

Thus, there is a great need in the art for the development of a therapeutic, which not only suppresses the inflammatory response in the mucous membranes of gastrointestinal tract, but that also acts in concert to restore the epithelial barrier function in an individual. The live biotherapeutic products, probiotics, and compositions thereof as taught herein prevent or treat mucositis and associated complications thereof in an individual.

### SUMMARY

In some aspects, the present disclosure addresses the important need in the medical community for a therapeutic which can effectively treat a subject suffering from a gastrointestinal disorder such as an inflammatory bowel disease (IBD) and various types of mucositis.

Accordingly, provided herein in one aspect is a recombinant host including a first nucleic acid comprising a promoter operably linked to a nucleic acid sequence encoding a signal peptide and a protein of interest, wherein the signal peptide is N-terminal to the protein of interest, wherein the promoter is selected from the group consisting of usp45 and thyA, wherein the first nucleic acid is integrated into the genome of the host, and wherein the host is a thymidylate synthase (thyA) auxotroph, a 4-hydroxy-tetrahydrodipicolinate synthase (dapA) auxotroph, or both.

Implementations can include one or more of the following features. The host can be a bacterium. The signal peptide can be a usp45 signal peptide. The host can further include a viability enhancement. The viability enhancement can include disruption of an endogenous gene encoding a protein involved in the catabolism of lactose, maltose, sucrose, trehalose, or glycine betaine. The protein involved in the catabolism of lactose, maltose, sucrose, trehalose, or glycine betaine cam be selected from the group consisting of a sucrose 6-phosphate, a maltose phosphorylase, a beta-galactosidase, a phospho-b-galactosidase, a trehalose 6-phosphate phosphorylase, and combinations thereof. The viability enhancement can include disruption of an endogenous gene encoding a protein involved in export of lactose, maltose, sucrose, trehalose, or glycine betaine. The protein involved in the export of lactose, maltose, sucrose, trehalose, or glycine betaine can be a permease IIC component. The viability enhancement can include an exogenous nucleic acid encoding a protein involved in the import of lactose, maltose, sucrose, trehalose, or glycine betaine. The protein involved in the import of lactose, maltose, sucrose, trehalose, or glycine betaine can be selected from the group consisting of a sucrose phosphotransferase, a maltose ABC-transporter permease, a maltose binding protein, a lactose phosphotransferase, a lactose permease, a glycine betaine/proline ABC transporter permease component, and combinations thereof. The viability enhancement can include an exogenous nucleic acid encoding a protein involved in the production of lactose, maltose, sucrose, trehalose, or glycine betaine. The protein involved in the production of lactose, maltose, sucrose, trehalose, or glycine betaine can be selected from the group consisting of a trehalose-6-phosphate synthase, a trehalose-6-phosphate phosphatase, and combinations thereof. The host can be a non-pathogenic bacterium. The bacterium can be a probiotic bacterium. The bacterium can be selected from the group consisting of Bacteroides, Bifidobacterium, Clostridium, Escherichia, Eubacterium, Lactobacillus, Lactococcus, and Roseburia. The host can be Lactococcus lactis. The Lactococcus lactis can be strain MG1363 or strain NZ9000. The protein of interest can include an amino acid sequence with at least about 90% sequence identity to SEQ ID NO: 19 and/or SEQ ID NO: 34. The protein of interest can include an amino acid sequence having at least about 95% sequence identity to SEQ ID NO: 19 or SEQ ID NO:34. The protein of interest can include an amino acid sequence having at least about 97% sequence identity to SEQ ID NO: 19 or SEQ ID NO: 34. The protein of interest can include an amino acid sequence having at least about 98% sequence identity to SEQ ID NO: 19 or SEQ ID NO: 34. The protein of interest can include an amino acid sequence having at least about 99% sequence identity to SEQ ID NO: 19 or SEQ ID NO: 34. The protein of interest can include the amino acid sequence of SEQ ID NO: 19 or SEQ ID NO: 34. The protein of interest can include an amino acid sequence having at least about 90% sequence identity to SEQ ID NO: 19, and wherein (i) the amino acid at position 147 of the protein of interest is valine, and/or (ii) the amino acid at position 151 of the protein of interest is serine, and/or (iii) the amino acid at position 84 of the protein of interest is aspartic acid, and/or (iv) the amino acid at position 83 of the protein of interest is serine, and/or (v) the amino acid at position 53 of the protein of interest is serine. The protein of interest can include an amino acid sequence having at least about 90% sequence identity to SEQ ID NO: 19, wherein the amino acid at position 147 of the protein of interest is valine and the amino acid at position 151 of the protein of interest is swine. The protein of interest can include an amino acid sequence having at least about 90% sequence identity to SEQ ID NO: 19, wherein the amino acid at position 84 of the protein of interest is aspartic acid, the amino acid at position 147 of the protein of interest is valine, and the amino acid at position 151 of de protein of interest is serine. The protein of interest can include an amino acid sequence having at least about 90%, sequence identity to SEQ ID NO: 19, wherein the amino acid at position 83 of the protein of interest is serine, the amino acid at position 147 of the protein of interest is valine, and the amino acid at position 151 of the protein of interest is serine. The protein of interest can include an amino acid sequence having at least about 90% sequence identity to SEQ ID NO: 19, wherein the amino acid at position 53 of the protein of interest is serine, the amino acid at position 84 of the protein of interest is aspartic acid, the amino acid at position 147 of the protein of interest is valine, and the amino acid at position 151 of the protein of interest is serine. The protein of interest can include an amino acid sequence having at least about 90% sequence identity to SEQ ID NO: 19, wherein the amino acid at position 53 of the protein of interest is serine, the amino acid at position 83 of the protein of interest is serine, the amino acid at position 147 of the protein of interest is valine, and the amino acid at position 151 of the protein of interest is serine. The protein of interest can include an amino acid sequence having at least about 90% sequence identity to SEQ ID NO: 19, wherein the amino acid at position 147 of the protein of interest is not cysteine, the amino acid at position 151 of the protein of interest is not cysteine the amino acid at position 83 of the protein of interest is not asparagine, and/or the amino acid at position 53 of the protein of interest is not asparagine. The protein of interest can include an amino acid sequence having at least about 90% sequence identity to SEQ ID NO: 34, wherein (i) the amino acid at position 76 of the protein of interest is valine, and/or (ii) the amino acid at position 80 of the protein of interest is serine; and/or (iii) the amino acid at position 13 of the protein of interest is aspartic acid; and/or (iv) the amino acid at position 12 of the protein of interest is serine. The protein of interest can include an amino acid sequence having at least about 90% sequence identity to SEQ ID NO: 34, wherein the amino acid at position 76 of the protein of interest is valine, and the amino acid at position 80 of the protein of interest is serine. The protein of interest can include an amino acid sequence having at least about 90% sequence identity to SEQ ID NO: 34, wherein the amino acid at position 13 of the protein of interest is aspartic acid, the amino acid at position 76 of the protein of interest is valine, and the amino acid at position 80 of the protein of interest is serine. The protein of interest can include an amino acid sequence having at least about 90% sequence identity to SEQ ID NO: 34, wherein the amino acid at position 12 of the protein of interest is serine, the amino acid at position 76 of the protein of interest is valine, and the amino acid at position 80 of the protein of interest is serine. The protein of interest can include an amino acid sequence having at least about 90% sequence identity to SEQ ID NO: 34, wherein the amino acid at position 76 of the protein of interest is not cysteine, the amino acid at position 90 of the protein of interest is not cysteine, and the amino acid at position 12 of the protein of interest is not asparagine. The protein of interest can include an amino acid sequence having at least about 90% sequence identity to SEQ ID NO:46, SEQ ID NO: 47, SEQ ID NO: 48, or SEQ ID NO: 49.

In one aspect, also provided is a pharmaceutical composition including a therapeutically effective amount of any of the recombinant hosts provided herein, and a pharmaceutically acceptable carrier. In some embodiments, the composition can include 10⁶-10¹² colony forming units of the recombinant host.

In an aspect, provided is a method of treating a gastrointestinal epithelial cell barrier function disorder, including administering to a subject in need thereof a pharmaceutical composition including a therapeutically effective amount of any of the recombinant hosts provided herein, and a pharmaceutically acceptable carrier.

Implementations can include one or more of the following features. The composition can include viable recombinant hosts. The composition can include non-viable recombinant hosts. The gastrointestinal epithelial cell barrier function disorder can be a disease associated with decreased gastrointestinal mucosal epithelium integrity. The disorder can be selected from the group consisting of: inflammatory bowel disease, ulcerative colitis, Crohn's disease, short bowel syndrome, GI mucositis, oral mucositis, chemotherapy-induced mucositis, radiation-induced mucositis, necrotizing enterocolitis, pouchitis, a metabolic disease, celiac disease, inflammatory bowel syndrome, and chemotherapy associated steatohepatitis (CASH). The disorder can be oral mucositis. The composition can be formulated for oral ingestion. The composition can be an edible product. The composition can be formulated as a pill, a tablet, a capsule, a suppository, a liquid, or a liquid suspension.

In an aspect, provided is a bacterium for treating a gastrointestinal epithelial cell barrier function disorder including at least one first heterologous nucleic acid, the first nucleic acid including a promoter operably linked to a nucleic acid sequence encoding a first polypeptide having at least about 90% sequence identity to SEQ ID NO: 19 and/or SEQ ID NO: 34.

Implementations can include one or more of the following features. The promoter can be a constitutive promoter or an inducible promoter. The constitutive promoter can be a usp45 promoter or a thyA promoter. The inducible promoter can be a nisA promoter. The first nucleic acid can encode a signal peptide N-terminal to the first polypeptide. The signal peptide can be a usp45 signal peptide. The bacterium can further include a second heterologous nucleic acid encoding at least one second polypeptide. The second polypeptide can include trehalose-6-phosphate synthase (otsA) or trehalose-6-phosphate phosphatase (otsB). The second nucleic acid can encode trehalose-6-phosphate synthase (otsA) and trehalose-6-phosphate phosphatase (otsB). The second nucleic acid can be integrated into a genome of the bacterium. The bacterium can be a non-pathogenic bacterium. The bacterium can be a probiotic bacterium. The bacterium can be selected from the group consisting of Bacteroides, Bifidobacterium, Clostridium, Escherichia, Eubacterium, Lactobacillus, Lactococcus, and Roseburia. The bacterium can be Lactococcus lactis. The first polypeptide can include an amino acid sequence having at least about 95% sequence identity to SEQ ID NO: 19. The first polypeptide can include an amino acid sequence having at least about 97% sequence identity to SEQ ID NO: 19. The first polypeptide can include an amino acid sequence having at least about 98% sequence identity to SEQ ID NO: 19. The first polypeptide can include an amino acid sequence having at least about 99% sequence identity to SEQ ID NO: 19. The first polypeptide can include the amino acid sequence of SEQ ID NO: 19. The first polypeptide can include an amino acid sequence having at least about 90% sequence identity to SEQ ID NO: 19, wherein the amino acid at position 147 of the first polypeptide is valine. The first polypeptide can include an amino acid sequence having at least about 90% sequence identity to SEQ ID NO: 19, wherein the amino acid at position 151 of the first polypeptide is serine. The first polypeptide can include an amino acid sequence having at least about 90% sequence identity to SEQ ID NO: 19, wherein the amino acid at position 147 of the first polypeptide is valine, and the amino acid at position 151 of the first polypeptide is serine. The first polypeptide can include an amino acid sequence having at least about 90% sequence identity to SEQ ID NO: 19, wherein the amino acid at position 84 of the first polypeptide is aspartic acid. The first polypeptide can include an amino acid sequence having at least about 90% sequence identity to SEQ ID NO: 19, wherein the amino acid at position 84 of the first polypeptide is aspartic acid, the amino acid at position 147 of the first polypeptide is valine, and the amino acid at position 151 of the polypeptide is serine. The first polypeptide can include an amino acid sequence having at least about 90% sequence identity to SEQ ID NO: 19, wherein the amino acid at position 83 of the first polypeptide is serine. The first polypeptide can include an amino acid sequence having at least about 90% sequence identity to SEQ ID NO: 19, wherein the amino acid at position 83 of the first polypeptide is serine, the amino acid at position 147 of the first polypeptide is valine, and the amino acid at position 151 of the first polypeptide is serine. The first polypeptide can include an amino acid sequence having at least about 90% sequence identity to SEQ ID NO: 19, wherein the amino acid at position 53 of the first polypeptide is serine. The first polypeptide can include on amino acid sequence having at least about 90% sequence identity to SEQ ID NO: 19, wherein the amino acid at position 53 of the fart polypeptide is serine, the amino acid at position 84 of the first polypeptide is aspartic acid, the amino acid at position 147 of the first polypeptide is valine, and the amino acid at position 151 is sine. The first polypeptide can include an amino acid sequence having at least about 90% sequence identity to SEQ ID NO: 19, wherein the amino acid at position 53 of the first polypeptide is serine, the amino acid at position 83 of the first polypeptide is swine, the amino acid at position 147 of the first polypeptide is valine, and the amino acid at position 151 of the first polypeptide is swine. The first polypeptide can include an amino acid sequence having at least about 90% sequence identity to SEQ ID NO: 19, wherein the amino acid at position 147 of the first polypeptide is not cysteine, the amino acid at position 151 of the first polypeptide is not cysteine, the amino acid at position 83 of the first polypeptide is not asparagine, and/or the amino acid at position 53 of the first polypeptide is not asparagine. The rust polypeptide can include an amino acid sequence having at least about 95% sequence identity to SEQ ID NO: 34. The first polypeptide can include an amino acid sequence having at least about 97% sequence identity to SEQ ID NO: 34. The first polypeptide can include an amino acid sequence having at least about 98% sequence identity to SEQ ID NO: 34. The first polypeptide can include an amino acid sequence having at least about 99% sequence identity to SEQ ID NO: 34. The first polypeptide can include the amino acid sequence of SEQ ID NO: 34. The first polypeptide can include an amino acid sequence having at least about 90% sequence identity to SEQ ID NO: 34, wherein the amino acid at position 76 of the first polypeptide is valine. The first polypeptide can include an amino acid sequence having at least about 90% sequence identity to SEQ ID NO: 34, wherein the amino acid at position 80 of the first polypeptide is serine. The first polypeptide can include an amino acid sequence having at least about 90% sequence identity to SEQ ID NO: 34, wherein the amino acid at position 76 of the first polypeptide is valine, and the amino acid at position 80 of the first polypeptide is serine. The first polypeptide can include an amino acid sequence having at least about 90% sequence identity to SEQ ID NO: 34, wherein the amino acid at position 13 of the first polypeptide is aspartic acid. The first polypeptide can include an amino acid sequence having at least about 90% sequence identity to SEQ ID NO: 34, wherein the amino acid at position 13 of the first polypeptide is aspartic acid, the amino acid at position 76 of the first polypeptide is valine, and the amino acid at position 80 of the first polypeptide is serine. The first polypeptide can include an amino acid sequence having at least about 90% sequence identity to SEQ ID NO: 34, wherein the amino acid at position 12 of the first polypeptide is swine. The first polypeptide can include an amino acid sequence having at least about 90% sequence identity to SEQ ID NO: 34, wherein the amino acid at position 12 of the first polypeptide is swine, the amino acid at position 76 of the first polypeptide is valine, and the amino acid at position 80 of the first polypeptide is swine. The rust polypeptide can include an amino acid sequence having at least about 90% sequence identity to SEQ ID NO-34 and wherein the amino acid at position 76 of the first polypeptide is not cysteine, the amino acid at position 80 of the first polypeptide is not cysteine, and the amino acid at position 12 of the first polypeptide is not asparagine. The first nucleic acid can be integrated into the genome of the bacterium. The first nucleic acid can be on a vector in the bacterium.

In an aspect, also provided herein is a pharmaceutical composition including a therapeutically effective amount of any of the bacteria provided herein and a pharmaceutically acceptable carrier.

In one aspect, also provided herein is a method of treating a gastrointestinal epithelial cell barrier function disorder, including administering to a subject in need thereof a pharmaceutical composition, including a therapeutically effective amount of any of the bacteria provided herein and a pharmaceutically acceptable carrier. The composition can include viable bacteria. The gastrointestinal epithelial cell barrier function disorder can be a disease associated with decreased gastrointestinal mucosal epithelium integrity. The disorder can be selected from the group consisting of: inflammatory bowel disease, ulcerative colitis, Crohn's disease, short bowel syndrome, GI mucositis, oral mucositis, chemotherapy-induced mucositis, radiation-induced mucositis, necrotizing enterocolitis, pouchitis, a metabolic disease, celiac disease, inflammatory bowel syndrome, and chemotherapy associated steatohepatitis (CASH). The disorder can be oral mucositis. The composition can be formulated for oral ingestion. The composition can be an edible product. The composition can be formulated as a pill, a tablet, a capsule, a suppository, a liquid, or a liquid suspension.

In one aspect, live biotherapeutic products, probiotics, and therapeutic compositions comprising live bacteria expressing therapeutic proteins are provided which can improve and/or maintain epithelial barrier integrity. These live biotherapeutic products and/or probiotics can also reduce inflammation of the gastrointestinal tract of the subject and/or decrease symptoms associated with inflammation of the gastrointestinal tract.

The live biotherapeutic products and/or probiotics provided herein can be useful in treating numerous diseases including IBD and various types of mucositis, and/or symptoms that may be associated with decreased gastrointestinal epithelial cell barrier function or integrity.

In some embodiments, the disclosure relates to a bacterium for treating a gastrointestinal epithelial cell barrier function disorder, Comprising: at least one first heterologous nucleic acid encoding a first polypeptide having at least about 90% sequence identity to SEQ ID NO: 19 and/or SEQ ID NO: 34. In some embodiments, the nucleic acid is operably linked to a promoter. In some embodiments, the promoter is a constitutive promoter or an inducible promoter. In some embodiments, the constitutive promoter is a usp45 promoter. In some embodiments, the inducible promoter is nisA promoter, which is directly or indirectly induced by nisin.

In some embodiments, the disclosure provides a novel bacterium for treating a gastrointestinal epithelial cell barrier function disorder, comprising: at least one first heterologous nucleic acid encoding a first polypeptide having at least about 90% sequence identity to SEQ ID NO: 19 and/or SEQ ID NO: 34. In some embodiments, the bacterium further comprises a signal peptide sequence, which is operably linked to said first nucleic acid. In some embodiments, the signal peptide is a USP45 signal peptide.

In some embodiments, the disclosure provides a novel bacterium for treating a gastrointestinal epithelial cell barrier function disorder, comprising: at least one first heterologous nucleic acid encoding a first polypeptide having at least about 90% sequence identity to SEQ ID NO: 19 and/or SEQ ID NO: 34. In some embodiments, the bacterium further comprises at least one second nucleic acid encoding a second polypeptide. In some embodiments, the second nucleic acid comprises trehalose-6-phosphate synthase (otsA) or trehalose-6-phosphate phosphatase (otsB). In some embodiments, the second nucleic acid comprises trehalose-6-phosphate synthase (otsA) and trehalose-6-phosphate phosphatase (otsB). In some embodiments, the second polypeptide comprises trehalose.

In some embodiments, the disclosure provides a novel bacterium is a non-pathogenic bacterium. In some embodiments, the bacterium is a probiotic bacterium. In some embodiments, the bacterium is selected from the group consisting of Bacteroides, Bifidobacterium, Clostridium. Escherichia, Eubacterium, Lactobacillus, Lactococcus, and Roseburia. In some embodiments, the bacterium is Lactococcus lactis (L. lactis).

In some embodiments, the disclosure provides a novel bacterium for treating a gastrointestinal epithelial cell barrier function disorder, comprising: at least one first heterologous nucleic acid encoding a first polypeptide having at least about 90% sequence identity to SEQ ID NO: 19 and/or SEQ ID NO: 34. In some embodiments, the first heterologous nucleic acid is integrated into a genome of said bacterium. In some embodiments, the first polypeptide is a therapeutic protein for treating a gastrointestinal epithelial cell barrier function disorder and/or disease.

In some embodiments, the disclosure provides that the first polypeptide comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%, sequence identity to SEQ ID NO:19. In some embodiments, the first polypeptide does not comprise an amino acid sequence identical to SEQ ID NO:3. In some embodiments, the first polypeptide comprises an amino acid sequence which is not naturally occurring.

In some embodiments, the first polypeptide comprises the amino acid sequence of SEQ ID NO:1, SEQ ID NO:3, SEQ ID NO:5, SEQ ID NO:7, SEQ ID NO:9, SEQ ID NO: 11, SEQ ID NO:13, SEQ ID NO:15, SEQ ID NO:17, or SEQ ID NO:19. In some embodiments, the first polypeptide comprises the amino acid sequence of SEQ ID NO:3. In some embodiments, the first polypeptide comprises the amino acid sequence of SEQ ID NO: 19.

In some embodiments, the first polypeptide comprises an amino acid sequence which is at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%, or 100% identical to SEQ ID NO:19, wherein the amino acid sequence has at leas 1, 2, 3 or 4 amino acid substitutions relative to SEQ ID NO:19 or to SEQ ID NO:3. In some embodiments, the amino acid sequence has at least 2 and less than 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acid substitutions relative to SEQ ID NO:3. In some embodiments, the first polypeptide comprises an amino acid sequence which is not naturally occurring.

In some embodiments, the first polypeptide comprises the amino acid sequence of SEQ ID NO:33. In some embodiments with respect to SEQ ID NO:33, X53 is N, S, T, M, R, Q and/or X83 is N, R or K, and/or X84 is O or A, and/or X147 is C, S, T, M, V, L, A, or G, and/or X151 is C, S, T, M, V, L, A, or In some embodiments, X53 is N, S or K and/or X83 is N or R and/or X84 is G or A nd/or X147 is C, V, L or A and/or X151 is C, S, V, L or A.

In some embodiments, the first polypeptide is about 200 to 250 amino acids, 210 to 250 amino acids, 220 to 250 amino acids, 220 to 240 amino acids, 230 to 250 amino acids, 230 to 240 amino acids, or 230 to 235 amino acids, 220 to 275 amino acids, 220 to 260 amino acids, 230 to 260 amino acids, 240 to 250 amino acids, 250 to 260 amino acids, 230 to 256 amino acids, 240 amino acids to 256 amino acids, 245 amino acids to 256 amino acids in length. In some embodiments, the first polypeptide is 220, 221, 222, 223, 224, 225, 226, 227, 228, 229, 230, 231, 232, 233, 234, 235, 236, 237, 238, 239, 240, 230, 251, 252, 253.254, 255, 256, 257, 258, 259 or 260 amino acids in length.

In some embodiments, the first polypeptide comprising an amino acid sequence having at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%, sequence identity to SEQ ID NO:34, SEQ ID NO:36, SEQ ID NO:38, SEQ ID NO:39, SEQ ID NO:40, SEQ ID NO:41, SEQ ID NO:42, SEQ ID NO:46, SEQ ID NO:47, SEQ ID NO:48 or SEQ ID NO:49 is provided. In some embodiments, the first polypeptide does not comprise an amino acid sequence identical to SEQ ID NO:3 or SEQ ID NO:34. In some embodiments, the first polypeptide comprises an amino acid sequence which is not naturally occurring. In some embodiments, the first heterologous nucleic acid is integrated into a genome of said bacterium. In some embodiments, the first polypeptide is a therapeutic protein for treating a gastrointestinal epithelial cell barrier function disorder and/or disease.

In some embodiments, the first polypeptide comprises the amino acid sequence of SEQ ID NO:34, SEQ ID NO:36, SEQ ID NO:38, SEQ ID NO:39, SEQ ID NO:40, SEQ ID NO:41, SEQ ID NO:42, SEQ ID NO:46, SEQ ID NO:47, SEQ ID NO:48 or SEQ ID NO:49. In some embodiments, the first polypeptide comprises the amino acid sequence of SEQ ID NO:3. In some embodiments, the first polypeptide comprises the amino acid sequence of SEQ ID NO:34 or SEQ ID NO:42.

In some embodiments, the rust polypeptide comprises an amino acid sequence which is at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to SEQ ID NO:34, wherein the amino acid sequence has at least 1, 2, 3 or 4 amino acid substitutions relative to SEQ ID NO:34 or to SEQ ID NO:36. In some embodiments, the amino acid sequence has at least 2 and less than 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 amino acid substitutions relative to SEQ ID NO:34. In some embodiments, the first polypeptide comprises an amino acid sequence which is not naturally occurring.

In some embodiments, the first polypeptide comprises the amino acid sequence of SEQ ID NO:50. In some embodiments, X11 is N, R or K, and/or X12 is O or A, and/or X75 is C, S, T, M, V, L, A, or G, and/or X79 is C, S, T, M, V, L, A, or G. In some embodiments, X11 is N or R and/or X12 is G or A and/or X75 is C, V, L or A and/or X79 is C, S, V, L or A.

In some embodiments, the first polypeptide is about 100 to 200 amino acids, 110 to 190 amino acids, 120 to 180 amino acids, 130 to 170 amino acids, 140 to 170 amino acids, 150 to 170 amino acids, 150 to 180 amino acids, 155 to 170 amino acids, 160 to 170 amino acids, 135 to 165 amino acids, or 160 to 165 amino acids in length. In some embodiments, the first polypeptide is 140, 141, 142, 143, 144, 145, 146, 147, 148, 149, 150, 151, 152, 153, 154, 155, 156, 157, 138, 159, 160, 161, 162, 163, 164, 165, 166, 167, 168, 169, 170, 171, 172 or 173 amino acids in length.

In some embodiments, the first polypeptide is a polypeptide which is about 30 to 80, 40 to 70, 45 to 55, 35 to 60, 40 to 60, or 35 to 55 amino acids in length. In some embodiments, the first polypeptide is about 35, 36, 37, 38, 39, 40, 41, 42, 43, 44, 45, 46, 47, 48, 49, 50, 51, 52, 53, 54, 55, 56, 57, 58, 59 or 60 amino acids in length.

In some embodiments, the bacterium comprises a first polypeptide that is a therapeutic protein provided herein. In some embodiments, the first polypeptide is a therapeutic protein for treating a gastrointestinal epithelial cell barrier function disorder and/or disease. In some embodiments, the bacterium comprising a therapeutic protein or variant is provided.

In some embodiments, the therapeutic protein reduces intestinal tissue pathology in a subject administered the protein. In some embodiments, the subject was induced to have intestinal tissue damage by treatment with a chemical. In some embodiments, the subject was treated with the chemical dextran sodium sulfate (DSS) to induce intestinal tissue damage. In some embodiments, the subject is a mammal. In some embodiments, the animal is a rodent. In some embodiments, the subject is a non human primate. In some embodiments, the subject can be a human, for example, after chemotherapy.

In some embodiments, the therapeutic protein reduces gastrointestinal inflammation in a subject administered the protein. In some embodiments, the therapeutic protein reduces intestinal mucosa inflammation in the subject. In some embodiments, the protein improves intestinal epithelial cell barrier function or integrity in the subject. In some embodiments, the therapeutic protein increases the amount of mucin in intestinal tissue in a subject administered said protein. In some embodiments, the therapeutic protein increases intestinal epithelial cell wound healing in a subject administered the protein. In some embodiments, the therapeutic protein increases intestinal epithelial cell proliferation in a subject administered the protein. In some embodiments, the therapeutic protein prevents or reduces colon shortening in a subject administered the protein. In some embodiments, the therapeutic protein modulates (e.g., increases or decreases) a cytokine in the blood, plasma, serum, tissue and/or mucosa of a subject administered the protein. In some embodiments, the therapeutic protein decreases the levels of at least one pro-inflammatory cytokine (e.g., TNF-α and/or IL-23) in the blood, plasma, serum, tissue and/or mucosa of the subject.

In some embodiments, the disclosure provides polynucleotides encoding the first polypeptide that is a therapeutic protein and methods of expressing said nucleic acids in a host bacterium. In some embodiments, the host bacterium is Lactococcus lactis. In some embodiments, the polynucleotide comprises a sequence which encodes a protein that is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 93%, 96%, 97%, 98%, 99%, 99.1%, 99.2%, 99.3%, 99.4%, 99.5%, 99.6%, 99.7%, 99.8%, or 99.9%, or 100% identical to SEQ ID NO:19, SEQ ID NO:34, SEQ ID NO:36, SEQ ID NO:38, SEQ ID NO:39, SEQ ID NO:40, SEQ ID NO:42, SEQ ID NO-A6, SEQ ID NO:47, SEQ ID NO:48 or SEQ ID NO:49. In some embodiments, the polynucleotide comprises a sequence which encodes a protein that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100% identical to SEQ ID NO:19, SEQ ID NO:36, SEQ ID NO:38, SEQ ID NO:39, SEQ ID NO:40, SEQ ID NO:42, SEQ ID NO:46, SEQ ID NO:47, SEQ ID NO:48 or SEQ ID NO:49, and less than 100% identical to SEQ ID NO:3 or SEQ ID NO:34. In some embodiments, the polynucleotide encodes a protein which is a non-naturally occurring variant of SEQ ID NO:1 or SEQ ID NO:3. In some embodiments, the polynucleotide is codon-optimized for expression in a recombinant host cell. In some embodiments, the polynucleotide is codon-optimized for expression in L. lactis and/or E. coli.

In some embodiments, the disclosure provides a nucleic acid which comprises a sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO:20, SEQ ID NO:35, SEQ ID NO:37, SEQ ID NO:41 or SEQ ID NO:42. In some embodiments, the nucleic acid comprises a sequence which is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO:20, SEQ ID NO:37, SEQ ID NO:41 or SEQ ID NO:42, and less than 100% identical to SEQ ID NO:4 or SEQ ID NO:35. In some embodiments, the nucleic acid comprises a sequence which is a non-naturally occurring variant of SEQ ID NO:2 or SEQ ID NO:4.

In some aspects, the disclosure provides a pharmaceutical composition for treating an inflammatory bowel disease. The composition can include a protein comprising an amino acid sequence having at least 70%, 75%, 80%, 85%, 90%, 91%, 92%, 93%94%, 95%, 96%, 97%, 98%, 99%, 99.5%, 99.6%, 99.7%, 99.8% or 100% sequence identity to SEQ ID NO:19, SEQ ID NO:34, SEQ ID NO:36, SEQ ID NO:38, SEQ ID NO:39, SEQ ID NO:40, SEQ ID NO:42, SEQ ID NO:46, SEQ ID NO:47, SEQ ID NO:48 or SEQ ID NO:49 and a pharmaceutically acceptable carrier. In some embodiments, the protein is purified or substantially purified. In some embodiments, the protein comprises the amino acid sequence of SEQ ID NO:19, SEQ ID NO:34, SEQ ID NO:36, SEQ ID NO:38, SEQ ID NO:39, SEQ ID NO:40, SEQ ID NO:42, SEQ ID NO:46, SEQ ID NO:47, SEQ ID NO:48 or SEQ ID NO:49. In some embodiments, the protein does not comprise a sequence which is identical to SEQ ID NO:34 or SEQ ID NO:36 or the protein is a non-naturally occurring variant of SEQ ID NO:3. In some embodiments, the protein comprises an amino acid sequence of SEQ ID NO:19 or SEQ ID NO:34. In some embodiments, the protein comprises an amino acid sequence of SEQ ID NO:36 or SEQ ID NO:44.

In some embodiments, the present disclosure provides a pharmaceutical composition, comprising: i) a therapeutically effective amount of the bacterium comprising at least one first heterologous nucleic acid encoding a first polypeptide having at least about 90% sequence identity to SEQ ID NO: 19 and/or SEQ ID NO: 34 and ii) a pharmaceutically acceptable carrier. In some embodiments, the pharmaceutical composition is formulated for rectal, parenteral, intravenous, topical, oral dermal, transdermal, or subcutaneous administration. In some embodiments, the pharmaceutical composition is a liquid, a gel, or a cream. In some embodiments, the pharmaceutical composition is a solid composition comprising an enteric coating. In some embodiments, the pharmaceutical composition is formulated to provide delayed release. In some embodiments, the delayed release is release into the gastrointestinal tract. In some embodiments, the delayed release is into the mouth, the small intestine, the large intestine and/or the rectum. In some embodiments, the pharmaceutical composition is formulated to provide sustained release. In some embodiments, the sustained release is release into the gastrointestinal tract. In some embodiments, the sustained release is into the mouth, the small intestine, the large intestine and/or the rectum. In some embodiments, the sustained release composition releases the therapeutic formulation over a time period of about 1 to 20 hours, 1 to 10 hours, 1 to 8 hours, 4 to 12 hours or 5 to 15 hours.

In some embodiments, the pharmaceutical composition further comprises a second therapeutic agent. In some embodiments, the second therapeutic agent is selected from the group consisting of an anti-diarrheal, a 5-aminosalicylic acid compound, an anti-inflammatory agent, an antibiotic, an anti-cytokine agent, an anti-inflammatory cytokine agent, a steroid, a corticosteroid, an immunosuppressant, a JAK inhibitor, an anti-integrin biologic, an anti-IL12/23R biologic, and a vitamin.

As aforementioned, these bacteria comprising (e.g., expressing or producing) protein therapeutics can, in some cases, promote epithelial barrier function and integrity in a subject. Additionally, the therapeutic effect of the proteins can include suppression of an inflammatory immune response in an IBD individual and a subject involved with various types of mucositis. The disclosure provides detailed guidance for methods of utilizing the taught bacteria comprising therapeutic proteins to treat a host of gastrointestinal inflammatory conditions and disease states involving compromised gastrointestinal epithelial barrier integrity.

In some embodiments, a method of treating a gastrointestinal epithelial cell barrier function disorder is provided. The disorder can be selected from the group consisting of: inflammatory bowel disease, ulcerative colitis, Crohn's disease, short bowel syndrome, GI mucositis, oral mucositis, chemotherapy-induced mucositis, radiation-induced mucositis, necrotizing enterocolitis, pouchitis, a metabolic disease, celiac disease, inflammatory bowel syndrome, and chemotherapy associated steatohepatitis (CASH). In some embodiments, the disorder is oral mucositis. The method can include administering to a subject in need thereof a pharmaceutical composition, comprising: i) a therapeutically effective amount of the bacterium comprising at least one first heterologous nucleic acid encoding a first polypeptide, which is a therapeutic protein comprising an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, %%, 97%, 98%, 99%, 99.1%, 99.2%, 99.3%, 99.4%, 99.5%, 99.6%, 99.7%, 99.8%, or 99.9%, or 100% sequence identity to SEQ ID NO:3, SEQ ID NO:9, SEQ ID NO:11, SEQ ID NO:13, SEQ ID NO:15, SEQ ID NO:17, SEQ ID NO:19, SEQ ID NO:34, SEQ ID NO:36, SEQ ID NO:38, SEQ ID NO:39, SEQ ID NO:40, SEQ ID NO:42, SEQ ID NO:46, SEQ ID NO:47, SEQ ID NO:48 or SEQ ID NO:49; and ii) a pharmaceutically acceptable carrier. In some embodiments of the method, the protein comprises an amino acid sequence identical to SEQ ID NO:3, SEQ ID NO:9, SEQ ID NO:11, SEQ ID NO:13, SEQ ID NO:15, SEQ ID NO:17, SEQ ID NO:19, SEQ ID NO:34, SEQ ID NO:36, SEQ ID NO:38, SEQ ID NO:39, SEQ ID NO:40, SEQ ID NO:42, SEQ ID NO:46, SEQ ID NO:47, SEQ ID NO:48 or SEQ ID NO:49. In some embodiments, the protein is not identical to SEQ ID NO:3 or is a non-naturally occurring variant of SEQ ID NO:3.

In some embodiments of the method, the bacterium is viable. In some embodiments of the method, the gastrointestinal epithelial cell barrier function disorder is a disease associated with decreased gastrointestinal mucosal epithelium integrity.

In some embodiments of the method, the composition can be formulated for oral ingestion. The composition can be an edible product. The composition can be formulated as a pill, a tablet, a capsule, a suppository, a liquid, or a liquid suspension.

In some embodiments, a genetically-engineered bacterium for treating a gastrointestinal epithelial cell barrier function disorder is provided, comprising: at least one first heterologous nucleic acid encoding a first polypeptide, which is a protein comprising an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%, or 100% sequence identity to SEQ ID NO:3, SEQ ID NO:9, SEQ ID NO: 11, SEQ ID NO:13, SEQ ID NO:15, SEQ ID NO:17, SEQ ID NO:19, SEQ ID NO:34, SEQ ID NO:36, SEQ ID NO:38, SEQ ID NO:39, SEQ ID NO:40, SEQ ID NO:42, SEQ ID NO:46, SEQ ID NO:47, SEQ ID NO:48 or SEQ ID NO:49 in a genome of said bacterium, wherein said nucleic acid is operably linked to a promoter.

In some embodiments, the protein comprises an amino acid sequence identical to SEQ ID NO:3, SEQ ID NO:9, SEQ ID NO:11, SEQ ID NO:13, SEQ ID NO:15, SEQ ID NO:17, SEQ ID NO:19, SEQ ID NO:34, SEQ ID NO:36, SEQ ID NO:38, SEQ ID NO:39, SEQ ID NO:40, SEQ ID NO:42, SEQ ID NO:46, SEQ ID NO:47, SEQ ID NO:48 or SEQ ID NO:49. In some embodiments, the protein is not identical to SEQ ID NO:3 or is a non-naturally occurring variant of SEQ ID NO:3.

In some embodiments, a subject administered with the bacterium taught herein has been diagnosed with mucositis. In some embodiments, the mucositis is oral mucositis. In some embodiments, the mucositis is chemotherapy-induced mucositis, radiation therapy-induced mucositis, chemotherapy-induced oral mucositis, or radiation therapy-induced oral mucositis. In some embodiments, the mucositis is gastrointestinal mucositis. In some embodiments, the gastrointestinal mucositis is mucositis of the small intestine, the large intestine, or the rectum.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF THE DRAWINGS

FIG. 1A and FIG. 1B show restoration, by SG-11, of epithelial barrier integrity following inflammation induced disruption, as described in Example 2.

FIG. 2 shows effects of SG-11 administration on epithelial cell wound healing, as described in Example 3.

FIG. 3 shows effects of SG-11 administration on epithelial centric barrier function readouts in a DSS model of inflammatory bowel disease, as described in Example 4.

FIG. 4 shows effects of SG-11 administration an inflammatory readouts responsive to impaired barrier function in a DSS model of inflammatory bowel disease, as described in Example 4.

FIG. 5 shows effects of SG-11 administration on body weight in a DSS model of inflammatory bowel disease, as described in Example 4.

FIG. 6 shows effects of SG-11 administration on gross pathology in a DSS model of inflammatory bowel disease, as described in Example 4.

FIG. 7A, FIG. 7B and FIG. 7C show results from histopathology analysis of proximal (FIG. 7A), distal (FIG. 7B) and both proximal and distal (FIG. 7C) tissue from a DSS model of inflammatory bowel disease, as described in Example 4.

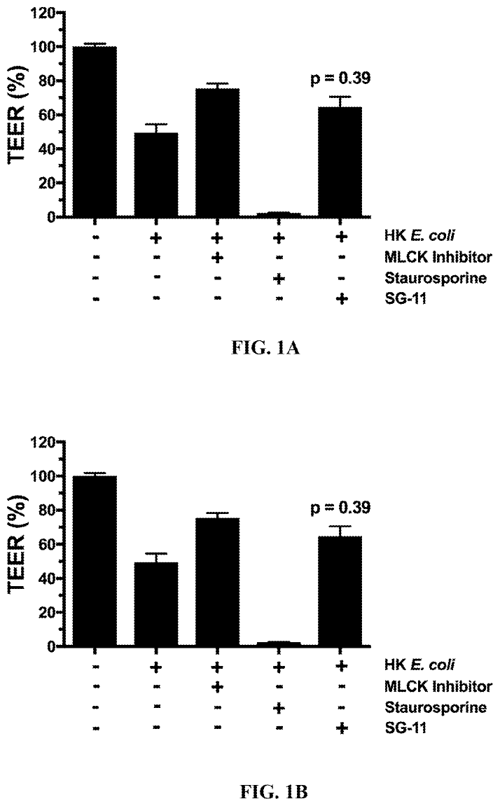

FIG. 1A and FIG. 8B show effects of SG-11 administration on colon length (FIG. 8A) and colon weight-to-length (FIG. 8B) in a DSS model of inflammatory bowel disease, as described in Example 4.

FIG. 9 shows epithelial barrier integrity following SG-11 treatment of a DSS model of inflammatory bowel disease, as described in Example 5.

FIG. 10 shows inflammation centric readouts of barrier function in a DSS model of inflammatory bowel disease, as described in Example 5.

FIG. 11 shows prevention of weight loss in a DSS model of inflammatory bowel disease, as described in Example 5.

FIG. 12A shows effects of SG-11 administration on colon length in a DSS model of inflammatory bowel disease, as described in Example 5. FIG. 12B shows effects of SG-11 administration on colon weight-to-length ratio in a DSS model of inflammatory bowel disease, as described in Example 5.

FIG. 13A, FIG. 13B and FIG. 13C show results from histopathology analysis of proximal (FIG. 13A), distal (FIG. 13B) and both proximal and distal (FIG. 13C) tissue from a DSS model of inflammatory bowel disease, as described in Example 5.

FIG. 14 shows the alignment of SG-11 (SEQ ID NO:7) with similar protein sequences from Roseburia species (WP_006857001, SEQ ID NO:21; WP_075679733, SEQ ID NO:22; WP_055301040, SEQ ID NO:23).

FIG. 15A, FIG. 15B, FIG. 15C, FIG. 15D, FIG. 15E, FIG. 15F, FIG. 15G, FIG. 15H, and FIG. 13I show effects of different conditions on SG-11 stability. See Example 11 for the conditions associated with FIG. 15A to FIG. 15I.

FIG. 16A, FIG. 16B, FIG. 16C, FIG. 16D, FIG. 16E, FIG. 16F, FIG. 16G, FIG. 16H, and FIG. 16I shows effects of conditions on SG-11V5 stability. See Example 11 for the conditions associated with FIG. 16A to FIG. 16I.

FIG. 17 shows restoration, by SG-11 and an SG-11 variant (SG11V5), of epithelial barrier integrity following inflammation induced disruption upon, as described in Example 12.

FIG. 18A and FIG. 18B show epithelial barrier integrity following treatment of a DSS model of inflammatory bowel disease with SG-11 and a variant of SG-11 (SG11V5), as described in Example 13.

FIG. 19A and FIG. 19B show inflammation centric readouts of barrier function in a DSS model of inflammatory bowel disease, as described in Example 13.

FIG. 20A and FIG. 20B show effects of treatment with SG-11 or a variant of SG-11 (SG11V5) on weight loss in a DSS model of inflammatory bowel disease, as described in Example 13.

FIG. 21 shows effects of administering SG-11 or a variant of SG-11 (SG11V5) on gross pathology in a DSS model of inflammatory bowel disease, as described in Example 13.

FIG. 22A and FIG. 22B show effects of treatment with SG-11 or a variant of SG-11 (SG11V5) on colon length in a DSS model of inflammatory bowel disease, as described in Example 13.

FIG. 23A and FIG. 23B show effects of treatment with SG-11 or a variant of SG-11 (SG11V5) on colon weight-to-length ratio in a DSS model of inflammatory bowel disease, as described in Example 13.

FIG. 24A shows the alignment of SG-11 (SEQ ID NO:7) with SG-11 variants (SEQ ID NO:11, SEQ ID NO:13, SEQ ID NO:15, SEQ ID NO:17, SEQ ID NO:19), and FIG. 24B shows results of the percent identity matrix based on the multiple sequence alignment analysis. The Clustal Omega program provided by EMBL-EBI was used for the multiple alignment analysis described herein.

FIG. 25 shows SDS-PAGE and Coomassie blue analysis of a protein product generated upon incubation of SG-11 protein in a fecal slurry as described in Example 14.

FIG. 26 shows SDS-PAGE and Coomassie blue analysis of a protein product generated upon incubation of SG-11 protein with trypsin as described in Example 14.

FIG. 27 shows SDS-PAGE and Coomassie blue analysis of a protein product generated upon incubation of SG-11 protein with trypsin in the presence or absence of a trypsin inhibitor as described in Example 14.

FIG. 28 shows restoration, by a product of SG-1 protein incubated in fecal slurry, of epithelial barrier integrity following inflammation induced disruption upon, as described in Example 15.

FIG. 29 shows expression cassettes in a L. lactis expression plasmid, pNZ8124. The pNZ124 plasmid is designed for expressing a gene of interest (e.g., SG-11V5) under control of an inducible nisin A promoter (PnisA) and the lactococcus usp45 secretion leader (aka signal peptide) sequence (see “before”). Alternatively, far the constitutive expression of gene of interest (e.g. SG-11V5), the PnisA promoter is replaced with a strong constitutive promoter (Pusp45) in the L. lactis expression plasmids (see first “after row, right column). To induce trehalose accumulation in the L. lactis strain, an additional expression cassette (PnisA-otsBA operon) comprising trehalose-6-phosphate phosphatase (otsB) and trehalose-6-phosphate synthase (otsA) genes placed downstream of an inducible nisin A promoter (PnisA) are cloned into a pNZ8124 plasmid (see “after” rows, left column). As a negative control, an expression vector having only PnisA-otsBA operon is used without expression of gene of interest (e.g. SG-11V5). PnisA, inducible nisA promoter; Pusp45. Lactococcus constitutive usp45 promoter; SPusp45, Lactococcus usp43 secretion leader (signal peptide); otsBA, trehalose-6-phosphate phosphatase gene (otsB) and trehalose-6-phosphate synthase gene (otsA).

FIG. 30 shows western blot analysis of in vitro SG-11V5 protein expressed from the L. lactis expression plasmids as described in Example 20.

FIG. 31A and FIG. 31B depict western blot analysis of SG-11V5 protein expressed in L. lactis strains comprising the SG-11V5 expression plasmids as described in Example 20. FIG. 31A shows that the L. lactis strains comprising the expression plasmids in which an inducible (Lanes 5-6) or constitutive (Lanes 7-8) promoter drives SG-11V5 expression, produced SG-11V5 protein in mice in vivo as described in Example 21. FIG. 31B shows that the L. lactis strains comprising the expression plasmids in which an inducible promoter is present upstream of both the otsBA and the SG-11V3 sequence (Lanes 5-6) or only upstream of the otsBA gene (Lane 7 wherein a constitutive promoter is upstream of the SG-V11 sequence), produced SG-11V5 protein in mice in vivo as described in Example 21.

FIG. 32A, FIG. 32B and FIG. 32C depict quality control results of L. lactis strains comprising the SG-11V5 expression plasmids as described in Example 20. FIG. 32A shows colonies of the L. lactis strains for functional analysis described in Example 22. FIG. 32B shows PCR amplification to confirm target genes cloned into the SG-11V5 expression plasmids as described in Example 22. FIG. 32C shows western blot analysis of in vitro SG-11V5 protein expressed from the L. lactis expression plasmids with the constitutive promoter and/or the inducible promoter, respectively for SG-11V5 expression as described in Example 22.

FIG. 33A shows effects of SG-11V5 administration ad SG-11V5-expressing L. lactis administration on epithelial centric barrier function readouts in a DSS model of inflammatory bowel disease, as described in Example 22. FIG. 33B shows effects of SG-11V5 administration and SG-11V5-expressing L. lactis administration on inflammatory readouts responsive to impaired barrier function in a DSS model of inflammatory bowel disease, as described in Example 22.

FIG. 34A and FIG. 34B show effects of SG-11V5 administration and SG-11V5-expressing L. lactis administration on colon length (FIG. 34A) and colon weight-to-length (FIG. 34B) in a DSS model of inflammatory bowel disease, as described in Example 22.

FIG. 35A and FIG. 358 show effects of SG-11V5 administration (FIG. 35A) and SG-11V5-expressing L. lactis administration (FIG. 35B) on body weight in a DSS model of inflammatory bowel disease, as described in Example 22.

FIG. 36A shows effects of SG-11V5 administration and SG-11V5-expressing L. lactis administration on gross pathology in a DSS model of inflammatory bowel disease, as described in Example 24. FIG. 36B shows images of the entire colon from cecum to rectum from mice tested with clinical scores, as described in Example 22.

FIG. 37A shows representative images of an oral mucositis model of hamsters induced by radiation, corresponding to mucositis score. FIG. 37B shows mean mucositis scores of SG-11-treated and multiple doses of SG-11V5 treated hamsters as an in vivo model of oral mucositis, as described in Example 23.

FIG. 38 shows effects of SG-11 and multiple doses of SG-11V5 administration on body weight in an in vivo model of oral mucositis, as described in Example 23.

FIG. 39 shows a Western blot, using an anti-SG11V5 antibody, in which SG-11V5 was detected from culture supernatants.

### DETAILED DESCRIPTION

In some aspects, the present disclosure addresses the important need in the medical community for a therapeutic that can effectively treat a subject suffering from a gastrointestinal barrier function disorder or disease such as Inflammatory Bowel Disease (IBD) and mucositis. In one aspect, therapeutics (e.g., probiotic therapeutics) are provided which can improve and/or maintain epithelial barrier integrity. These probiotic therapeutics can also reduce inflammation of the gastrointestinal tract of the subject and/or decrease symptoms associated with inflammation of the mucous membranes lining the digestive tract. In another aspect, the probiotic therapeutics comprise protein therapeutics. The probiotics are bacterial strains having proteins that can improve and/or maintain epithelial barrier integrity as well as reduce inflammation of the digestive tract. In one aspect, the bacterial strain is a Lactococcus lactis strain. In one aspect, the probiotics are recombinant bacteria expressing proteins that can improve and/or maintain epithelial barrier integrity as well as reduce inflammation of the digestive tract. In one aspect, the recombinant bacteria have at least one recombinant vector comprising at least one expression cassette to produce a protein. In another aspect, the recombinant bacteria have at least one polynucleotide construct encoding a protein within a genome of the bacteria. In another aspect, the probiotics are also genetically-engineered bacteria expressing proteins that can improve and/or maintain epithelial barrier integrity as well as reduce inflammation of the digestive tract. In another aspect, the genetically-engineered bacteria have at least one expression cassette to produce protein within a genome of the bacteria.

In some aspects, the present disclosure provides therapeutics (e.g., probiotic therapeutics) that are useful in the treatment of subjects suffering from symptoms associated with gastrointestinal disorders. For example, these probiotic therapeutics can promote or enhance epithelial barrier function and/or integrity. The probiotic therapeutics may also suppress the inflammatory immune response in an individual suffered from IBD and/or mucositis. The probiotic therapeutics provided herein are useful in treating the numerous diseases that are associated with decreased gastrointestinal epithelial cell barrier function or integrity and inflammation of the mouse and gastrointestinal tract.

In some aspects, provided are also therapeutics (e.g., probiotic bacterial strains) expressing a heterologous protein that have therapeutic activity comparable to or superior to an similar (e.g., parental) strain, but the bacterial strains have enhanced viability compared to the similar strain through the expression of another protein related to trehalose accumulation.

### Definitions

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art. Thus, while the following terms are believed to be well understood by one of ordinary skill in the art, the following definitions are set forth to facilitate explanation of the presently disclosed subject matter.

Throughout this specification, the word “comprise” or variations such as “comprises” or “comprising” will be understood to imply the inclusion of a stated component, or group of components, but not the exclusion of ay other components, or group of components.

The term “a” or “an” refers to one or more of that entity, e.g. can refer to a plural referents. As such, the terms “a” or “an,” “one or more,” and “at least one” are used interchangeably herein. In addition, reference to “an element” by the indefinite article “a” or “an” does not exclude the possibility that more than one of the elements is present, unless the context clearly requires that there is one and only one of the elements.

The term “including” is used to mean “including but not limited to.” “Including” and “including but not limited to” are used interchangeably.

The term “about” as used herein with respect to % sequence identity, or % sequence homology, of a nucleic acid sequence, or amino acid sequence, means up to and including ±1.0% in 0.1% increments. For example, “about 90%” sequence identity includes 89.0%, 89.1%, 89.2%, 89.3%, 89.4%, 89.5%, 89.6%, 89.7%, 89.8%, 89.9%, 90%, 90.1%, 90.2%, 90.3%, 90.4%, 90.5%, 90.6%, 90.7%, 90.8%, 90.9%, and 91%. If not used in the context of % sequence identity, then “about” means±1%, 2%, 3%, 4%, 5%, 6%, 7%, 8%, 9%, or 10%, depending upon context of the value in question.

As used herein, a “synthetic protein” means a protein that comprises an amino acid sequence that contains one or more amino acids substituted with different amino acids relative to a naturally occurring amino acid sequence. That is, a “synthetic protein” comprises an amino acid sequence that has been altered to contain at least one non-naturally occurring substitution modification at a given amino acid position(s) relative to a naturally occurring amino acid sequence.

The terms “gastrointestinal” or “gastrointestinal tract,” “alimentary canal,” and “intestine,” as used herein, may be used interchangeably to refer to the series of hollow organs extending from the mouth to the anus and including the mouth, esophagus, stomach, small intestine, large intestine, rectum and anus. The terms “gastrointestinal” or “gastrointestinal tract,” “alimentary canal,” and “intestine” am not always intended to be limited to a particular portion of the alimentary canal.

The term “SG-11” as used herein refers to a protein comprising the amino acid sequence of SEQ ID NO:3 and also to variants thereof having the same or similar functional activity as described herein. For example, variants can include one or more mutations. In some embodiments, variants can include an initial methionine. Accordingly, SG-11 can refer herein to proteins comprising or consisting of SEQ ID NO:1, SEQ ID NO:3, SEQ ID NO:5, SEQ ID NO:7, or SEQ ID NO:9, or variants or fragments thereof. Examples of SG-11 variants include but are not limited to SEQ ID NO:11 (SG-11V1), SEQ ID NO:13 (SG-11V2), SEQ ID NO:15 (SG-11V3), SEQ ID NO:17 (SG-11V4), and SEQ ID NO:19 (SG-11V5). In U.S. provisional patent applications Nos. 62/482,963 and 62/607,706, U.S. patent application Ser. No. 15/947,263 and PCT application no. PCT/US2018/026447, are incorporated herein by reference in its entirety, the term “Experimental Protein 1” and variants thereof was used, and it is synonymous with SG-11 as used herein or variants thereof.

The term “SG-21” as used herein refers to a protein comprising the amino acid sequence of SEQ ID NO:34 and also to variants thereof having the same or similar functional activity as described herein. Accordingly, SG-21 can refer herein to proteins comprising or consisting of SEQ ID NO-34 or SEQ ID NO:36, or variants thereof. Examples of SG-21 variants include but are not limited to SEQ ID NO:38 (SG-21V1), SEQ ID NO:39 (SG-21V2), and SEQ ID NO:40 (SG-21V5). In some of U.S. provisional patent applications 62/482,963, filed Apr. 7, 2017; 62/607,706, filed Dec. 19, 2017; 62/611,334, filed Dec. 28, 2017, 62/654,083, filed Apr. 6, 2018, and PCT application no. PCT/US2019/026412, filed on Apr. 8, 2019, which describe the related proteins, SG-11 and SG-21, and each of which is incorporated herein by reference in its entirety, the term “Experimental Protein 1” and variants thereof was used, and it is synonymous with SG-11 as used herein or variants thereof.

A “signal sequence” (also termed “presequence,” “signal peptide,” “leader sequence,” or “leader peptide”) refers to a sequence of amino acids located at the N-terminus of a nascent protein, and which can facilitate the secretion of the protein from the cell. The resultant mature form of the extracellular protein lacks the signal sequence, which is cleaved off during the secretion process.

The recitations “sequence identity,” “percent identity,” “percent homology,” or for example, comprising a “sequence 50% identical to,” as used herein, refer to the extent that sequences are identical on a nucleotide-by-nucleotide or amino acid-by-amino acid basis, over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Vat, Leu, Ile, Phe, Tyr, Trp, Lys, Arg, His, Asp, Glu, Asn, Gin, Cys and Met) occurs in both sequences to yield the number of matched positions, dividing the number of matched positions by the total number of positions in the window of comparison (e.g., the window size), and multiplying the result by 100 to yield the percentage of sequence identity.

The phrases “substantially similar” and “substantially identical” in the context of at least two nucleic acids or polypeptides typically means that a polynucleotide or polypeptide comprises a sequence that has at least about 70%, 75%, 80%, 85%, 86%, 87%, 88%, 89%, 90%, 91%, 92%, 93%, 94%, 95%, 96%, 97%, 98%, 99%, or even 99.5% sequence identity, in comparison with a reference polynucleotide or polypeptide. In some embodiments, substantially identical polypeptides differ only by one or more conservative amino acid substitutions. In some embodiments, substantially identical polypeptides are immunologically cross-reactive. In some embodiments, substantially identical nucleic acid molecules hybridize to each other under stringent conditions (e.g., within a range of medium to high stringency).

As used herein, the term “nucleotide change” refers to, e.g., nucleotide substitution, deletion, and/or insertion, as is well understood in the art. For example, in some embodiments, mutations contain alterations that can produce silent substitutions, additions, or deletions, but do not alter the properties or activities of the encoded protein or how the proteins are made.

Related (and derivative) proteins encompass “variant” proteins. Variant proteins differ from another (e.g., parental) protein and/or from one another by a small number of amino acid residues. A variant may include one or more amino acid mutations (e.g., amino acid deletion, insertion or substitution) as compared to the parental protein from which it is derived.

“Conservatively modified variants” applies to both amino acid and nucleic acid sequences. With respect to particular nucleic acid sequences, a conservatively modified variant refers to nucleic acids encoding identical amino acid sequences, or amino acid sequences that have one or more “conservative substitutions.” An example of a conservative substitution is the exchange of an amino acid in one of the following groups for another amino acid of the same group (see U.S. Pat. No. 5,767,063; Kyte and Doolittle (1982) J. Mol. Biol. 157:105-132). (1) Hydrophobic: Norleucine, Ile, Val, Leu, Phe, Cys, Met; (2) Neutral hydrophilic: Cys, Ser, Thr; (3) Acidic: Asp, Glu; (4) Basic: Asn, Gin, His, Lys, Arg; (5) Residues that influence chain orientation: Gly. Pro; (6) Aromatic: Trp, Tyr, Phe; and (7) Small amino acids: Gly, Ala, Ser. Thus, the term “conservative substitution” with respect to an amino acid denotes that one or more amino acids are replaced by another, chemically similar residue, wherein said substitution does not generally affect the functional properties of the protein. In some embodiments, the disclosure provides for proteins that have at least one non-naturally occurring, conservative amino acid substitution relative to the amino acid sequence identified in SEQ ID NO:3, SEQ ID NO:19 or SEQ ID NO:34.

The term “amino acid” or “any amino acid” refers to any and all amino acids, including naturally occurring amino acids (e.g., α-amino acids), unnatural amino acids, modified amino acids, and unnatural or non-natural amino acids. It includes both D- and L-amino acids. Natural amino acids include those fund in nature, such as, e.g., the 23 amino acids that combine into peptide chains to form the building-blocks of a vast array of proteins. These are primarily L stereoisomers, although a few D-amino acids occur, e.g., in bacterial envelopes and some antibiotics. There are 20 “standard” natural amino acids. The “non-standard,” natural amino acids include pyrrolysine (found in methanogenic organisms and other eukaryotes), selenocysteine (present in many noneukaryotes as well as most eukaryotes), and N-formylmethionine (encoded by the start codon AUG in bacteria, mitochondria and chloroplasts). “Unnatural” or “non-natural” amino acids include non-proteinogenic amino acids (e.g., those not naturally encoded or found in the genetic code) that either occur naturally or are chemically synthesized. Over 140 unnatural amino acids are known and thousands of more combinations are possible. Examples of“unnatural” amino acids include β-amino acids (β3 and β2), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid.

As used herein, “polypeptide” and “protein” are typically used interchangeably.

As used herein, “polynucleotide” and “nucleic acid” arm typically used interchangeably.

As used herein, a “synthetic nucleotide sequence” or “synthetic polynucleotide sequence” is a nucleotide sequence that is not known to occur in nature, or that is not naturally occurring. Generally, such a synthetic nucleotide sequence will comprise at least one nucleotide difference when compared to any other naturally occurring nucleotide sequence. As used herein, a “synthetic amino acid sequence” or “synthetic peptide sequence” or “synthetic polypeptide sequence” or “synthetic protein sequence” is an amino acid sequence that is not known to occur in nature, or that is not naturally occurring. Generally, such a synthetic amino acid sequence will comprise at least one amino acid difference when compared to any other naturally occurring amino acid sequence.

For the most part, the names of natural and non-natural aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader.

Among sequences disclosed herein are sequences incorporating a “Hy-” moiety at the amino terminus (N-terminus) of the sequence, and either an “—OH” moiety or an “—NH₂” moiety at the carboxy terminus (C-terminus) of the sequence. In such cases, and unless otherwise indicated, a “Hy-” moiety at the N-terminus of the sequence in question indicates a hydrogen atom, corresponding to the presence of a free primary or secondary amino group at the N-terminus, while an “—OH” or an “—NH₂” moiety at the C-terminus of the sequence indicates a hydroxy group or an amino group, corresponding to the presence of an amino (CONH₂) group at the C-terminus, respectively. In each sequence of the disclosure, a C-terminal “—OH” moiety may be substituted for a C-terminal “—NH₂” moiety, and vice-versa.

The term “NH₂,” a used herein, can refer to a free amino group present at the amino terminus of a polypeptide. The term “OH,” as used herein, can refer to a free carboxy group present at the carboxy terminus of a peptide. Further, the term “Ac,” as used herein, refers to acetyl protection through acylation of the C. or N-terminus of a polypeptide. In certain peptides shown herein, the NH₂ locates at the C-terminus of the peptide indicates an amino group. The term “carboxy,” as used herein, refers to —CO₂H. The term “cyclized,” as used herein, refers to a reaction in which one part of a polypeptide molecule becomes linked to another part of the polypeptide molecule to form a closed ring, such as by forming a disulfide bridge or other similar bond.

The term “pharmaceutically acceptable salt,” as used herein, represents salts or zwitterionic forms of the peptides, proteins, or compounds of the present disclosure, which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethanesulfonate (isethionate), lactate, maleate, mesitylenesulfonate, methanesulfonate, napthalenesulfonate, nicotinate, 2-naphthalenesulfonate, oxalate, pamoate, pectinate, persulfate, 3-phenylpropionate, picrate, pivalate, propionate, succinate, tartrate, trichloroacetate, trifluoroacetate, phosphate, glutamate, bicarbonate, para-toluenesulfonate, and undecanoate. Also, amino groups in the compounds of the present disclosure can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. A pharmaceutically acceptable salt may suitably be a salt chosen, e.g., among acid addition salts and basic salts. Examples of acid addition salts include chloride salts, citrate salts and acetate salts. Examples of basic salts include salts where the cation is selected among alkali metal cations, such as sodium or potassium ions, alkaline earth metal cations, such a calcium or magnesium ions, as well as substituted ammonium ions. Other examples of pharmaceutically acceptable salts are described in “Remington's Pharmaceutical Sciences”, 17th edition, Alfonso R. Gennaro (Ed.), Mark Publishing Company, Easton, Pa., USA, 1985 (and more recent editions thereof), in the “Encyclopedia of Pharmaceutical Technology”, 3rd edition, James Swarbrick (Ed.), Informa Healthcare USA (Inc.), NY, USA, 2007, and in J. Pharm. Sci. 66: 2 (1977). Also, for a review on suitable salts, see Handbook of Pharmaceutical Salt: Properties, Selection, and Use by Stahl and Wermuth (Wiley-VCH, 2002). Other suitable base salts are formed from bases which form non-toxic salts. Representative examples include the aluminum, arginine, benzathine, calcium, choline, diethylamine, diolamine, glycine, lysine, magnesium, meglumine, olamine, potassium, sodium, tromethamine, and zinc salts. Hemisalts of acids and bases may also be formed, e.g., hemisulphate and hemicalcium salts.

As used herein, the term “at least a portion” or “fragment” of a nucleic acid or polypeptide means a portion having the minimal size characteristics of such sequences, or any larger fragment of the full length molecule, up to and including the full length molecule. In some embodiments, a fragment can include any subsequence of a parent molecule, for example, any consecutive 10, 20, 30, 40, 50, or more amino acids of a parent protein or any consecutive 30, 60, 90, 120, 150, or more nucleotides of a parent polynucleotide.

As used herein, the term “host cell” refers to a cell or cell line into which a recombinant expression vector for production of a polypeptide may be introduced for expression of the polypeptide.

The term “isolated,” “purified,” “separated,” and “recovered” as used herein refer to a material (e.g., a protein, nucleic acid, or cell) that is removed from at least one component with which it is naturally associated, for example, at a concentration of at least 90% by weight, or at least 95% by weight, or at least 98% by weight of the sample in which it is contained. For example, these terms may refer to a material which is substantially or essentially free from components which normally accompany it as found in its native state, such as, for example, an intact biological system.

As used herein, a “heterologous” or “non-native” nucleic acid sequence refers to a nucleic acid sequence not normally present in a microorganism, e.g., an extra copy of an endogenous sequence, or a heterologous sequence such as a sequence from a different organism (e.g., an organism from a different species, strain, or substrain of a prokaryote or eukaryote), or a sequence that is modified and/or mutated as compared to the unmodified native or wild-type sequence. In some embodiments, the non-native nucleic acid sequence is a synthetic, non-naturally occurring sequence. The non-native nucleic acid sequence may be a regulatory region, a promoter, a gene, and/or one or more genes (e.g., genes in a gene cassette or operon). In some embodiments, “heterologous” or “non-native” refers to two or more nucleic acid sequences that are not found in the same relationship to each other in nature. The non-native nucleic acid sequence may be present on a plasmid or chromosome. In some embodiments, the genetically engineered bacteria of the disclosure comprise a gene that is operably linked to a directly or indirectly inducible promoter that is not associated with said gene in nature, e.g., an inducible nisinA promoter (or other promoter described herein) operably linked to a gene encoding a protein provided herein.

“Microorganism” or “microbe” refers to an organism or microbe of microscopic, submicroscopic, or ultramicroscopic size that typically consists of a single cell. Examples of microorganisms include bacteria, viruses, parasites, fungi, certain algae and protozoa. In some aspects, the microorganism is engineered (“engineered microorganism”) to produce one or more polypeptide molecules. In certain embodiments, the recombinant microorganism or microbe is a recombinant bacterium. In certain embodiments, the engineered microorganism is an engineered bacterium.

“Non-pathogenic bacteria” refer to bacteria that are not capable of causing disease or harmful responses in a host. In some embodiments, non-pathogenic bacteria are commensal bacteria. Examples of non-pathogen bacteria include, but are not limited to Bacillus, Bacteroides Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis. Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, and Lactococcus lactis (see, for example, Sonnenborn et al., 2009; Dinleyici et al., 2014; U.S. Pat. Nos. 6,835,376; 6,203,797; 5,589,168; 7,731,976). In some embodiments, naturally pathogenic bacteria may be genetically engineered to reduce or eliminate pathogenicity.

The terms “patient,” “subject,” and “individual” may be used interchangeably and refer to either a human or a non-human animal. These terms include mammals such as humans, non-human primates, livestock animals (e.g., bovines, porcines), companion animals (e.g., canines, felines) and rodents (e.g., mice and rats). In some embodiments, the terms refer to a human patient. In some embodiments, the terms refer to a human patient that suffers from a gastrointestinal inflammatory condition.

As used herein, “improved” should be taken broadly to encompass improvement in an identified characteristic of a disease state (e.g., a symptom), said characteristic being regarded by one of skill in the art to generally correlate, or be indicative of, the disease in question, as compared to a control, or as compared to a known average quantity associated with the characteristic in question. For example, “improved” epithelial barrier function associated with application of a protein of the disclosure can be demonstrated by comparing the epithelial barrier integrity of a human treated with a protein of the disclosure, as compared to the epithelial barrier integrity of a human not treated. Alternatively, one could compare the epithelial barrier integrity of a human treated with a protein of the disclosure to the average epithelial barrier integrity of a human, as represented in scientific or medical publications known to those of skill in the art. In the present disclosure, “improved” does not necessarily demand that the data be statistically significant (e.g., p<0.05); rather, any quantifiable difference demonstrating that one value (e.g. the average treatment value) is different from another (e.g, the average control value) can rise to the level of“improved.”

As used herein, the term “IBD” or “inflammatory bowel disease” refers to conditions in which individuals have chronic or recurring immune response and inflammation of the gastrointestinal (GI) tract. The two most common inflammatory bowel diseases are ulcerative colitis (UC) and Crohn's disease (CD).

As used herein, the term “mucositis” refers to very painful disorder involving inflammation of the mucous membrane, with the inflammation often accompanied by infection and/or ulceration. Mucositis can occur at any of the different mucosal sites in the body. A non-limiting list of examples of locations where mucositis can occur include mucosal sites in the oral cavity, esophagus, gastrointestinal tract, bladder, vagina, rectum, lung, nasal cavity, ear and orbita. Mucositis often develops as a side effect of cancer therapy, and especially as a side effect of chemotherapy and radiation therapy for the treatment of cancer. While cancerous cells are the primary targets of cancer therapies, other cell types can be damaged as well. Exposure to radiation and/or chemotherapeutics often results in significant disruption of cellular integrity in mucosal epithelium, leading to inflammation, infection and/or ulceration at mucosal sites.

As used herein, the tem “therapeutically effective amount” refers to an amount of a therapeutic agent (e.g., a bacterium, a peptide, polypeptide, or protein of the disclosure), which confers a therapeutic effect on the treated subject, at a reasonable benefit/risk ratio applicable to any medical treatment. Such a therapeutic effect may be objective (e.g., measurable by some test or marker) or subjective (e.g., subject gives an indication of, or feels an effect). In some embodiments. “therapeutically effective amount” refers to an amount of a therapeutic agent or composition effective to treat, ameliorate, or prevent (e.g., delay onset of) a relevant disease or condition, and/or to exhibit a detectable therapeutic or preventative effect, such as by ameliorating symptoms associated with the disease, preventing or delaying onset of the disease, and/or also lessening severity or frequency of symptoms of the disease. In some embodiments, a therapeutically effective amount can be measured in colony forming units (CFU). In some embodiments, a therapeutically effective amount can be about 10⁶-10¹² CFU, 10⁸-10¹² CFU, 10¹⁰-10¹² CFU, 10⁸-10¹⁰ CFU, or 10⁸-10¹¹ CFR of a bacterial species. A therapeutically effective amount is commonly administered in a dosing regimen that may comprise multiple unit doses. For any particular therapeutic agent, a therapeutically effective amount (and/or an appropriate unit dose within an effective dosing regimen) may vary, for example, depending on route of administration, or on combination with other therapeutic agents. Alternatively or additionally, a specific therapeutically effective amount (and/or unit dose) for any particular patient may depend upon a variety of factors including the particular form of disease being treated; the severity of the condition or pre-condition; the activity of the specific therapeutic agent employed; the specific composition employed; the age, body weight, general health, sex and diet of the patient; the time of administration, mute of administration, and/or rate of excretion or metabolism of the specific therapeutic agent employed; the duration of the treatment; and like factors as is well known in the medical arts. The current disclosure utilizes therapeutically effective amounts of novel proteins, and compositions comprising same, to treat a variety of diseases, such as: gastrointestinal inflammatory diseases or diseases involving gastrointestinal epithelial barrier malfunction. The therapeutically effective amounts of the administered protein, or compositions comprising same, will in some embodiments reduce inflammation associated with IBD or repair gastrointestinal epithelial barrier integrity and/or function.

As used herein, the term “treatment” (also “treat” or “treating”) refers to any administration of a therapeutic agent (e.g., a bacterium, a peptide, polypeptide, or protein of the disclosure), according to a therapeutic regimen that achieves a desired effect in that it partially or completely alleviates, ameliorates, relieves, inhibits, delays onset of, reduces severity of and/or reduces incidence of one or more symptoms or features of a particular disease, disorder, and/or condition (e.g., chronic or recurring immune response and inflammation of the gastrointestinal (GI) tract); In some embodiments, administration of the therapeutic agent according to the therapeutic regimen is correlated with achievement of the desired effect. Such treatment may be of a subject who does not exhibit signs of the relevant disease, disorder and/or condition and/or of a subject who exhibits only early signs of the disease, disorder, and/or condition. Alternatively or additionally, such treatment may be oft subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

“Pharmaceutical” implies that a composition, reagent, method, and the like, are capable of a pharmaceutical effect, and also that the composition is capable of being administered to a subject safely. “Pharmaceutical effect,” without limitation, can imply that the composition, reagent, or method, is capable of stimulating a desired biochemical, genetic, cellular, physiological, or clinical effect, in at least one individual, such as a mammalian subject, for example, a human, in at least 5% of a population of subjects, in at least 10%, in at least 20%, in at least 30%, in at least 50% of subjects, and the like. “Pharmaceutically acceptable” means approved by a regulatory agency of the Federal or a state government or listed in the U.S. Pharmacopoeia or other generally recognized pharmacopoeia for safe use in animals, and more particularly safe use in humans. “Pharmaceutically acceptable vehicle” or “pharmaceutically acceptable excipient” refers to a diluent, adjuvant, excipient or carrier with which a protein as described herein is administered.

“Preventing” or“prevention” refers to a reduction in risk of acquiring a disease or disorder (e.g., causing at least one of the clinical symptoms of the disease not to develop in a subject that may be exposed to or predisposed to the disease but does not yet experience or display symptoms of the disease, or causing the symptom to develop with less severity than in absence of the treatment). “Prevention” or “prophylaxis” may refer to delaying the onset of the disease or disorder.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. Some species, strains, and/or subtypes of non-pathogenic bacteria are currently recognized as probiotic bacteria. Examples of probiotic bacteria include, but we not limited to, Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, and Lactococcus lactis (Sonnenborn et al., 2009; Dinleyici et al., 2014; U.S. Pat. Nos. 6,833,376; 6,203,797; 5,589,168; 7,731,976). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, the term “recombinant bacterial cell”, “recombinant bacteria” or “genetically modified bacteria” refers to a bacterial cell or bacteria that have been genetically modified from their native state. For instance, a recombinant bacterial cell may have nucleotide insertions, nucleotide deletions, nucleotide rearrangements, and nucleotide modifications introduced into their DNA. These genetic modifications may be present in the chromosome of the bacteria or bacterial cell, or on a plasmid in the bacteria or bacterial cell. Recombinant bacterial cells of the disclosure may comprise exogenous nucleotide sequences on plasmids. Alternatively, recombinant bacterial cells may comprise exogenous nucleotide sequences stably incorporated into their chromosome. In some embodiments, recombinant bacterial cells of the disclosure are Lactococcus lactis bacterial cells comprising exogenous nucleotide sequences on plasmids. In some embodiments, recombinant bacterial cells of the disclosure are Lactococcus lactis bacterial cells having nucleotide insertions, nucleotide deletions, nucleotide rearrangements, and nucleotide modifications introduced into their DNA. In further embodiments, recombinant bacterial cells of the disclosure are genetically-engineered Lactococcus lactis bacterial cells.

As used herein, the term “transform” or “transformation” refers to the transfer of a nucleic acid fragment into a host bacterial cell, resulting in genetically-stable inheritance. Host bacterial cells comprising the transformed nucleic acid fragment are referred to as “recombinant” or “transgenic” or “transformed” organisms.

The therapeutic pharmaceutical compositions taught herein may comprise one or more natural products. However, in certain embodiments, the therapeutic pharmaceutical compositions themselves do not occur in nature. Further, in certain embodiments, the therapeutic pharmaceutical compositions possess markedly different characteristics, as compared to any individual naturally occurring counterpart, or composition component, which may exist in nature. That is, in certain embodiments, the pharmaceutical compositions taught herein—which comprise a therapeutically effective amount of a purified protein—possess at least one structural and/or functional property that impart markedly different characteristics to the composition as a whole, as compared to any single individual component of the composition as it may exist naturally. The courts have determined that compositions comprising natural products, which possess markedly different characteristics as compared to any individual component as it may exist naturally, are statutory subject matter. Thus, the taught therapeutic pharmaceutical compositions as a whole possess markedly different characteristics. These characteristics are illustrated in the data and examples taught herein.

Details of the disclosure are set forth herein. Although methods and materials similar or equivalent to those described herein can be used in the practice or testing of the present disclosure, illustrative methods and materials are now described. Other features, objects, and advantages of the disclosure will be apparent from the description and from the claims.

Therapeutic Proteins from the Microbiome

Numerous diseases and disorders are associated with decreased gastrointestinal epithelial cell barrier function or integrity. These diseases and disorders are multifaceted and present diagnostically in a myriad of ways. One such disease is inflammatory bowel disease (IBD), the incidence and prevalence of which is increasing with time and in different regions around the world, indicating its emergence as a global disease. (Molodecky et al., Gastroenterol 142:46-54, 2012). IBD is a collective term that describes conditions with chronic or recurring immune response and inflammation of the gastrointestinal (GI) tract. The two most common inflammatory bowel diseases are ulcerative colitis (UC) and Crohn's disease (CD). Both art marked by an abnormal response of the GI immune system. Normally, immune cells protect the body from infection. In people with IBD, however, this immune system mistakes food, bacteria, and other materials in the intestine for pathogens and an inflammatory response is launched into the lining of the intestines, creating chronic inflammation. When this happens, the patient experiences the symptoms of IBD.

IBD involves chronic inflammation of all, or part, of the digestive tract Both UC and CD usually involve, for example, severe diarrhea, abdominal pain, fatigue, and weight loss. IBD and associated disorders can be debilitating and sometimes lead to life-threatening complications.

With respect to intestinal barrier integrity, loss of integrity of the intestinal epithelium plays a key pathogenic role in IBD. Maloy, Kevin J.; Powrie, Fiona, 2011, Nature. 474 (7351): 298-306; Coskun, 2014, Front Med (Lausanne), 1:24; Martìní at al., 2017, Cell Mol Gastroenterol Hepatol, 4:33-46. It is hypothesized that detrimental changes in the intestinal microbiota induce an inappropriate or uncontrolled immune response that results in damage to the intestinal epithelium. Breaches in this critical intestinal epithelium barrier allow further infiltration of microbiota that, in turn, elicit further immune responses. Thus, IBD is a multifactorial disease that is driven in part by an exaggerated immune response to gut microbiota that can cause defects in epithelial barrier function.

Microbiome profiling of IBD patients has revealed distinct profiles such as increased Proteobacteria, including adherent-invasive E. coli, often at the expense of potentially beneficial microbes such as Roseburia spp (Machiels et al., 2014, Cut, 63:1275-1283; Patterson et al., 2017, Front Immunol, 8:1166; Shawki and McCole, 2017. Cell Mol Gastroenterol Hepatol, 3:41-50). Moreover, a decrease in Roseburia hominis was linked with dysbiosis in patients with ulcerative colitis. IBD affected individuals have been found to have 30-50 percent reduced biodiversity of commensal bacteria, such as decreases in Firmicutes (namely Lachnospiraceae) and Bacteroidetes. Further evidence of the role of gut flora in the cause of inflammatory bowel disease is that IBD affected individuals are more likely to have been prescribed antibiotics in the 2-5 year period before their diagnosis than unaffected individuals. See, Aroniadis O C, Brandt L J, “Fecal microbiota transplantation: past, present and future.” (2013) Curr. Opin. Gastroaterol. 29 (1)(2013): 79-84.

Protective bacterial communities, probiotics and bacterially derived metabolites have been demonstrated to improve disease in various clinical and pre-clinical studies. For example, fecal microbial transfer (FMT) experiments have shown some success in IBD patients, although challenges still exist with FMT (Moayyedi et al., 2015, Gastroenterology, 149:102-109 e106; Qazi et al., 2017. Out Microbes, 8-574-588; Narula et al., 2017, Inflamm Bowel Dis, 23:1702-1709). In other studies treatment with probiotics including VSL #3, Lactobacillus spp. and Bifidobacterium spp. have also shown to have beneficial effects in humans and animal models (Srutkova et al, 2015, PLoS One, 10:e0134050; Pan et al., 2014, Benef Microbes, 5:315-322; Huynh et al., 2009, Inflamm Bowel Dis, 15:760-768; Bibiloni et al., 2005. Am J Gastroenterol, 100:1539-1546). Furthermore, bacterial products such as p40 from L. rhamnosus GG and Amuc-1100 from A. muciniphila have been shown to promote barrier function and protect in animal models of IBD and metabolic disease, receptively (Yan et al., 2011, J Clin Invest. 121:2242-2253; Plovier et al., Nat Mod, 23:107-113).

While uses of live microbial populations to treat diseases is increasingly common, such methods rely on the ability of the administered bacteria to survive in the host or patient and to interact with the host tissues in a beneficial and therapeutic way. An alternative approach, provided here, is to identify microbially-encoded proteins and variants thereof which can affect cellular functions in the host and provide therapeutic benefit. Such proteins can be administered, for example, as pharmaceutical compositions comprising a substantially isolated or purified therapeutic, bacterially-derived protein or as a live biotherapeutic (bacterium) engineered to express the therapeutic protein as an exogenous protein. Moreover, methods of treatment comprising administration of the therapeutic protein am not limited to the gut (small intestine, large intestine, rectum) but may also include treatment of other disorders within the alimentary canal such as oral mucositis.

To identify microbially-derived proteins which may have therapeutic application in gastrointestinal inflammatory disorders, fecal samples from humans who were healthy or who were diagnosed with UC or CD were analyzed to determine the microbial compositions of fecal samples collected from these individuals. A comparison of the bacterial profiles from healthy vs. diseased subjects identified bacteria that were either likely to be beneficial (greater numbers in healthy vs. diseased) or detrimental (lower numbers in healthy vs. diseased). Among the bacterial species identified as beneficial was Roseburia hominis, consistent with studies referenced above. Extensive bioinformatics analysis was then performed to predict proteins encoded by the bacterium and then to identify those proteins which are likely to be secreted by the bacterium. Proteins which were predicted to be secreted proteins were then characterized using a series of in vitro assays to study the effect of each protein on epithelial barrier integrity, cytokine production and/or release, and wound healing. Proteins identified as functioning to increase epithelial barrier integrity were then assessed in an in vivo mouse model for colitis. One such protein, identified herein as “SG-11,” demonstrated both in vitro and in vivo activity indicative of its ability to provide therapeutic benefit for increasing epithelial barrier integrity and for treating diseases and disorders associated with epithelial barrier integrity as well as treating inflammatory gastrointestinal diseases such as IBDs. The amino acid and polynucleotide sequences of SG-11 and variants thereof, as well as functional activity of the SG-11 protein and variants thereof was described in U.S. provisional patent applications 62/482,963, filed Apr. 7, 2017; 62/607,706, filed Dec. 19, 2017; 62/611,334, filed Dec. 28, 2017. The disclosure of each of these three provisional applications is incorporated herein by reference in their entirety. The SG-11 protein, variants thereof, and functional activity is summarized below and in Examples 1-13.

### The S-11 Protein

The SG-11 protein is encoded within a 768 nucleotide sequence (SEQ ID NO:2) present in the genome of Roseburia hominis. A complete genomic sequence for R. hominis strain can be found at GenBank accession number CP003040 (the sequence incorporated herein by reference in its entirety). A 16S rRNA gene sequence for the Roseburia hominis strain can be found at GenBank accession number AJ270482. The full-length protein encoded by the R. hominis genomic sequence is 256 amino acids in length (SEQ ID NO:1), wherein residues 1-25 are predicted to be a signal peptide that is cleaved in vivo to produce a mature protein of 232 amino acids (SEQ ID NO:3; encoded by SEQ ID NO:4). Recombinant SG-11 can be expressed with an N-terminal methionine (encoded by the codon ATG) to produce a mature protein of 233 amino acids (SEQ ID NO:7)

SG-11 was recombinantly expressed in different commercially available and routinely used expression vectors. For example, SG-11 (a protein comprising SEQ ID NO:3) was expressed using a pGEX expression vector which expresses the protein of interest with a GST tag and protease site which is cleaved after expression and purification, a pET-28 expression vector which adds an N-terminal FLAG tag, and a pD451 expression vector which was used to express the SG-11 protein consisting of SEQ ID NO:7 and having no N-terminal tag. Experiments performed and repeated with these proteins showed that the minor N-terminal and/or C-terminal variations resulting from the use of the different protein expression systems and DNA constructs retained equivalent functional activity in in vivo and in vitro assays. It is understood that unless otherwise indicated, the term “SG-11” refers herein to the amino acid sequence depicted herein as SEQ ID NO:3 and such variants of the protein comprising the amino acid sequence of SEQ ID NO:3 (including but not limited to SEQ ID NO:1, SEQ ID NO:5, SEQ ID NO:7). SG-11 variants can include variations in amino acid residues (e.g., substitutions, insertions, and/or deletions) as well as modifications such as fusion constructs and post-translational modifications (e.g., phosphorylation, glycosylation, etc.). Some exemplary embodiments of the SG-11 protein and encoding nucleic acids are provided in Table 1 below.

TABLE 1

| Amino Add Sequence | Encoding Nucleic Acid Sequence |
|---|---|
| SEQ ID NO: 1 | SEQ ID NO: 2 |
| MKRLVCTVCSVLLCAGLLSGCGT | ATGAAGAGATTAGTGTGCACGGTCTGCAGTGTACTGTTGTGTGC |
| SLEGEESVVYVGKKGVIASLDVET | GGGACTTCTCTCCGGATGCGGTACCT |
| LDQSYYDETELKSYVDAEVEDYTA | CGCTGGAGGGAGAGGAAAGTGTCGTGTACGTGGGAAAGAAAGG |
| EHGKNAVKVESLKVEDGVAKLK | CGTGATAGCGTCGCTGGATGTGGAGAC |
| MKYKTPEDYTAFNGIELYQGKVV | GCTCGATCAGTCCTACTACGATGAGACGGAACTGAAGTCCTATGT |
| ASLAAGYVYDGEFARVEEGKVVG | GGATGCAGAGGTGGAAGATTACACC |
| AATKQDIYSEDDLKVAIIRANTDV | GCGGAGCATGGTAAAAATGCAGTCAAGGTGGAGAGCCTTAAGG |
| KVDGEICYVSCQNVKLTGKDSVSI | TGGAAGACGGTGTGGCGAAGCTTAAGA |
| RDGYYLETGSVTASVDVTGQESV | TGAAGTACAAGACACCGGAGGATTATACCGCATTTAATGGAATT |
| GTEQLSGTEQMEMTGEPVNAD | GAACTCTATCAGGGGAAAGTCGTTGC |
| DTEQTEAAAGDGSFETDVYTFIV | ATTCCCTGGCGGCAGGATACGTCTACGACGGGGAGTTCGCCCGCG |
| YK | TGGAGGAAGGCAAGGTTGTGGGAGCT |
| | GCCACAAAACAGGATATTTACTCTGAGGATGATTTGAAAGTTGCC |
| | ATCATCCGTGCCAATACGGATGTGA |
| | AGGTGGACGGTGAGATCTGCTATGTCTCCTGTCAGAATGTGAAG |
| | CTGACCGGAAAAGACAGTGTGTCGAT |
| | CCGTGACGGATATTATCTTGAGACGGGAAGCGTGACGGCATCCG |
| | TGGATGTGACCGGACAGGAGAGCGTC |
| | GGGACCGAGCAGCTTTCGGGAACCGAACAGATGGAGATGACCG |
| | GGGAGCCGGTGAATGCGGATGATACCG |
| | AGCAGACAGAGGCGGCGGCCGGTGACGGTTCGTTCGAGACAGA |
| | CGTATATACTTTCATTGTCTACAAA |
| SEQ ID NO: 3 | SEQ ID NO: 4 |
| LEGEESVVYVGKKGVIASLDVETL | TTGGAGGGTGAAGAGTCTGTTGTCTATGTGGGTAAGAAAGGTGT |
| DQSYYDETELKSYVDAEVEDYTA | GATCGCGTCCCTGGACGTCGAGACTCTGGACCAGTCTTACTATGA |
| EHGKNAVKVESLKVEDGVAKLK | TGAAACCGAGCTGAAGTCGTATGTGGACGCCGAAGTTGAGGATT |
| MKYKTPEDYTAFNGIELYQGKVV | ACACGGCCGAGCACGGCAAAAATGCCGTCAAAGTTGAGAGCTTG |
| ASLAAGYVYDGEFARVEEGKVVG | AAAGTTGAGGACGGCGTGGCAAAGCTGAAGATGAAATACAAGA |
| AATKQDIYSEDDLKVAIIRANTDV | CCCCAGAGGACTACACGGCGTTCAATGGTATCGAGCTGTATCAG |
| KVDGEICYVSCQNVKLTGKDSVSI | GGCAAAGTCGTCGCATCCCTGGCAGCGGGCTATGTGTACGACGG |
| RDGYYLETGSVTASVDVTGQESV | TGAGTTTGCGCGCGTCGAAGAAGGCAAAGTTGTGGGTGCGGCTA |
| GTEQLSGTEQMEMTGEPVNAD | CGAAACAAGATATCTACAGCGAAGATGACCTGAAAGTCGCGATT |
| DTEQTEAAAGDGSFETDVYTFIV | ATTCGTGCTAACACCGATGTTAAAGTTGATGGCGAGATTTGCTAC |
| YK | GTTAGCTGTCAAAACGTAAAGCTGACGGGTAAAGATAGCGTGAG |
| | CATTCGTGATGGCTATTATCTGGAAACCGGTAGCGTTACGGCGA |
| | GCGTCGATGTTACCGGTCAAGAGAGCGTGGGTACCGAACAGCTG |
| | AGCGGCACCGAACAGATGGAAATGACCGGTGAACCGGTTAACGC |
| | AGACGACACGGAACAAACCGAAGCCGCGGCAGGCGACGGTAGC |
| | TTCGAGACTGACGTGTACACCTTTATCGTGTACAAG |
| SEQ ID NO: 7 | SEQ ID NO: 8 |
| MLEGEESVVYVGKKGVIASLDVE | ATGTTGGAGGGTGAAGAGTCTGTTGTCTATGTGGGTAAGAAAGG |
| TLDQSYYDETELKSYVDAEVEDYT | TGTGATCGCGTCCCTGGACGTCGAGACTCTGGACCAGTCTTACTA |
| MKYKTPEDYTAFNGIELYQGKVV | TGATGAAACCGAGCTGAAGTCGTATGTGGACGCCGAAGTTGAGG |
| ASLAAGYVYDGEFARVEEGKVVG | ATTACACGGCCGAGCACGGCAAAAATGCCGTCAAAGTTGAGAGC |
| AATKQDIYSEDDLKVAIIRANTDV | TTGAAAGTTGAGGACGGCGTGGCAAAGCTGAAGATGAAATACAA |
| KVDGEICYVSCQNVKLTGKDSVSI | GACCCCAGAGGACTACACGGCGTTCAATGGTATCGAGCTGTATC |
| RDGYYLETGSVTASVDVTGQESV | AGGGCAAAGTCGTCGCATCCCTGGCAGCGGGCTATGTGTACGAC |
| GTEQLSGTEQMEMTGEPVNAD | GGTGAGTTTGCGCGCGTCGAAGAAGGCAAAGTTGTGGGTGCGG |
| DTEQTEAAAGDGSFETDVYTFIV | CTACGAAACAAGATATCTACAGCGAAGATGACCTGAAAGTCGCG |
| YK | ATTATTCGTGCTAACACCGATGTTAAAGTTGATGGCGAGATTTGC |
| | TACGTTAGCTGTCAAAACGTAAAGCTGACGGGTAAAGATAGCGT |
| | GAGCATTCGTGATGGCTATTATCTGGAAACCGGTAGCGTTACGG |
| | CGAGCGTCGATGTTACCGGTCAAGAGAGCGTGGGTACCGAACAG |
| | CTGAGCGGCACCGAACAGATGGAAATGACCGGTGAACCGGTTAA |
| | CGCAGACGACACGGAACAAACCGAAGCCGCGGCAGGCGACGGT |
| | AGCTTCGAGACTGACGTGTACACCTTTATCGTGTACAAG |
| SEQ ID NO: 9 | SEQ ID NO: 10 |
| MDYKDDDDKGSSHMLEGEESVV | ATGGACTACAAAGACGATGACGACAAGGGCAGCAGCCATATGCT |
| YVGKKGVIASIDVETLDQSYYDET | GGAGSGAGAGGAAAGTGTCGTGTACGTGGGAAAGAAAGGCGT |
| ELKSYVDAEVEDYTAEHGKNAVK | GATAGCGTCGCTGGATGTGGAGACGCTCGATCAGTCCTACTACG |
| VESLKVEDGVAKLKMKYKTPEDY | ATGAGACGGAACTGAAGTCCTATGTGGATGCAGAGGTGGAAGAT |
| TAFNGIELYQGKVVASLAAGYVY | TACACCGCGGAGCATGGTAAAAATGCAGTCAAGGTGGAGAGCCT |
| DGEFARVEEGKVVGAATKQDIYS | TAAGGTGGAAGACGGTGTGGCGAAGCTTAAGATGAAGTACAAG |
| EDDLKVAIIRANTDVKVDGEICYV | ACACCGGAGGATTATACCGCATTTAATGGAATTGAACTCTATCAG |
| SCQNVKLTGKDSVSIRDGYYLET | GGGAAAGTCGTTGCTTCCCTGGCGGCAGGATACGTCTACGACGG |
| GSVTASVDVTGQESVGTEQLSGT | GGAGTTCGCCCGCGTGGAGGAAGGCAAGGTTGTGGGAGCTGCC |
| EQMEMTGEPVNADDTEQTEAA | ACAAAACAGGATATTTACTCTGAGGATGATTTGAAAGTTGCCATC |
| AGDGSFETDVYTFIVYK | ATCCGTGCCAATACGGATGTGAAGGTGGACGGTGAGATCTGCTA |
| | TGTCTCCTGTCAGAATGTGAAGCTGACCGGAAAAGACAGTGTGT |
| | CGATCCGTGACGGATATTATCTTGAGACGGGAAGCGTGACGGCA |
| | TCCGTGGATGTGACCGGACAGGAGAGCGTCGGGACCGAGCAGC |
| | TTTCGGGAACCGAACAGATGGAGATGACCGGGGAGCCGGTGAA |
| | TGCGGATGATACCGAGCAGACAGAGGCGGCGGCCGGTGACGGT |
| | TCGirCGAGACAGACGTATATACTTTCATTGTCTACAAA |

### Epithelial Barrier Function in Disease

Studies in recent years have identified a major role of both genetic and environmental factors in the pathogenesis of IBD. Markus Neurath, Nature Reviews Immunology, Vol. 14, 329-342 (2014). A combination of these IBD risk factors seems to initiate detrimental changes in epithelial barrier function, thereby allowing the translocation of luminal antigens (for example, bacterial antigens from the commensal microbiota) into the bowel wall. Id. Subsequently, aberrant and excessive responses, such as increased pro-inflammatory cytokine release, to such environmental triggers cause subclinical or acute mucosal inflammation in a genetically susceptible host. Id. Thus, the importance of proper epithelial barrier function in IBD is apparent, for in subjects that fail to resolve acute intestinal inflammation, chronic intestinal inflammation develops that is induced by the uncontrolled activation of the mucosal immune system. In particular, mucosal immune cells, such as macrophages, T cells, and the subsets of innate lymphoid cells (ILCs), seem to respond to microbial products or antigens from the commensal microbiota by, e.g., producing cytokines that can promote chronic inflammation of the gastrointestinal tract. Consequently, restoring proper epithelial barrier function to patients may be critical in resolving IBD.

The therapeutic activity of SG-11 was identified in part by its beneficial effects on epithelial barrier function both in vitro and in vivo. SG-11 was shown to be active in increasing epithelial barrier integrity as shown by an in vitro trans-epithelial electrical resistance (TEER) assay (see Example 2). A TEER may is a well-known method for measuring effects on the structural and functional integrity of an epithelial cell layer (Srinivasan et al., 2015, J Lab Autom, 20:107-126; Beduneau et al., 2014, Eur J Pharm Biopharm, 87:290-298; Zolotarevsky et al., 2002, Gastroenterology, 123:163-172, Dewi, e al, 2004, J. Virol. Methods. 121:171-180, Dewi, et al., 2004, J. Virol. Methods. 121: 171-180, and Mandic, et al., 2004. Clin. Exp. Metast. 21:699-704). The assay performed and described herein consists of an epithelial monolayer made up of enterocyte and goblets cells to more accurately model the structural and functional components of the intestinal epithelium. The cells are cultured until tight junction formation occurs and barrier function capacity is assessed by a measurement of trans-epithelial electrical resistance. Upon addition of an insult, such as heat killed E. coli, there is a decrease in electrical resistance across the epithelial layer. Control reagents useful in the TEER assay include staurosporine and a myosin light chain kinase inhibitor. Staurosporine is a broad spectrum kinase inhibitor, originating from Streptomyces staturosporeus, which induces apoptosis. This reagent disrupts about 98% of the gap junctions leading to a decrease in electrical resistance in a TEER assay. Myosin light chain kinase (MLCK) is the terminal effector in a signaling cascade induced by pro-inflammatory cytokines, which results in contraction of the perijunctional actomyosin ring, resulting in separation of the gap junctions. By inhibiting MLCK, disruption of tight, junctions is prevented. MLCK inhibitor in a TEER assay should reduce or prevent the reduction of electrical resistance in a TEER assay.

As noted above. IBDs and other gastrointestinal disorders including inflammatory disorders are believed to be associated with decreased epithelial barrier integrity which leads inter alia to bacteria crossing the barrier and inciting an immune response. Example 3 shows that SG-11 protein can enhance or facilitate epithelial wound healing, an activity that can play a role in the maintenance or repair of and epithelial barrier such as an intestinal or mucosal epithelial barrier.

In view of the effect of SG-11 to repair barrier function integrity, SG-11 was analyzed in vivo for its ability to reduce damage in a rodent model of BD. Examples 4 and 5 (SG-11) and 13 (SG-11 variant) describe studies done using a DSS (dextran sodium sulfate) animal model, a model well accepted for the study of agents on IBDs (Classaign et al., 2014. Curr Protoc Immunol, 104:Unit-15.25; Kiesler et al., 2015, Cell Mol Gastroenterol Hepatol). DSS is a sulfated polysaccharide that is directly toxic to colonic epithelium and causes epithelial cell injury leading to loss of barrier function due to disrupted gap junctions. In these experiments, animals were treated with SG-11 either prior to (Example 4) or after (Example 5) induction of colitis in the mouse. As a positive control, the mice were also treated with Gy2-GLP2, a stable analog of glucagon-like peptide 2 (GLP2). Gly2-GLP2 is known to promote epithelial cell growth and reduce colonic injury in experiment mouse colitis models. Results of the DSS studies show that SG-11 protein was effective in reducing weight loss in DSS models, an important indicator of clinical efficacy for IBD therapeutics. SG-11 treatment also reduced scores in gross pathology and intestinal histopathology analyse.

It is noted that while SG-11 treatment improved the 4Kda-FITC intestinal permeability readout and reduced serum levels of LPS binding protein (LBP—a marker of LPS exposure) in Example 7, no significant effects upon treatment with SG-11 or Gly2-GLP2 were observed in Example 8. This is not surprising when considering that animals in Example 8 were treated with DSS for 7 days prior to replacement with normal drinking water and treatment with SG-11 or Gy2-GLP2. This prior exposure to DSS results in damage to the intestinal epithelium, translocation of LPS across a disrupted epithelial barrier, and induction of LBP secretion. However, based on 4KDa-FITC dextran measurements, epithelial barrier repair appears to occur rapidly, within 3-4 days, following replacement of DSS with normal drinking water (data not shown, FIG. 12). Accordingly, it is difficult to detect improvements in 4KDa-FITC permeability readouts in treated vs. untreated animals at the time of measurement (after 6 days of treatment). Additionally, levels of LBP in the serum may be independent of barrier function repair in animals exposed to DSS for an extended period of time prior to therapeutic treatment (Example g). For instance, hepatocytes activated by translocating LPS during the DSS exposure produce and secrete large amounts of LPB. Accordingly, and without being bound by theory, the short time period of the study may not allow sufficient time for inactivation of the hepatocytes and clearance of LBP from the serum of the DSS-treated animals. It is considered, therefore, that continuation of the study with measurement of serum LBP at later time points would show a decrease in serum LBP levels, however, the decrease in serum LBP may be similar in both treated and untreated animals if barrier function is restored in both animals before LBP can be cleared from the serum.

### Amino Acid Variants of SG-11

In view of the therapeutic value of SG-11 and its use for treating disease, the protein was further characterized and its sequence modified to change its primary structure in ways that would optimize pharmaceutical formulation and long-term storage of the protein.

As described in Example 6, SG-11 (SEQ ID NO:7) was used to perform a BLAST search of the GenBank non-redundant protein database to identify proteins with similar amino acid sequences and which may be functional homologs or have function(s) similar to those of SG-11. Three such proteins were identified and the predicted mature sequence of each (without an N-terminal signal peptide) was aligned with SEQ ID NO:3 to identify regions and individual positions within the proteins which were relatively conserved. See FIG. 14. These three proteins are disclosed herein as SEQ ID NO:21 (derived from GenBank Acc. No. WP_006857001), SEQ ID NO:22 (derived from GenBank Acc. No. WP_075679733), and SEQ ID NO:23 (derived from GenBank Acc. No. WP_055301040). Accordingly, provided herein are pharmaceutical compositions comprising any one of these three proteins or variants or fragments thereof. Also provided herein are methods for treating diseases associated with barrier function disorders and/or gastrointestinal diseases or disorders comprising administering to a subject in need thereof a pharmaceutical composition comprising any one of SEQ ID NO:21, SEQ ID NO:22 and SEQ ID NO:23 or variant or fragment thereof. In some embodiments, provided is a protein comprising an amino acid sequence that is at least 90%, 95%, 97%, 98% or 99% identical to the sequence of residues 73 to 227 of SEQ ID NO:21 or fragment thereof, residues 72 to 215 of SEQ ID NO:22 or fragment thereof, or residues 72 to 236 of SEQ ID NO:23 or fragment thereof. Also provided herein are bacteria expressing a protein comprising an amino acid sequence that is at least 90%, 95%, 97%, 98% or 99% identical to the sequence of SEQ ID NO:21, SEQ ID NO:22 or SEQ ID NO:23. Also provided herein are bacteria expressing a protein that is at least 90%, 95%, 97%, 98% or 99% identical to the sequence of residues 73 to 227 of SEQ ID NO:21 or fragment thereof, residues 72 to 215 of SEQ ID NO:22 or fragment thereof, or residues 72 to 236 of SEQ ID NO:23 or fragment thereof.

In the interest of enhancing the stability of SG-11 proteins for use in pharmaceutical formulations and clinical applications, studios were performed to identify and characterize post translational modifications of purified SG-11 protein. These experiments are described in Examples 7-9. Such analysis shows that the SG-11 protein can undergo at least the PTMs of methionine oxidation and asparagine deamidation. Moreover, experiments described in Example 10 the cysteines in SG-11 are unlikely to form disulfide bonds in the native, functional conformation of the active protein, suggesting that the fee sulfhydryl groups in SG-11 may cause aggregation in a solution containing the purified protein. Based on these stability studies and despite the conserved nature of the residues in SG-11 as seen in the multiple sequence alignment (FIG. 14) it was decided to test whether or not the cysteines at positions 147 and/or 151 (with reference to SEQ ID NO:7) could be substituted with a different amino acid. Also, substitution of conserved asparagines at positions 53 and 83 were considered. In an exemplary embodiment, the SG-11 sequence of SEQ ID NO:7 is modified to introduce the substitutions of C147V and C151S to generate SEQ ID NO:11 (SG-11V1). The C147V and C151S substitutions are also present in the provided SG-11 variants SG-11V2 (SEQ ID NO:13; comprising G84D, C147V, C151S), SG-11V3 (SEQ ID NO:15; comprising N83S, C147V, C151S), SG-11V4 (SEQ ID NO:17; comprising N53S, GMD, C147V, C151S) and SG-11V5 (SEQ ID NO:19; comprising N53S, N83S, C147V, C151S).

An embodiment of SG-11V5 and an encoding nucleic acid sequence is provided in Table 2 below.

TABLE 2

| Amino Acid Sequence | Encoding Nucleic Acid Sequence |
|---|---|
| SEQ ID NO: 19 (SG-11V5) | SEQ ID NO: 20 |
| MLEGEESVVYVGKKGVIASLDVE | ATGTTGGAGGGTGAAGAGTCTGTTGTCTATGTGGGTAAGAAAGG |
| TLDQSYYDETELKSYVDAEVEDYT | TGTGATCGCGTCCCTGGACGTCGAGACTCTGGACCAGTCTTACTA |
| AEHGKSAVKVESIKVEDGVAKIK | TGATGAAACCGAGCTGAAGTCGTATGTGGACGCCGAAGTTGAGG |
| MKYKTPEDYTAFSGIELYQGKVV | ATTACACGGCCGAGCACGGCAAATCCGCCGTCAAAGTTGAGAGC |
| ASLAAGYVYDGEFARVEEGKVVG | TTGAAAGTTGAGGACGGCGTGGCAAAGCTGAAGATGAAATACAA |
| AATKQDIYSEDELKVAIIRANTDV | GACCCCAGAGGACTACACGGCGTTCAGCGGTATCGAGCTGTATC |
| KVDGEIVYVSSQNVKLTGKDSVSI | AGGGCAAAGTCGTCGCATCCCTGGCAGCGGGCTATGTGTACGAC |
| RDGYYLETGSVTASVDVTGQESV | GGTGAGTTTGCGCGCGTCGAAGAAGGCAAAGTTGTGGGTGCGG |
| GTEQLSGTEQMEMTGEPVNAD | CTACGAAACAAGATATCTACAGCGAAGATGACCTGAAAGTCGCG |
| DTEQTEAAAGDGSFETDVYTFIV | ATTATTCGTGCTAACACCGATGTTAAAGTTGATGGCGAGATTGTG |
| YK | TACGTTAGCAGCCAAAACGTAAAGCTGACGGGTAAAGATAGCGT |
| | GAGCATTCGTGATGGCTATTATCTGGAAACCGGTAGCGTTACGG |
| | CGAGCGTCGATGTTACCGGTCAAGAGAGCGTGGGTACCGAACAG |
| | CTGAGCGGCACCGAACAGATGGAAATGACCGGTGAACCGGTTAA |
| | CGCAGACGACACGGAACAAACCGAAGCCGCGGCAGGCGACGGT |
| | AGCTTCGAGACTGACGTGTACACCTTTATCGTGTACAAG |

Example 10 shows that PTMs (methionine oxidation and asparagine deamidation) is significantly reduced in SG-11V5 as compared to SG-11 (SEQ ID NO:7). The reductions were observed both at different temperatures and in different storage buffers. Example 11 describes an experiment performed to determine if an SG-11 variant comprising the cysteine substitutions (SG-11V5, SEQ ID NO:19) would affect aggregation of the protein in a storage buffer. The results show that the SG-11V5 variant has reduced aggregation compared to SG-11 (SEQ ID NO:7) when tested in different storage buffers.

Notably, ashough the amino acids substituted to generate SG-11V5 are present in a relatively conserved region of the SG-11 protein, it was possible to substitute these 4 residues without losing functional activity (Examples 12 and 13, described in more detail below).

Based on the experimental data and analysis described herein, variants of SG-11 (e.g, SEQ ID NO:3 or SEQ ID NO:5) were designed to substitute any one or more of amino acids N53. N83, C147 and C151 of SEQ ID NO:7 (wherein noted substitutions are at residue positions with respect to SEQ ID NO:7). An embodiment of this variant is provided below in Table 3, as SEQ ID NO:31, wherein the residue at each of positions 53, 83, 147 and 151 is denoted as X indicating that one or more of these 4 residues can each be substituted for any of the other 19 amino acids. In some embodiments, the protein comprises the amino acid sequence of SEQ ID NO:33. In some embodiments, X53 is N, S, T, M, R, Q and/or X83 is N, R or K, and/or X84 is G or A, and/or X147 is C, S, T, M, V, L, A, or G, and/or X151 is C, S, T, M, V, L, A, or G. In some embodiments, X53 is N, S or K and/or X83 is N or R and/or X84 is G or A and/or X147 is C, V, L or A and/or X151 is C, S, V, L or A. In some embodiments, X53 is any amino acid other than N, X83 is any amino acid other than N, X84 is any amino acid other than G, X147 is any amino acid other than C, and/or X151 is any amino acid other than C.

TABLE 3
Amino Add Sequence for SEQ ID NO: 33

MLEGEESVVYVGKKGVIASLDVETLDQSYYDETELKSYVDAEVEDYTAE
HGKXAVKVESLKVEDGVAKLKMKYKTPEDYTAFXXIELYQGKVVASLAA
GYVYDGEFARVEEGKVVGAATKQDIYSEDDLKVAIIRANTDVKVDGEIX
YVSXQNVKLTGKDSVSIRDGYYLETGSVTASVDVTGQESVGTEQLSGTE
QMEMTGEPVNADDTEQTEAAAGDGSFETDVYTFIVYK

In another example, certain amino acids of the taught proteins may be substituted for other amino acids in a protein structure without appreciable loss of interactive binding capacity with structures such as, for example, binding sites on substrate molecules, receptors, antigen-binding regions of antibodies, and the like. Thus, these proteins would be biologically functional equivalents of the disclosed proteins (e.g. comprising SEQ ID NO:3 or variants thereof.) So-called “conservative” changes do not disrupt the biological activity of the protein, as the structural change is not one that impinges on the protein's ability to carry out its designed function. It is thus contemplated by the inventors that various changes may be made in the sequence of genes and proteins disclosed herein, while still fulfilling the goals of the present disclosure.

Also described herein are variants of SG-11: SEQ ID NO:11 (C147V, C151S, “SG11-V1”), SEQ ID NO:13 (G84D, C147V, C151S “SG11-V2”), SEQ ID NO:15 (N83S, C147V, C151S “SG11-V3”), SEQ ID NO:17 (N53S, G84D, C147V, C151S “SG11-V4”), and SEQ ID NO:19 (N53S, N83S, C147V, C151“SG11-V5”).

Importantly, the SG-11 variant protein comprising SEQ ID NO:19 maintained its activity both with respect to the TEER assay (Example 12) and in vivo DSS mouse models (Example 13), showing that variants of SG-11 can maintain therapeutic function equivalent to that of wild type SG-11. Specifically, in vitro TEER and in vivo DSS model experiments were performed in which SG-11 (SEQ ID NO:7) and SG-11V5 (SEQ ID NO19) were used in parallel. Example 12 shows that SG-11 and SG-11V5 has essentially the same functional ability to reduce TEER in vitro. As described in Examples 4 and 5 in which DSS model mice were treated with SG-11 before or after DSS treatment, Example 13 was performed to compare in vivo efficacy of SG-11 and the SG-11 variant. Example 13 also compares administration to the mice with the protein before DSS (described as Example 13A) and after DSS (described as Example 13B) treatment. SG-11 and the SG-11 variant reduced weight loss (FIGS. 20A and 20B) as well as gross pathology clinical scores (FIG. 21). Again, SG-11 reduced intestinal permeability and serum LBP levels while SG-11V5 is shown to reduce intestinal permeability (FIG. 18A) and serum LBP levels in a dose-dependent manner (FIG. 19A) in Example 13A. Similar to results observed in Examples 4 and 5, SG-11 and the SG-11 variant protein did not reduce intestinal permeability or serum LBP levels in Example 13B where the therapeutic protein was administered after a prolonged assault with DSS and results observed over a limited period of time. As discussed above, it is considered that continuation of the study would show a decrease in both permeability and serum LBP levels.

In view of these data, provided herein is a therapeutic protein that is at last 90%, 95%, 96%, 97%, 98%, 99% or 100% identical to a protein comprising the amino acid sequence of SEQ ID NO:3 or a fragment thereof. In an alternative embodiment, the therapeutic protein has at least 70%, 75%, 80%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or 100% sequence identity to SEQ ID NO:19 or to SEQ ID NO-7 or a fragment thereof. In some embodiments, the therapeutic protein comprises an amino acid sequence that is identical to SEQ ID NO:19 or SEQ ID NO:5. The therapeutic protein alternatively can be one which is a variant of SEQ ID NO:3 or SEQ ID NO:7, wherein the therapeutic protein has 1, 2, 3, 4, 5, 6, 7, 8, 9 or 10 amino acid substitutions relative to SEQ ID NO:7. In some embodiments, the variant therapeutic protein comprises a non-naturally occurring variant of SEQ ID NO:3. Alternatively stated, the therapeutic protein comprises 1, 2, 3, 4, 5, 6, 7, 8, 9 or 10 non-naturally occurring amino acid substitutions relative to SEQ ID NO:3. In some embodiments, the therapeutic protein does not comprise an amino acid sequence identical to the sequence of residues 2 to 233 of SEQ ID NO:7.

In some embodiments, the SG-11 protein can be modified or varied by one or more amino acid insertions or deletions. An insertion can be the addition of 1 or more (e.g., 1, 2, 3, 4, 5, 6, 7, 8, 9 or 1 to 10, 1 to 20, 1 to 30, 1 to 40 or 1 to 50) amino acids to the N-terminus and/or C-terminus of the protein and/or can be an inset of 1 or more (e.g., 1, 2, 3, 4, 5, 6, 7, 8, 9 or 1 to 10, 1 to 20, 1 to 30, 1 to 40 or 50) amino acids at a position located between the N- and C-terminal amino acids. Similarly, the deletion of the 1 or more (e.g., 1, 2, 3, 4, 5, 6, 7, 8, 9 or 1 to 10, 1 to 20, 1 to 30, 1 to 40 or 1 to 50) amino acids can occur at any of the N- and C-terminus and in the internal portion.

In some embodiments, a modified or variant protein is provided which contains at least one non-naturally occurring amino acid substitution relative to SEQ ID NO:3. In some embodiments, the variant protein comprises 1, 2, 3, 4, 5, 6, 7, 8, 9 or 10 amino acid substitutions relative to SEQ ID NO:3 or SEQ ID NO:7. In further embodiments, the modified protein contains the amino acid sequence as depicted in SEQ ID NO:5, SEQ ID NO:7, SEQ ID NO:9, SEQ ID NO:11 (SG-11V1), SEQ ID NO:13 (SG-11V2), SEQ ID NO:15 (SG-11V3), SEQ ID NO:17 (SG-11V4), or SEQ ID NO:19 (SG-11V5).

In some embodiments, a therapeutic protein according to the present disclosure encompasses any one of the variant proteins (e.g., SEQ ID NO:11, SEQ ID NO:13, SEQ ID NO:15, SEQ ID NO:17; or SEQ ID NO:19) that also retains one or more activities of the full length mature protein depicted in, for example, SEQ ID NO:3 or SEQ ID NO:7.

Also envisioned are polynucleotide sequences which encodes these proteins. It is well known to the ordinarily skilled artisan that 2 polynucleotide sequences which encode a single polypeptide sequence can share relatively low sequence identity due to the degenerative nature of the genetic code. For example, if every codon in the polynucleotide encoding a 233-amino acid sequence contained at least 1 substitution in its third position, that would calculate to about 67% sequence identity between the 2 polynucleotides. A polynucleotide of the present disclosure comprises a sequence that encodes a protein that is at least 70%, 75%, 80%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or 100% identical to SEQ ID NO:19. Accordingly. In some embodiments, the polynucleotide comprises a sequence that is at least 67% identical to SEQ ID NO:4 or SEQ ID NO:8, or is about 67% to 100%, 70% to 100%, 75% to 100%, 80% to 100%, 90% to 100% or 95% to 100% identical to SEQ ID NO:20 or a fragment thereof. In some embodiments, the polynucleotide comprises the sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, or SEQ. ID NO20 or a fragment thereof.

In some embodiments, the taught proteins have markedly different structural and/or functional characteristics, as compared to a protein comprising or consisting of SEQ ID NO3.

The term “SG-11 variant” as used herein can include SG-11 proteins that are, e.g., identical to not identical to a protein comprising the sequence of SEQ ID NO:3 and which are further modified such as by a PTM or fusion or linkage to a second agent, e.g., a protein or peptide.

Protein PTMs occur in vivo and can increase the functional diversity of the proteome by the covalent addition of functional groups or proteins, proteolytic cleavage of regulatory subunits or degradation of entire proteins. Isolated proteins prepared according to the present disclosure can undergo 1 or more PTMs in vivo or in vitro. The type of modification(s) depends on host cell in which the protein is expressed and includes but is not limited to phosphorylation, glycosylation, ubiquitination, nitrosylation (e.g., S-nitrosylation), methylation, acetylation (e.g., N-acetylation), lipidation (myristoylation, N-myristoylation, S-palmitoylation, farnesylation. S-prenylation, S-palmitoylation) and proteolysis may influence almost all aspects of normal cell biology and pathogenesis. The isolated and/or purified SG-11 proteins or variants or fragments thereof as disclosed herein may comprise one or more the above recited post-translational modifications.

The SG-11 protein or variant or fragment thereof may be a fusion protein in which the N- and/or C-terminal domain is fused to a second protein via a peptide bond. Commonly used fusion partners well known to the ordinarily skilled artisan include but are not limited to human serum albumin and the crystallizable fragment, or constant domain of IgG, Fc. In some embodiments, the SG-11 protein or variant or fragment thereof is linked to a second protein or peptide via a disulfide bond, wherein the second protein or peptide comprises a cysteine residue.

### SG-21—a Functional Fragment of SG-11

Without being bound by theory, it is considered that a protein comprising SEQ ID NO3 or functional variant thereof (e.g., SEQ ID NO:19) can impart therapeutic effect when present in the lumen of the alimentary canal, such as the mouth, small intestine and/or large intestine. Accordingly, experiments were performed to test the stability of purified or isolated SG-11 protein in a fecal slurry as a means of assessing stability of the protein in the intestine. As shown in Example 14 (and FIG. 25), incubation of purified SG-11 in a fecal slurry results in a protein having an apparent molecular weight of 25 kDa when analyzed by SDS-PAGE. Furthermore, digestion of purified SG-11 protein with trypsin, which can cleave after lysine residues results in a predominant product, also with an apparent molecular weight of 25 kDa as determined by SDS-PAGE. The fecal slurry-treated SG-11 protein was then shown to maintain the ability to enhance epithelial barrier function integrity in a TEER assay (Example 12). Peptide mapping of the apparent 25 kDa band excised from an SDS-PAGE provides evidence that the 25 kDa protein is a C-terminal portion of the SG-11 protein, herein referred to as SG-21, wherein the N-terminus is likely to be an amino acid at a position within about residues 70 to 75, 65 to 85, or 65 to 75.

In an exemplary embodiment, a C-terminal fragment of SG-11 or variant thereof is provided which comprises residues 72 to 232 of SEQ ID NO:3 or SEQ ID NO:19, wherein each of SEQ ID NO3 or SEQ ID NO:19 can further comprise a methionine at the N-terminus (SEQ ID NO:36 or SEQ ID NO:42, respectively). A C-terminal fragment comprising at least a C-terminal portion of SG-11 (e.g., at least 40, 50, 75, 100, 125, 150 or 160 amino acids of residues 50 to 232 of SEQ ID NO:7), or variant or fragment thereof, which has functional activity equivalent to that of SG-11 is taught herein and referred to as SG-21 or a variant or fragment thereof. Amino acid sequences for SG-21 SEQ ID NO:34 and the SG-21V5 variant SEQ ID NO:40 are provided in Table 4A below.

TABLE 4A
SEQ ID NO: 34 (SG-21)
YKTPEDYTAFNGIELYQGKVVASLAAGYVYDGEFARVEEGKVVGAATKQ
DIYSEDDLKVAIIRANTDVKVDGEICYVSCQNVKLIGKDSVSIRDGYYL
ETGSVTASVDVTGQESVGTEQLSGTEQMEMTGEPVNADDTEQTEAAAGD
GSFETDVYTFIVYK
SEQ ID NO: 40 (SG-21V5)
YKTPEDYTAFSGIELYQGKVVASLAAGYVYDGEFARVEEGKVVGAATKQ
DIYSEDDLKVAIIRANTDVKVDGEIVYVSSQNVKLTGKDSVSIRDGYYL
ETGSVTASVDVTGQESVGTEQLSGTEQMEMTGEPVNADDTEQTEAAAGD
GSFETDVYTFIVYK

In view of these data, provided herein is a therapeutic protein is at least 90%, 95%, 96%, 97%, 98%, 99% or 100% identical to a protein comprising a fragment of the SG-11 protein (e.g., SEQ ID NO:3) which is functionally active as demonstrated by the ability to increase epithelial barrier function as determined by an in vitro TEER assay as described herein or by the ability to improve pathology in an animal model of IBD such as a DSS model. For example, a functional fragment of SG-11 is a fragment which, when administered to a mouse treated with DSS, reduces weight loss as compared to a control DSS mouse not treated with the fragment. A non-limiting example of a functional fragment of SG-11 is SG-21. In some embodiments, an SG-21 protein comprises amino acids 80 to 220, 75 to 225, 75 to 232, 74 to 232, 73 to 232, 72 to 232, 71 to 232, 70 to 232, 69 to 232, 68 to 232, 67 to 232 or 66 to 232 of SEQ ID NO:3 or a fragment thereof. The SG-21 protein may have a length of about 1 to 200, 1 to 190, 1 to 180, 1 to 175, 1 to 170, 1 to 165, 1 to 164, 1 to 163, 1 to 163, 1 to 161, 1 to 160, 1 to 150, 150 to 180, 155 to 180, 150 to 170, 155 to 170, 150 to 165, 155 to 165, or 160 to 165 amino acids in length. In an alternative embodiment, the functional fragment has at least 70%, 75%, 80%, 85%, 86%, 87%, 88%, 89%, 90%, 91%, 92%, 93%, 94%, 95%, 96%, 97%, 98%, 99%, 99.5%, 99.6, 99.7%, 99.8%, 99.9%, or 100% sequence identity to SEQ ID NO:34, SEQ ID NO: 36, SEQ ID NO:38, SEQ ID NO:39, SEQ ID NO:40, SEQ ID NO:42, SEQ ID NO46, SEQ ID NO:47 SEQ ID NO:48 or SEQ ID NO:49 or a fragment thereof. In some embodiments, the therapeutic protein comprises an amino acid sequence that is identical to SEQ ID NO:34, SEQ ID NO:36, SEQ ID NO:38. SEQ ID NO:39, SEQ ID NO:40, SEQ ID NO:42, SEQ ID NO:46, SEQ ID NO:47 SEQ ID NO:48 or SEQ ID NO:49. The therapeutic protein alternatively can be one which is a variant of SEQ ID NO:3, wherein the therapeutic protein has 1, 2, 3, 4, 5, 6, 7, 8, 9 or 10 amino acid substitutions relative to SEQ ID NO:34. Alternatively stated, the therapeutic protein comprises 1, 2, 3, 4, 5, 6, 7, 8, 9 or 10 non-naturally occurring amino acid substitutions relative to the sequence of residues 72 to 232 of SEQ ID NO:3. In some embodiments, the therapeutic protein does not comprise an amino acid sequence identical to the sequence of residues 72 to 232 of SEQ ID NO:3.

In some embodiments, the SG-21 protein can be modified or varied by one or more amino acid insertions or deletions. An insertion can be the addition of 1 or more (e.g., 1, 2, 3, 4, 5, 6, 7, 8, 9 or 1 to 10, 1 to 20, t to 30, 1 to 40 or 1 to 50) amino acids to the N-terminus and/or C-terminus of the protein and/or can be an insert of t or more (e.g., 1, 2, 3, 4, 5, 6, 7, 8, 9 or 1 to 10, 1 to 20, 1 to 30, 1 to 40 or 1 to 50) amino acids at a position located between the N- and C-terminal amino acids. Similarly, the deletion of the 1 or more (e.g., 1, 2, 3, 4, 5, 6, 7, 8, 9 or 1 to 10, 1 to 20, 1 to 30, 1 to 40 or 1 to 50) amino acids can occur at any of the N- and C-terminus and in the internal portion.

In some embodiments, a modified or variant protein is provided which contains at least one non-naturally occurring amino acid substitution relative to SEQ ID NO:3. In some embodiments, the variant protein comprises 1, 2, 3, 4, 5, 6, 7, 8, 9 or 10 amino acid substitutions relative to SEQ ID NO:3. In further embodiments, the variant protein contains the amino acid sequence as depicted in SEQ ID NO:38 (SG-21V1), SEQ ID NO:39 (SG-21V2), or SEQ ID NO:40 (SG-21V5).

In some embodiments, a therapeutic protein according to the present disclosure encompasses anyone of the variant proteins (e.g., SEQ ID NO:38, SEQ ID NO:39, SEQ ID NO:40, SEQ ID NO:42, SEQ ID NO:46, SEQ ID NO:47 SEQ ID NO:48 or SEQ ID NO:49) that also retains one or more activities of the full length mature protein depicted in, for example, SEQ ID NO:3, SEQ ID NO:7 or SEQ ID NO:19 or of the so-21 protein, for example, SEQ ID NO:34 or SEQ ID NO:36.

An embodiment of this variant is provided below in Table 4B, as SEQ ID NO:50, wherein the residue at each of positions 12, 13, 76, and 80 is denoted as X indicating that one or more of these 3 residues can each be substituted for any of the other 19 amino acids. The X at position 1 of SEQ ID NO:50 can be any of the 20 amino acids or is not present. In some embodiments, the protein comprises the amino acid sequence of SEQ ID NO:50. In some embodiments, X12 is N, R or K, and/or X13 is G or A, and/or X76 is C, S, T, M, V, L, A, or G, and/or X80 is C, S, T, M, V, L, A, or G. In some embodiments, X12 is N or R and/or X13 is G or A and/or X76 is C, V, L or A and/or X80 is C, S, V, L or A. In some embodiments, X12 is any amino acid other than N, X13 is any amino acid other than G, X76 is any amino acid other than C, and/or X80 is any amino acid other than C.

TABLE 4B
Amino Add Sequence for SEQ ID NO: 50

XYKTPEDYTAFXXIELYQGKVVASLAAGYVYDGEFARVEEGKVVGAATKQ
DIYSEDDLKVAIIRANTDVKVDGEIXYVSXQNVKLTGKDSVSIRDGYYLE
TGSVTASVDVTGQESVGTEQLSGTEQMEMTGEPVNADDTEQTEAAAGDGS
FETDVYTFIVYK

Also envisioned are polynucleotide sequences which encodes these proteins. It is well known to the ordinarily skilled artisan that two polynucleotide sequences which encode a single polypeptide sequence can share relatively low sequence identity due to the degenerate nature of the genetic code. For example, if every codon in the polynucleotide encoding a 161-amino acid sequence contained at least 1 substitution in its third position, that would calculate to about 67% sequence identity between the 2 polynucleotides. A polynucleotide of the present disclosure comprises a sequence that encodes a protein that is at least 7%, 75%, 80%, 85%, 86%, 87%, 88%, 89%, 90%, 91%, 92%, 93%, 94%, 95%, 96%, 97%, 98%, 99%, 99.5%, 99.6%, 99.7%, 99.8%, 99.9%, or 100% identical to SEQ ID NO:35 or SEQ ID NO:41.

The term “SG-21 variant” as used herein can include SG-21 proteins that are, e.g., identical to not identical to a protein comprising the sequence of SEQ ID NO:34 and/or which we further modified such as by a PTM or fusion or linkage to a second agent, e.g. a protein or peptide.

Protein PTMs occur in vivo and can increase the functional diversity of the proteome by the covalent addition of functional groups or proteins, proteolytic cleavage of regulatory subunits or degradation of entire proteins. Isolated proteins prepared according to the present disclosure can undergo one or more PTMs in vivo or in vitro. The type of modification(s) depends on host cell in which the protein is expressed and includes but is not limited to phosphorylation, glycosylation, ubiquitination, nitrosylation (e.g., S-nitrosylation), methylation, acetylation (e.g., N-acetylation), lipidation (myristoylation, N-myristoylation, S-palmitoylation, farnesylation, S-prenylation, S-palmitoylation) and proteolysis may influence almost all aspects of normal cell biology and pathogenesis. The isolated and/or purified SG-21 proteins or variants or fragments thereof as disclosed herein may comprise one or more the above recited post-translational modifications.

The SG-11 protein or variant or fragment thereof may be a fusion protein in which the N- and/or C-terminal domain is fused to a second protein via a peptide bond. Commonly used fusion partners well known to the ordinarily skilled artisan include but are not limited to human serum albumin and the crystallizable fragment, or constant domain of IgG, Fc. In some embodiments, the SG-21 protein or variant or fragment thereof is linked to a second protein or peptide via a disulfide bond, wherein the second protein or peptide comprises a cysteine residue.

As aforementioned, modifications and/or changes (e.g., substitutions, insertions, deletions) may be made in the structure of proteins disclosed herein. Thus, the present disclosure contemplates variation in sequence of these proteins, and nucleic acids coding therefore, where they are nonetheless able to retain substantial activity with respect to the functional activities assessed in various in vitro and in vivo assays as well as in therapeutic aspects of the present disclosure. In terms of functional equivalents, it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity.

In some embodiments, the SG-11 protein or variant or fragment thereof can be characterized by its ability to increase epithelial barrier function integrity as assessed by an in vitro TEER assay. The TEER assay can comprise a layer of colonic epithelial cells consisting of a mixture of enterocytes and goblet cells which are cultured until the cells obtain tight junction formation and barrier function capacity as assessed by a measurement of trans-epithelial electrical resistance. The protein may increase electrical resistance in a TEER assay by at least about 10%, 20%, 30%, 40%, 50%, 60%, 70%, S0% or 90% as compared to the TEER assay performed in the absence of the protein.

It is also contemplated that the SG-11 protein or variant or fragment thereof is one which, when administered to a subject, can reduce weight loss, reduce the clinical pathology score, or reduce colon shortening in the subject. In some embodiments, the subject is a mammal which has genetically or clinically induced inflammatory disorder or dysfunctional epithelial barrier function. Alternatively, the animal has an idiopathic gastrointestinal disorder involving a decrease in epithelial barrier function or intestinal inflammatory disorder. In some embodiments, the mammal is a human, non-human primate, or a rodent. The rodent may be a mouse or rat.

The SG-11 protein or variant or fragment thereof according to the present disclosure is one, when administered to a subject (e.g., rodent, non-human primate, or human), which can improve gastrointestinal epithelial cell barrier function, induce or increase mucin gene expression (e.g., muc2 expression), increase the structural integrity and/or functionality of a gastrointestinal mucous barrier (e.g., in the small intestine, large intestine, mouth and/or esophagus), and/or reduce inflammation in the gastrointestinal tract.

In some embodiments, the SG-11 protein or variant or fragment thereof resulting from such a substitution, insertion and/or deletion of amino acids relative to SEQ ID NO:3 or SEQ ID NO:7 maintains a level of functional activity which is substantially the same as that of a protein comprising SEQ ID NO:7 or SEQ ID NO:19 or SEQ ID NO:34 (e.g., is able to increase electrical resistance in a TEER assay wherein an epithelial cell layer was disrupted by, e.g., heat-killed E. coli). The variant protein may be useful as a therapeutic for treatment or prevention of a variety of conditions, including, but not limited to inflammatory conditions and/or barrier function disorders, including, but not limited to, inflammation of the gastrointestinal (including oral, esophageal, and intestinal) mucosa, impaired intestinal epithelial cell gap junction integrity. In some embodiments, the modified protein has one or more of the following effects when administered to an individual suffering from, or predisposed to, an inflammatory condition and/or barrier function disorder: improvement of epithelial barrier integrity, e.g., following inflammation induced disruption; suppression of production of at least one pro-inflammatory cytokine (e.g., TNF-α and/or IL-23) by one or more immune cell(s); induction of mucin production in epithelial cells; improvement of epithelial wound healing; and/or increase in epithelial cell proliferation. Moreover, the modified or variant protein may be used for treatment or prevention of a disorder or condition such as, but not limited to, inflammatory bowel disease, ulcerative colitis, Crohn's disease, short bowel syndrome, GI mucositis, oral mucositis, chemotherapy-induced mucositis, radiation-induced mucositis, necrotizing enterocolitis, pouchitis, a metabolic disease, celiac disease, inflammatory bowel syndrome, or chemotherapy associated steatohepatitis (CASH).

As demonstrated, e.g., in Example 3, the SG-11 protein can enhance epithelial wound healing. Accordingly, provided herein is a therapeutic protein comprising the amino acid sequence of SEQ ID NO:3 or SEQ ID NO:7 or SEQ ID NO: 19 or a variant or fragment thereof, wherein the protein can increase wound healing in an in vitro assay. Accordingly, provided herein is a therapeutic protein comprising the amino acid sequence of SEQ ID NO:34 or SEQ ID NO:40 or a variant or fragment thereof wherein the protein can increase wound healing in an in vitro assay. In some embodiments, the protein has a length of about 150 to 170 or 165 to 175 amino acids. Also envisioned are fragments of SG-11 ranging in length from about 30 to 70, 40 to 60, or 45 to 55 amino acids in length. Examples of such fragments include but are not limited to SEQ ID NO:46, SEQ ID NO:47, SEQ ID NO:48 and SEQ ID NO:49, and variants thereof, wherein such fragments have activity similar to that of SEQ ID NO:7 d/or SEQ ID NO:19.

### Recombinant Bacterial Delivery Systems

In some aspects, the present disclosure contemplates utilizing delivery systems outside of the traditional pharmaceutical formulations that comprise a purified protein. In some embodiments, the disclosure utilizes recombinant bacterial delivery systems, phage-mediated delivery systems, chitosan-DNA complexes, or AAV delivery systems.

One particular recombinant bacterial delivery system is based upon Lactococcus lactis. In some embodiments, the present disclosure teaches the cloning of heterologous nucleic acid encoding the therapeutic protein (e.g., SEQ ID NO:19 or SEQ ID NO:34) into an expression vector, and then transforming the vector into L. lactis. Subsequently, the transformed L. lactis is administered to a subject. See, e.g. Bratt, et al., A phase 1 trial with transgenic bacteria expressing interleukin-10 in Crohn's disease,” Clinical Gastroenterology and Hepatology, 2006, Vol. 4, pgs. 754-759 (“We treated Crohn's disease patients with genetically modified Lactococcus lactis (LL-Thy12) in which the thymidylate synthase gene was replaced with a synthetic sequence encoding mature human interleukin-10.”); Shigemori, et al., “Oral delivery of Lactococcus lactis that secretes bioactive home oxygenase-1 alleviates development of acute colitis in mice,” Microbial Cell Factories, 2015, Vol. 14:189 (“Mucosal delivery of therapeutic proteins using genetically modified strains of lactic acid bacteria (gmLAB) is being investigated as a new therapeutic strategy.”); Steidler, et al., “Treatment of murine colitis by Lactococcus lactis secreting interleukin-10,” Science, 2000, Vol. 289, pgs. 1352-1355 (“The cytokine interleukin-10 (IL-10) has shown promise in clinical trials for treatment of inflammatory bowel disease (IBD). Using two mouse models, we show that the therapeutic dose of IL-10 can be reduced by localized delivery of a bacterium genetically engineered to secrete the cytokine. Intragastric administration of IL-10-secreting Lactococcus lactis caused a 30% reduction in colitis in mice treated with dextran sulfate sodium and prevented the onset of colitis in IL-102/2 mice. This approach may lead to better methods for cost effective and long-term management of IBD in humans.”); Hanniffy, et al., “Mucosal delivery of a pneumococcal vaccine using Lactococcus lactis affords protection against respiratory infection,” Journal of Infectious Diseases, 2007, Vol. 195, pgs. 185-193 (“Here, we evaluated Lactococcus lactis intracellularly producing the pneumococcal surface protein A (PspA) as a mucosal vaccine in conferring protection against pneumococcal disease.”); and Vandenbroucke, et al., “Active delivery of trefoil factors by genetically modified Lactococcus lactis prevents and heals acute colitis in mice,” Gastroenterology, 2004, Vol. 127, pgs. 502-513 (“We have positively evaluated a new therapeutic approach for acute and chronic colitis that involves in situ secretion of murine TFF by orally administered L. lactis. This novel approach may lead to effective management of acute and chronic colitis and epithelial damage in humans.”).

In another embodiment, a “synthetic bacterium” may be used to deliver an SG-11 protein or variant or fragment thereof wherein a probiotic bacterium is engineered to express the SG-11 therapeutic protein (see, e.g., Durrer and Allen, 2017, PLoS One, 12:00176286).

Phages have been genetically engineered to deliver specific DNA payloads or to alter host specificity. Transfer methods, such as phages, plasmids, and transposons, can be used to deliver and circulate engineered DNA sequences to microbial communities, via processes such as transduction, transformation, and conjugation. For purposes of the present disclosure, it is sufficient to understand that an engineered phage could be one possible delivery system for a protein of the disclosure, by incorporating the nucleic acid encoding said protein into the phage and utilizing the phage to deliver the nucleic acid to a host microbe that would then produce the protein after having the phage deliver the nucleic acid into its genome.

Similar to the aforementioned engineered phage approach, one could utilize a transposon delivery system to incorporate nucleic acids encoding a therapeutic protein into a host microbe that is resident in a patient's microbiome. See, Sheth, et al., “Manipulating bacterial communities by in situ microbiome engineering” Trends in Genetics, 2016, Vol. 32, Issue 4, pgs. 189.200.

### Therapeutically Effective Live Bacteria

Lactococcus lactis a widely used Lactic Acid Bacterium (LAB) in the production of fermented milk products and is considered as the model LAB because many genetic tools lave been developed and its complete genome has been completely sequenced (Bolotin, Wincker et al. 2001. Genome Res, 11, 731-753). Thus, this food-grade Gram-positive bacterium may represent a good candidate to produce and deliver therapeutic proteins to the mucosa immune system. Also, the potential of live recombinant Lactococci to deliver such proteins to the mucosal immune system has been widely investigated (Steidler, Robinson et al, 1998, Infect Immun, 66, 3183-3189; Bermudez-Humaran, Cortes-Perez et al. 2004, J Med Microbiol, 53, 427-433; Hanniffy, Wiedermann et al 2004, Adv Appl Microbiol, 56, 1-64; Wells and Mercenier 2008, Nat Rev Microbiol, 6, 349-362; Bermudez-Humaran, Kharrat et al. 2011, Microb Cell Fact, 10 suppl 1, S4). This approach can offer several advantages over the traditional systemic injection, such as easy administration and the ability to elicit both systemic and mucosal immune responses (Mielcarek, Alonso et al. 2001, Adv Drug Deliv Rev, 51, 55-69; Eriksson and Holmgren 2002, Curr Opin Immunol, 14, 666-672).

In some aspects, the present disclosure provides a recombinant Lactococcus lactis bacterium expressing a therapeutic protein using any of the bacterial expression systems described herein, for instance, expression from a bacterial chromosome or a nisin-induced gene expression (e.g. NICE) system. In some embodiments, recombinant Lactococcus lactis bacteria as disclosed herein are able to express and secrete a therapeutic protein in a biologically active form. In some aspects, the present disclosure provides that the recombinant Lactococcus lactis bacterium expressing a therapeutic protein is able to diminish treat one or more conditions or symptoms thereof (e.g., inflammation and/or mucositis).

In some aspects, the present disclosure also provides a recombinant Lactococcus lactis bacterium expressing SG-11 or variants thereof, using any of the bacterial expression systems described herein, for instance, expression from a bacterial chromosome or a nisin-induced gen expression (NICE) system. In some embodiments, recombinant Lactococcus lactis bacteria as disclosed herein are able to express and secrete SG-11 protein or variants thereof in a biologically active form. In some aspects, the present disclosure provides that the recombinant Lactococcus lactis bacterium expressing either SG-11 or variants thereof is able to diminish inflammation and/or treat mucositis.

Therefore, in some aspects, the present disclosure provides a recombinant Lactococcus lactis bacterium wherein the bacterium comprises an expression cassette comprising a heterologous nucleotide sequence encoding a SG-11 protein or variants thereof selected from the group consisting of SEQ ID NOs: 1, 3, 5, 7, 9, 11, 13, 15, 17, 19, 34, 36, 38, 39, 40, 42, 44, 45, 46, 47, 48, 49, and 50. In some aspects, the present disclosure teaches provides a recombinant Lactococcus lactis bacterium recombinant, wherein the bacterium comprises an expression cassette comprising a heterologous nucleotide sequence encoding a polypeptide comprising an amino acid sequence with at least 90% sequence identity to a sequence selected from the group consisting of SEQ ID NOs: 1, 3, 5, 7, 9, 11, 13, 15, 17, 19, 34, 36, 38, 39, 40, 42, 44, 45, 46, 47, 48, 49, and 50. In some aspects, the present disclosure teaches provides a recombinant Lactococcus lactis bacterium recombinant, wherein the bacterium comprises an expression cassette comprising a heterologous nucleotide sequence encoding a polypeptide comprising an amino acid sequence with at least 90% sequence identity to a sequence selected from the group consisting of SEQ ID NOs: 21, 22, and 23. The heterologous nucleotide sequence can be expressed under the control of a constitutive promoter or an inducible promoter. The promoter can be the promoter of the usp45 operon of Lactococcus lactis or a nisin-inducible nisA promoter. In some embodiments, the expression cassette further comprises a nucleotide sequence encoding a secretion leader peptide, especially the signal peptide of the usp45 protein of Lactococcus lactis.

In some aspects, the present disclosure further provides any of the recombinant Lactococcus lactis bacteria as disclosed herein for use as a probiotic or as an anti-inflammatory agent.

In addition, in some aspects, the present disclosure provides a pharmaceutical, veterinary or probiotic composition comprising a recombinant Lactococcus lactis bacterium as disclosed herein. In some embodiments, the composition comprises a recombinant Lactococcus lactis bacterium capable of secreting a therapeutic protein. In some embodiments, the composition comprises a recombinant Lactococcus lactis bacterium capable of secreting a therapeutic protein (e.g., a SG-11 protein) and/or a recombinant Lactococcus lactis bacterium capable of secreting one or more SG-11 variants. The composition can further comprise an additional active ingredient, for example a drug such as an anti-inflammatory or immune-modulatory drug.

In some aspects, the present disclosure provides a food composition comprising a recombinant Lactococcus lactis bacterium as disclosed herein or a combination thereof, preferably a diary product.

Also, in some aspects, the present disclosure provides a recombinant Lactococcus lactis bacterium as disclosed herein or a combination thereof for use for the prophylaxis or treatment of an inflammatory condition. It also relates to the use of a recombinant Lactococcus lactis bacterium as disclosed herein or a combination thereof for die manufacture of a medicament for the treatment of an inflammatory condition. In some embodiments, provided is a method for treating an inflammatory condition in a subject in need thereof comprising administering a therapeutically effective amount of a recombinant Lactococcus lactis bacterium as disclosed herein or a combination of one or more thereof. In some embodiments, the inflammatory condition is a gastrointestinal epithelial cell barrier function disorder or a disease associated with decreased gastrointestinal mucosal epithelium integrity. In some embodiments, the epithelial cell barrier (unction disorder or disease is selected from the group consisting of: inflammatory bowel disease, ulcerative colitis. Crohn's disease, short bowel syndrome, GI mucositis, oral mucositis, chemotherapy-induced mucositis, radiation-induced mucositis, necrotizing enterocolitis, pouchitis, a metabolic disease, celiac disease, inflammatory bowel syndrome, and chemotherapy associated steatohepatitis (CASH). In some embodiments, the disorder or disease is mucositis including oral mucositis.

Also, the recombinant Lactococcus lactis bacterium can be intended for oral administration. A composition including recombinant Lactococcus lactis bacterium can be an edible product. The composition can be formulated as a pill, a tablet, a capsule, a suppository, a liquid, or a liquid suspension. In some embodiments, the recombinant Lactococcus lactis bacterium is intended to be administered in the early phase of inflammation. In some embodiments, the recombinant Lactococcus lactis bacterium is intended to be administered in the intermediate phase of inflammation. In some embodiments, the recombinant Lactococcus lactis bacterium is intended to be administered in the late phase of inflammation. In some embodiments, the recombinant Lactococcus lactis bacterium is intended to be administered during more than one phase of inflammation (e.g., early phase and intermediate phase, intermediate phase and late phase, or early, intermediate, and late phase).

In some embodiments, a composition comprising recombinant Lactococcus lactis bacteria useful, for example, for treating a subject suffering from an inflammation condition described above, can include viable recombinant Lactococcus lactis bacteria. In some embodiments, a composition comprising recombinant Lactococcus lactis bacteria useful, for example, for treating a subject suffering from an inflammation condition described above, can include non-viable recombinant Lactococcus lactis bacteria. In some embodiments, a composition comprising recombinant Lactococcus lactis bacteria useful, for example, for treating a subject suffering from an inflammation condition described above, can include both viable and non-viable recombinant Lactococcus lactis bacteria.

In some embodiments, the present disclosure provides that the recombinant Lactococcus lactis bacterium is a Lactococcus lactis bacterial cell comprising heterologous nucleotide sequences (e.g., encoding a therapeutic protein such as the SG-11 protein and/or variant thereof) on one or more plasmids. In some embodiments, the present disclosure provides that the recombinant Lactococcus lactis bacterium is a generically-engineered Lactococcus lactis bacterial cell having nucleotide insertions and/or modifications of heterologous nucleotide sequences (e.g., encoding a therapeutic protein such as the SG-11 protein and/or variant thereof), introduced into their DNA using genetic engineering techniques that are well known in the art.

### Expression Systems and Host Cells

Provided herein are expression systems (e.g., expression vectors and/or recombinant cells (e.g. Lactococcus lactis bacteria)) for the expression of one or more proteins of interest (e.g., SG-11 and/or one or more variants thereof) in a host cell. Typically, an expression system includes a nucleic acid comprising a promoter operably linked to a nucleic acid sequence encoding a protein of interest (e.g., a therapeutic protein such as SG-11 or one or more variants or fragments thereof)). In some embodiments, the nucleic acid encoding a protein of interest can further encode a signal peptide (e.g., N-terminal to the protein of interest). A host cell can optionally further include a ‘kill switch’. In some embodiments, a host cell can optionally further include one or more viability-enhancing mutations, additions, or deletions. In some embodiments, all or part of an expression system can be integrated into the host genome (e.g., bacterial chromosome). In some embodiments, all or part of an expression system can be present on one or more vectors (e.g., plasmids).

It will be appreciated that in order to produce an expression system integrated into the host genome, one or more vectors can be used, and portions of such vector (e.g., nucleotide sequences from a plasmid backbone) may or may not be present in the host genome after integration. Any appropriate gene editing techniques can be used to integrate a nucleic acid into a genome, including, for example, homologous recombination, site-specific recombination, transposon mediated gene transposition, zinc finger nucleases, transcription activator-like effector nucleases (e.g., TALEN®), and CRISPR.

Any method can be used to introduce an exogenous nucleic acid molecule into a cell. In fact, many methods for introducing nucleic acid into microorganisms such as bacteria are known, including, for example, heat shock, lipofection, electroporation, conjugation, fusion of protoplasts, and biolistic delivery.

An exogenous nucleic acid molecule contained within a host cell can be maintained within that host cell in any form. For example, exogenous nucleic acid molecules can be integrated into the genome of the host cell or maintained in an episomal state. In other words, a host cell can be a stable or transient transformant. A host cell described herein can contain a single copy, or multiple copies (e.g., about 5, 10, 20, 35, 50, 75, 100 or 150 copies), of a particular exogenous nucleic acid molecule as described herein.

Polynucleotide sequences encoding the proteins of the disclosure can be obtained using standard recombinant techniques. Desired encoding polynucleotide sequences may be amplified from the genomic DNA of the source bacterium. e.g., R. hominis. Alternatively, polynucleotides can be synthesized using a nucleotide synthesizer.

In some embodiments, the nucleic acid encoding the protein of interest (e.g., a therapeutic protein such as SG-11 or one or more variants or fragments thereof)) can be codon-optimized. A codon optimization algorithm can be applied to a polynucleotide sequence encoding a protein in order to choose an appropriate codon for a given amino acid based on the expression host's codon usage bias. Many codon optimization algorithms also take into account other factors such as mRNA structure, host GC content, ribosomal entry sites. Some examples of codon optimization algorithms and gene synthesis service providers are: AUTM: www.atum.bio/services/genegps; GenScript: www.genscript.com/codon-opt.html; ThermoFisher: www.thermofisher.com/us/en/home/life-science/cloning/gene-synthesis/geneart-gene-synthesis/geneoptimizer.html; and Integrated DNA Technologies: www.idtdna.com/CodonOpt.

In some embodiments, a protein of interest (e.g., a therapeutic protein such as SG-11 or one or more variants or fragments thereof)), can be expressed from a vector. Accordingly, provided herein are expression vectors which comprise a polynucleotide sequence that encodes a protein of interest (e.g., a therapeutic protein such as SG-11 or one or more variants or fragments thereof)). Once obtained, sequences encoding the protein of interest can be inserted into a recombinant vector capable of replicating and expressing heterologous (exogenous) proteins in a host cell. In some embodiments, the host cell is a Lactococcus lactis bacterium, Many vectors that are available and known in the art can be used for the purpose of the present disclosure. Selection of an appropriate vector will depend mainly on the size of the nucleic acids to be inserted into the vector and the particular host cell to be transformed with the vector. Each vector contains various components, depending on its function (amplification or expression of heterologous polynucleotide, or both) and its compatibility with the particular host cell in which it resides. The vector components generally include, but am not limited to: an origin of replication, a selection marker gene, a promoter, a ribosome binding site (RBS), a signal sequence, the heterologous nucleic acid insert and a transcription termination sequence. In some embodiments, the expression vector is a nisin-controlled gene expression system (e.g., NICE®) for Lactococcus lactis.

In general, plasmid vectors containing replicon and control sequences which are derived from species compatible with the host cell are used in connection with these hosts. The vector ordinarily carries a replication site, as well as marking sequences which are capable of providing phenotypic selection in transformed cell. For example, E. coli is typically transformed using a pBR322, pUC, pET or pGEX vector, a plasmid derived from an E. coli species. Another example is L. lactis, typically transformed using a pNZ8008, pNZ8148, pNZ8149, pNZ8150, pNZ8151, pNZ8152, pNZ8120, pNZ8121, pNZ8122, pNZ8123, pNZ8124, pND632, pND648, or pND969 vector, a plasmid derived from an L. lactis species. Such vectors can contain genes encoding ampicillin (Amp) and tetracycline (Tet) resistance and thus provides easy means for identifying transformed cells. These vectors, as well as their derivatives or other microbial plasmids or bacteriophage, may also contain, or be modified to contain, promoters which can be used by the microbial organism for expression of endogenous proteins.

An expression vector of the present disclosure may comprise a promoter, an untranslated regulatory sequence located upstream (5′) and an operably linked protein-encoding nucleotide sequence such that the promoter regulates transcription of that coding sequence.

Further useful plasmid vectors include pIN vectors (Inouye et al., 195); and pGEX vectors, for use in generating glutathione S-transferase (GST) soluble fusion proteins for later purification and separation or cleavage. Other suitable fusion proteins are those with β-galactosidase, ubiquitin, and the like. Suitable vectors for expression in both prokaryotic and eukaryotic host cells are known in the art, and some am further described herein.

Promoters typically fall into two classes, inducible and constitutive. An inducible promoter is a promoter that initiates increased levels of transcription of the protein-encoding polynucleotide under its control in response to changes in the culture condition, e.g., the presence or absence of a nutrient or a change in temperature. In some embodiments, the inducible promoter is a nisin-inducible nisA promoter. In some embodiments, an inducible promoter can be used without concomitant use of the inducing agent, for example, a nisin-inducible promoter can be used without the addition of nisin. A large number of promoters recognized by a variety of potential host cells are well known and a skilled artisan can choose the promoter according to desired expression levels. Additional promoters suitable for use with prokaryotic hosts include E. coli promoters such as lac, trp, tac, trc and ara, viral promoters recognized by E. coli such as lambda and T5 promoters, and the T7 and T7lac promoters derived from T7 bacteriophage. A host cell harboring a vector comprising a T7 promoter, e.g., is engineered to express a T7 polymerase. Such host cells include E. coli BL21(DE3), Lemo21(DE3), and NiCo21(DE3) cells. In some embodiments, the promoter is an inducible promoter which is under the control of chemical or environmental factors.

One or more promoters native to a host cell (e.g., Lactococcus lactis) can be used in an expression system. In some embodiments, a vector can include a promoter native to the host cell. In some embodiments, a nucleotide construct encoding a protein of interest (e.g., a therapeutic protein (e.g., SG-11 or one or more variants or fragments thereof)) can be engineered to be expressed from the host cell genome from a native promoter.

In some embodiments, when a nucleotide construct encoding a protein of interest (e.g., a therapeutic protein (e.g., SG-11 or one or more variants or fragments thereof)) is engineered to be expressed from the host cell genome from a native promoter, the native promoter can be in a location other than its native location (e.g., a second copy of the promoter can be inserted into the host genome).

In some embodiments, when a nucleotide construct encoding a protein of interest (e.g., a therapeutic protein (e.g., SG-11 or one or more variants or fragments thereof)) is engineered to be expressed from the host cell genome from a native promoter, the native promoter can be in its native location. In some embodiments, a gene normally expressed from the native promoter in the host can be deleted. In some embodiments, the nucleotide construct encoding a protein of interest (e.g., a therapeutic protein (e.g., SG-11 or one or more variants or fragments thereof)) can disrupt (e.g., diminish or eliminate) expression of a gene normally expressed from the promoter in the host. In some embodiments, the nucleotide construct encoding a protein (e.g., a protein of interest) can be expressed as a polycistonic transcript with a gene normally expressed from the promoter.

A disruption of an endogenous gene in a host cell can be accomplished by any appropriate method, including deleterious mutation or partial or complete substitution or deletion of a gene or promoter thereof. In some embodiments, a gene is disrupted in a cell if activity of the gene product is less than 20% (e.g., less than 15%, 10%, 5%, 3%, or 1%, or the activity of the gene product is 0%) of the activity of the gene product in a wild-type cell.

In some embodiments, the nucleotide construct encoding a protein (e.g., a protein of interest (e.g., a SG-11 protein, variant or fragment thereof)) can be under the control of the promoter of the GroESL operon of Lactococcus lactis. Such expression system has been disclosed in detail in US201510139940, incorporated herein by reference in its entirety. Other Lactococcus promoters have been identified in International Patent Application Publications WO2008084115 and WO2013175358, incorporated herein by reference in its entirety and include those of the genes rpoB, dpsA, glnA, glnR, pepV, atpD, pgk, fabF, fabG, rpoA, pepQ, rpsD, sodA, luxS, rpsK, rpIL, usp45, thyA, trePP, and hIIA (named as such in L. lactis MG1363). In some embodiments, a nucleotide construct encoding a protein of interest can be under the control of a usp45 promoter (e.g., the native usp45 promoter from L. lactis, e.g., with a sequence with at least 85%, 90%, 95% or 99% sequence identity to SEQ ID NO: 70 in Table 5). In some embodiments, a nucleotide construct encoding a protein of interest can be under the control of a thyA promoter (e.g., the native thyA promoter from L. lactis, e.g., with a sequence with at least 85%, 90%, 95%, or 99% sequence identity to SEQ ID NO: 71 in Table 5). In some embodiments, a nucleotide construct encoding a protein of interest can be under the control of a trePP promoter (e.g., the native trePP promoter from L. lactis, e.g., with a sequence with at least 85%, 90%, 95%, or 99/sequence identity to the promoter from SEQ ID NO: 90, which is a trehalose operon from L. lactis).

Nucleotide constructs encoding a protein of interest (e.g., a therapeutic protein (e.g., SG-11 or one or more variants or fragments thereof)) of the present disclosure may further encode a signal sequence which allows the translated recombinant protein to be recognized and processed (e.g., secreted or cleaved by a signal peptidase) by the host cell. For example, a nucleotide construct can further encode a signal peptide, which can be N-terminal to the protein of interest. In some embodiments, a signal peptide can be immediately N-terminal to the protein of interest. In some embodiments, a linker (e.g., including a cleavage site) can be present between a signal peptide and the protein of interest. In some embodiments, prokaryotic host cells may not recognize and process the signal sequences native to a eukaryotic heterologous polypeptide (e.g., a heterologous protein of interest), and the encoded signal sequence can substituted by a prokaryotic signal sequence selected, for example, from the group consisting of the alkaline phosphatase, penicillinase, Ipp, or heat-stable enterotoxin II (STII) leaders, LamB, PhoE, PcIB, OmpA and MBP. Examples of signal sequences that can be used in eukaryotic host cells include but are not limited to interleukin-2, CD5, the Immunoglobulin Kappa light chain, trypsinogen, scrum albumin, and prolactin.

In some embodiments, the encoded signal sequence is a secretion leader from the usp45 gene of L. lactis (e.g., a nucleotide encoding a polypeptide with at least 85%, 90%, 95%, or 99% percent identity to SEQ ID NO: 67).

Proteins of interest (e.g., a therapeutic protein (e.g., SG-11 or one or mere variants or fragments thereof)) as described herein can. In some embodiments, be expressed as a fusion protein or polypeptide. Commonly used fusion partners include but are not limited to human serum albumin and the crystallizable fragment, or constant domain of IgG, Fc. A histidine tag or FLAG tag can also be used to simplify purification of recombinant protein from the expression media or recombinant cell lysate. The fusion partners can be fused to the N- and/or C-terminus of the protein of interest. When used in combination with a signal sequence N-terminal to the protein of interest, the signal sequence is typically N-terminal to the fusion partner.

In some embodiments, a host cell can include a kill switch. “Kill switches” (sometimes also called containment systems) are defined as artificial systems that result in cell death under certain conditions. Several kill switches have been explored for containment of engineered microbes. See, for example, Wright, et al. Microbiology. 2013 July; 159(Pt 7):1221-35. doi: 10.1099/mic.0.066308-0, incorporated herein by reference in its entirety. In some embodiments, a kill switch can include lethal genes that are induced in designated non-permissive conditions. In some embodiments, a kill switch can include disruption of a gene that is necessary for cellular survival, for example, resulting the generation of an artificial auxotroph. In some embodiments, a kill switch can include disruption of a promoter of a gene that is necessary for cellular survival, for example, resulting in the generation of an artificial auxotroph. In some embodiments, a gene that is necessary for cellular survival is thymidylate synthase (e.g., thyA, e.g., a polynucleotide encoding a protein with at least 85%, 90%, 95%, or 99% sequence identity to SEQ ID NO: 72 in Table 5) or 4-hydroxy-tetrahydrodipicolinate synthase (e.g., dapA, e.g., a polynucleotide encoding a protein with at least 85%, 90%, 95%, or 99% sequence identity to SEQ ID NO: 73 in Table 5). For example, an organism lacking a functional thyA is a thyA auxotroph and can be referred to as having a thyA kill switch. For example, an organism lacking a functional dapA is a dapA auxotroph and can be referred to as having a dapA kill switch. In some embodiments, an organism can have more than one kill switch, for example, a thyA kill switch and a dapA kill switch.

It has been reported that the development of strategies to control genetically engineered bacteria can involve modular, reprogrammable genetic circuits. One strategy, dubbed ‘Deadman’, relies on a circuit in which the LacI and TetR transcription factors are reciprocally repressive, but in which the expression of TetR is favored owing to modifications in the strength of the ribosomal binding sites of the two transcription factors. Inhibition of TetR expression by anhydrotetracycline (ATc), a compound that is not normally found in nature, is necessary for expression of LacI. LacI directly inhibits expression of a lethal toxin and/or indirectly prevents inhibition of the expression of an essential gene; these effects, either alone or combined in a single circuit, keep the cells alive. Removal of ATc from the environment activates the expression of TetR, which leads to cell death. A ‘fail-safe’ mechanism was also added to the system, whereby production of the toxin and cell death are independently activated by isopropyl β-d-1-thiogalactopyranoside (IPTG). Another strategy, named ‘Passcode’, is based on the construction of fusions of environmental sensing modules of specific transcription fetors and DNA-recognition modules of different transcription factors. Hybrid transcription factors with the same DNA-recognition module but with different environmental sensing modules can therefore be built. The researchers used three different hybrid transcription factors to build a circuit in which the concomitant presence of two distinct environmental cues and the absence of another environmental cue are simultaneously required for preventing expression of a toxin and, thus, for cell survival. These kill switch strategies would be known to one of ordinary skill in the art (see, for example, Chan et al., nature chemical biology, 12:82-86 (2016). Osorio. Nat. Rev. Genet. 17(2):67 (2016), each of which is incorporated herein by reference in its entirety). Although a host of effective kill switches have been described, they can sometimes evolve to lose functionality within days. Another approach has been developed for varying the level of expression in a toxin/antitoxin system as well described in Stirling et al Molecular Cell 68:686-697(2017), which is incorporated herein by reference in its entirety. In some embodiments, the present disclosure provides the use and implementation of kill switch system to engineer the bacteria disclosed in this disclosure, which can be administered to a subject. This kill switch system can be used for preventing uncontrolled or undesired proliferation of the recombinant and/or genetically-engineered bacterium comprising SG-11 protein or variants thereof when desired.

Host cells as described herein (e.g., including an expression system as described herein) can also include enhancements to viability, for example, to remain at least partially viable when preserved, stored, and/or ingested. In some cases, viability can be determined by a host cell's ability to produce a protein of interest (e.g., a therapeutic protein (e.g., SG-11 or one or more variants or fragments thereof)). Such viability enhancements can, for example, allow the host cells to actively produce protein when present in the digestive system (e.g., stomach or intestines). One way that viability during preservation, storage, and/or ingestion can be enhanced is to increase the concentration of a small molecule (e.g., a sugar such as lactose, maltose, sucrose, or trehalose, an amino acid or derivative thereof such as glycine betaine (also called trimethylglycine), or combinations thereof) during preservation (e.g., a lyophilization process). Without being bound by any particular theory, it is believed that some small molecules can protect cells from the damaging effects of cold, desiccation, and/or acid (e.g., stomach acid or bile acids).

In some embodiments, a small molecule (e.g., a sugar such as lactose, maltose, sucrose, or trehalose, an amino acid or derivative thereof such as glycine betaine, or combinations thereof) can be supplemented to a mixture comprising the host cell, for example, prior to preservation (e.g., lyophilization). A small molecule can be supplemented to a mixture comprising the expression system in any appropriate amount, for example, about 5% to about 25% (e.g., about 5% to about 20%, about 5% to about 15%, about 5% to about 10%, about 10% to about 25%, about 15% to about 25%, about 20% to about 25%, or about 10% to about 20%) by weight of the mixture. In some embodiments, a mixture comprising the expression system can be supplemented with a salt (e.g., sodium chloride) at a concentration of about 0.1 M to about 1 M (e.g., about 0.1 M to about 0.8 M, about 0.1 M to about 0.6 M, about 0.1 M to about 0.4 M, about 0.1 M to about 0.2 M, about 0.2 M to about 1 M, about 0.4 M to about 1 M, about 0.6 M to about 1 M, about 0.8 M to about 1 M, or about 0.4 to about 0.6 M), either instead of or in addition supplementation with a small molecule.

In some embodiments, the concentration of a small molecule (e.g., a sugar such as lactose, maltose, sucrose, or trehalose, an amino acid or derivative thereof such as glycine betaine, or combinations thereof) can be increased by engineering the host cell to decrease catabolism of the small molecule. One way of decreasing catabolism is to disrupt one or more genes encoding a protein involved in catabolism of the small molecule. For example, one or more of the following genes can be disrupted: a sucrose 6-phosphate hydrolase such as sacA (also called scrB, e.g., a polynucleotide encoding a polypeptide with at least 85%, 90%, 95%, or 99% sequence identity to SEQ U) NO: 75 in Table 5), a maltose phosphorylase such as mapA (e.g., a polynucleotide encoding a polypeptide with at least 85%, 90%, 95%, or 99% sequence identity to SEQ ID NO: 75 in Table 5), a beta-galactosidase such as lacZ (e.g., a polynucleotide encoding a polypeptide with at least 85%, 90%, 95%, or 99% sequence identity to SEQ ID NO:76 in Table 5), a phospho-b-galactosidase such as lacG (e.g., a polynucleotide encoding a polypeptide with at least 85%, 90%, 95%, or 99% sequence identity to SEQ ID NO:77 in Table 5), or a trehalose 6-phosphate phosphorylase such as trePP (e.g., a polynucleotide encoding a polypeptide with at least 85%, 90%, 95%, or 99% sequence identity to SEQ ID NO: 78 in Table 5). In some embodiments, a host cell can include the disruption of trePP as a viability enhancement.

In some embodiments, the concentration of a small molecule (e.g., a sugar such as lactose, maltose, sucrose, or trehalose, an amino acid or derivative thereof such as glycine betaine, or combinations thereof) can be increased by engineering the host cell to decrease export of the small molecule. One way of decreasing export is to disrupt one or more genes encoding a protein involved in export of the small molecule. For example, a permease TIC component (e.g., ptcC, such as that from L. lactis (e.g., a polynucleotide encoding a polypeptide with at least 85%, 90%, 95%, or 99% sequence identity to SEQ ID NO: 79 in Table 5)) can be disrupted.

In some embodiments, the concentration of a small molecule (e.g., a sugar such as lactose, maltose, sucrose, or trehalose, an amino acid or derivative thereof such as glycine betaine, or combinations thereof) can be increased by engineering the host cell to activate import of the small molecule. One way of activating import is to engineer the cell by introducing into the cell one or more exogenous polynucleotides including one or more copies of a gene encoding a protein that imports the small molecule. For example, the following genes can be activated to increase the import of small molecules: a sucrose phosphotransferase such as sacB (e.g., a polynucleotide encoding a polypeptide with at least 85%, 90%, 95%, or 99% sequence identity to SEQ ID NO: 80 in Table 5), one or more components of a maltose transport operon such as malEFG (e.g., a polynucleotide encoding a polypeptide with at least 85%, 90%, 95%, or 99% sequence identity to SEQ ID NO: 81 (malE), a polynucleotide encoding a polypeptide with at least 85%, 90%, 95%, or 99% sequence identity to SEQ ID NO: 82 (malF), and/or a polynucleotide encoding a polypeptide with at least 85%, 90%, 95%, or 99% sequence identity to SEQ ID NO: 83 (malG) in Table 5), a lactose phosphotransferase such as lacFE (e.g., a polynucleotide encoding a polypeptide with at least 85%, 90%, 95%, or 99% sequence identity to SEQ ID NO: 84 and/or 85 in Table 5), a lactose permease such as lacY (e.g., a polynucleotide encoding a polypeptide with at least 85%, 90%, 95%, or 99% sequence identity to SEQ ID NO: 86 in Table 5) or a glycine betaine/proline ABC transporter permease component such as busAB (e.g., a polynucleotide encoding a polypeptide with at least 85%, 90%, 95%, or 99% sequence identity to SEQ ID NO: 87 in Table 5). It will be appreciated that a gene encoding a protein that imports the small molecule can be expressed using any of the strategies described herein for a protein of interest, or any other appropriate method.

In some embodiments, the concentration of a small molecule (e.g., a sugar such as lactose, maltose sucrose, or trehalose, an amino acid or derivative thereof such as glycine betaine, or combinations thereof) can be increased by engineering the host cell to activate production of the small molecule. One way of activating production of the smalt molecule is to engineer the cell by introducing into the cell one or more exogenous polynucleotides including one or more copies of a gene encoding a protein that is involved in the production of the small molecule. For example, copies of one or more of the following genes can be added: a trehalose-6-phosphate synthase such as otsA (e.g., a polynucleotide encoding a polypeptide with at least 85%, 90%, 95%, or 99% sequence identity to SEQ ID NO: 88 in Table 5) or a trehalose-6-phosphate phosphatase such a otsB (e.g., a polynucleotide encoding a polypeptide with at least 85%, 90%, 95%, or 99% sequence identity to SEQ ID NO: 89 in Table 5). It will be appreciated that a gene encoding a protein that is involved in the production of the small molecule can be expressed using any of the strategies described herein for a protein of interest, or any other appropriate method.

In some embodiments, one or more of the viability enhancement strategies can be combined. For example, one or more copies of a gene encoding a protein that is involved in the production of a small molecule (e.g., otsA and/or otsB) can be used to disrupt a gene involved in the catabolism of a small molecule (e.g., the same small molecule), for example, trePP. As another example, one or more copies of a gene encoding a protein that is involved in the production of a small molecule (e.g., otsA and/or otsB) can be used to disrupt a gene involved in the export of a small molecule (e.g., the same small molecule), for example, pteC.

TABLE 5

| | |
|---|---|
| SEQ ID NO: 70 | TGTTTTGTAATCATAAAGAAATATTAAGGTGGGGTAGGAATAGTATAATATGT |
| | TTATTCAACCGAACTTAATGGGAGGAAAAATTAAAAAAGAACAGTT |
| SEQ ID NO: 71 | TGGATATTTTTTATAAATCTGGTTTGRACAAATTATATTGACATCTCTTTTTCTA |
| | TCCTGATAATTCTGAGAGGTTATTTTGGGAAATACTATTGAACCATATCGAGG |
| | TGGTGTGGTATAATGAAGGGAATTAAAAAAGATAGGAAAATTTC |
| SEQ ID NO: 72 | MTYADKIFKQNIQNILDNGVFSENARPKYKDGQTANSKYVTGSFVTYDLQKGEFP |
| | ITTLRPIPIKSAIKELMWIYQDQTSELAILEEKYGVKYWGEWGIGDGTIGQRYGATV |
| | KKYNIISKLLDGLAKNPWNRRNIINLWQYEDFEETEGLLPCAFQTMFDVRREQDG |
| | QIYLDATLIQRSNDMLVAHHINAMQYVALQMMIAKHFSWKVGKFFYFVNNLHIY |
| | DNQFEQANELVKRTASDKEPRLVLNVPDGTNFFDIKPEDFELVDYEPVKPQLKFDL |
| | AI |
| SEQ ID NO: 73 | MSAKETIEKLQNARIITALVTPFKENGQINFGAFPKLIEDLIANHTEGLILAGTTAES |
| | PTLTHDEELAIFAAVNKIVDGRIPLIAGVGTNDTRDSVEFVKEVAELGYIDAGLAVT |
| | PYYNKPSQEGIYQHFKAIATASDLPIILYNIPGRVVTEIQVETILRLAELENVIAIKECT |
| | NTDNLAYIEKLPKDFLVYTGEDGLAFHTKALGGQGVISVASHILGQEFFEMFAEID |
| | QGSIQKAAAIQRKILPKINALFSVTSPAPIKTVLNAKGYEVGGLRLPLVACTTEESKII |
| | LEKIGN |
| SEQ ID NO: 74 | MKWSTKQRYRTYDSYSESDLESLRKLALKSPWKSNFHIEPETGLLNDPNGFSYFNE |
| | KWHLFYQHFPFGPVHGLKSWVHLVSDDLVHFEKTGLVLYPDTKYDNAGVYSGSA |
| | LAFENFLFLIYTGNHRGEDWVRTPYQLGAKIDKNNNQLVKFTEPLIYPDFSQTTDHF |
| | RDPQIFSFQGQIYCLIGAQSSQKNGIIKLYKAIENNLTDWKDLGNLDFSKEKMGY |
| | MIECPNLIFINGRSVLVFCPQGLDKSIVKYDNIYPNVYVIADDFTTGSKNQLKNAG |
| | QLINLDEGFDCYATQSFNAPDGSAYAISWLGLPETSYPTDKYNVQGVLSMVKKLSI |
| | KDNKLYQYPVEKMKELRQMEQDLLLADNNIITSNSYELEVDFRQQTSTLLSLMTN |
| | EKGDSALKVEIDKENNTITLIRNYEKRLAHVKIEKMNVFIDQSIFEIFINDGEKVLSDC |
| | RVFPNKNQYSIRSQNPIKIKLWELKK |
| SEQ ID NO: 75 | MKQIKRIMGIDPWKITSNQIEKEDRRLQESLTSIGNGYMGMRGNFSETYSGDSH |
| | QGTYIAGVWFPDKTRVGWWKNGYPEYFGKAINALNFASVRVFIDDKEVDLAAS |
| | HVTDFNLSLDMQKGVLTYTYVAYGVRVTAERFFSIAQQELAVFAFMFESLDGEIH |
| | QIRTVSVIDANVRNEDSNYDEKFWTVKNLDNTATGSFIVTETIPNPFGVEQFTVA |
| | AKQSFAGDFARVKQETRETSVLDVYEAKLVENAPLTFIKNVVLVVTSRDIKPSNLTKV |
| | LSNLTLEISKKTYNKFYKEQEEAWAKRWEIADVQIDGSAEAQQGIRFNLFQLFSTY |
| | YGEDERLNIGPKGFTGEKYGGATYWDTEAYAVPLYLALSDEKVAKNLLKYRHNQL |
| | PQAQHNARQQGLKGALYPMVTFTGVECHNEWEITFEEIHRNGAMAYAIYNYTN |
| | YTGDETYLAQEGLEVLVEIARFWADRVHYSQRNDKYMIHGVTGPNEYENNINNN |
| | WYTNKLAAWVLTYTAESLEKYPRTDLISSEEVAHWGEIVDKMYYPEDKELGIFVQ |
| | HDGYLDKDLTPVAQLDPKNLPLNQNWSWDKVLRSPYIKQADVLQGIYFFGNQFS |
| | MAEKQRNFDFYEPLTVHESSLSPSIHAILAAELGMEDKAVEMYERTARLDLDNYN |
| | NDTEDGLHITSMTGSWLAIVHGFAQMKTWEAQLSFAPFLPQAWIGYAFHINYR |
| | GCLLKISVGQEVKIELLRGQALSLKIYDETVELSDSYITKTR |
| SEQ ID NO: 76 | MAMMTMIDVLERKDWENPVVSNWNRLPMHTPMDLLEKQSLNGLWNFDHFS |
| | RISDVPKNWLELTESKTEIIVPSNWQIEFKDKSDVPIYTNVTYPIPIQPPYVPEANPV |
| | GAYSRYFDITKEWLESGHVHLTFEGVGSAFHFWLNGEYGGYSEDSRLPAEFDISNL |
| | AKEGQNCLKVLVFRWSKVTYFEDQDMWRMSGIFRSVNLQWLPDNYLLDFSIKT |
| | DLDEDLDFANVKLQAYAKNIDDACLEFKLYDDEQLIGECHGFDAEIGVVNPKLWS |
| | DEIPYLYRLELTLMDRSGAVFHKETKKIGIRKIAIEKGQLKINGKALLVRGVNKHEFT |
| | PEHGYVVSEEVMIKDIKLMKEHNFNAVRCSHYPNDSRWYELCDEYSLYVMDEA |
| | NIETHGMTPMNRLTNDPTYLPLMSERVTRMVMRDRNHPSIIIWSLGNESGYSS |
| | NHQALYDWCKSFDSSRPVHYEGGDDASRGATDATDIICPMYARVDSPSINAPYSL |
| | KTWMGVAGENRPLILCEYAHDMSNSLGGFGKYWQAFREIDRLQGGFIWDWV |
| | DQGLLKDGNYAYGGDFGDKPNDRQESLNGLVFPNRQAKPALREAKYWQQYYQ |
| | FELEKTPLGQVFAFTVTNEYLFRSTDNEKLCYQLINGLEVLWENELILNMPAGGSM |
| | RIDLSELPIDGTDNLFLNIQVKTIEKCNLLESDFEVAHQQFVLQEKINFTDRIDSNEEI |
| | TLFEDEELLTVRSAKQKFIFNKSNGNLSRWLDEKGNEKLLHELSEQFTRAPLDNDIG |
| | VSEVEHIDPNAWLERWKGIGFYELKTLLKTMIIQATENEVIISVQTDYEAKGKIAFS |
| | TIREYHIFRNGELLLKVDFKRNIEFPEPARIGLSLQLAEKAENVTYFGLGPDENYPDR |
| | RGASLFGQWNLRITDMTTPYIRSENGLRMETRELNYDRLKVRAMGQSFAFNLS |
| | PYSQNQLAKKGHWHLLEEEAGTWLNIDGFHMGVGGDDSWSPSVAQEYLLTKG |
| | NYHYEVSFKLT |
| SEQ ID NO: 77 | MKSTFKQDLYYMFHSKIIIIFLSISTLLVFLGALQAINLQKSSIIQFEQTKLIYKNKNDF |
| | LKDLNKNYTENNITDDESNINTEVNNSARYSYDYVKKSNYQLTSLGFPLFILKYLGLI |
| | FLPIVMGILGILLSTTDYKYGTYKRRLSTNSWKEIITGKIVGLSSVIFGLYFYILILSMAV |
| | GLFLPKFSKFIDLKQYNIDSPNPSLFSAFTLVLCVLVLALITGILSFLISLSIKNLFVSLIG |
| | FLLYYLALPNLGKFDYKNVVMNIFSNAGKDVLGQPIPYIPLDIKVSLILFAIYVALIST |
| | GVLIIFNKYTKYSM |
| SEQ ID NO: 78 | MTEKDWIIQYDKKEVGKRSYGQESLMSLGNGYLGLRGAPLWSTCSDNHYPGLYV |
| | AGVFNRTSTEVAGHDVINEDMVNWPNPQLIKVYIDGELVDFEASVEKQATIDFK |
| | NALQIERYQVKLAKGNLTLVTTKFVDPINFHDFGFVGEIIADFSCKLRIETFTDGSVL |
| | NQNVERYRAFDSKEFEVTKISKGLLVAKTRTSEIELAIASKSFLNGLAFPKIDSENDEI |
| | LAEAIEIDLQKNQEVQFDKTIVIASSYESKNPVEFVLTELSATSVSKIQENNTNYWEK |
| | VWSDADIVIESDHEDLQRMVRMNIFHIRQAAQHGANQFLDASVGSRGLTGEGY |
| | RGHIFWDEIFVLPYYAANEPETARDLLLYRINRLTAAQENAKVDGEKGAMFPWQ |
| | SGLIGDEQSQFVHLNTVNNEWEPDNSRRQRHVSLAIVYNLWIYSQLTEDESILTD |
| | GGLDLIIETTKFWLNKAELGDDGRYHIDGVMGPDEYHEAYPGQEGGICDNAYTN |
| | LMLTWQLNWLTELSEKGFEIPKELLEKAQKVRKKLYLDIDENGVIAQYAKYFELKE |
| | VDFAAYEAKYGDIHRIDRLMKAEGISPDEYQVAKQADTLMLIYNLGQEHVTKLVK |
| | QLAYELPENWLKVNRDYYLARTVHGSTTSRPVFAGIDVKLGDFDEALDFLITAIGS |
| | DYYDIQGGTTAEGVHIGVMGETLEVIQNEFAGLSLREGQFAIAPYLPKSWTKLKFN |
| | QIFRGTKVEILIENGQLLLTASADLLTKVYDDEVQLKAGVQTKFDLK |
| SEQ ID NO: 79 | MNNFIQNKIMPPMMKFLNTRAVTAIKNGMIYPIPFIIIGSVFLILGQLPFQAGQDP |
| | MNKIKLGPLFLQINNASFGIMALLAVFGIAYAWVRDAGYEGVPAGLTGVIVHILLQ |
| | PDTIHQVTSVTDPTKTSTAFQVGGVIDRAWLGGKGMVLSIIVGLLVGWIYTGFM |
| | RRNITIKMPEQVPENVAASFTSLVPAGAIITMAGVVHGITTIGFNTTFIELVYKWIQ |
| | TPLQHVTDGPVGVFVIAFMPVFIWWFGVHGATIIGGIMGPLLQANSADNAALYK |
| | AGHLSLSNGAHIVTQSFMDQYLTVTGSGLTGLVIFLLVSAKSVQGKTLGRMEIGP |
| | AVFNINEPILFGLPIVLNPILAIPFILAPLISGILTYLVIYLGIIPPFNGAYVPWTTPAVLS |
| | GYLVGGWQGMVWQIIILALTTVLYWPFAKAYDNILLKEEAETEAGINAAE |
| SEQ ID NO: 80 | MNHKQVAERILNAVGRDNIQGARHCATRLRLVLKDTGVIDQEALDNDPDLKGTF |
| | EAAGQYQIIVGPGDVNTVYEEFIKLTSISEASTADLKEIAGSQKKQNPVMALVKLLS |
| | DIFVPLIPALVAGGLLMALNNALTAEHLFATKSLVEMFPMWKGFADIVNTMSAA |
| | PFTFMPILIGYSATKRFGGNPYLGAVVGMIMVMPGLINGYNVAEAISNHTMTYW |
| | DIFSFKVAQAGYQGQVLPVIGVAFILAKLERFFHKYLNDAIDFTFTPLLSVIITSFLTF |
| | TIVGPALRFVSNGLTDGLVGLYNTLGALGMLVFGGFYSAIVVTGLHQSFPAIETML |
| | ITNYQHSGIGGDFIFPVAACANMAQAGATFAILFVTKNIKTKALAAPAGVSAILGIT |
| | EPALFGINLKLKYPFFIALGASAIGSLFMGLFHVLAVSLGSAGLIGFISIKAGYNLQF |
| | MISIFISFLIAFVVTSIYGRRMEAKSITKEKNKQNATTQYQPEVIIDPVKSGELLAPI |
| | NGFVIPLSDVSDPVFSKEIMGKGIAIKPKSGELFSPADGEIIIAYETGHAYGIKTKNG |
| | GEVLLHIGIDTVSMNGNGFIQNVKVGQKVKAGDLLGSFDKEEIKKSGLDDTVIIVIT |
| | NSASYNEILPLSENVDIKVGKILLLN |
| SEQ ID NO: 81 | MKSWKKVALGGASVLALATLAACGSSASSNKSSSSSSSDSIKGTVRVYVDTQQKA |
| | TYTDVAKGLTSKYPDLKVQIIANASGSANAKTDIAKDPSKYADVFAVPNDQLGDM |
| | ADKGFISPVATKFADEIKNDNSKITVAGVTYKDKVYAFPKSTEAQVLFYNKSKLSAD |
| | DVKSWDTMTSKAVFATDFTNAYNFYPVFFSAGTQLFGASGEDVTGTNVASDKG |
| | VTAMKWFADQKANKNVMQTSNALNQLQSGKADAIIDGPWDTANIKKILGDNF |
| | AVAPYPTITLNGEQKQLEAFQGIKGFAVNSATKDQAASQTVAQYLTTKAAQLKLF |
| | NSQGDVPTNLDAQKDDAVKSSDATKAVITMAKEGNSVVMPKLPQMATFWNN |
| | AAPLINGAYTGSIKATDYQAQLQKFQDSISK |
| SEQ ID NO: 82 | MTKKKKRKQTESNVSPEEKSIKLREVFQKGNTVTKLTFFVMGLNQIKNKQWVKG |
| | FTFLILEIAFIGWLLFSGLSAFSLLSSLGPNKTLKETTDANGFPVIIQPDHSVLILLWGL |
| | IACLVVVLFILLYWFNYRSNKHLYYLEREGKHIPTNREELASLLDEKLYATLMAVPLI |
| | GVLAFTVLPTVYMISMAFTNYDRLHATAFSWTGFQAFGNVLTGDLAGTFFPVLG |
| | WTLVWAIVATATTFLGGVLLALLIESTGIKFKGFWRTVFVIVFAVPQFVTILMMA |
| | QFLDQQGAFNGILMNLHLISNPINFIGAASDPMVARITVIFVNMWIGIPVSMLVS |
| | TAIIQNLPQDQIEAARIDGANSLNIFRSITFPQILFVMTPALIQQFIGNINNFNVIYLL |
| | TQGWPMNPNYQGAGSTDLLVTWLYNLVFGQTQRYNAAAVLGILIFIVNASISLV |
| | AYRRTNAFKEG |
| SEQ ID NO: 83 | MKSYKTQRRISITLRYILLALLAIVWIFPIIWIVLASLTQNNTGFVSTIIPKTFTFENYI |
| | QLFQNKSGSFPFVSWIINTFIVAVISATLSTFITIIMSYILSRLRFAFRKPFLQIALVLG |
| | MFPGFMSMIALYYILKAMNMLNIGGLILVYVGGAGLGFYIAKGFFDTIPRSIDEAA |
| | TIDGANKWQVFTHITLPLSRPIIVYTALMAFIAPWTTDFIFSGIILGNNQAHPETFTIA |
| | YGLYSMVHSQKGAATAFFTQFIAGCVIIAIPITILFVIMQKFYVNGITAGADKG |
| SEQ ID NO: 84 | MHKLIELIEKGKPFFEKISRNIYLRAIRDGFIAGMPVILFSSIFILIAYVPNAWGFHWS |
| | KDIETFLMTPYSYSMGILAFFVGGTTAKALTDSKNRDLPATNQINFLSTMLASMV |
| | GFLLMAAEPAKEGGFLTAFMGTKGLLTAFIAAFVTVNVYKVCVKNNVTIRMPED |
| | VPPNISQVFKDLIPFTVSVVLLYGLELLVKGTLGVTVAESIGTLIAPLFSAADGYLGIT |
| | LIFGAYAFFWFVGIHGPSIVEPAIAAITYANIDVNLHLIQAGQHADKVITSGTQMFI |
| | ATMGGTGATLIVPFLFMWICKSDRNRAIGRASVVRTFFGVNEPILFGAPIVLNPIFF |
| | VPFIFTPIVNVWIFKFFVDTLNMNSFSANLPWVTPGPLGIVLGTNFQVLSFILAGLL |
| | VVVDTIIYYPFVKVYDEQILEEERSGKTNDALKEKVAENFNTAKADAVLGKAGVAK |
| | EDVAANNNITKETNVLVLCAGGGTSGLLANALNKAAAEYNVPVKAAAGGYGAH |
| | REMLPEFDLVILAPQVASNFDDMKAETDKLGIKLVKTEGAQYIKLTRDGKGALAFV |
| | QQQFD |
| SEQ ID NO: 85 | MTIKFKHAYKSFGKKIIFKDASININRNSIYFIMAPNGSGKTTFFKIITNLQTLDKGKV |
| | YNDCSNRKQFSIFDDLSLYKNLTGYQNIQLFTNFKFNKFEIEQHSKKYEMLSKLNQ |
| | KVSTYSLGEGKKISLLLWELLNPDLVIMDEVTNGLDHNTLKELKSSLLKAKEDSIIILT |
| | GHELLFYEEIIDDLYILNNGKLLKELNWKEEGLTKTYEKYF |
| SEQ ID NO: 86 | MKEGKMKQRLSYAFGALGHDVYYYSISTFFIAFVTAQMFAGTPHEDAMIALVTSL |
| | VVIIRLIEIIFDPIIGSIIDNTHTRWGKFKPWLVVGGIMSSLMIMLMFSDFFGLAKSD |
| | NRTLFAIVFIIAPIILDAFYSFKDIAFWSMIPALSEKNSERETLGTFARFGSAIGAQGA |
| | TIIAIPITIFFTKGGHAQGARGFFAFGVIAALVQGISALVTAWGTKEQKSVIRQEGT |
| | KTNTLDVFKALLKNDQLMWLSLSYILFAIAYVATTATLILNFTFVIGNASLYSITGIVG |
| | FIGSIILVPMFPILAKKFGRRKVLTGAIISMLLGYLLEVLGSSVAMTVAGLIFLTAPYQ |
| | LVFLSVLMTITDSVEYGQWKNGVRNEAVTLAMRPLLDKIAGAFSNGIYGFVAISA |
| | GMTGSKYIAGHTYGVATFKLYSFVVPAILMIIALAVYLFKVKLTEKRHEEIVAELEER |
| | LK |
| SEQ ID NO: 87 | MIDLVIGKIPLANWVSSATDWITSTFSSGFDVIQKSGTVLMNGITGALTAVPFWL |
| | MIAVVTILAILVSGKKFAFPLFAFIGLCLIANQGMSDLMSTITLVLLSSWSWGVPL |
| | GIWMAKSELVAKIVQPILDFMQTMPGFVYLIPAVAFFGIGVVPGVFASVIFALPPT |
| | VRMTNLGIRQVSTELVEAADSFGSTARQKLFKLEFPLAKGTIMAGVNQTIMLALS |
| | MVVIASMIGARGLGRGVMAVQSADIGKGFVSGISIVILAIIIDRFTQKLNVSPLEK |
| | QGNPKLKKWKRWIAIVSLLALIVGAFSGMSFGKKSSDKKVDLVYMNWDSEVASI |
| | NVLTQAMEEHSFDVTTTALDNAVAWQTVANSQADGMVSAWLPNTHKTQWK |
| | KYGKSVELLGPNLKGAKVGFVVPSYMNVNSIEDLTNQANKTITGIEPGAGVMAAS |
| | ENTLKSYSNLKDWKLVPSSSGAMTVALGEAIKQHKDIVITGWSPHWIFNKYDLKY |
| | LADPKGTMGTSENINTIVRKGLKKENPEAYKVLNNFNWTTKDMESVMLDIQNG |
| | KTPEAAAKAWIKDHQKQVDKWFK |
| SEQ ID NO: 88 | MSRLVVVSNRIAPPDEHARSAGGLAVGILGALKAAGGLWFGWSGETGNEDQPL |
| | KKVKKGNITWASFNLSEQDLDEYYNQFSNAVLWPAFHYRLDLVQFQRPAWDGY |
| | LRVNALLADKLLPLLQDDDIIWIHDYHLLPFAHELRKRGVNNRIGFFLHIPFPTPEIF |
| | NALPTYDTLLEQLCDYDLLGFQTENDRLAFLDCLSNLTRVTTRSAKSHTAWGKAFR |
| | TEVYPIGIEPKEIAKQAAGPLPPKLAQLKAELKNVQNIFSVERLDYSKGLPERFLAYE |
| | ALLEKYPQHHGKIRYTQIAPTSRGDVQAYQDIRHQLENEAGRINGKYGQLGWTPL |
| | YYLNQHFDRKLLMKIFRYSDVGLVTPLRDGMNLVAKEYVAAQDPANPGVLVLSQ |
| | FAGAANELTSALIVNPYDRDEVAAALDRALTMSLAERISRHAEMLDVIVKNDINH |
| | WQECFISDLKQIVPRSAESQQRDKVATFPKLA |
| SEQ ID NO: 89 | MTEPLTETPELSAKYAWFFDLDGTLAEIKPHPDQVVVPDNILQGLQLLATASDGA |
| | LALISGRSMVELDALAKPYRFPLAGVHGAERRDINGKTHIVHLPDAIARDISVQLHT |
| | VIAQYPGAELEAKGMAFALHYRQAPQHEDALMTLAQRITQIWPQMALQQGKC |
| | VVEIKPRGTSKGEAIAAFMQEAPFIGRTPVFLGDDLTDESGFAVVNRLGGMSVKI |
| | GTGATQASWRLAGVPDVWSWLEMITTALQQKRENNRSDDYESFSRSI |

Suitable host cells for cloning or expressing nucleotide constructs as described herein include prokaryote, yeast, or higher eukaryote cells. Numerous cell lines and cultures are available for use as a host cell, and they can be obtained for example through the American Type Culture Collection (ATCC), which is an organization that serves as an archive for living cultures and genetic materials. Cell types available for vector replication and/or expression include, but are not limited to, bacteria, such as E. coli (e.g., E. coli strain RR1, E. coli LE392, E. coli B, E. coli X 1776 (ATCC No. 31337) well as E. coli W3110 (F-, lambda-, prototrophic, ATCC No. 273325). BL21(DE3), Lemo21(DE3), and NiCo21(DE3), E. coli Nissel (EcN), DH5α, JM109, TOP10 and KC8, bacilli such as Bacillus subtilis; and other enterobacteriaceae such as Salmonella typhimurium. Serratia marcescens, various Pseudomonas species, various Lactococcus species as well as a number of commercially available bacterial hosts such as SURE® Competent Cells and SOLOPACK™ Gold Cells (STRATAGENE®, La Jolla). In certain embodiments, bacterial cells such as E. coli are particularly contemplated a host cells. In some embodiments, bacterial cells such as L. lactis are particularly comtemplated as host cells. A number of commercially available Lactococcus lactis bacterial strains include MG1363, IL1403, NZ9000, NZ9100, NZ3900, NZ3910, LM0230. In some embodiments, the MG1363 strain is used. In some embodiments, the NZ9000 strain is used.

In some embodiments, the Lactococcus lactis bacterium is prepared rom a bacterium selected among Lactococcus lactis subsp. cremoris (for example, strain A76, GE214, HP, IBB477, KW2, MG1363, HB60, HB61, HB63, NBRC 100676, NZ9000, SK11, TIFN1, TIFN3, TIFN5, TIFN6, TIFN7, DSM14797, CNCM I-2807, DN030066 (CNCM I-1631), DN030087 (CNCM I-2807), CNCM I-1631, NCC2287 (CNCM I-4157) or UC509.9), Lactococcus lactis subsp. lactis (for example, strain 1AA59, A12, CNCM I-1631, CV56, Delphy I, II1403, IO-1, DPC3901, LD61, TIFN2, TIFN4, JCM 5805 also called NBRC 100933, JCM 7638, K214, KF147, KLDS 4.0325, NCDO 2118 or YF11), Lactococcus lactis subsp. hordinae (such as NBRC 100931) or Lactococcus lactis subsp. tructae. In some embodiments, the Lactococcus lactis bacterium is selected from Lactococcus lactis subsp. cremoris and Lactococcus lactis subsp. lactis, especially Lactococcus lactis subsp. lactis by. Diacetylactis. In a particular embodiment, the Lactococcus lactis bacterium is prepared from Lactococcus lactis subsp. Cremoris, preferably MG1363 (GenBank NC_009004). The Lactococcus lactis bacterium that can be used as a host cell is provided in U.S. Patent Application Publication US 2018/0104285, which is herein incorporated by reference in its entirety.

Examples of eukaryotic host cells for replication of a vector and/or expression of a nucleotide construct include, but are not limited to, HeLa, NIH3T3, Jurkat, 293, Cos, CHO, Saos, and PC12. Additional eukaryotic host cells include yeasts (e.g., Pichia pastoris and Saccharomyces cerevisiae) and cells derived from insects (e.g., Spodoptera frugiperda or Trichoplusia ni). Many host cells from various cell types and organisms are available and would be known to one of skill in the art. Similarly, a viral vector may be used in conjunction with either a eukaryotic or prokaryotic host cell, particularly one that is permissive for replication or expression of the vector. The selection of an appropriate host cell is deemed to be within the skill in the art.

Methods are well known for introducing recombinant DNA, e.g., an expression vector, into a host cell so that the DNA is replicable, either as an extrachromosomal element or as a chromosomal integrant, thereby generating a host cell which harbors the nucleotide construct of interest. Methods of transfection are known to the ordinarily skilled artisan, for example, by CaPO₄ and electroporation. Depending on the host cell used, transformation is performed using standard techniques appropriate to such cells. The calcium treatment employing calcium chloride, as described in Sambrook et al., supra, or electroporation is generally used for prokaryotes or other cells that contain substantial cell-wall barriers. General aspects of mammalian cell host system transformations have been described in U.S. Pat. No. 4,399,216. Transformations into yeast are typically carried out according to the method of Van Solingen et al., J. Bact, 130:946 (1977) and Hsiao et al., Proc. Natl. Acad. Sci. (USA), 76:3829 (1979). Other methods for introducing DNA into cells include nuclear microinjection, electroporation, bacterial protoplast fusion with intact cells, or introduction using polycations, e.g., polybrene, polyornithine. For various techniques for transforming mammalian cells, see Keown et al., Methods in Enzymology. 185:527-537 (1990) and Mansour et al., Nature, 336:348-352 (1989).

Accordingly, provided herein is a recombinant vector or expression vector and comprising a nucleotide construct which encodes a SG-11 therapeutic protein sequence of interest (e.g., SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19 or variant thereof, and/or fragment thereof as described herein). Also, provided herein is a recombinant vector or expression vector as described above and comprising a nucleotide construct which encodes a SG-21 therapeutic protein sequence of interest (e.g., SEQ ID NO:35, SEQ ID NO:37, SEQ ID NO:41, SEQ ID NO:43, or which encodes the protein of SEQ ID NO:34, SEQ ID NO:36, SEQ ID NO:38, SEQ ID NO:39, SEQ ID NO:40, SEQ ID NO:42, SEQ ID NO:46, SEQ ID NO47, SEQ ID NO:48, SEQ ID NO:49 or variant thereof, and/or fragment thereof as described herein). Moreover, the present disclosure provides a host cell harboring the vector. The host cell can be a eukaryotic or prokaryotic cell as detailed above. In a preferred embodiment, the host cell is a prokaryotic cell. In a further preferred embodiment, the hoot cell is L. lactis. In some embodiments, the bot cell is E. coli.

In some embodiments, the protein of interest is expressed from a vector (e.g., NZ8124) with a signal peptide (e.g., a usp45 signal peptide) using a promoter from the vector (e.g., nisA), a thymidylate synthase kill switch, and viability enhancements of expression of neither otsA nor otsB and disruption of neither trePP nor pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch, and viability enhancements of expression of neither otsA nor otsB and disruption of trePP but not pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch, and viability enhancements of expression of neither otsA nor otsB and disruption of pteC but not trePP.

In some embodiments, the protein of interest is expressed from a vector (e.g., NZ8124) with a signal peptide (e.g, a usp45 signal peptide) using a promoter from the vector (e.g., nisA), a thymidylate synthase kill switch, and viability enhancements of expression of neither otsA nor otsB and disruption of trePP and pteC.

In some embodiments, the protein of interest is expressed from a vector (e.g., NZ8124) with a signal peptide (e.g., a usp45 signal peptide) using a promoter rom the vector (e.g., nisA), a thymidylate synthase kill switch, and viability enhancements of expression of otsA but not otsB and disruption of neither trePP nor pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch, and viability enhancements of expression of otsA but not otsB and disruption of trePP but not pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch, and viability enhancements of expression of otsA but not otsB and disruption of pteC but not trePP.

In some embodiments, the protein of interest is expressed from a vector (e.g., NZ8124) with a signal peptide (e.g., a usp45 signal peptide) using a promoter from the vector (e.g., nisA), a thymidylate synthase kill switch, and viability enhancements of expression of otsA but not otsB and disruption of trePP and pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch, and viability enhancements of expression of otsB but not otsA and disruption of neither trePP nor pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch, and viability enhancements of expression of otsB but not otsA and disruption of trePP but not pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch, and viability enhancements of expression of otsB but not otsA and disruption of pteC but not trePP.

In some embodiments, the protein of interest is expressed from a vector (e.g., NZ8124) with a signal peptide (e.g., a usp45 signal peptide) using a promoter from the vector (e.g., nisA), a thymidylate synthase kill switch, and viability enhancements of expression of otsB but not otsA and disruption of trePP and pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch, and viability enhancements of expression of otsA and otsB and disruption of neither trePP nor pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch, and viability enhancements of expression of otsA and otsB and disruption of trePP but not pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch, and viability enhancements of expression of otsA and otsB and disruption of pteC but not trePP.

In some embodiments, the protein of interest is expressed from a vector (e.g., NZ8124) with a signal peptide (e.g., a usp45 signal peptide) using a promotor from the vector (e.g., nisA), a thymidylate synthase kill switch, and viability enhancements of expression of otsA and otsB and disruption of trePP and pteC.

In some embodiments, the protein of interest is expressed from a vector (e.g., NZ8124) with a signal peptide (e.g., a usp45 signal peptide) using a promoter from the vector (e.g., nisA), a dapA kill switch, and viability enhancements of expression of neither otsA nor otsB and disruption of neither trePP nor pteC.

In some embodiments, the protein of interest is expressed from a vector (e.g., NZ8124) with a signal peptide (e.g., a usp45 signal peptide) using a promoter from the vector (e.g., nisA), a dapA kill switch, and viability enhancements of expression of neither otsA nor otsB and disruption of trePP but not pteC.

In some embodiments, the protein of interest is expressed from a vector (e.g., NZ8124) with a signal peptide (e.g., a usp45 signal peptide) using a promoter from the vector (e.g., nisA), a dapA kill switch, and viability enhancements of expression of neither otsA nor otsB and disruption of pteC but not trePP.

In some embodiments, the protein of interest is expressed from a vector (e.g., NZ8124) with a signal peptide (e.g., a usp45 signal peptide) using a promoter from the vector (e.g., nisA), a dapA kill switch, and viability enhancements of expression of neither otsA nor otsB and disruption of trePP and pteC.

In some embodiments, the protein of interest is expressed from a vector (e.g., NZ8124) with a signal peptide (e.g., a usp45 signal peptide) using a promoter from the vector (e.g., nisA), a dapA kill switch, and viability enhancements of expression of otsA but not otsB and disruption of neither trePP nor pteC.

In some embodiments, the protein of interest is expressed rom a vector (e.g., NZ8124) with a signal peptide (e.g., a usp45 signal peptide) using a promoter from the vector (e.g., nisA), a dapA kill switch, and viability enhancements of expression of otsA but not otsB and disruption of trePP but not pteC.

In some embodiments, the protein of interest is expressed from a vector (e.g., NZ8124) with a signal peptide (e.g., a usp45 signal peptide) using a promoter from the vector (e.g., nisA), a dapA kill switch, and viability enhancements of expression of otsA but not otsB and disruption of pteC but not trePP.

In some embodiments, the protein of interest is expressed from a vector (e.g., NZ8124) with a signal peptide (e.g., a usp45 signal peptide) using a promoter tom the vector (e.g., nisA), a dapA kill switch, and viability enhancements of expression of otsA but not otsB and disruption of trePP and pteC.

In some embodiments, the protein of interest is expressed from a vector (e.g., NZ8124) with a signal peptide (e.g., a usp45 signal peptide) using a promoter from the vector (e.g., nisA), a dapA kill switch, and viability enhancements of expression of otsB but not otsA and disruption of neither trePP nor pteC.

In some embodiments, the protein of interest is expressed from a vector (e.g., NZ8124) with a signal peptide (e.g., a usp45 signal peptide) using a promoter from the vector (e.g., nisA), a dapA kill switch, and viability enhancements of expression of otsB but not otsA and disruption of trePP but not pteC.

In some embodiments, the protein of interest is expressed from a vector (e.g., NZ8124) with a signal peptide (e.g., a usp45 signal peptide) using a promoter from the vector (e.g., nisA), a dapA kill switch, and viability enhancements of expression of otsB but not otsA and disruption of pteC but not trePP.

In some embodiments, the protein of interest is expressed from a vector (e.g., NZ8124) with a signal peptide (e.g., a usp45 signal peptide) using a promoter from the vector (e.g., nisA), a dapA kill switch, and viability enhancements of expression of otsB but not otsA and disruption of trePP and pteC.

In some embodiments, the protein of interest is expressed from a vector (e.g., NZ8124) with a signal peptide (e.g., a usp45 signal peptide) using a promoter from the vector (e.g., nisA), a dapA kill switch, and viability enhancements of expression of otsA and otsB and disruption of neither trePP nor pteC.

In some embodiments, the protein of interest is expressed from a vector (e.g., NZ8124) with a signal peptide (e.g., a usp45 signal peptide) using a promoter from the vector (e.g., nisA), a dapA kill switch, and viability enhancements of expression of otsA and otsB and disruption of trePP but not pteC.

In some embodiments, the protein of interest is expressed from a vector (e.g., NZ8124) with a signal peptide (e.g., a usp45 signal peptide) using a promoter from the vector (e.g., nisA), a dapA kill switch, and viability enhancements of expression of otsA and otsB and disruption of pteC but not trePP.

In some embodiments, the protein of interest is expressed from a vector (e.g., NZ8124) with a signal peptide (e.g., a usp45 signal peptide) using a promoter from the vector (e.g., nisA), a dapA kill switch, and viability enhancements of expression of otsA and otsB and disruption of trePP and pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch and a dapA kill switch, and viability enhancements of expression of neither otsA nor otsB and disruption of neither TrePP nor pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch and a dapA kill switch, and viability enhancements of expression of neither otsA nor otsB and disruption of TrePP but not pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch and a dapA kill switch, and viability enhancements of expression of neither otsA nor otsB and disruption of pteC but not TrePP.

In some embodiments, the protein of interest is expressed from a vector (e.g., NZ8124) with a signal peptide (e.g., a usp45 signal peptide) using a promoter from the vector (e.g., nisA), a thymidylate synthase kill switch and a dapA kill switch, and viability enhancements of expression of neither otsA nor otsB and disruption of TrePP and pteC.

In some embodiments, the protein of interest is expressed from a vector (e.g., NZ8124) with a signal peptide (e.g., a usp5 signal peptide) using a promoter from the vector (e.g., nisA), a thymidylate synthase kill switch and a dapA kill switch, and viability enhancements of expression of otsA but not otsB and disruption of neither TrePP nor pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch and a dapA kill switch, and viability enhancements of expression of otsA but not otsB and disruption of TrePP but not pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch and a dapA kill switch, and viability enhancements of expression of otsA but not otsB and disruption of pteC but not TrePP.

In some embodiments, the protein of interest is expressed from a vector (e.g, NZ8124) with a signal peptide (e.g., a usp45 signal peptide) using a promoter from the vector (e.g., nisA), a thymidylate synthase kill switch and a dapA kill switch, and viability enhancements of expression of otsA but not otsB and disruption of TrePP and pteC.

In some embodiments, the protein of interest is expressed from a vector (e.g, NZ8124) with a signal peptide (e.g., a usp45 signal peptide) using a promoter from the vector (e.g., nisA), a thymidylate synthase kill switch and a dapA kill switch, and viability enhancements of expression of otsB but not otsA and disruption of neither TrePP nor pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch and a dapA kill switch, and viability enhancements of expression of otsB but not otsA and disruption of TrePP but not pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch and a dapA kill switch, and viability enhancements of expression of otsB but not otsA and disruption of pteC but not TrePP.

In some embodiments, the protein of interest is expressed from a vector (e.g., NZ8124) with a signal peptide (e.g., a usp45 signal peptide) using a promoter from the vector (e.g., nisA), a thymidylate synthase kill switch and a dapA kill switch, and viability enhancements of expression of otsB but not otsA and disruption of TrePP and pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch and a dapA kill switch, and viability enhancements of expression of otsA and otsB and disruption of neither TrePP nor pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch and a dapA kill switch, and viability enhancements of expression of otsA and otsB and disruption of TrePP but not pteC.

In some embodiments, the protein of interest is expressed from a vector (e.g., NZ8124) with a signal peptide (e.g., a usp45 signal peptide) using a promoter from the vector (e.g., nisA), a thymidylate synthase kill switch and a dapA kill switch, and viability enhancements of expression of otsA and otsB and disruption of pteC but not TrePP.

In some embodiments, the protein of interest is expressed from the bacterial chromosome with a signal peptide (e.g., a usp45 signal peptide) from the thyA promoter, using a thymidylate synthase kill switch and viability enhancements of expression of neither otsA nor otsB and disruption of neither trePP nor pteC.

In some embodiments, the protein of interest is expressed from the bacterial chromosome with a signal peptide (e.g., a usp45 signal peptide) from the thyA promoter, using a thymidylate synthase kill switch and viability enhancements of expression of neither otsA nor otsB and disruption of trePP but not pteC.

In some embodiments, the protein of interest is expressed from the bacterial chromosome with a signal peptide (e.g., a usp45 signal peptide) from the thyA promoter, using a thymidylate synthase kill switch and viability enhancements of expression of neither otsA nor otsB and disruption of pteC but not trePP.

In some embodiments, the protein of interest is expressed from the bacterial chromosome with a signal peptide (e.g., a usp45 signal peptide) from the thyA promoter, using a thymidylate synthase kill switch and viability enhancements of expression of neither otsA nor otsB and disruption of trePP and pteC.

In some embodiments, the protein of interest is expressed from the bacterial chromosome with a signal peptide (e.g., a usp45 signal peptide) from the thyA promoter, using a thymidylate synthase kill switch and viability enhancements of expression of otsA but not otsB and disruption of neither trePP nor pteC.

In some embodiments, the protein of interest is expressed from the bacterial chromosome with a signal peptide (e.g., a usp45 signal peptide) from the thyA promoter, using a thymidylate synthase kill switch and viability enhancements of expression of otsA but not otsB and disruption of trePP but not pteC.

In some embodiments, the protein of interest is expressed from the bacterial chromosome with a signal peptide (e.g., a usp45 signal peptide) from the thyA promoter, using a thymidylate synthase kill switch and viability enhancements of expression of otsA but not otsB and disruption of pteC but not trePP.

In some embodiments, the protein of interest is expressed from the bacterial chromosome with a signal peptide (e.g., a usp45 signal peptide) from the thyA promoter, using a thymidylate synthase kill switch and viability enhancements of expression of otsA but not otsB and disruption of trePP and pteC.

In some embodiments, the protein of interest is expressed from the bacterial chromosome with a signal peptide (e.g., a usp45 signal peptide) from the thyA promoter, using a thymidylate synthase kill switch and viability enhancements of expression of otsB but not otsA and disruption of neither trePP nor pteC.

In some embodiments, the protein of interest is expressed from the bacterial chromosome with a signal peptide (e.g., a usp45 signal peptide) from the thyA promoter, using a thymidylate synthase kill switch and viability enhancements of expression of otsB but not otsA and disruption of trePP but not pteC.

In some embodiments, the protein of interest is expressed Brom the bacterial chromosome with a signal peptide (e.g., a usp45 signal peptide) from the thyA promoter, using a thymidylate synthase kill switch and viability enhancements of expression of otsB but not otsA and disruption of pteC but not trePP.

In some embodiments, the protein of interest is expressed from the bacterial chromosome with a signal peptide (e.g., a usp45 signal peptide) from the thyA promoter, using a thymidylate synthase kill switch and viability enhancements of expression of otsB but not otsA and disruption of trePP and pteC.

In some embodiments, the protein of interest is expressed from the bacterial chromosome with a signal peptide (e.g., a usp45 signal peptide) from the thyA promoter, using a thymidylate synthase kill switch and viability enhancements of expression of otsA and otsB and disruption of neither trePP nor pteC.

In some embodiments, the protein of interest is expressed from the bacterial chromosome with a signal peptide (e.g., a usp45 signal peptide) from the thyA promoter, using a thymidylate synthase kill switch and viability enhancements of expression of otsA and otsB and disruption of trePP but not pteC.

In some embodiments, the protein of interest is expressed from the bacterial chromosome with a signal peptide (e.g., a usp45 signal peptide) from the thyA promoter, using a thymidylate synthase kill switch and viability enhancements of expression of otsA and otsB and disruption of pteC but not trePP.

In some embodiments, the protein of interest is expressed from the bacterial chromosome with a signal peptide (e.g., a usp45 signal peptide) from the thyA promoter, using a thymidylate synthase kill switch and viability enhancements of expression of otsA and otsB and disruption of trePP and pteC.

In some embodiments, the protein of interest is expressed from the bacterial chromosome with a signal peptide (e.g., a usp45 signal peptide) from the thyA promoter, using a dapA kill switch and viability enhancements of expression of neither otsA nor otsB and disruption of neither trePP nor pteC.

In some embodiments, the protein of interest is expressed from the bacterial chromosome with a signal peptide (e.g., a usp43 signal peptide) from the thyA promoter, using a dapA kill switch and viability enhancements of expression of neither otsA nor otsB and disruption of trePP but not pteC.

In same embodiments, the protein of interest is expressed from the bacterial chromosome with a signal peptide (e.g., a usp45 signal peptide) from the thyA promoter, using a dapA kill switch and viability enhancements of expression of neither otsA nor otsB and disruption of pteC but not trePP.

In some embodiments, the protein of interest is expressed from the bacterial chromosome with a signal peptide (e.g., a usp45 signal peptide) from the thyA promoter, using a dapA kill switch and viability enhancements of expression of neither otsA nor otsB and disruption of trePP and pteC.

In some embodiments, the protein of interest is expressed from the bacterial chromosome with a signal peptide (e.g., a usp45 signal peptide) from the thyA promoter, using a dapA kill switch and viability enhancements of expression of otsA but not otsB and disruption of neither trePP nor pteC.

In some embodiments, the protein of interest is expressed from the bacterial chromosome with a signal peptide (e.g., a usp45 signal peptide) from the thyA promoter, using a dapA kill switch and viability enhancements of expression of otsA but not otsB and disruption of trePP but not pteC.

In some embodiments, the protein of interest is expressed from the bacterial chromosome with a signal peptide (e.g., a usp45 signal peptide) from the thyA promoter, using a dapA kill switch and viability enhancements of expression of otsA but not otsB and disruption of pteC but not trePP.

In some embodiments, the protein of interest is expressed from the bacterial chromosome with a signal peptide (e.g. a usp45 signal peptide) from the thyA promoter, using a dapA kill switch and viability enhancements of expression of otsA but not otsB and disruption of trePP and pteC.

In some embodiments, the protein of interest is expressed from the bacterial chromosome with a signal peptide (e.g., a usp45 signal peptide) from the thyA promoter, using a dapA kill switch and viability enhancements of expression of otsB but not otsA and disruption of neither trePP nor pteC.

In some embodiments, the protein of interest is expressed from the bacterial chromosome with a signal peptide (e.g., a usp45 signal peptide) from the thyA promoter, using a dapA kill switch and viability enhancements of expression of otsB but not otsA and disruption of trePP but not pteC.

In some embodiments, the protein of interest is expressed from the bacterial chromosome with a signal peptide (e.g., a usp45 signal peptide) from the thyA promoter, using a dapA kill switch and viability enhancements of expression of otsB but not otsA and disruption of pteC but not trePP.

In some embodiments, the protein of interest is expressed from the bacterial chromosome with a signal peptide (e.g., a usp45 signal peptide) from the thyA promoter, using a dapA kill switch and viability enhancements of expression of otsB but not otsA and disruption of trePP and pteC.

In some embodiments, the protein of interest is expressed from the bacterial chromosome with a signal peptide (e.g., a usp5 signal peptide) from the thyA promoter, using a dapA kill switch and viability enhancements of expression of otsA and otsB and disruption of neither trePP nor pteC.

In some embodiments, the protein of interest is expressed from the bacterial chromosome with a signal peptide (e.g., a usp45 signal peptide) from the thyA promoter, using a dapA kill switch and viability enhancements of expression of otsA and otsB and disruption of trePP but not pteC.

In some embodiment the protein of interest is expressed from the bacterial chromosome with a signal peptide (e.g., a usp45 signal peptide) from the thyA promoter, using a dapA kill switch and viability enhancements of expression of otsA and otsB and disruption of pteC but not trePP.

In some embodiments, the protein of interest is expressed from the bacterial chromosome with a signal peptide (e.g, a usp45 signal peptide) from the thyA promoter, using a dapA kill switch and viability enhancements of expression of otsA and otsB and disruption of trePP and pteC.

In some embodiments, the protein of interest is expressed from the bacterial chromosome with a signal peptide (e.g., a usp45 signal peptide) from the usp45 promoter, using a thymidylate synthase kill switch and viability enhancements of expression of neither otsA nor otsB and disruption of neither trePP nor pteC.

In some embodiments, the protein of interest is expressed from the bacterial chromosome with a signal peptide (e.g., a usp45 signal peptide) from the usp45 promoter, using a thymidylate synthase kill switch and viability enhancements of expression of neither otsA nor otsB and disruption of trePP but not pteC.

In some embodiments, the protein of interest is expressed from the bacterial chromosome with a signal peptide (e.g., a usp45 signal peptide) from the usp4 promoter, using a thymidylate synthase kill switch and viability enhancements of expression of neither otsA nor otsB and disruption of pteC but not trePP.

In some embodiments, the protein of interest is expressed from the bacterial chromosome with a signal peptide (e.g., a usp45 signal peptide) from the usp45 promoter, using a thymidylate synthase kill switch and viability enhancements of expression of neither otsA nor otsB and disruption of trePP and pteC.

In some embodiments, the protein of interest is expressed from the bacterial chromosome with a signal peptide (e.g., a usp45 signal peptide) from the usp45 promoter, using a thymidylate synthase kill switch and viability enhancements of expression of otsA but not otsB and disruption of neither trePP nor pteC.

In some embodiments, the protein of interest is expressed from the bacterial chromosome with a signal peptide (e.g., a usp45 signal peptide) from the usp45 promoter, using a thymidylate synthase kill switch and viability enhancements of expression of otsA but not otsB and disruption of trePP but not pteC.

In some embodiments, the protein of interest is expressed from the bacterial chromosome with a signal peptide (e.g., a usp45 signal peptide) from the usp45 promoter, using a thymidylate synthase kill switch and viability enhancements of expression of otsA but not otsB and disruption of pteC but not trePP.

In some embodiments, the protein of interest is expressed from the bacterial chromosome with a signal peptide (e.g., a usp45 signal peptide) from the usp45 promoter, using a thymidylate synthase kill switch and viability enhancements of expression of otsA but not otsB and disruption of trePP and pteC.

In some embodiments, the protein of interest is expressed from the bacterial chromosome with a signal peptide (e.g., a usp45 signal peptide) from the usp45 promoter, using a thymidylate synthase kill switch and viability enhancements of expression of otsB but not otsA and disruption of neither trePP nor pteC.

In some embodiments, the protein of interest is expressed from the bacterial chromosome with a signal peptide (e.g., a usp45 signal peptide) from the usp45 promoter, using a thymidylate synthase kill switch and viability enhancements of expression of otsB but not otsA and disruption of trePP but not pteC.

In some embodiments, the protein of interest is expressed from the bacterial chromosome with a signal peptide (e.g., a usp45 signal peptide) from the usp45 promoter, using a thymidylate synthase kill switch and viability enhancements of expression of otsB but not otsA and disruption of pteC but not trePP.

In some embodiments, the protein of interest is expressed from the bacterial chromosome with a signal peptide (e.g., a usp45 signal peptide) from the usp45 promoter, using a thymidylate synthase kill switch and viability enhancements of expression of otsB but not otsA and disruption of trePP and pteC.

In some embodiments, the protein of interest is expressed from the bacterial chromosome with a signal peptide (e.g. a usp45 signal peptide) from the usp45 promoter, using a thymidylate synthase kill switch and viability enhancements of expression of otsA and otsB and disruption of neither trePP nor pteC.

In some embodiments, the protein of interest is expressed from the bacterial chromosome with a signal peptide (e.g., a usp45 signal peptide) from the usp45 promoter, using a thymidylate synthase kill switch and viability enhancements of expression of otsA and otsB and disruption of trePP but not pteC.

In some embodiments, the protein of interest is expressed from the bacterial chromosome with a signal peptide (e.g., a usp45 signal peptide) from the usp45 promoter, using a thymidylate synthase kill switch and viability enhancements of expression of otsA and otsB and disruption of pteC but not trePP.

In some embodiments, the protein of interest is expressed from the bacterial chromosome with a signal peptide (e.g., a usp45 signal peptide) from the usp45 promoter, using a thymidylate synthase kill switch and viability enhancements of expression of otsA and otsB and disruption of trePP and pteC.

In some embodiments, the protein of interest is expressed from the bacterial chromosome with a signal peptide (e.g., a usp45 signal peptide) from the usp45 promoter, using a dapA kill switch and viability enhancements of expression of neither otsA nor otsB and disruption of neither trePP nor pteC.

In some embodiments, the protein of interest is expressed from the bacterial chromosome with a signal peptide (e.g., a usp45 signal peptide) from the usp45 promoter, using a dapA kill switch and viability enhancements of expression of neither otsA nor otsB and disruption of trePP but not pteC.

In some embodiments, the protein of interest is expressed from the bacterial chromosome with a signal peptide (e.g., a usp45 signal peptide) from the usp45 promoter, using a dapA kill switch and viability enhancements of expression of neither otsA nor otsB and disruption of pteC but not trePP.

In some embodiments, the protein of interest is expressed from the bacterial chromosome with a signal peptide (e.g., a usp43 signal peptide) from the usp45 promoter, using a dapA kill switch and viability enhancements of expression of neither otsA nor otsB and disruption of trePP and pteC.

In some embodiments, the protein of interest is expressed from the bacterial chromosome with a signal peptide (e.g., a usp45 signal peptide) from the usp45 promoter, using a dapA kill switch and viability enhancements of expression of otsA but not otsB and disruption of neither trePP nor pteC.

In some embodiments, the protein of interest is expressed from the bacterial chromosome with a signal peptide (e.g., a usp45 signal peptide) from the usp45 promoter, using a dapA kill switch and viability enhancements of expression of otsA but not otsB and disruption of trePP but not pteC.

In some embodiments, the protein of interest is expressed from the bacterial chromosome with a signal peptide (e.g., a usp45 signal peptide) from the usp45 promoter, using a dapA kill switch and viability enhancements of expression of otsA but not otsB and disruption of pteC but not trePP.

In some embodiments, the protein of interest is expressed from the bacterial chromosome with a signal peptide (e.g., a usp45 signal peptide) from the usp45 promoter, using a dapA kill switch and viability enhancements of expression of otsA but not otsB and disruption of trePP and pteC.

In some embodiments, the protein of interest is expressed from the bacterial chromosome with a signal peptide (e.g., a usp45 signal peptide) from the usp45 promoter, using a dapA kill switch and viability enhancements of expression of otsB but not otsA and disruption of neither trePP nor pteC.

In some embodiments, the protein of interest is expressed from the bacterial chromosome with a signal peptide (e.g., a usp45 signal peptide) from the usp45 promoter, using a dapA kill switch and viability enhancements of expression of otsB but not otsA and disruption of trePP but not pteC.

In some embodiments, the protein of interest is expressed from the bacterial chromosome with a signal peptide (e.g., a usp45 signal peptide) from the usp45 promoter, using a dapA kill switch and viability enhancements of expression of otsB but not otsA and disruption of pteC but not trePP.

In some embodiments, the protein of interest is expressed from the bacterial chromosome with a signal peptide (e.g., a usp45 signal peptide) from the usp45 promoter, using a dapA kill switch and viability enhancements of expression of otsB but not otsA and disruption of trePP and pteC.

In some embodiments, the protein of interest is expressed from, the bacterial chromosome with a signal peptide (e.g., a usp45 signal peptide) from the usp45 promoter, using a dapA kill switch and viability enhancements of expression of otsA and otsB and disruption of neither trePP nor pteC.

In some embodiments, the protein of interest is expressed from the bacterial chromosome with a signal peptide (e.g., a usp45 signal peptide) from the usp45 promoter, using a dapA kill switch and viability enhancements of expression of otsA and otsB and disruption of trePP but not pteC.

In some embodiments, the protein of interest is expressed from the bacterial chromosome with a signal peptide (e.g., a usp45 signal peptide) from the usp45 promoter, using a dapA kill switch and viability enhancements of expression of otsA and otsB and disruption of pteC but not trePP.

In some embodiments, the protein of interest is expressed from the bacterial chromosome with a signal peptide (e.g., a usp45 signal peptide) from the usp45 promoter, using a dapA kill switch and viability enhancements of expression of otsA and otsB and disruption of trePP and pteC.

In some embodiments, the protein of interest is expressed from the bacterial chromosome with a signal peptide (e.g., a usp45 signal peptide) from the thyA promoter, using a thymidylate synthase kill switch and a dapA kill switch and viability enhancements of expression of neither otsA nor otsB and disruption of neither TrePP nor PtcC.

In some embodiments, the protein of interest is expressed from the bacterial chromosome with a signal peptide (e.g., a usp45 signal peptide) from the thyA promoter, using a thymidylate synthase kill switch and a dapA kill switch and viability enhancements of expression of neither otsA nor otsB and disruption of TrePP but not PtcC.

In some embodiments, the protein of interest is expressed from the bacterial chromosome with a signal peptide (e.g., a usp45 signal peptide) from the thyA promoter, using a thymidylate synthase kill switch and a dapA kill switch and viability enhancements of expression of neither otsA nor otsB and disruption of PtcC but not TrePP.

In some embodiments, the protein of interest is expressed from the bacterial chromosome with a signal peptide (e.g., a usp45 signal peptide) from the thyA promoter, using a thymidylate synthase kill switch and a dapA kill switch and viability enhancements of expression of neither otsA nor otsB and disruption of TrePP and PtcC.

In some embodiments, the protein of interest is expressed from the bacterial chromosome with a signal peptide (e.g., a usp45 signal peptide) from the thyA promoter, using a thymidylate synthase kill switch and a dapA kill switch and viability enhancements of expression of otsA but not otsB and disruption of neither TrePP nor PtCC.

In some embodiments, the protein of interest is expressed from the bacterial chromosome with a signal peptide (e.g., a usp45 signal peptide) from the thyA promoter, using a thymidylate synthase kill switch and a dapA kill switch and viability enhancements of expression of otsA but not otsB and disruption of TrePP but not PtcC.

In some embodiments, the protein of interest is expressed from the bacterial chromosome with a signal peptide (e.g., a usp45 signal peptide) from the thyA promoter, using a thymidylate synthase kill switch and a dapA kill switch and viability enhancements of expression of otsA but not otsB and disruption of PtcC but not TrePP.

In some embodiments, the protein of interest is expressed from the bacterial chromosome with a signal peptide (e.g., a usp45 signal peptide) from the thyA promoter, using a thymidylate synthase kill switch and a dapA kill switch and viability enhancements of expression of otsA but not otsB and disruption of TrePP and PtcC.

In some embodiments, the protein of interest is expressed from the bacterial chromosome with a signal peptide (e.g., a usp45 signal peptide) from the thyA promoter, using a thymidylate synthase kill switch and a dapA kill switch and viability enhancements of expression of otsB but not otsA and disruption of neither TrePP nor PtCC.

In some embodiments, the protein of interest is expressed from the bacterial chromosome with a signal peptide (e.g., a usp45 signal peptide) from the thyA promoter, using a thymidylate synthase kill switch and a dapA kill switch and viability enhancements of expression of otsB but not otsA and disruption of TrePP but not PtcC.

In some embodiments, the protein of interest is expressed from the bacterial chromosome with a signal peptide (e.g., a usp45 signal peptide) from the thyA promoter, using a thymidylate synthase kill switch and a dapA kill switch and viability enhancements of expression of otsB but not otsA and disruption of PtcC but not TrePP.

In some embodiments, the protein of interest is expressed from the bacterial chromosome with a signal peptide (e.g., a usp45 signal peptide) from the thyA promoter, using a thymidylate synthase kill switch and a dapA kill switch and viability enhancements of expression of otsB but rot otsA and disruption of TrePP and PtcC.

I3611 In some embodiments, the protein of interest is expressed from the bacterial chromosome with a signal peptide (e.g., a usp45 signal peptide) from the thyA promoter, using a thymidylate synthase kill switch and a dapA kill switch and viability enhancements of expression of otsA and otsB and disruption of neither TrePP nor PtCC.

In some embodiments, the protein of interest is expressed from the bacterial chromosome with a signal peptide (e.g., a usp45 signal peptide) from the thyA promoter, using a thymidylate synthase kill switch and a dapA kill switch and viability enhancements of expression of otsA and otsB and disruption of TrePP but not PtcC.

In some embodiments, the protein of interest is expressed from the bacterial chromosome with a signal peptide (e.g., a usp45 signal peptide) from the thyA promoter, using a thymidylate synthase kill switch and a dapA kill switch and viability enhancements of expression of otsA and otsB and disruption of PtcC but not TrePP.

In some embodiments, the protein of interest is expressed from the bacterial chromosome with a signal peptide (e.g., a usp45 signal peptide) from the thyA promoter, using a thymidylate synthase kill switch and a dapA kill switch and viability enhancements of expression of otsA and otsB and disruption of TrePP and PtcC.

In some embodiments, the protein of interest is expressed from the bacterial chromosome with a signal peptide (e.g., a usp45 signal peptide) from the usp45 promoter, using a thymidylate synthase kill switch and a dapA kill switch and viability enhancements of expression of neither otsA nor otsB and disruption of neither TrePP nor PtCC.

In some embodiments, the protein of interest is expressed from the bacterial chromosome with a signal peptide (e.g., a usp45 signal peptide) from the usp45 promoter, using a thymidylate synthase kill switch and a dapA kill switch and viability enhancements of expression of neither otsA nor otsB and disruption of TrePP but not PtcC.

In some embodiments, the protein of interest is expressed from the bacterial chromosome with a signal peptide (e.g., a usp45 signal peptide) from the usp45 promoter, using a thymidylate synthase kill switch and a dapA kill switch and viability enhancements of expression of neither otsA nor otsB and disruption of PWC but not TrePP.

In some embodiments, the protein of interest is expressed from the bacterial chromosome with a signal peptide (e.g., a usp45 signal peptide) from the usp45 promoter, using a thymidylate synthase kill switch and a dapA kill switch and viability enhancements of expression of neither otsA nor otsB and disruption of TrePP and PtcC.

In some embodiments, the protein of interest is expressed from the bacterial chromosome with a signal peptide (e.g., a usp45 signal peptide) from the usp45 promoter, using a thymidylate synthase kill switch and a dapA kill switch and viability enhancements of expression of otsA but not otsB and disruption of neither TrePP nor PtCC.

In some embodiments, the protein of interest is expressed from the bacterial chromosome with a signal peptide (e.g., a usp45 signal peptide) from the usp45 promoter, using a thymidylate synthase kill switch and a dapA kill switch and viability enhancements of expression of otsA but not otsB and disruption of TrePP but not PtcC.

In some embodiments, the protein of interest is expressed from the bacterial chromosome with a signal peptide (e.g., a usp45 signal peptide) from the usp45 promoter, using a thymidylate synthase kill switch and a dapA kill switch and viability enhancements of expression of otsA but not otsB and disruption of PtcC but not TrePP.

In some embodiments, the protein of interest is expressed from the bacterial chromosome with a signal peptide (e.g., a usp45 signal peptide) from the usp45 promoter, using a thymidylate synthase kill switch and a dapA kill switch and viability enhancements of expression of otsA but not otsB and disruption of TrePP and PtcC.

In some embodiments, the protein of interest is expressed from the bacterial chromosome with a signal peptide (e.g., a usp45 signal peptide) from the usp45 promoter, using a thymidylate synthase kill switch and a dapA kill switch and viability enhancements of expression of otsB but not otsA and disruption of neither TrePP nor PtCC.

In some embodiments, the protein of interest is expressed from the bacterial chromosome with a signal peptide (e.g., a usp45 signal peptide) from the usp45 promoter, using a thymidylate synthase kill switch and a dapA kill switch and viability enhancements of expression of otsB but not otsA and disruption of TrePP but not PtcC.

In some embodiments, the protein of interest is expressed from the bacterial chromosome with a signal peptide (e.g., a usp45 signal peptide) from the usp45 promoter, using a thymidylate synthase kill switch and a dapA kill switch and viability enhancements of expression of otsB but not otsA and disruption of PtcC but not TrePP.

In some embodiments, the protein of interest is expressed from the bacterial chromosome with a signal peptide (e.g., a usp45 signal peptide) from the usp45 promoter, using a thymidylate synthase kill switch and a dapA kill switch and viability enhancements of expression of otsB but not otsA and disruption of TrePP and PtcC.

In son embodiments, the protein of interest is expressed from the bacterial chromosome with a signal peptide (e.g., a usp45 signal peptide) from the usp45 promoter, using a thymidylate synthase kill switch and a dapA kill switch and viability enhancements of expression of otsA and otsB and disruption of neither TrePP nor PtCC.

In some embodiments, the protein of interest is expressed from the bacterial chromosome with a signal peptide (e.g., a usp45 signal peptide) from the usp45 promoter, using a thymidylate synthase kill switch and a dapA kill switch and viability enhancements of expression of otsA and otsB and disruption of TrePP but not PtcC.

In some embodiments, the protein of interest is expressed from the bacterial chromosome with a signal peptide (e.g., a usp45 signal peptide) from the usp45 promoter, using a thymidylate synthase kill switch and a dapA kill switch and viability enhancements of expression of otsA and otsB and disruption of PtcC but not TrePP.

In some embodiments, the protein of interest is expressed from the bacterial chromosome with a signal peptide (e.g., a usp45 signal peptide) from the usp45 promoter, using a thymidylate synthase kill switch and a dapA kill switch and viability enhancements of expression of otsA and otsB and disruption of TrePP and PtcC.

### Methods of Treatment

The recombinant Lactococcus lactis bacterium comprising a protein of interest (e.g., a therapeutic protein (e.g., SG-11 or one or more variants or fragments thereof)) described herein including variants (e.g., amino acid substitutions, deletions, insertions), modifications (e.g., glycosylation, acetylation), and fragments and fusions thereof is contemplated for use in treating a subject diagnosed with or suffering from a disorder related to inflammation within the gastrointestinal tract and/or malfunction of epithelial barrier function within the gastrointestinal tract.

Provided herein am methods for treating a subject in need thereof comprising administering to the subject a pharmaceutical composition comprising the recombinant Lactococcus lactis bacterium comprising a protein of interest (e.g., a therapeutic protein (e.g., SG-11 or one or more variants or fragments thereof)) as described in the present disclosure. The subject can be one who has been diagnosed with inflammatory bowel disease, ulcerative colitis, pediatric UC, Crohn's disease, pediatric Crohn's disease, short bowel syndrome, mucositis GI mucositis, oral mucositis, mucositis of the esophagus, stomach, small intestine (duodenum, jejunum, ileum), large intestine (colon), and/or rectum, chemotherapy-induced mucositis, radiation-induced mucositis, necrotizing enterocolitis, pouchitis, a metabolic disease, celiac disease, irritable bowel syndrome, or chemotherapy associated steatohepatitis (CASH). In some aspects, the present disclosure provides that the subject is suffered from various types of mucositis. Administration of pharmaceutical compositions comprising the recombinant bacterium comprising the protein of interest (e.g., a therapeutic protein (e.g., SG-11 or one or more variants or fragments thereof)) may also be useful for wound healing applications. The mucositis can be healed by pharmaceutical compositions described herein.

### Inflammatory Bowel Disease

Inflammatory bowel disease (IBO) classically includes ulcerative colitis (UC) and Crohn's disease (CD). The pathogenesis of inflammatory bowel disease is not known. A genetic predisposition has been suggested, and a host of environmental factors, including bacterial, viral and, perhaps, dietary antigens, can trigger an ongoing enteric inflammatory cascade. Id. IBD can cause severe diarrheas, pain, fatigue, and weight loss. IBD can be debilitating and sometimes leads to life-threatening complications. Accordingly. In some embodiments, the method of treatment as described herein is effective to reduce, prevent or eliminate any one or more of the symptoms described above wherein the method comprises administering to a patient in need thereof a therapeutically effective amount of a pharmaceutical composition comprising the recombinant bacterium comprising a protein of interest (e.g., a therapeutic protein (e.g., SG-1 or one or more variants or fragments thereof)). In some embodiments, the method of treatment results in remission.

### Ulcerative Colitis

Ulcerative colitis is an inflammatory bowel disease that causes long-lasting inflammation am sores (ulcers), in the innermost lining of your large intestine (colon) and rectum.

Ulcerative colitis typically presents with shallow, continuous inflammation extending from the rectum proximally to include, in many patients, the entire colon. Fistulas, fissures, abscesses and small-bowel involvement are absent. Patients with limited disease (e.g., proctitis) typically have mild but frequently recurrent symptoms, while patients with pancolitis more commonly have severe symptoms, often requiring hospitalization. Botoman et al., “Management of Inflammatory Bowel Disease,” Am. Fam. Physician, Vol. 57 (1):57-68 (Jan. 1, 1998) (internal citations omitted). Thus, ulcerative colitis is an IBD that causes long-lasting inflammation and sores (ulcers) in the innermost lining of your large intestine (colon) and rectum.

### Crohn's Disease

Unlike ulcerative colitis, Crohn's disease can involve the entire intestinal tract, from the mouth to the anus, with discontinuous fecal ulceration, fistula formation and perianal involvement. The terminal ileum is most commonly affected, usually with variable degrees of colonic involvement. Subsets of patients have perianal disease with fissures and fistula formation. Only 2 to 3 percent of patients with Crohn's disease have clinically significant involvement of the upper gastrointestinal tract. Botoman et al., “Management of Inflammatory Bowel Disease,” Am. Fam, Physician, Vol. 57(1):57-68 (Jan. 1, 1998) (internal citations omitted). Thus, Crohn's disease is an IRD that causes inflammation of the lining of your digestive tract. In Crohn's disease, inflammation often spreads deep into affected tissues. The inflammation can involve different ureas of the digestive tract, e.g, the large intestine, small intestine, or both. Collagenous colitis and lymphocytic colitis also are considered inflammatory bowel diseases, but are usually regarded separately from classic inflammatory bowel disease.

### Clinical Parameters of Inflammatory Bowel Disease

As previously discussed, inflammatory bowel disease encompasses ulcerative colitis and Crohn's disease. There are numerous scores and clinical markers known to one of skill in the art that can be utilized to access the efficacy of the administered proteins described herein in treating these conditions.

There are two general approaches to evaluating patients with IBD. The first involves the visual examination of the mucosa and relies on the observation of signs of damage to the mucosa, in view of the fact that IBD is manifested by the appearance of inflammation and ulcers in the GI tract. Any procedure that allows an assessment of the mucosa can be used. Examples include barium enemas, x-rays, and endoscopy. An endoscopy may be of the esophagus, stomach and duodenum (esophagogastroduodenoscopy), small intestine (enteroscopy), or large intestine/colon (colonoscopy, sigmoidoscopy). These techniques we used to identify areas of inflammation, ulcers and abnormal growths such as polyps.

Scoring systems based on this visual examination of the GI tract exist to determine the status and severity of IBD, and these scoring systems are intended to ensure that uniform assessment of different patients occur, despite the fact that patients may be assessed by different medical professionals, in diagnosis and monitoring of these diseases as well as in clinical research evaluations. Examples of evaluations based on visual examination of UC are discussed and compared in Daperno Metal (J Crohns Colitis. 2011 5:484-98).

Clinical scoring systems also exist, with the same purpose. The findings on endoscopy or other examination of the mucosa can be incorporated into these clinical scoring systems, but those scoring systems also incorporate data based on symptoms such as stool frequency, rectal bleeding and physician's global assessment. IUD has a variety of symptoms that affect quality of life, so certain of these scoring systems also take into account a quantitative assessment of the effect on quality of life as well as the quantification of symptoms. Both UC and CD, when present in the colon, generate a similar symptom profile which can include diarrhea, rectal bleeding, abdominal pain, and weight loss. See, Sands, B. E., “From symptom to diagnosis: clinical distinctions among various forms of intestinal inflammation.” (Gastroenterology. Vol. 126, pp. 1518-1532 (2004).

One example of a scoring system for UC is the Mayo scaring system (Schroeder et al., N Eng J Med, 1987, 317:1625-1629), but others exist that have less commonly been used and include the Ulcerative Colitis Endoscopic Index of Severity (UCEIS) score (Travis et al, 2012, Gut, 61:535-542), Baron Score (Baron et al., 1964, BMJ, 1:89), Ulcerative Colitis Colonocopic Index of Severity (UCCIS) (This et al., 2011, Inflamm Bowel Dis, 17:1757-1764), Rachmilewitz Endoscopic Index (Rachmilewitz, 1989, BMJ, 298:82-86), Sutherland Index (also known as the UC Disease Activity Index (UCDA1) scoring system; Sutherland et al., 1987, Gastroenterology, 92:1994-1998), Matts Score (Matts, 1961, QJM, 30:393-407), and Blackstone Index (Blackstone, 1984, inflammatory bowel disease. In Blackstone M O (ed.) Endoscopic interpretation: normal and pathologic appearances of the gastrointestinal tract, 1984, pp. 464-494). For a review, see Paine, 2014, Gastroenterol Rep 2:161-168. Accordingly, also contemplated herein is a method for treating a subject diagnosed with and suffering from UC, wherein the treatment comprises administering pharmaceutical compositions comprising the recombinant bacterium comprising a SG-11 protein or variant or fragment thereof as described herein and wherein the treatment results in a decrease in the UC pathology as determined by measurement of the UCEIS score, the Baron score, the UCCIS score, the Rachmilewitz Endoscopic Index, the Sutherland Index, and/or the Blackstone Index.

An example of a scoring system for CD is the Crohns Disease Activity Index (CDAI) (Sands B et al 2004, N Engl J Med 350 (9): 876-85; Best, et al. (1976) Gastroenterol. 70:439-444); most major studies use the CDAI in order to define response or remission of disease. Calculation of the CDAI score includes scoring of the number of liquid stools over 7 days, instances and severity of abdominal pain over 7 days, general well-being over 7 days, extraintestinal complications (e.g., arthritis/arthralgia, iritis/uveitis, erythema nodosum, pyoderma gangrenosum, aphthous stomatitis, anal fissure/fistula/abscess, and/or fever >37.8° C.), use of antidiarrheal drugs over 7 days, present of abdominal mass, hematocrit, and body weight as a ratio of ideal/observed or percentage deviation from standard weight. Based on the CDAI score, the CD is classified as either asymptomatic remission (0 to 149 points), mildly to moderately active CD (130 to 220 points), moderately to severely active CD (221 to 450 points), or severely active fulminant disease (451 to 1000 points). In some embodiments, the method of treatment comprising administering to a patient diagnosed with CD a therapeutically effective amount of pharmaceutical compositions comprising the recombinant bacterium comprising a protein of interest (e.g., a therapeutic protein (e.g., SG-11 or one or more variants or fragments thereof)) results in a decrease in a diagnostic score of CD. For example, the score nay change the diagnosis from severely active to mildly or moderately active or to asymptomatic remission.

The Harvey-Bradshaw index is a simpler version of the CDAI which consists of only clinical parameters (Harvey et al., 1980, Lancet 1(8178):1134-1135). The impact on quality of life is also addressed by the Inflammatory Bowel Disease Questionnaire (IBDQ) (Irvine at al., 1994, Gastroenterology 106: 287-2%). Alternative methods further include CDEIS and SES CD (see, e.g., Levesque, et al. (2015) Gastroenterol. 148:37 57). Additionally or alternatively, diagnosis includes assessment on a histological scale. Goblet depletion score and loss of crypts score are described in Johannson, et al. (2014) Gut 63:281-291. Parameters and definitions for crypt architecture distortion are described in Simmonds, et al. (2014) BMC Gastroenterol. 14:93. Distinctions between acute inflammation and chronic inflammation are described, e.g., in Simmonds, supra, and Gassier (2001) Am. J. Physiol. Gastrointest. Liver Physiol. 281:G216. G228.

In some embodiments, a method of testing an IBD, e.g., UC, is provided wherein the treatment is effective in reducing the Mayo Score. The Mayo Score is a combined endoscopic and clinical scale used to assess the severity of UC and has a scale of 1-12 The Mayo Score is a composite of subscores for stool frequency, rectal bleeding, findings of flexible proctosigmoidoscopy or colonoscopy, and physician's global assessment (Paine, 2014, Gastroenterol Rep 2:161-168). With respect to rectal bleeding, blood streaks seen in the stool less than half the time is assigned 1 point, blood in most stools is assigned 2 points and pure blood passed is assigned 3 point. Regarding stool frequency, a normal number of daily stools is assigned 0 points, 1 or 2 more stools than normal is assigned 1 point, 3 or 4 more stools than normal is assigned 2 points, and 5 or more stools than usual is assigned 3 points. With respect to the endoscopy component, a score of 0 indicates normal mucosa or inactive UC, a score of 1 is given for mild disease with evidence of mild friability, reduced vascular pattern, and mucosal erythema, a score of 2 is given for moderate disease with friability, erosions, complete loss of vascular pattern, and significant erythema, and a score of 3 is given for ulceration and spontaneous bleeding (Schroeder et al., 1987, N Engl J Med, 317:1623-1629). Global assessment by a physician assigns 0 points for a finding of normal, 1 point for mild colitis, 2 points for moderate colitis and 3 points for severe colitis. Accordingly. In some embodiments, a patient treated with a SG-11 therapeutic protein or variant or fragment thereof is successfully treated when the patient experiences a reduction in the Mayo Score by at least 1, 2 or 3 points in at least one of rectal bleeding, blood streaks seen in the stool, endoscopy subscore and physician's global assessment. In some embodiments, the method of treatment comprising administering to a patient diagnosed with UC a therapeutically effective amount of pharmaceutical compositions comprising the recombinant bacterium comprising a protein of interest (e.g., a therapeutic protein (e.g., SG-11 or one or more variants or fragments thereof)) results in a decrease in a diagnostic score of UC. For example, the score may change a diagnostic score, e.g. Mayo Score, by at least 1, 2, 3, 4, 6, 7, 8, 9, 10 or 11 points.

### Pouchitis

Additionally or alternatively, the compositions comprising the recombinant bacterium comprising a SG-11 therapeutic protein or variant and methods of administration as described herein can be used to treat pouchitis. Pouchitis is an inflammation of the lining of a pouch that is surgically created in the treatment of UC. Specifically, subjects having serious UC may have their diseased colon removed and the bowel reconnected by a procedure called ileoanal anastomosis (IPAA) or J-pouch surgery. Pouchitis cases can recur in many patients, manifesting either as acute relapsing pouchitis or chronic, unremitting pouchitis. Accordingly, provided herein are methods for treating pouchitis, acute pouchitis or recent pouchitis.

Pouchitis activity can be classified as remission (no active pouchitis), mild to moderately active (increased stool frequency, urgency, and/or infrequent incontinence), or severely active (frequent incontinence and/or the patient is hospitalized for dehydration). The duration of pouchitis can be defined as acute (loss than or equal to four weeks) or chronic (four weeks or more) and the pattern classified as infrequent (1-2 acute episodes), relapsing (three or fewer episodes) or continuous. The response to medical treatment can be labeled as treatment responsive or treatment refractory, with the medication for either case being specified. Accordingly, in some embodiments, a method for treating a subject diagnosed with pouchitis is provided wherein treatment with a pharmaceutical composition comprising the recombinant bacterium comprising a protein of interest (e.g., a therapeutic protein (e.g., SG-11 or one or more variants or fragments thereof)) results in a decrease in the severity of the pouchitis and/or results in remission.

### Mucositis and Mucosal Barriers

The mucosa of the gastrointestinal (GI) tract is a complex microenvironment involving an epithelial barrier, immune cells, and microbes. A delicate balance is maintained in the healthy colon. Luminal microbes are physically separated from the host immune system by a barrier consisting of epithelium and mucus. The pathogenesis of IBD, although not fully elucidated, may involve an inappropriate host response to an altered commensal flora with a dysfunctional mucous barrier. See, Boltin et al., “Mucin Function in Inflammatory Bowel Disease An Update,”. Clin. Gastroenterol., Vol. 47(2):106-111 (February 2013).

Mucositis occurs when cancer treatments (particularly chemotherapy and radiation) break down the rapidly divided epithelial cells lining the gastro-intestinal tract (which goes from the mouth to the anus), leaving the mucosal tissue open to ulceration and infection. Mucosal tissue, also known as mucosa or the mucous membrane, lines all body passages that communicate with the air, such as the respiratory and alimentary tracts, and have cells and associated glands that secrete mucus. The part of this lining that covers the mouth, called the oral mucosa, is one of the most sensitive parts of the body and is particularly vulnerable to chemotherapy and radiation. The oral cavity is the most common location for mucositis. While the oral mucosa is the most frequent site of mucosal toxicity and resultant mucositis, it is understood that mucositis can also occur along the entire alimentary tract including the esophagus, stomach, small intestine (duodenum, jejunum, ileum), large intestine (colon), and rectum. In some embodiments, pharmaceutical compositions comprising the recombinant bacterium comprising a protein of interest (e.g., a therapeutic protein (e.g., SG-11 or one or more variants or fragments thereof)) are therapeutically effective to treat mucositis of the mouth, esophagus, stomach, small intestine (duodenum, jejunum, ileum), large intestine (colon), and/or rectum

Oral mucositis can lead to several problems, including pain, nutritional problems as a result of inability to eat, and increased risk of infection due to open sores in the mucosa. It has a significant effect on the patient's quality of life and can be dose-limiting (e.g., requiring a reduction in subsequent chemotherapy doses). The World Health Organization has an oral toxicity scale for diagnosis of oral mucositis: Grade 1: soreness±erythema, Grade 2: erythema, ulcers; patient can swallow solid food; Grade 3: ulcers with extensive erythema; patient cannot swallow solid food; Grade 4: mucositis to the extent that alimentation is not possible. Grade 3 and Grade 4 oral mucositis is considered severe mucositis. Accordingly, provided herein is a method for treating a subject diagnosed with oral mucositis, wherein administration of a pharmaceutical composition comprising the recombinant bacterium comprising a protein of interest (e.g., a therapeutic protein (e.g., SG-11 or one or more variants or fragments thereof)) reduces the grade of oral toxicity by at least 1 point of the grade scale of 1 to 4.

In some embodiments the recombinant Lactococcus lactis bacterium comprising a protein of interest (e.g., a therapeutic protein (e.g., SG-11 or one or more variants or fragments thereof)) is used for treating mucositis, such as oral mucositis.

In some embodiment % a subject administered with the recombinant bacterium taught herein has been diagnosed with intestinal inflammation. In some embodiments, the intestinal inflammation is in the small intestine and/or the large intestine. In some embodiments, the intestinal inflammation is in the rectum. In some embodiments, the subject has been diagnosed with pouchitis.

In some embodiments, the subject has been diagnosed with intestinal ulcers. In some embodiments, the subject has been diagnosed with draining enterocutaneous and/or rectovaginal fistulas.

In some embodiments, the subject has been diagnosed with Crohn's disease (CD). In some embodiments, the CD is mildly active CD. In some embodiments, the CD is moderately to severely active CD. In some embodiments, the subject has been diagnosed with pediatric CD.

In some embodiments, the subject has been diagnosed with short bowel syndrome or irritable bowel syndrome.

In some embodiments, the subject has been diagnosed with mucositis. In some embodiments, the mucositis is oral mucositis. In some embodiments, the mucositis is chemotherapy-induced mucositis, radiation therapy-induced mucositis, chemotherapy-induced oral mucositis, or radiation therapy-induced oral mucositis. In some embodiments, the mucositis is gastrointestinal mucositis. In some embodiments, the gastrointestinal mucositis is mucositis of the small intestine, the large intestine, or the rectum.

In some embodiments, the administering to a subject diagnosed with CD resulted in a reduced number of draining enterocutaneous and/or rectovaginal fistulas. In some embodiments, the administering maintains fistula closure in adult subjects with fistulizing disease.

In some embodiments, the subject has been diagnosed with ulcerative colitis (UC). In some embodiments, the UC is mildly active UC. In some embodiments, the UC is moderately to severely active UC. In some embodiments, the subject has been diagnosed with pediatric UC.

In some embodiments, the subject is in clinical remission from an IBID. In some embodiments, the subject is in clinical remission from UC, pediatric UC, CD or pediatric CD.

In some embodiments, the subject has an inflammatory bowel disease or disorder other than Crohn's disease or ulcerative colitis. In some embodiments, the subject has at least one symptom associated with inflammatory bowel disease.

In some embodiments, the administering refers to the administering of the bacterium comprising at least one first heterologous nucleic acid encoding a first polypeptide, which is a therapeutic protein comprising an amino acid sequence having at least 90% sequence identity to SEQ ID NO:19 and/or SEQ ID NO:34.

In some embodiments, the administering reduces gastrointestinal inflammation and/or reduces intestinal mucosa inflammation associated with inflammatory bowel disease in the subject. In some embodiments, the administering improves intestinal epithelial cell barrier function or integrity in the subject.

In some embodiments, after the administering the subject experiences a reduction in at least one symptom associated with an inflammatory bowel disease or disorder. In some embodiments, the at least one symptom is selected from the group consisting of abdominal pain, blood in stool, pus in stool, fever, weight loss, frequent diarrhea, fatigue, reduced appetite, nausea, cramps, anemia, tenesmus, and metal bleeding. In some embodiments, after the administering the subject experiences reduced frequency of diarrhea, reduced blood in stool and/or reduced rectal bleeding.

In some embodiments, the subject has experienced inadequate response to conventional therapy. In some embodiments, the conventional therapy is treatment with an aminosalicylate, a corticosteroid, a thiopurine, methotrexate, a JAK inhibitor, a sphingosine 1-phosphate (SIP) receptor inhibitor, an anti-integrin biologic, an anti-IL12/23R or anti-IL23/p10 biologic, and/or an anti-tumor necrosis factor agent or biologic.

In some embodiments, the administering modulates (e.g. increases or decreases) levels of a cytokine in the blood, plasma, serum, mucus or tissue of the subject.

In some embodiments, the administering increases the amount of mucin in intestinal lumen of the subject.

In some embodiments, the administering increases intestinal epithelial cell wound healing in the subject.

In some embodiments, the administering prevents or reduces colon shortening in the subject.

In some embodiments, the administering comprises rectal, intravenous, parenteral, oral, topical, dermal, transdermal or subcutaneous administering or the pharmaceutical composition to the subject. In some embodiments, the administering is to the gastrointestinal lumen.

In some embodiments, the subject is also administered at least one second therapeutic agent. In some embodiments, the at least one second therapeutic agent is selected from the group consisting of anti-diarrheal, an anti-inflammatory agent, an antibody, an antibiotic, or an immunosuppressant. In some embodiments, the at least one second therapeutic agent is an aminosalicylate, a steroid, or a corticosteroid. In some embodiments, the at least one second therapeutic agent is selected from the group consisting of adalimumab, pegol, golimumab, infliximab, vedolizumab, ustekinumab, tofacitinib, and certolizumab or certolizumab pegol.

### Epithelial Barrier Function in IBD

Studies in recent years have identified a major role of both genetic and environmental factors in the pathogenesis of IBD. Markus Neurath, “Cytokines in inflammatory Bowel Disease,” Nature Reviews Immunology, Vol. 14, 329-342 (2014). A combination of these IBD risk factors seems to initiate alterations in epithelial barrier function, thereby allowing the translocation of luminal antigens (for example, bacterial antigens from the commensal microbiota) into the bowel wall. Id. Subsequently, aberrant and excessive cytokine responses to such environmental triggers cause subclinical or acute mucosal inflammation in a genetically susceptible host. Id. Thus, the importance of proper epithelial barrier function in IBD is apparent, for in patients that fail to resolve acute intestinal inflammation, chronic intestinal inflammation develops that is induced by the uncontrolled activation of the mucosal immune system. In particular, mucosal immune cells, such as macrophages, T cells, and the subsets of innate lymphoid cells (ILCs) seem to respond to microbial products or antigens from the commensal microbiota by producing cytokines that can promote chronic inflammation of the gastrointestinal tract. Consequently, restoring proper epithelial barrier function to patients may be critical in resolving IBD.

### Colon Shortening

Ulcerative colitis is an idiopathic inflammatory bowel disease that affects the colonic mucosa and is clinically characterized by diarrhea, abdominal pain and hematochezia. The extent of disease is variable and may involve only the rectum (ulcerative proctitis), the left side of the colon to the splenic flexure, or the entire colon (pancolitis). The severity of the disease may also be quite variable histologically, ranging from minimal to florid ulceration and dysplasia. Carcinoma may develop. The typical histological (microscopic) lesion of ulcerative colitis is the crypt abscess, in which the epithelium of the crypt breaks down and the lumen fills with polymorphonuclear cells. The lamina propria is infiltrated with leukocytes. As the crypts are destroyed, normal mucosal architecture is lost and resultant scarring shortens and can narrow the colon. Thus, colon shortening can be a consequence of colitis disease and is often used diagnostically. For example, non-invasive plain abdominal x-rays can demonstrate the gaseous outline of the transverse colon in the acutely ill patient. Shortening of the colon and loss of haustral markings can also be demonstrated by plain films, as well as a double-contrast barium enema. Indications of ulcerative disease include loss of mucosal detail, cobblestone filling defects, and segmental areas of involvement. See, “Ulcerative Colitis: Introduction—Johns Hopkins Medicine,” found at: www.hopkinsmedicine.org/gastroenterology_hepatology/_pdfs/small_large_intestine/ulcerative-colitis.pdf.

Further, art recognized in vivo models of colitis will utilize shortening of colon length in scoring the severity of colitis in the model. See. Kim et al., “Investigating Intestinal Inflammation in DSS-induced Model of IBD,” Journal of Visualized Experiments, Vol. 60, pages 2-6 (February 2012).

### Epithelial Barrier Function in Non-IBD Diseases

An improperly functioning epithelial barrier is increasingly implicated in, e.g., IBDs and mucositis. Moreover, them are numerous other diseases that studies have shown are also caused, linked, correlated, and/or exacerbated by, an improperly functioning epithelial barrier. These diseases include: (1) metabolic diseases, including-obesity, type 2 diabetes, non-alcoholic steatohepatitis (NASH), non-alcoholic fatty liver disease (NAFLD), liver disorders, and alcoholic steatohepatitis (ASH); (2) celiac disease; (3) necrotizing enterocolitis; (4) irritable bowel syndrome (IBS); (5) enteric infections (e.g. Clostridium difficile); (6) other gastro intestinal disorders in general; (7) interstitial cystitis; (8) neurological disorders or cognitive disorders (e.g. Alzheimer's, Parkinson's, multiple sclerosis, and autism); (9) chemotherapy associated steatohepatitis (CASH); and (10) pediatric versions of the aforementioned diseases. See, e.g.: Everard et al., “Responses of Gut Microbiota and Glucose and Lipid Metabolism to Prebiotics in Genetic Obese and Diet-Induced Leptin-Resistant Mie,” Diabetes, Vol. 60, (November 2011), pgs. 2775-2786; Everard et al., “Cross-talk between Akkermansia muciniphila and intestinal epithelium controls diet-induced obesity.” PNAS, Vol. 110, No. 22, (May 2013), pgs. 9066-9071; Cani et al., “Changes in Gut Microbiota Control Metabolic Endotoxemia-Induced Inflammation in High-Fat Diet-Induced Obesity and Diabetes in Mice,” Diabetes, Vol. 57, (June 2008), pgs. 1470-1481; Delzenne et al., “Targeting gut microbiota in obesity: effects of probiotics and probiotics,” Nate Reviews, Vol. 7. (November 2011), pgs. 639-646. Consequently, restoring proper epithelial barrier function to patients may be critical in resolving the aforementioned disease states.

A properly functioning epithelial barrier in the lumen of the alimentary canal, including the mouth, esophagus, stomach, small intestine, large intestine, and rectum, is critical in controlling and maintaining the microbiome within the gastrointestinal tract and alimentary canal. The ecosystem for the microbiome includes the environment, barriers, tissues, mucus, mucin, enzymes, nutrients, food, and communities of microorganism, that reside in the gastrointestinal tract and alimentary canal. The integrity and permeability of the intestinal mucosal barrier impacts heath in many critical ways.

A loss of integrity of the mucosal barrier in gastro-intestinal disorders due to changes in mucin secretion may be related to host immune changes, luminal microbial factors, or directly acting genetic or environmental determinants. Thus, the disequilibrium of the mucous barrier may be central to the pathogenesis of IBD. Boltin et al., “Mucin Function in Inflammatory Bowel Disease An Update,” J. Clin. Gastroenterol., Vol. 47(2):106-111 (February 2013).

Mucins am the primary constituent of the mucous layer lining the GI tract. There are at least 21 mucin (MUC) genes known in the human genome, encoding either secreted or membrane-bound mucins. The predominant mucins in the normal colorectum are MUC1, MUC2, MUC3A, MUC3B, MUC4, MUC13, and MUC17.1. MUC2 is the primary secretory, gel-forming component of intestinal mucus, produced in goblet cells. See, Boltin el al., “Mucin Function in Inflammatory Bowel Disease An Update” J. Clin. Gastroenterol., Vol. 47(2):106-11 (February 2013). Along with additional secreted mucins such as MUC1, 3A, 3B, 4, 13 and 17.1, goblet cell secretion of MUC2 forms a protective barrier on colonic epithelial cells reducing exposure to intestinal contents which may damage epithelial cells or prime immune responses.

The dosing regimen used for treatment depends upon the desired therapeutic effect, on the route of administration, and on the duration of the treatment. The dose will vary from patient to patient, depending upon the nature and severity of disease, the patient's weight, special diets then being followed by a patient, concurrent medication, and other factors which those skilled in the art will recognize.

Generally, dosage levels of therapeutic protein between 0.0001 to 10 mg/kg of body weight daily are administered to the patient, e.g., patients suffering from inflammatory bowel disease. The dosage range will generally be about 0.5 mg to 100.0 g per patient per day, which may be administered in single or multiple doses.

In some aspects, the dosage range will be about 0.5 mg to 10 g per patient per day, or 0.5 mg to 9 g per patient per day, or 0.5 mg to 8 g per patient per day, or 0.5 mg to 7 g per patient per day, or 0.5 mg to 6 g per patient per day, or 0.5 mg to 5 g per patient per day, or 0.5 mg to 4 g per patient per day, or 0.5 mg to 3 g per patient per day, or 0.5 mg to 2 g per patient per day, or 0.5 mg to 1 g per patient per day.

In some aspects, the dosage range will be about 0.5 mg to 900 mg per patient per day, or 0.5 mg to 800 mg per patient per day, or 0.5 mg to 700 mg per patient per day, or 0.5 mg to 600 mg per patient per day, or 0.5 mg to 500 mg per patient per day, or 0.5 mg to 400 mg per patient per day, or 0.5 mg to 300 mg per patient per day, or 0.5 mg to 200 mg per patient per day, or 0.5 mg to 100 mg per patient per day, or 0.5 mg to 50 mg per patient per day, or 0.5 mg to 40 mg per patient per day, or 0.5 mg to 30 mg per patient per day, or 0.5 mg to 20 mg per patient per day, or 0.5 mg to 10 mg per patient per day, or 0.5 mg to 1 mg per patient per day.

### Compositions Comprising a Recombinant Bacterium

In some embodiments, the recombinant bacterium compositions of the present disclosure can be administered to a subject in need thereof to enhance general health and well-being and/or to treat or prevent a disease or disorder such as a gastrointestinal barrier function disorder or disease associated with reduced intestinal epithelial barrier function as described herein. In some embodiments, the composition is a live biotherapeutic product (LBP) while in some embodiments, the composition is a probiotic. In some embodiments, the recombinant Lactococcus lactis bacterium is isolated and has been cultured outside of a subject to increase the number or concentration of the bacteria, thereby enhancing the therapeutic efficacy of a composition comprising the bacterial population.

In some embodiments, the composition is in the form of alive bacterial population. The live population may be, e.g., frozen, cryoprotected or lyophilized. In some embodiments, the composition comprises a non-viable bacterial preparation, or the cellular components thereof. In some embodiments, where the composition is in the form of a non-viable bacterial preparation, it is selected from, for example, het-killed bacteria, irradiated bacteria and lysed bacteria.

In some embodiments, the bacterial species is in biologically pure form, substantially fee from other species of organism. In some embodiments, the bacterial species is in the form of a culture of a single species of organism.

Compositions comprising the recombinant Lactococcus lactis bacterium comprising a protein of interest (e.g., a therapeutic protein (e.g., SG-11 or one or more variants or fragments thereof)) in accordance with the present disclosure can be any of a number of accepted probiotic or live biotherapeutic product (LBP) delivery systems suitable for administration to a subject. Importantly, a composition for delivery of a live population of recombinant Lactococcus lactis bacterium must be formulated to maintain viability of the microbe. In some embodiments, thecompositioncompriseselementswhichprotectthebacteriafromtheacidicenvironmentof the stomach. In some embodiments, the composition includes an enteric coating.

In some embodiments, the composition is a food-based product. A food-based product can be, for example, a yogurt, cheese, milk, meat, cream, or chocolate. Such food-based products can be considered edible which means that it is approved for human or animal consumption.

One aspect of the disclosure relates to a food product comprising the bacterial species defined above. The term “food product” s intended to cover all consumable products that can be solid, jellied or liquid. Suitable food products may include, for example, functional food products, food compositions, pet food, livestock food, health foods, feedstuffs, and the like. In some embodiments, the food product is a prescribed health food.

As used herein, the term “functional food product” means food that is capable of providing not only a nutritional effect, but is also capable of delivering a further beneficial effect to the consumer. Accordingly, functional foods are ordinary foods that have components or ingredients (such as those described herein) incorporated into them that impart to the food a specific functional—e.g. medical or physiological benefit—other than a purely nutritional effect.

Examples of specific food products that are applicable to the preset disclosure include milk-based products, ready to cat desserts, powders for re-constitution with, e.g., milk or water, chocolate milk drinks, malt drinks, ready-to-eat dishes, instant dishes or drinks for humans or food compositions representing a complete or a partial diet intended for humans, pets, or livestock.

In some embodiments, the composition according to the present disclosure is a food product intended for humans, pets or livestock. The composition nay be intended for animals selected from the group consisting of non-hu man primates, dogs, cats, pigs, cattle, hoses, goats, sheep, or poultry. In another embodiment, the composition is a food product intended for adult species, in particular human adults.

Another aspect of the disclosure relates to food products, dietary supplements, nutraceuticals, nutritional formulae, drinks and medicaments containing the bacterial species as defined above, and use thereof.

In the present disclosure, “milk-based product” means any liquid or semi-solid milk or whey based product having a varying fat content. The milk-based product can be, e.g., cow's milk, goat's milk, sheep's milk, skimmed milk, whole milk, milk recombined from powdered milk and whey without any processing, or a processed product, such as yoghurt, curdled milk, curd, sour milk, sour whole milk, butter milk and other sour milk products. Another important group includes milk beverages, such as whey beverages, fermented milks, condensed milks, infant or baby milks; flavored milks, ice cream; milk-containing food such a sweets.

Compositions comprising recombinant Lactococcus lactis bacterium comprising SG-11 or a variant or fragment thereof can be a tablet, a chewable tablet, a capsule, a stick pack, a powder, or effervescent powder. The composition can comprise coated beads which contain the bacteria. A powder may be suspended or dissolved in a drinkable liquid such as water for administration.

In some embodiments, the composition comprises a microbe and/or a bacterium which is isolated. The isolated microbe may be included in a composition with one or more additional substance(s). For example, the isolated microbe may be included in a pharmaceutical composition with one or more pharmaceutically acceptable excipient(s).

In some embodiments, the composition may be used to promote or improve human health. In some aspects, the composition may be used ID improve gut health, gastrointestinal tract health and mouth health.

The microbes and/or recombinant bacteria described herein may also be used in prophylactic applications. In prophylactic applications, bacterial species or compositions according to the disclosure are administered to a patient susceptible to, or otherwise at risk of, a particular disease in an amount that is sufficient to at least partially reduce the risk of developing a disease. The precise amounts depend on a number of patient specific factors such as the patient's state of health and weight.

In some embodiments, the disclosure provides for various immediate and controlled release formulations comprising the taught microbes, recombinant bacteria and combinations thereof. Controlled release formulations sometimes involve a controlled release coating disposed over the bacteria. In particular embodiments, the controlled release coatings may be enteric coatings, semi-enteric coatings delayed release coatings, or pulsed release coatings may be desired. In particular, a coating will be suitable if it provides an appropriate lag in active release (e.g. release of the therapeutic microbes and combinations thereof). It can be appreciated that in some embodiments one does not desire the therapeutic microbes, recombinant bacteria and combinations thereof to be released into the acidic environment of the stomach, which could potentially degrade and/or destroy the taught microbes and recombinant bacteria, before it reaches a desired target in the intestines.

In some embodiments, the compositions of this disclosure encompass the recombinant Lactococcus lactis bacterium comprising a protein of interest (e.g., a therapeutic protein (e.g., SG-11 or one or more variants or fragments thereof)) as described above.

In some embodiments, the composition of the present disclosure further comprises a prebiotic in an amount of from about 1 to about 30% by weight, respect to the total weight composition, preferably from 5 to 20% by weight. Preferred carbohydrates are selected from, fructooligosaccharides (or FOS), short-chain fructo-oligosaccharides, inulin, isomalt-oligosaccarides, pectins, xylo-oligosaccharides (or XOS), chitosan-oligosaccharides (or COS), beta-glucans, arable gum modified and resistant starches, polydextrose, D-tagatose, acacia fibers, carob, oats, and citrus fibers. Particularly preferred prebiotics are the short-chain fructo-oligosaccharides (for simplicity shown herein below as FOSs-c.c); said FOSs-c.c. are not digestible carbohydrates, generally obtained by the conversion of the beet sugar and including a saccharose molecule to which three glucose molecules are bonded.

In some embodiments, the composition further comprises at least one other kind of other food grade bacterium, wherein the food grade bacterium is preferably selected from the group consisting of lactic acid bacteria, bifidobacteria, propionibacterium or mixtures thereof.

In some embodiments, microbe compositions comprise 10⁶-10¹² CFU (colony forming units), 10⁸-10¹² CFU, 10¹⁰-10¹² CFU, 10⁸-10¹⁰ CFU, or 10⁸-10¹¹ CFU of a bacterial species. In some embodiments, microbial combinations comprise about 10⁶, about 10⁷, about 10⁸, about 10⁹, about 10¹⁰, about 10¹¹, or about 10¹² CFU of a bacterial species. In some embodiments, the bacterial species is a recombinant Lactococcus lactis bacterium comprising a protein of interest (e.g., a therapeutic protein (e.g., SG-11 or one or more variants or fragments thereof)) or a variant or fragment thereof.

Compositions comprising a recombinant Lactococcus lactis bacterium comprising a protein of interest (e.g., a therapeutic protein (e.g., SG-11 or one or more variants or fragments thereof)) according to the present disclosure can be formulated for delivery to a desired site of action within an individual to whom it is administered. For example, the composition may be formulated for oral and/or rectal administration. Additionally, the compositions may be formulation for administration to the gastrointestinal lumen, or for delayed release in the intestine, terminal ileum, or colon.

When employed as a pharmaceutical, e.g., for treatment or prophylaxis of a disease, disorder, or condition, the compositions described herein are typically administered in the form of a pharmaceutical composition. Such compositions can be prepared in a manner well known in the pharmaceutical art and include at least one active compound, e.g., a live strain as described herein. Generally, the compostions are administered in a pharmaceutically effective amount, e.g., a therapeutically or prophylactically effective amount. The amount of the active agent, e.g., a microbe and/or bacterium as described herein, administered will typically be determined by a physician, in the light of the relevant circumstances, including the condition to be treated, the chosen route of administration, the activity of the microbes and/or bacteria administered, the age, weight, and response of the individual patient, the severity of the patient's symptoms, and the like.

The compositions can be administered by a variety of routes including oral, rectal, and intranasal. Depending on the intended route of delivery, the compositions are formulated as either injectable or oral compositions or as salves, as lotions, or as patches.

The compositions for oral administration can take the form of bulk liquid solutions or suspensions, or bulk powders. More commonly, however, the compositions are presented in unit dosage forms to facilitate accurate dosing. Typical unit dosage forms include prefilled, premeasured ampules or syringes of the liquid compositions or pills, tablets, capsules or the like in the case of solid compositions. The above-described components for orally administrable or injectable administrable compositions are merely representative. Other materials, as well as processing techniques and the like are set forth in Part 8 of Remington's The Science and Practice of Pharmacy, 21^{st} edition, 2005, Publisher Lippincott Williams & Wilkins, which is incorporated herein by reference.

For oral administration, particular use is made of compressed tablets, pills, tablets, gellules, drops, and capsules. In some embodiments, the composition comprising the recombinant Lactococcus lactis bacterium comprising a protein of interest (e.g., a therapeutic protein (e.g., SG-11 or one or more variants or fragments thereof)) is formulated as a pill, a tablet, a capsule, a suppository, a liquid, or a liquid suspension.

The compositions may be formulated in unit dosage form, e.g., in the form of discrete portions containing a unit dose, or a multiple or sub-unit of a unit dose.

In another embodiment, the compositions of the disclosure are adminstered in combination with one or more other active agents. In such cases, the compositions of the disclosure may be administered consecutively, simultaneously or sequentially with the one or more other active agents.

### Pharmaceutical Compositions Comprising the Recombinant Lactococcus Lactis Bacterium Comprising a Protein of Interest

Pharmaceutical compositions are provided herein which comprise the recombinant Lactococcus lactis bacterium comprising a protein of interest (e.g., a therapeutic protein (e.g., SG-11 or one or more variants or fragments thereof)) according to the present disclosure or pharmaceutically acceptable salt thereof and a pharmaceutically acceptable excipient. In some embodiments, the pharmaceutical composition is formulated for administration to the gastrointestinal lumen, including the mouth, esophagus, small intestine, large intestine, rectum and/or anus.

In some embodiments, the composition comprises one or more other substances which are associated with the recombinant bacterium comprising source of the protein, for example, cellular components from a production host cell, or substance associated with chemical synthesis of the protein. In some embodiments, the pharmaceutical composition is formulated to include one or more second active agents as described herein. Moreover, the composition may comprise ingredients that preserve the structural and/or functional activity of the active agent(s) or of the composition itself. Such ingredients include but are not limited to antioxidants and various antibacterial and antifungal agents, including but not limited to parabens (e.g., methylparabens, propylparabens), chlorobutanol, phenol, sorbic acid, thimerosal or combinations thereof.

The terms “pharmaceutical” or pharmaceutically acceptable” refers compositions that do not or preferably do not produce an adverse, allergic, or other untoward reaction when administered to an animal, such as, for example, a human, as appropriate. The preparation of a pharmaceutical composition or additional active ingredient will be known to those of skill in the art in light of the present disclosure, as exemplified by Remington's Pharmaceutical Sciences, 18^{th} Ed. Mack Printing Company, 1990, incorporated herein by reference. Moreover, for animal (e.g., human) administration, it will be understood that preparations should meet sterility, pyrogenicity, general safety and purity standards as required by the FDA Office of Biological Standards.

The pharmaceutical compositions of the disclosure are formulated according to the intended route of administration and whether it is to be administered, e.g., in solid, liquid or aerosol form. In a preferred embodiment, the composition can be administered rectally, but may also be administered topically, by injection, by infusion, orally, intrathecally, intranasally, subcutaneously, mucosally, localized perfusion bathing target cells directly, via a catheter, via a lavage, or by other method or any combination of the foregoing as would be known to one of ordinary skill in the art. Liquid formulations comprising a therapeutically effective amount of the protein can be administered rectally by enema, catheter, use of a bulb syringe. A suppository is an example of a solid dosage form formulated for rectal delivery. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and or about 1% to about 2%. Injectable liquid compositions are typically based upon injectable sterile saline or phosphate-buffered saline or other injectable harriers known in the art. Other liquid compositions include suspensions and emulsions. Solid compositions such as for oral administration may be in the form of tablets, pills, capsules (e.g., hard or soft-shelled gelatin capsules), buccal compositions, troches, elixirs, suspensions, syrups, wafers, or combinations thereof. The active agent in such liquid and solid compositions, e.g., a protein as described herein, is typically a component, being about 0.05% to 10% by weight, with the remainder being the injectable carrier and the like.

The pharmaceutical composition comprising the recombinant bacterium comprising a protein of interest (e.g., a therapeutic protein (e.g., SG-11 or one or more variants or fragments thereof)) may be formulated as a controlled or sustained release composition which provide release of the active agent(s) including the therapeutic protein of the present disclosure over an extended period of time, e.g., over 30-60 minutes, or over 1-10 hours, 2-8 hours, 8-24 hours, etc. Alternatively or additionally, the composition is formulated for release to a specific site in the host body. For example, the composition may have an enteric costing to prevent release of the active agent(s) in an acidic environment such as the stomach, allowing release only in the more neutral or basic environment of the small intestine, colon or rectum. Alternatively or additionally, the composition may be formulated to provide delayed release in the mouth, small intestine or large intestine.

Each of the above-described formulations may contain at least one pharmaceutically acceptable excipient or carrier, depending up the intended route of administration, e.g., a solid for rectal administration or liquid for intravenous or parenteral administration or administration via cannula. As used herein. “pharmaceutically acceptable carrier” includes any and ail solvents, dispersion media, coatings, surfactants, antioxidants, preservatives (e.g., antibacterial agents, antifungal agents), isotonic agents, absorption delaying agents salts, preservative, drugs, drug stabilizers, gels, binders, excipient, disintegration agents, lubricants, sweetening agents, flavoring agents, dyes, such like materials and combinations thereof, as would be known to one of ordinary skill in the art (see, for example, Remington's Pharmaceutical Sciences, 18^{th} Ed. Mack Printing Company, 1990, pgs. 1289-1329, incorporated herein by reference).

The pharmaceutical compositions for administration ca be present in unit dosage forms to facilitate accurate dosing. Typical unit dosage forms include prefilled, premeasured ampules or syringes of the liquid compositions or suppositories, pills, tablets, capsules or the like in the case of solid compositions. In some embodiments of such compositions, the active agent, e.g., a protein as described herein, may be a component (about 0.1 to 50 wt/wt %, 1 to 40 wt/wt %, 0.1 to 1 wt/wt % A or 1 to 10 wt/wt %) with the remainder being various vehicles or carriers and processing aids helpful for forming the desired dosing form.

The actual dosage amount in a unit dosage form of the present disclosure administered to a patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

### Dosage and Administration Schedule

The dosages disclosed herein arm exemplary of the average case. There can of course be individual instances where higher or lower dosage ranges are merited, and such are within the scope of this disclosure. The term “unit dosage form” refers to a physically discrete unit suitable as a unitary dosage for an individual to whom administered, each unit containing a predetermined quantity of active material calculated to produce the desired therapeutic or prophylactic effect, and may be in association with a suitable pharmaceutical excipient.

In some embodiments, the effective daily dose in a subject is from about 1×10⁶ to about 1×11¹² colony forming units (CFUs), 1×10⁷ to 1×10¹² CFUs, 1×10⁸ to 1×10¹² CFUs, 1×10⁸ to 1×10¹¹ CFUs, 1×10⁸ to 1×10¹⁰ CFUs, 1×10⁸ to 1×10⁹ CFUs, 1×10⁹ to 1×10¹² CFUs, 1×10¹⁰ to 1×10¹² CFUs, or 1×10¹⁰ to 1×10¹¹ CFUs. The subject may be a human or non-human primate. Alternatively, the subject may be another mammal such as a rat, mouse, rabbit, etc.

In some embodiments, the daily dose is administered to the subject daily for about 1 to 2 weeks, to 4 weeks, 1 to 2 months, 1 to 6 months, 1 to 12 months.

Alternatively, the dose which ranges from about 1×10⁶ to about 1×10¹² colony forming units (CFUs), 1×10⁷ to 1×10¹² CFUs, 1×10⁸ to 1×10¹² CFU, 1×10⁸ to 1×10¹¹ CFUs, 1×10⁸ to 1×10¹⁰ CFUs, 1×10⁸ to 1×10⁹ CFUs, 1×10⁹ to 1×10¹² CFUs, 1×10¹⁰ to 1×10¹² CFUs, or 1×10¹⁰ to 1×10¹¹ CFUs is administered to a subject three times a day, twice a day, once a day, every other day, once per week, 3 times per week, 5 times per week, once per month, twice per month, 3 times per month, one every 2 months, or 3 times, 4 times or 6 times per year. In these embodiments, the dose can be administered to the subject for a period extending from about 0 to 2 weeks, 1 to 2 weeks, 1 to 4 weeks, 1 to 2 months, 1 to 6 months, 1 to 12 months.

The dose administered to a subject should be sufficient to treat a disease and/or condition, partially reverse a disease and/or condition, fully reverse a disease and/or condition, or establish a healthy-state microbiome. In some aspects, the dose administered to a subject should be sufficient to prevent the onset of symptoms associated with an inflammation condition. In some embodiments, the dose is effective to treat or ameliorate the symptoms of an inflammatory disorder. In some embodiments, the inflammatory is an inflammatory bowel disease and/or mucositis.

Dosing may be in one or a combination of two or more administrations, e.g., daily, bi-daily, weekly, monthly, or otherwise in accordance with the judgment of the clinician or practitioner, taking into account factors such as age, weight, severity of the disease, and the dose administered in each administration.

In another embodiment, an effective amount can be provided in from 1 to 500 ml or from 1 to 500 grams of the bacterial composition having from 10⁷ to 10¹¹ bacteria per ml or per gram, or a capsule, tablet or suppository having from 1 mg to 1000 mg lyophilized powder having from 10⁷ to 10¹¹ bacteria. Those receiving acute treat-ment can receive higher doses than those who are receiving chronic administration (such as hospital workers or those admitted into long-term care facilities).

The effective dose as described above, can be administered, for example, orally, rectally, intravenously, via a subcutaneous injection, or transdermally. The effective dose can be provided as a solid or liquid, and can be present in one or more dosage form units (e.g., tablets or capsules).

### Combination Therapies Comprising Therapeutic Proteins

The pharmaceutical compositions taught herein comprising a therapeutic protein may be combined with other treatment therapies and/or pharmaceutical compositions. For example, a patient suffering from an inflammatory bowel disease, may already be taking a pharmaceutical prescribed by their doctor to treat the condition. In embodiments, the pharmaceutical compositions taught herein, are able to be administered in conjunction with the patient's existing medicines.

For example, the therapeutic proteins taught herein may be combined with one or more of: an anti-diarrheal, a 5-aminosalicylic acid compound, an anti-inflammatory agent, an antibiotic, an antibody (e.g. antibodies targeting an inflammatory cytokine, e.g. antibodies targeting an anti-cytokine agent such as anti-TNF-α, (e.g., adalimumab, certolizumab pegol, golimumab, infliximab, V565) or anti-IL-121IL-23 (e.g., ustekinumab, risankizumab, brazikumab, ustekinumab), a JAK inhibitor (e.g., tofacitinib, PF06700841, PF06651600, filgotinib, upadacitinib), an anti-integrin agent (e.g., vedolizumab, etrolizumab), a SIP inhibitor (e.g., etrasimod, onnimod, amiselimod), a recombinant cell-based agent) e.g., Cx601), a steroid, a corticosteroid, an immunosuppressant (e.g., azathioprine and mercaptopurine), vitamins, and/or specialized diet.

Cancer patients undergoing chemotherapy or radiation therapy and suffering from or at risk of developing may be administered a pharmaceutical composition according to the present disclosure in combination with an agent used to treat mucositis such as oral mucositis. In some embodiments, a method of treatment comprises administering to a patient suffering from mucositis a combination of a pharmaceutical composition comprising the recombinant Lactococcus lactis bacterium comprising SG-11 or a variant or fragment thereof and one or more second therapeutic agents selected from the group consisting of amifostine, benzocaine, benzydamine, ranitidine, omeprazole, capsaicin, glutamine, prostaglandin E2, Vitamin E, sucralfate, and allopurinol.

In some embodiments, a synergistic effect is achieved upon combining the disclosed therapeutic proteins with one or more additional therapeutic agents.

In some embodiments of the methods herein, the second therapeutic agent is administered in conjunction with the recombinant Lactococcus lactis bacterium comprising a protein of interest (e.g., a therapeutic protein (e.g., SG-11 or one or more variants or fragments thereof)) described herein, either simultaneously or sequentially. In some embodiments, the protein and the second agent act synergistically for treatment or prevention of the disease, or condition, or symptom. In some embodiments, the protein and the second agent act additively for treatment or prevention of the disease, or condition, or symptom.

### Protein Expression Systems and Protein Production

Provided herein are compositions and methods for producing proteins of the present disclosure as well as expression vectors which contain polynucleotide sequence encoding the proteins and host cells which harbor the expression vectors.

The proteins of the present disclosure can be prepared by routine recombinant methods, e.g., culturing cells transformed or transfected with an expression vector containing a nucleic acid encoding protein of interest (e.g., a therapeutic protein (e.g., SG-11 or one or more variants or fragments thereof)). Host cells comprising any such vector are also provided. Host cells can be prokaryotic or eukaryotic and examples of host cells include L. lactis, E. coli, yeast, or mammalian cells. A method for producing any of the herein described proteins is further provided and comprises culturing host cells under conditions suitable for expression of the desired protein and recovering the desired protein from the cell culture. The recovered protein can then be isolated and/or purified for use in in vitro and in vivo methods, as well as for formulation into a pharmaceutically acceptable composition. In some embodiments, the protein is expressed in a prokaryotic cell such as L. lactis and E. coli, and the isolation and purification of the protein includes step to reduce endotoxin to levels acceptable for therapeutic use in humans or other animals.

In some embodiments, a method for producing any of the herein described recombinant cell comprising proteins taught in the disclosure is further provided and comprises culturing host cells under conditions suitable for expression of the desired protein and secreting the desired protein from the host cell. Host cells can be prokaryotic or eukaryotic and examples of host cells include L. lactis, E. coli, yeast, or mammalian cells. The recombinant cell can then be isolated and/or purified for use in in vitro and in vivo methods, as well as for formulation into a pharmaceutically acceptable composition. In some embodiments, the secreted protein is expressed in a prokaryotic cell such as L. lactis and E. coli, and the host cell expressing the protein can be utilized for therapeutic use in humans or other animals.

### Method to Produce the Protein

Methods are provided for producing the proteins described herein but are well known to the ordinarily skilled artisan. Host cells transformed or transfected with expression or cloning vectors described herein for protein production are cultured in conventional nutrient media modified as appropriate for inducing promoter, selecting and/or maintaining transformants, and/or expressing the gene encoding the desired protein sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991) and Molecular Cloning: A Laboratory Manual (Sambrook, et al, 1989, Cold Spring Harbor Laboratory Press).

Generally, “purified” will refer to a specific protein composition that has been subjected to fractionation to remove nonproteinaceous components and various other proteins, polypeptides, or peptides, and which composition substantially retains its activity, as may be assessed, for example, by the protein assays, as described herein below, or as would be known to one of ordinary skill in the art for the desired protein, polypeptide or peptide.

Where the term “substantially purified” is used, this will refer to a composition in which the specific protein, polypeptide, or peptide forms the major component of the composition, such as constituting about 50% of the proteins in the composition or more. In preferred embodiments, a substantially purified protein will constitute more than 60%, 70%, 80%, 90%, 95%, 99% or even more of the proteins in the composition.

A peptide, polypeptide or protein that is “purified to homogeneity,” as applied to the present disclosure, means that the peptide, polypeptide or protein has a level of purity where the peptide, polypeptide or protein is substantially free from other proteins and biological components. For example, a purified peptide, polypeptide or protein will often be sufficiently free of other protein components so that degradative sequencing may be performed successfully.

Although preferred for use in certain embodiments, there is no general requirement that the protein, polypeptide, or peptide always be provided in their most purified state. Indeed, it is contemplated that less substantially purified protein, polypeptide or peptide, which are nonetheless enriched in the desired protein compositions, relative to the natural state, will have utility in certain embodiments.

Various methods for quantifying the degree of purification of proteins, polypeptides, or peptides will be known to those of skill in the art in light of the present disclosure. These include, for example, determining the specific protein activity of a fraction, or assessing the number of polypeptides within a fraction by gel electrophoresis.

Another example is the purification of a specific fusion protein using a specific binding partner. Such purification methods are routine in the art. As the present disclosure provides DNA sequences for the specific proteins, any fusion protein purification method can now be practiced. This is exemplified by the generation of a specific protein-glutathione S-transferase fusion protein, expression in E. coli, and isolation to homogeneity using affinity chromatography on glutathione-agarose or the generation of a poly-histidine tag on the N- or C-terminus of the protein, and subsequent purification using Ni-affinity chromatography. However, given many DNA and proteins are known, or may be identified and amplified using the methods described herein, any purification method can now be employed.

In some embodiments, a preparation enriched with the peptides may be used instead of a purified preparation. In this document, whenever purified is used, enriched may be used also. A preparation may not only be enriched by methods of purification, but also by the over-expression or over-production of the peptide by bacteria when compared to wild-type. This can be accomplished using recombinant methods, or by selecting conditions which will induce the expression of the peptide from the wild type cells.

Recombinantly expressed polypeptides of the present disclosure can be recovered from culture, medium or from host cell lysates. The suitable purification procedures include, for example, by fractionation on an ion-exchange (anion or cation) column; ethanol precipitation; reverse phase HPLC; chromatography on silica or on a cation-exchange resin such as DEAE; chromatofocusing; SDS-PAGE; ammonium sulfite precipitation; gel filtration or size exclusion chromatograph (SEC) using, for example, Sephadex G-75; and metal chelating columns to bind epitope-tagged forms of a polypeptide of the present disclosure. Various methods of protein purification can be employed and such methods are known in the art and described for example in Deutscher, Methods in Enzymology, 182 (1990); Scopes, Protein Purification: Principles and Practice, Springer-Verlag New York (1982). The purification step(s) selected will depend, for example, on the nature of the production process used and the particular polypeptide produced.

Alternative methods, which are well known in the art, can be employed to prepare a polypeptide of the present disclosure. For example, a sequence encoding a polypeptide or portion thereof, can be produced by direct peptide synthesis using solid-phase techniques (see, e.g., Stewart et al., 1969, Solid-Phase Peptide Synthesis, W.H. Fireman Co., San Francisco, Calif.; Merrifield. J. 1963, Am. Chem. Soc., 85:2149-2154. In vitro protein synthesis can be performed using manual techniques or by automation. Automated synthesis can be accomplished, for instance, using an Applied Biosystems Peptide Synthesizer (Foster City, Calif.) using manufacturer's instructions. Various portions of a polypeptide of the present disclosure or portion thereof can be chemically synthesized separately and combined using chemical or enzymatic methods to produce the full-length polypeptide or portion thereof.

In some embodiments, the disclosure provides chimeric molecules comprising any of the herein described polypeptides fused to a heterologous polypeptide or amino acid sequence and the polynucleotides encoding the chimeric molecules. Examples of such chimeric molecules include, but are not limited to, any of the herein described polypeptides fused to an epitope tag sequence, an Fc region of an immunoglobulin.

The following examples are intended to illustrate, but not limit, the disclosure.

### EXAMPLES

The following experiments utilize a robust mixture of in vitro experiments combined with in vivo models of IBD and epithelial barrier function disorders to demonstrate the therapeutic ability of the described proteins and methods.

### Example 1

### Expression of SG-11 and Variants Thereof

For experiments described in the examples below, a polynucleotide encoding SG-II (SEQ ID NO:3) was obtained by PCR amplification of genomic DNA obtained from Roseburia hominis (A2-183; DSM 16839 type strain; See. e.g. Duncan, et al. (2006). Int. J. Syst. Evol. Microbiol. Vol. 56, pgs. 2437-2441). The encoding polynucleotide was then subcloned into an inducible expression vector and used to transform E. coli BL21(DE3) cells for expression and purification of SG-11 or variants thereof as detailed below, using culturing and purification methods routine in the art.

### Expression of SG-11 (Comprising SEQ ID NO:3).

Expression and purification of proteins comprising the amino acid sequence of SG-11 (SEQ ID NO:5) for use in various experiments is described below using a pGEX vector system, which is designed for inducible, high-level intracellular expression of genes or gene fragments. Expression in E. coli yields tagged proteins with the GST moiety at the amino terminus and the protein of interest at the carboxyl terminus. The vector has a tac promoter for chemically inducible, high-level expression and an internal laq1^{S} gene for use in any E. coli host.

A polynucleotide comprising a nucleotide sequence encoding SG-11 (SEQ ID NO:3 from R. hominis DSM 16839) was inserted into the multiple-cloning site (BamHI and NotI sites) of pGEX-6P-1 (GE Healthcare Life Science, Pittsburgh, Pa.) to express SG-11 as a GST fusion protein, which was then cleaved at the PreScission protease site, generating SG-11 having the amino acid sequence of SEQ ID NO:5 (encoded by SEQ ID NO:6), provided in Table 6 below. This protein was expressed and purified by two alternate methods. In the first, E. coli BL21(DE3) cells were transformed with the pGEX-6P-1 expression construct, and the BL21(DE3) transformants were grown at 30° C. in LB with 100 μg/ml carbenicillin and 1 μg/ml chloramphenicol. Expression was induced when a culture density of 0.6 OD₆₀₀ was reached, with OA mM IPTG for 4 h. Cells were harvested by centrifugation then lysed by sonication, and the soluble lysate was applied to a GST-resin column. Bound protein was washed with PBS and then purified tag-fire SG-11C was eluted by adding PreScission Protease to cleave the protein C-terminal to the GST-tag.

In the second method, the same pGEX expression construct was used and the transformed BL21(DE3) ells were grown at 37° C. in LB with 50 μg/ml carbenicillin. When cultures reached a density of 0.7 OD₆₀₀, they were chilled to 16° C., and expression was induced with 1 mM isopropyl β-D-1-thiogalactopyranoside (IPTG) at 16° C. for 15 h. Cells were harvested and lysed by sonication, and the soluble lysate was applied to a GSTrap column. Bound protein was washed with HEPES buffer and then purified tag-free SG-11 (SEQ ID NO:5) was eluted by adding HRV3C protease to cleave the protein C-terminal to the GST-tag. Eluted fractions containing protein as determined by SDS-PAGE and Coomasie Blue staining were identified and pooled, then applied to a HiTrap Q HP anion exchange column then to a Superdex 75 (26/60) preparative size exclusion column (SEC) to obtain a final preparation.

TABLE 6

| Amino Add Sequence | Encoding Nucleic Acid Sequence |
|---|---|
| SEQ ID NO: 5 | SEQ ID NO: 6 |
| GPLGSLEGEESVVYVGKKGVIASL | GGGCCCCTGGGATCCCTGGAGGGAGAGGAAAGTGTCGTGTACGT |
| DVETLDQSYYDETELKSYVDAEV | GGGAAAGAAAGGCGTGATAGCGTCGCTGGATGTGGAGACGCTC |
| EDYTAEHGKNAVKVESLKVEDG | GATCAGTCCTACTACGATGAGACGGAACTGAAGTCCTATGTGGAT |
| VAKLKMKYKTPEDYTAFNGIELY | GCAGAGGTGGAAGATTACACCGCGGAGCATGGTAAAAATGCAGT |
| QGKVVASLAAGYVYDGEFARVE | CAAGGTGGAGAGCCTTAAGGTGGAAGACGGTGTGGCGAAGCTT |
| EGKVVGAATKQDIYSEDDLKVAII | AAGATGAAGTACAAGACACCGGAGGATTATACCGCATTTAATGG |
| RANTDVKVDGEICYVSCQNVKLT | AATTGAACTCTATCAGGGGAAAGTCGTTGCTTCCCTGGCGGCAGG |
| GKDSVSIRDGYYLETGSVTASVD | ATACGTCTACGACGGGGAGTTCGCCCGCGTGGAGGAAGGCAAG |
| VTGQESVGTEQLSGTEQMEMT | GTTGTGGGAGCTGCCACAAAACAGGATATTTACTCTGAGGATGAT |
| GEPVNADDTEQTEAAAGDGSFE | TTGAAAGTTGCCATCATCCGTGCCAATACGGATGTGAAGGTGGAC |
| TDVYTFIVYKAAAS | GGTGAGATCTGCTATGTCTCCTGTCAGAATGTGAAGCTGACCGGA |
| | AAAGACAGTGTGTCGATCCGTGACGGATATTATCTTGAGACGGG |
| | AAGCGTGACGGCATCCGTGGATGTGACCGGACAGGAGAGCGTC |
| | GGGACCGAGCAGCTTTCGGGAACCGAACAGATGGAGATGACCG |
| | GGGAGCCGGTGAATGCGGATGATACCGAGCAGACAGAGGCGGC |
| | GGCCGGTGACGGTTCGTTCGAGACAGACGTATATACTTTCATTGT |
| | CTACAAAGCGGCCGCATCG |

Expression and purification of the mature SG-11 protein having no signal peptide was done using a pD451-SR vector system (AUTM, Newark, Calif.). This expression vector utilizes an IPTG-inducible T7 promoter. The polynucleotide (SEQ ID NO:4) encoding SG-11 was codon-optimized for expression in E. coli at AUTM (Newark, Calif.) to generate the codon-optimized coding sequence provided herein as SEQ ID NO:8. This codon-optimized coding sequence was inserted into the pD451-SR vector and the resultant construct provides expression of the 233-amino acid SG-11 protein provided herein as SEQ ID NO:7.

BL21(DE3) cells transformed with the construct were grown in auto-induction media, MagicMedia (ThermoFisher). The cultures were incubated with shaking at 25° C. for 8 hours then at 16° C. for up to 72 hours. Cells were pelleted by centrifugation, re-suspended in 100 mM Tris-HCl, pH 8.0 containing 50 mM NaCl, 2 mg/ml lysozyme and protease inhibitor, then Triton X-100 was added to the suspension. Cells were then sonicated and clear lysate was prepared by centrifugation for purification of the protein by standard column chromatography techniques.

SG-11 (SEQ ID NO:7) was purified with two anion exchange columns, HiTrap Q followed by Mono Q. Fractions containing partially purified proteins as determined by SDS-PAGE and Coomassie Blue staining were further purified with Mono Q. The purification protocol for MonoQ was the same as that for HiTrapQ. The fraction containing SG-11 were pooled and dialyzed in buffer (50 mM sodium phosphate, 150 mM NaCl and 10% glycerol). Purity and uniformity was analyzed with SDS-PAGE and analytical SEC, Superdex 200 Increase 3.2/300. The preparation was assessed to have about 92.7% purity.

The pD451-SR vector system was also used to express and purify the SG-11 variant SG-11V5 (SEQ ID NO:19). To generate the expression construct, the codon-optimized sequence (SEQ ID NO:8) was modified to generate the polynucleotide of SEQ ID NO:20, which encodes SG-11V5 (SEQ ID NO:19). The SG-11V5 encoding sequence was cloned into the pD451-SR vector.

BL21(DE3) cells transformed with the construct were grown and processed for preparation of clear lysate as described above for expression of SG-11 (SEQ ID NO:7).

SG-11V5 protein was purified from clear lysate by HiTrap Q purification, followed by hydrophobic interaction chromatography (HIC) HiTrap Butyl HP. Fractions containing SG-11V5 as determined by SDS-PAGE and Coomassie Blue staining, were pooled and dialyzed in buffer in buffer (50 mM sodium phosphate, 150 mM NaCl and 10% glycerol). All column chromatography described for preparation of 0-11 (SEQ ID NO:7) and SG-11V5 (SEQ ID NO:19 was performed using ÄKTA protein purification systems (GE Healthcare Life Sciences, Pittsburgh, Pa.).

Purified proteins were quantified by densitometry using bovine serum albumin as a reference following SDS-PAGE and Coomassie Blue staining. Endotoxin levels were measured with Endosafe® Nexgen-MCS™ (Charles River, Wilmington, Mass.) according to the manufacturer's instruction. Endotoxin levels of proteins used for the assays described herein were lower than 1 Endotoxin Unit/mg.

An expression construct was generated in which a pET-28 vector was used to express a polynucleotide sequence encoding SG-11 (SEQ ID NO:3) with a FLAG-tag (DYKDDDDK; SEQ ID NO:32) at the N-terminus of SG-11. The full FLAG-tagged SG-11 protein sequence is provided herein as SEQ ID NO:9 (and is encoded by codon-optimized polynucleotide SEQ ID NO:10). Protein expression using this construct is under the control of the T7 promoter, which can be induced with IPTG. The FLAG-tag at the N-terminus was incorporated into the construct using PCR and oligonucleotides encoding DYKDDDDK (SEQ ID NO:32). The transformed host cells were grown in 2xYT media overnight at 37° C. The overnight culture was then inoculated into fresh 2xYT media and incubated at 37° C. for 4 hours. The 4-hour culture was then inoculated (1% inoculation) into MagicMedia™ E. coli Expression Medium (ThermoFisher). Cells were grown at 25° C. for 8 h and then 16° C. for up to 72 h prior to harvesting by centrifugation. The protein was expressed as a soluble form allowing recovery from a clear lysate. The expressed protein was purified using a HiTrapQ anion exchange column followed by a Superdex 200 Increase 10/300 GL SEC. Purity and uniformity was analyzed with SDS-PAGE and analytical SEC, Superdex 200 increase 3.2/300, and the preparation was assessed to have about 93.3% purity.

### Preparation of SG-11 Proteins for Stability Analysis

SG-11 (SEQ ID NO:7) and a variant, SG-11V5 (SEQ ID NO:19) were purified with two anion exchange columns, HiTrap Q followed by Mono Q. Fractions containing partially purified proteins as determined by SDS-PAGE and Coomassie Blue staining were further purified with Mono Q. Purification protocol for MonoQ was the same as that for HiTrapQ. The fractions containing SG-11 were pooled and dialyzed in buffer (50 mM sodium phosphate, 150 mM NaCl and 10% glycerol).

For SG-11V5, following HiTrap Q purification, the protein was further purified with hydrophobic interaction chromatography (HIC), HiTrap Butyl HP. Fractions containing SG-11V5 and determined by SDS-PAGE and Coomassie Blue staining were pooled and dialyzed in buffer in buffer (50 mM sodium phosphate, 150 mM NaCl and 10% glycerol). All column chromatography described for preparation of and was performed using ÄKTA protein purification systems (GE Healthcare Life Sciences, Pittsburgh, Pa.).

Purified proteins were quantified by densitometry using bovine serum albumin as a reference following SDS-PAGE and Coomassie Blue staining. Endotoxin levels were measured with Endosafe® Nexgen-MCSS™ (Charles River, Wilmington, Mass.) according to the manufacturer's instructions. Endotoxin levels of proteins used for the assays described herein were lower than 1 EU/mg.

### Example 2

### Effect of SG-11 on Restoration of Epithelial Barrier Integrity Following Inflammation Induced Disruption

The following experiment demonstrates the therapeutic ability of a protein as disclosed herein to restore gastrointestinal epithelial barrier integrity. The experiment demonstrates the functional utility of a therapeutic such a SG-11 to treat a gastrointestinal inflamatory disorder or disease involving impaired epithelial barrier integrity/function.

Assays were performed as described below in trans-well plates where co-cultures of multiple cell types were performed utilizing a permeable membrane to separate cells. In the apical (top) chamber, human colonic epithelial cells, consisting of a mixture of enterocytes and goblet cells, were cultured until cells obtained tight junction formation and barrier function capacity as assessed by measurement of trans-epithelial electrical resistance (TEER). In the basolateral chamber, monocytes were cultured separately. Epithelial cell % were primed with inflammatory cytokines. The assays measured the effect of a therapeutic protein, e.g., SG-11, on epithelial barrier function, muc2 gene expression, and production of cytokines.

Cell culture. The HCT8 human enterocyte cell line (ATCC Cat. No. CCL-244) was maintained in RPMI-1640 medium supplemented with 10% fetal bovine seam, 100 IU/ml penicillin, 100 [symbol] g/ml streptomycin, 10 [symbol] g/ml gentamicin and 0.25 μg/ml amphotericin (cRPMI). HT29-MTX human goblet cells (Sigma-Aldrich (St. Louis, Mo.; Cat. No. 12040401) were maintained in DMEM medium with 10% fetal bovine serum, 100 IU/ml penicillin, 100 [symbol] g/ml streptomycin, 10 [symbol] g/ml gentamicin and 0.25 μg/ml amphotericin (cDMEM). Epithelial cells were passaged by trypsinization and were used between 5 and 15 passages following thawing from liquid nitrogen stocks. U937 monocytes (ATCC Cat. No. 700928) were maintained in cRPMI medium as a suspension culture, and split by dilution as needed to maintain cells between 5×10⁵ and 2×10⁶ cell/ml. U937 cells were used up to passage 18 following thawing from liquid nitrogen stocks.

Epithelial cell culture. A mixture of HCT8 enterocytes and HT29-MTX goblet cells were plated at a 9:1 ratio, respectively, in the apical chamber of the transwell plate as described previously (Berget et al., 2017, Int J Mol Sci, 18:1573; Beduneau et al., 2014. Eur J Pharm Biopharm, 87:290-298). A total of 10⁵ cells were plated in each well (9×10⁴ HCT cells and 1×10⁴ HT29-MTX cells per well). Epithelial cells were trypsinized from culture flasks and viable cells determined by trypan blue counting. The correct volumes of each cell type were combined in a single tube and centrifuged. The cell pellet was resuspended in cRPMI and added to the apical chamber of the transwell plate. Celle were cultured for 8 to 10 days at 37° C.+5% CO₂, and media was changed every 2 days.

Monocyte culture. On day 6 of epithelial cell culture 2×10⁵ cells/well U937 monocytes were plated into a 96 well receiver plate. Cells were cultured at 37° C.+5% CO₂ and media was changed every 24 hours for four days.

Co-culture assay. Following 8-10 days of culture, 10 ng/ml IFN-© was added to the basolateral chamber of the transwell plate containing enterocytes, for 24 hours at 37° C.+5% CO₂. After 24 hours, fresh cRPMI was added to the epithelial cell culture plate. TEER readings were measured after the IFN-© treatment and were used as the pre-treatment TEER value. SG-11 was then added to the apical chamber of the transwell plate at a final concentration of 1 [symbol] g/ml (40 nM). The myosin light chain kinase (MLCK) inhibitor peptide 18 (BioTechne, Minneapolis, Minn.) was used at 50 nM as a positive control to prevent inflammation induced barrier disruption (Zolotarevsky et al., 2002, Gastroenterology. 123:163-172). The bacterially derived molecule staurosporine was used at 100 nM as a negative control to induce apoptosis and exacerbate barrier disruption (Antonsson and Persson, 2009, Anticancer Res, 29:2893-2898). Compounds were incubated on enterocytes for 1 hour or 6 hours. Following pre-incubation with test compounds, the transwell insert containing the enterocytes was transferred on top of the receiver plate containing U937 monocytes. Heat killed E. coli (HK E. coli) (bacteria heated to 80° C. for 40 minutes) was then added to both the apical and basolateral chambers at a multiplicity of infection (MOI) of 10. Transwell plates were incubated at 37° C.+5% CO₂ for 24 hours and a post treatment TEER measurement was made. The TEER assays were performed with mature SG-11 protein (SEQ ID NO:5 or SEQ ID NO:9).

Data analysis. Raw electrical resistance values in ohms (\hat{ }) We converted to ohms per square centimeter (\hat{ }cm²) based on the surface area of the transwell insert (0.143 cm²). To adjust for differential resistances developing over 10 days of culture, individual well post treatment \hat{ }cm² readings were normalized to pre-treatment \hat{ }cm² readings. Normalized \hat{ }cm² values were then expressed as a percent change from the mean \hat{ }cm² values of untreated samples.

SG-11 protein was added 30 minutes (FIG. 1A) or 6 hours (FIG. 1B) prior to exposure of both epithelial cells and monocytes to heat killed Escherichia coli (HK E. coli), inducing monocytes to produce inflammatory mediators resulting in disruption of the epithelial monolayer as indicated by a reduction in TEER. A MLCK inhibitor was utilized as a control compound, which has been shown to prevent barrier disruption and/or reverse barrier loss triggered by the antibacterial immune response. Staurosporine was used as a control compound that caused epithelial cell apoptosis and/or death, thus resulting in a drastic decrease in TEER, which indicates disruption and/or loss of epithelial cell barrier integrity/function. In FIG. A SG-11 increased TEER from 55.8% disruption by HK E. coli to 62%. In FIG. 1B, SG-11 increased TEER from a 53.5% disruption by HK E. coli to 60.6%. The graphs in FIGS. 1A-1B represent data pooled from two individual experiments (n=6).

### Example 3

### Effects of SG-11 on Epithelial Cell Wound Healing

The following experiment demonstrates the therapeutic ability of a protein as disclosed herein to increase gastrointestinal epithelial cell wound healing. The experiment demonstrates the functional utility of the therapeutic protein SG-11 to treat a gastrointestinal inflammatory disease, or disease involving impaired epithelial barrier integrity/function, where increased epithelial cell wound healing would be beneficial.

The 96 well Oris Cell Migration assay containing plugs preventing cell attachment in the center of each well was used according to the manufacturer's instructions (Platypus Technologies. Madison, Wis.).

The migration assay plates were warmed to room temperature prior to use and plugs were removed rom 100% confluence wells prior to cell addition. The HCT8 enterocyte and HT29-MTX goblet cell lines were used at a 9:1 ratio with a total of 5×10⁴ total cells added per well (4.5×10⁴ HCT8 cells and 0.5×10⁴ HT29-MTX cells). Cells were incubated at 37° C.+5% CO₂ for 24 hours. Plugs were then removed from all control and sample wells. Control wells included cells treated with the diluent vehicle as the blank, 30 ng/ml epidermal growth factor (EGF) as the positive control, and 100 nM staurosporine as the negative control, all diluted in cRPMI. Sample wells contained SG-11 protein (SEQ ID NO:9) at a concentration of 1 μg/ml diluted in eRPMI. 100% and 0% wells were cultured in cRPMI. Treatments were added to cells and incubated at 37° C.+5% CO₂ for 48 hours. Prior to staining for viable cells, plugs were removed from the 0% wells. Treatment media was removed and cells were washed in PBS containing 0.9 mM CaCl₂ and 0.5 mM MgCl₂. The green fluorescent viability dye Calcenin AM was added to all wells at a concentration of 0.5 μg/min PBS containing 0.9 mM CaCl₂ and 0.5 mM MgCl₂, incubated for 30 min at 37° C.+5% CO₂, the dye was removed and cells were washed in PBS containing 0.9 mM CaCl₂ and 0.5 mM MgCl₂, and fluorescence was measured. Relative fluorescent values from 100% wells where plugs were removed prior to cell plating were set as the max effect, and 0% wells where plugs remained in place until immediately before staining were used as the baseline. Samples were normalized between 100% and 0% samples and values expressed as a percent growth.

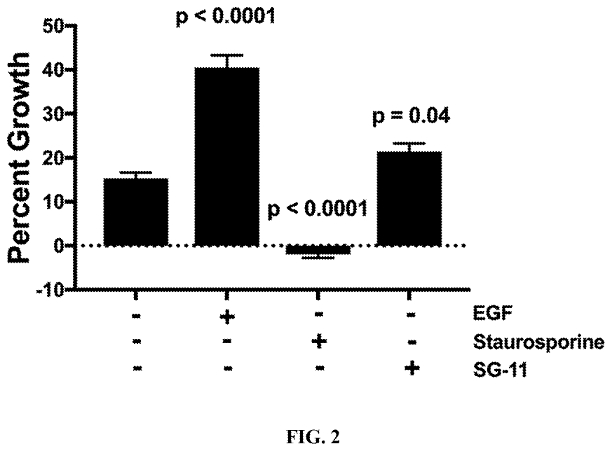

As shown in FIG. 2, a significant increase in growth was observed upon treatment with SG-11. Control compounds modulated wound healing as expected with EGF increasing proliferation, and staurosporine suppressing cell proliferation. The graph in FIG. 2 represents data pooled from 5 experiments (n=15). The data represent 5 independent replicate experiments wherein SEQ ID NO:5 was used in 1 experiment and SEQ ID NO:9 was used in 4 experiments.

### Example 4

### SG-11 Demonstrates Therapeutic Activity in a Concurrent DSS Model of Inflammatory Bowel Disease

Examples 4 and 5 demonstrate the ability of a protein as disclosed herein to treat inflammatory bowel disease in an in vi model. The experiment demonstrate that the aforementioned in vitro models, which described important functional and possible mechanistic modes of action, will translate into an in vivo model system of inflammatory bowel disease. Specifically, the mice in Examples 4 and 5 were treated with dextran sodium sulfate (DSS), a chemical known to induce intestinal epithelial damage and thereby reduce intestinal barrier integrity and function. DSS mice are well-accepted models of colitis. In Example 4, mice were treated with SG-11 protein approximately concurrent with (6 hours prior to) administration of DSS. In Example 5, mice were treated with DSS for 6 days prior to treatment with SG-1 protein.

The graphs presented in Example 4 represent data pooled from 3 independent experiments, each using 10 mice (n=30). The SG-11 protein used in these experiments was the mature protein (no signal peptide) without an N-terminal tag and comprising the amino acid sequence of SEQ ID NO3. For 2 experiments, the SG-11 protein consisted of SEQ ID NO:5; for the third experiment, the SG-11 protein consisted of SEQ ID NO:7.

Eight-week old C57BL/6 mice were housed 5 animals per cage and given food and water ad libitum for 7 days. Following the 7-day acclimation period, treatments were initiated concurrently with addition of 2.5% DSS to the drinking water. Preliminary tracking studies with fluorescently labeled bovine serum albumin following intraperitoneal (i.p.) injection of protein demonstrated proteins reached the colon at 6 hours after i.p. delivery. Based on these results, 6 hours prior to addition of 2.3% DSS to the drinking water, mice were treated with 50 nmoles/kg SG-11 (1.3 mg/kg) or Gly2-GLP2 (0.2 mg/kg) i.p. Six hours after the initial treatment, the drinking water was changed to water containing 2.3% DSS. The mice were treated with 2.5% DSS in their drinking water for 6 days. Treatments were continued with SG-11 or Gly2-GLP2 twice a day (b.i.d.) in the morning and evening (every 8 and 16 hr) with i.p. injections at 50 nmoles/kg. Fresh 2.5% DSS drinking water was prepared every 2 days.

On day six, mice were fasted for four hours and then orally gavaged with 600 mg/kg 4KDa dextran labeled with fluorescein isothiocyanate (FITC) [4KDa-FITC]. One hour after the 4KDa-FITC gavage, mice were euthanized, blood was collected, and FITC signal was measured in serum. A significant increase in 4KDa-FITC dextran translocation across the epithelial barrier was observed in untreated mice, in comparison to vehicle treated DSS mice. Additionally, a significant reduction in 4KDa-FITC dextran was observed in mice receiving DSS and treated with SG-11, as compared to DSS mice treated with vehicle. The magnitude of 4KDa-FITC dextran translocation observed for SG-11 was similar to the positive control of Gly2-GLP2. Results are shown in FIG. 3, and are presented as mean±SEM. The graph in FIG. 3 represents data pooled from 3 independent experiments, each using 10 mice (n=30).

### SG-11 Improves Inflammation Centric Readouts of Barrier Function in a Concurrent DSS Model of Inflammatory Bowel Disease

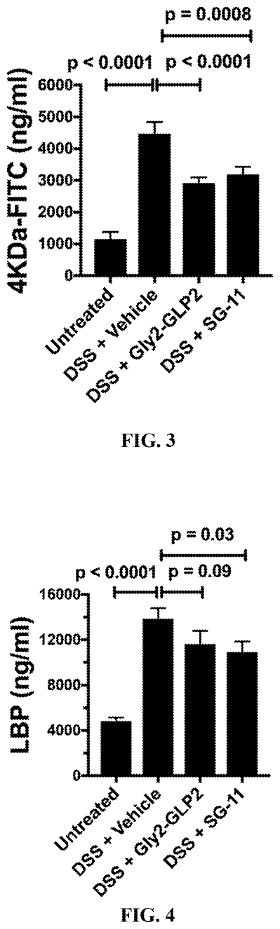

SG-11 was also assessed for its effects on the levels of lipopolysaccharide (LPS) binding protein (LBP) in the blood of the DSS animal with and without SG-11 administration. LBP, which has been linked to clinical disease activity in subjects with inflammatory bowel disease, was also measured by ELA in the serum of mice tested in the DSS model described in this Example. A significant increase in LBP concentration was observed in response to DSS. Additionally, a significant reduction in LBP was observed in SG-11 treated mice given DSS as compared to DSS mice treated with vehicle. Furthermore, SG-11 had a greater impact on LBP concentration as compared to the control peptide Gly2-GLP2, as a significant difference between DSS mice treated with Gly2-GLP2 and DSS mice treated with SG-11 was observed. Results are shown in FIG. 4, and are presented as mean±SEM. The graph in FIG. 4 represents data pooled from 3 independent experiments (n=30; each experiment using 10 mice).

### SG-11 Prevents Weight Loss in a Concurrent DSS Model of Inflammatory Bowel Disease

Also assessed was the therapeutic ability of a SG-11 protein as disclosed herein to ameliorate weight loss in an animal suffering from an inflammatory intestinal disorder. Weight loss is a significant and potentially dangerous side effect of inflammatory bowel disease.

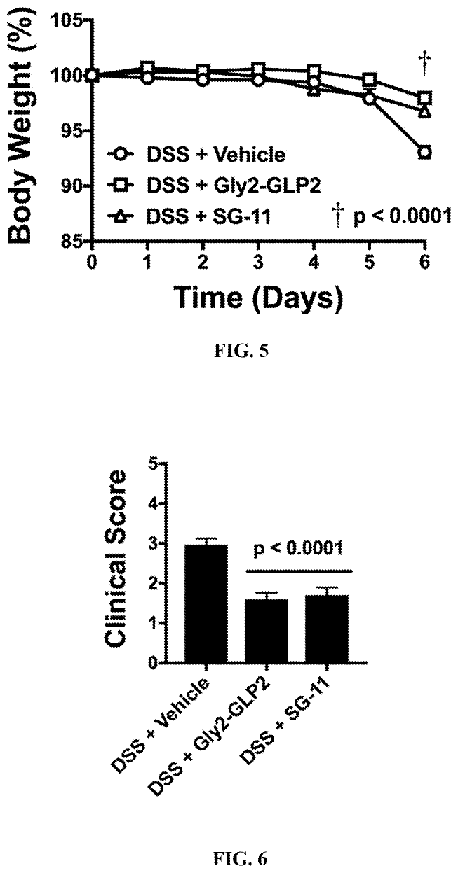

Body weight was measured daily from mice included in the DSS model described in this Example. Percent change from starting weight on day 0 was determined for each mouse. SG-11 administration to DSS treated mice significantly improved body weight as compared to vehicle treated DSS mice. Weight loss in mice treated with SG-11 at day 6 was similar to weight loss observed with Gly2-GLP2. Results are shown in FIG. 5. The graph in FIG. 5 represents data pooled from two independent experiments (n=20; each experiment using 10 mice).

### SG-11 Significantly Reduces Gross Pathology in a DSS Model of Inflammatory Bowel Disease

Gross pathology observations were made in mice included in the concurrent DSS model performed in this Example. SG-11 administration to DSS treated mice significantly improved gross pathology as compared to vehicle treated DSS mice. No differences in clinical scores were observed between mice given DSS and treated with either Gly2-GLP2 or SG-11. The scoring system used was: (0)=no gross pathology, (1)=streaks of blood visible in feces, (2)=completely bloody fecal pellets, (3) bloody fecal material visible in cecum, (4) bloody fecal material in cecum and loose stool, (5)=rectal bleeding. Results are shown in FIG. 6. The graph in FIG. 6 represents data pooled from 3 independent experiments (n=30; each experiment using 10 mice). These data show that SG-11 is therapeutically effective in improving symptoms of IBDs such as blood in the feces.

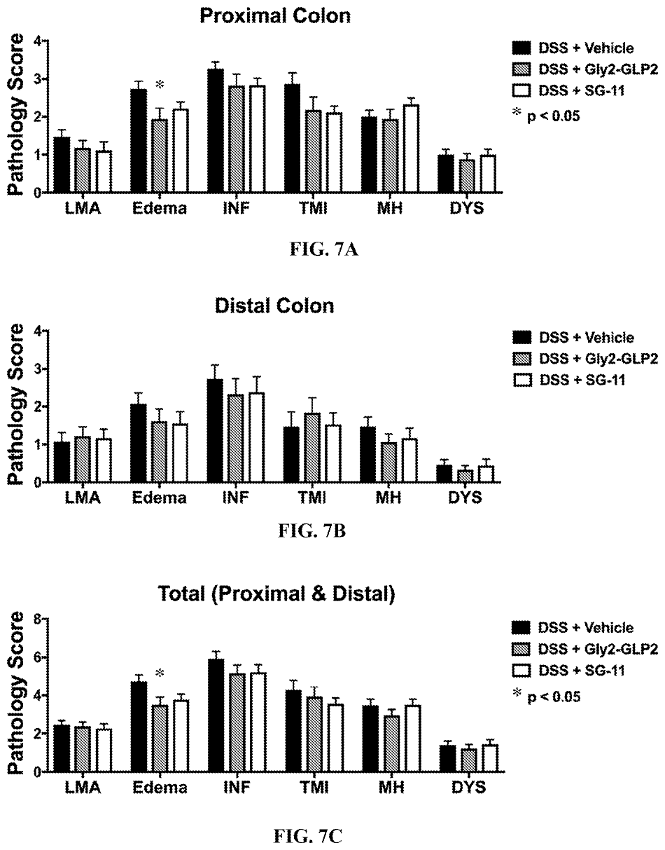

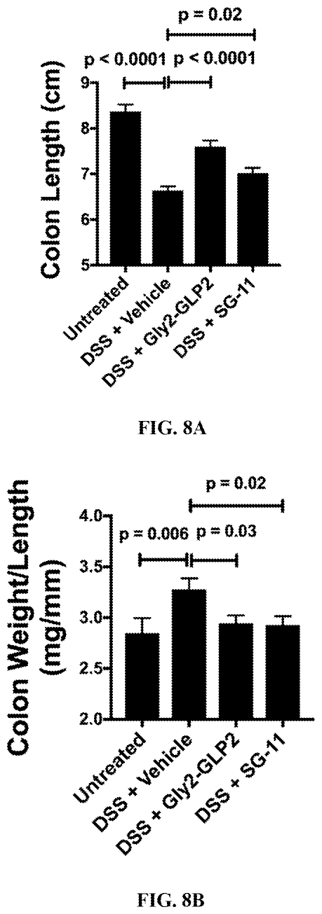

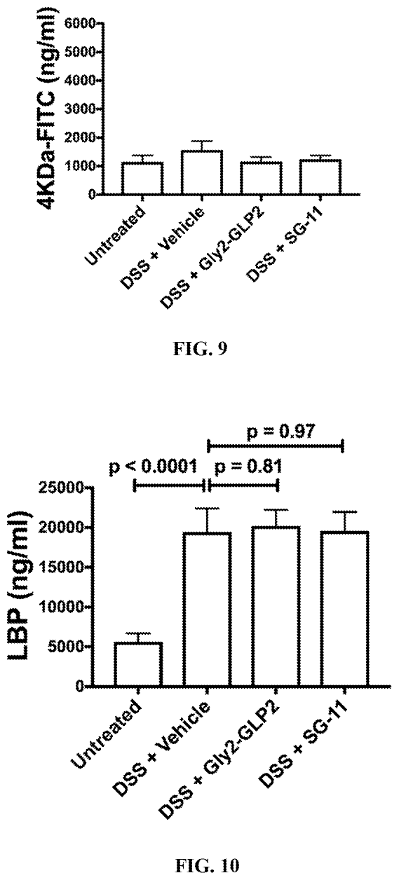

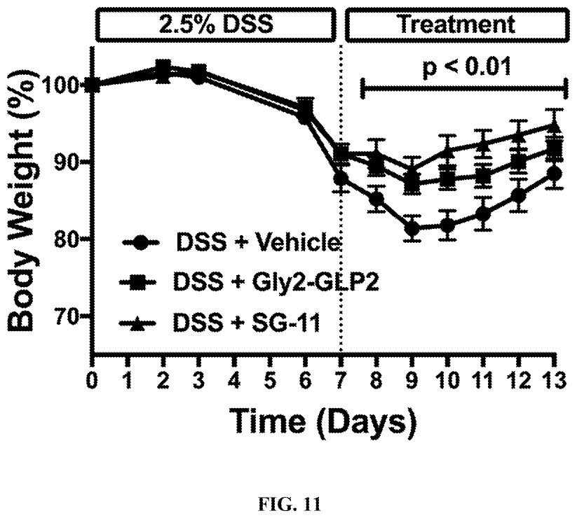

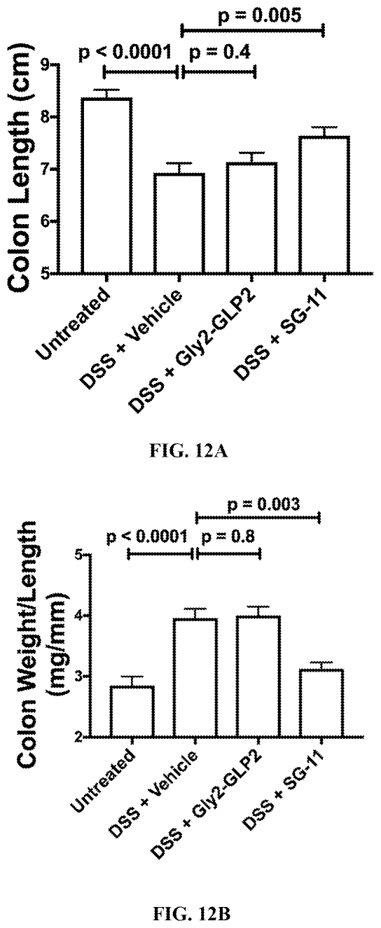

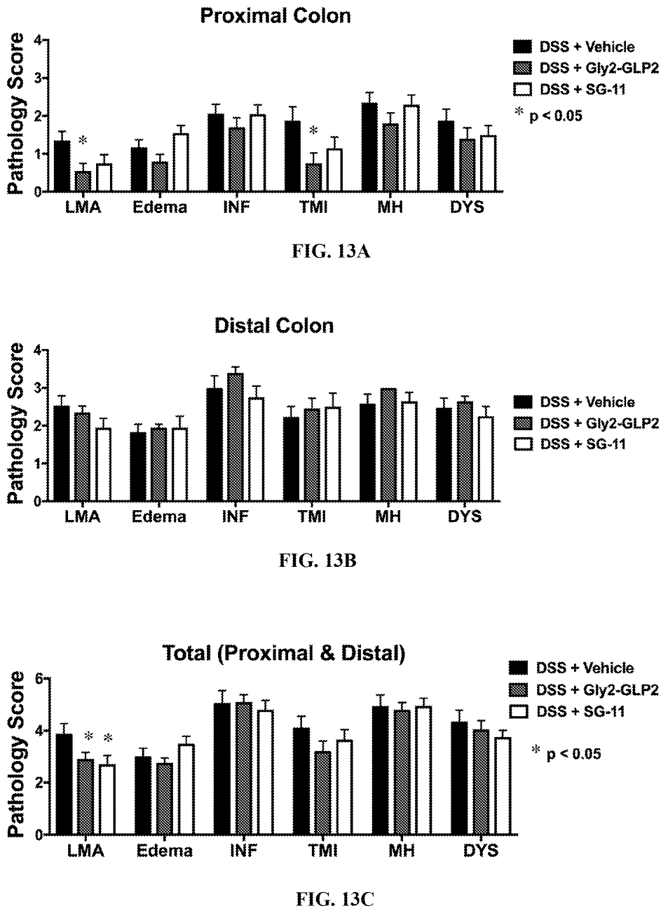

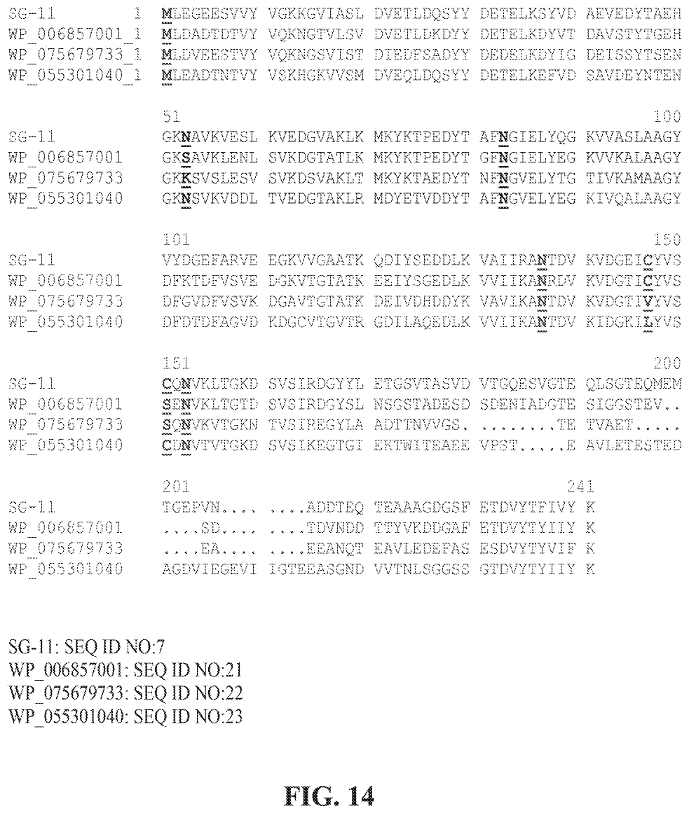

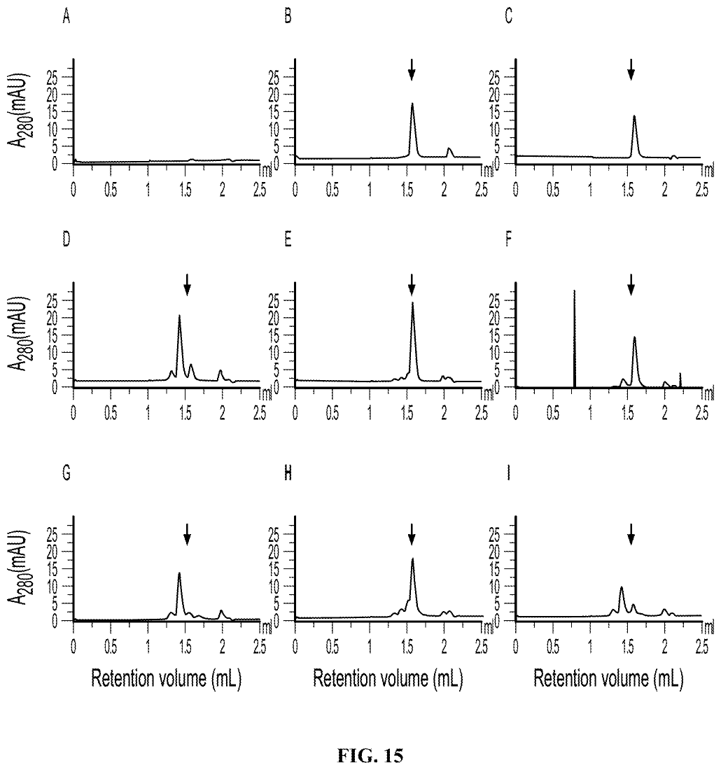

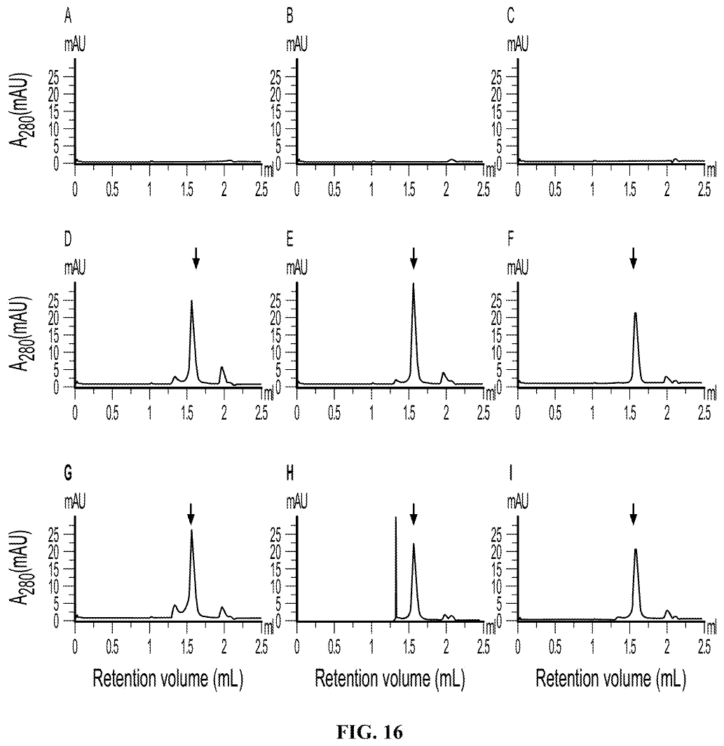

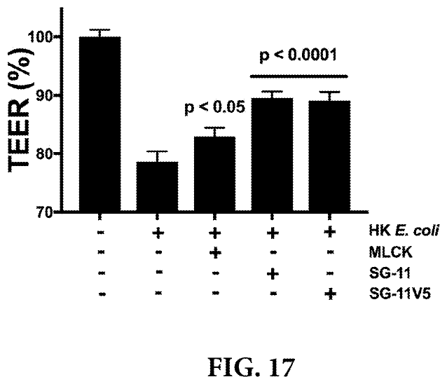

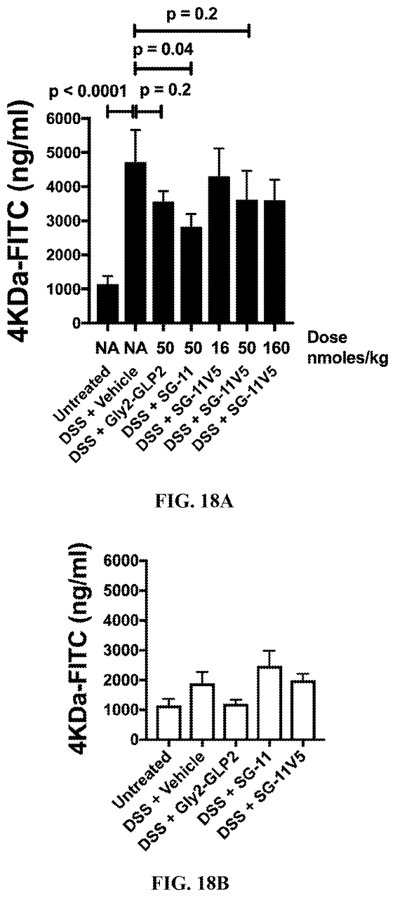

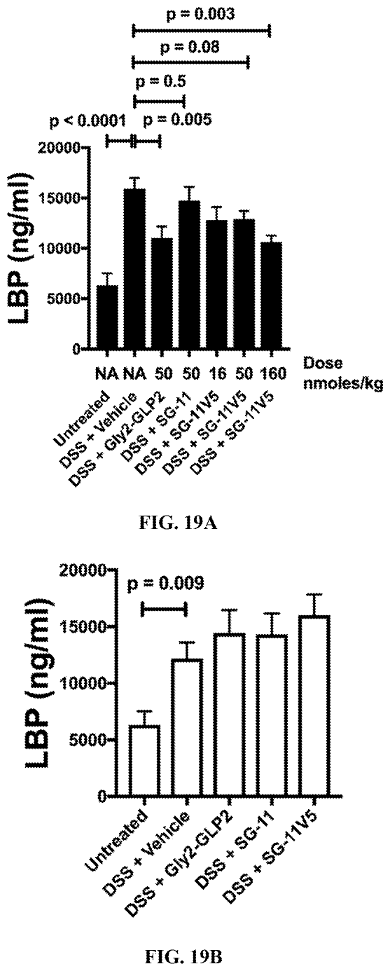

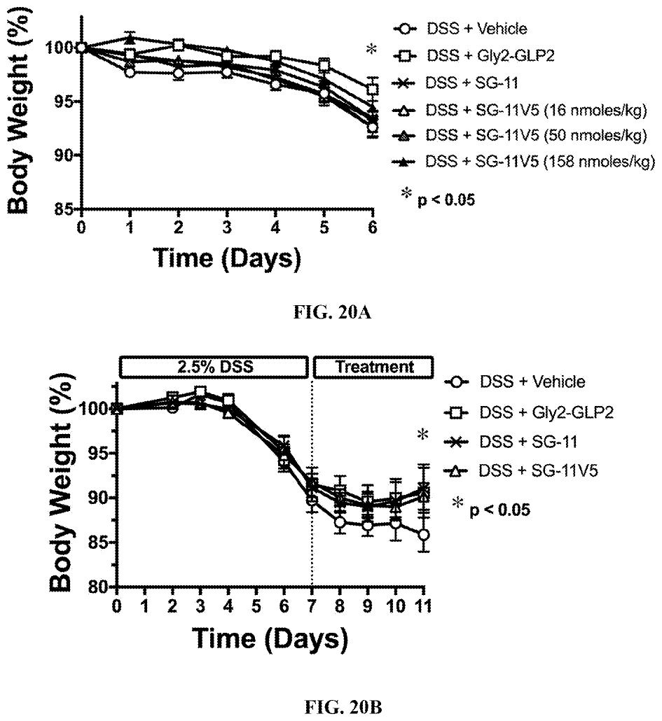

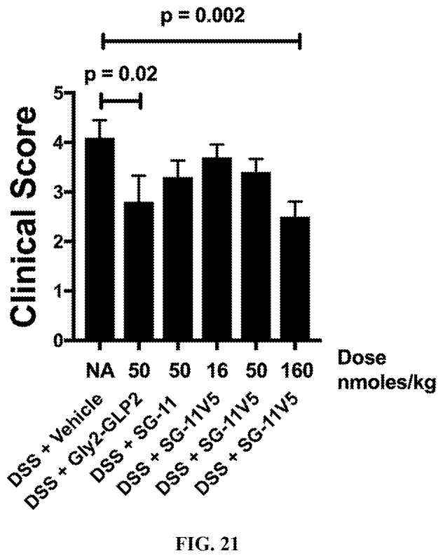

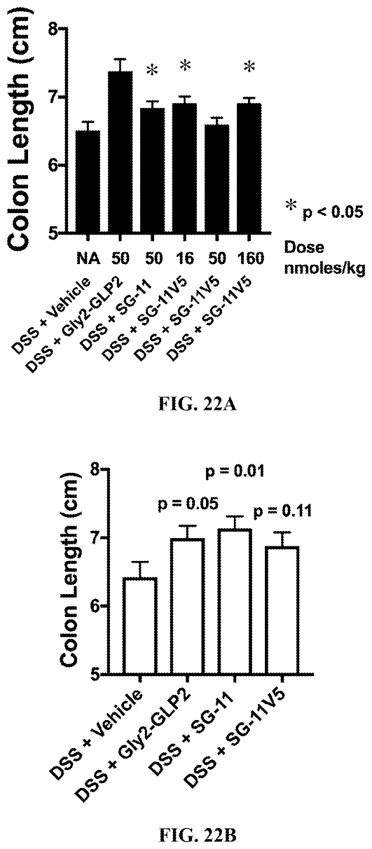

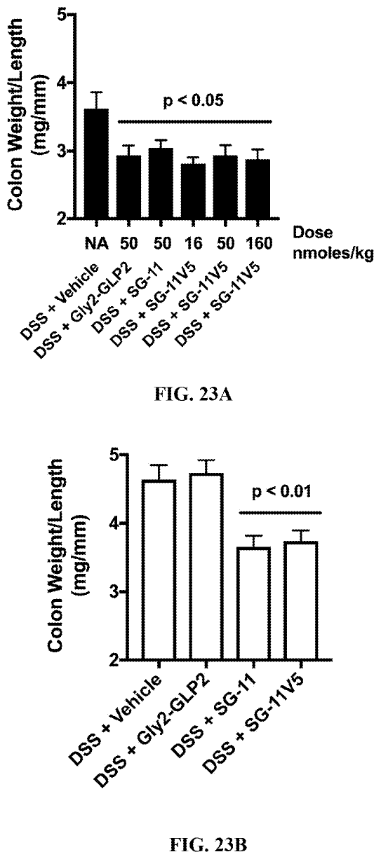

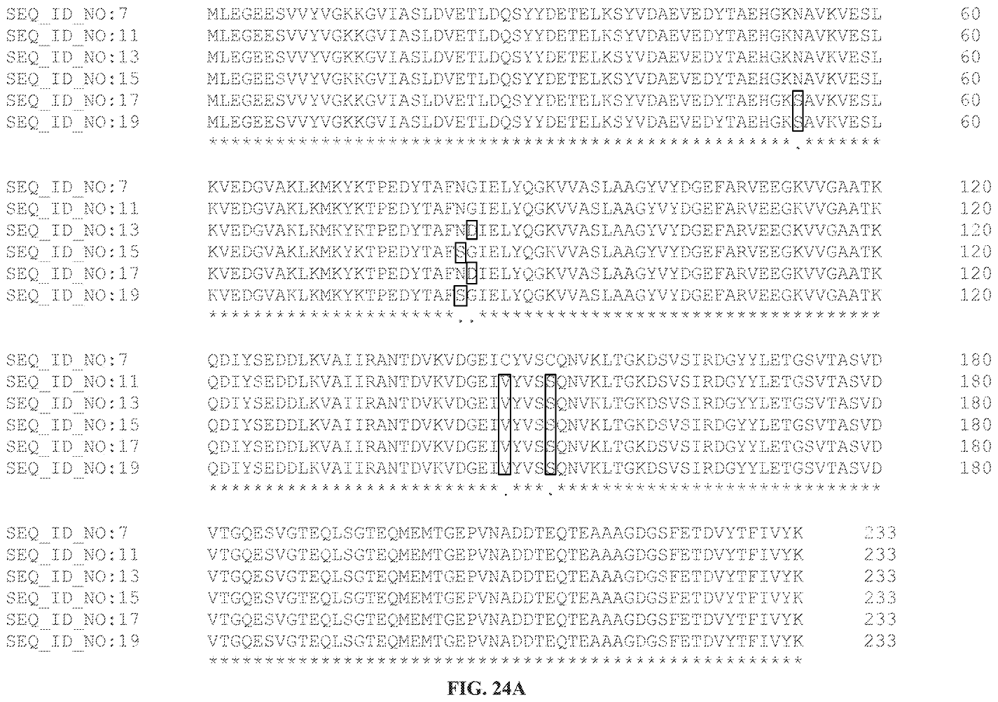

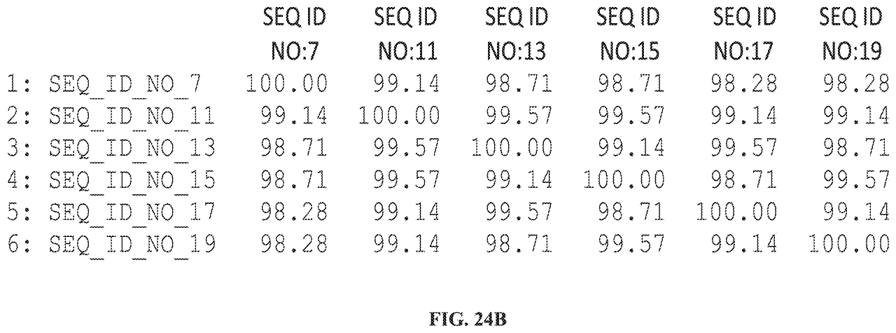

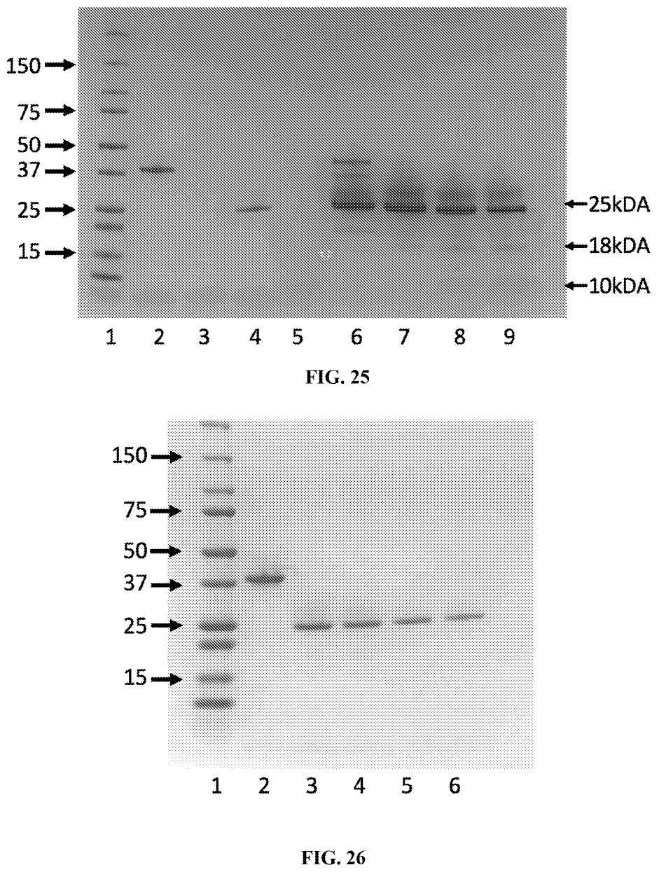

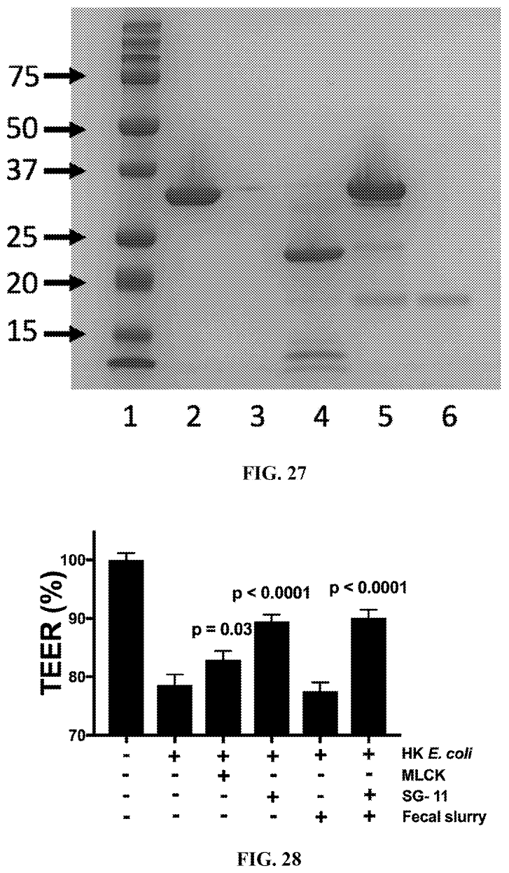

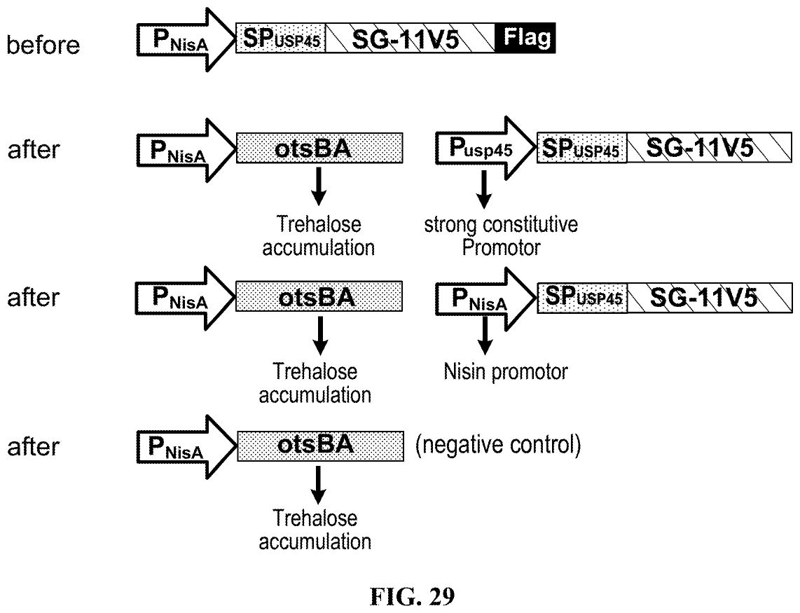

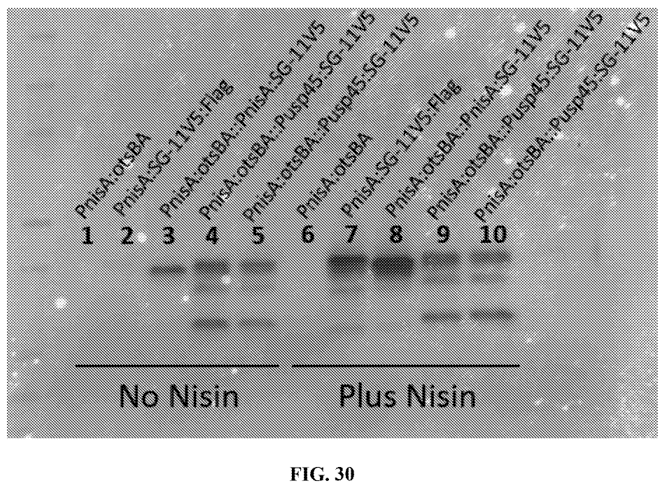

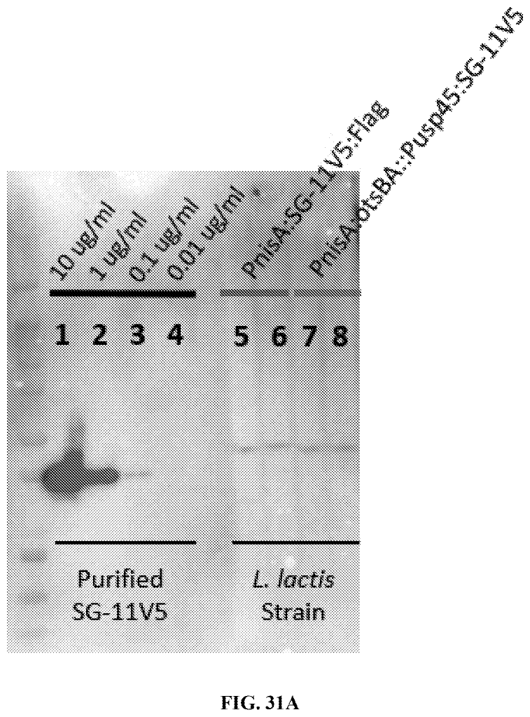

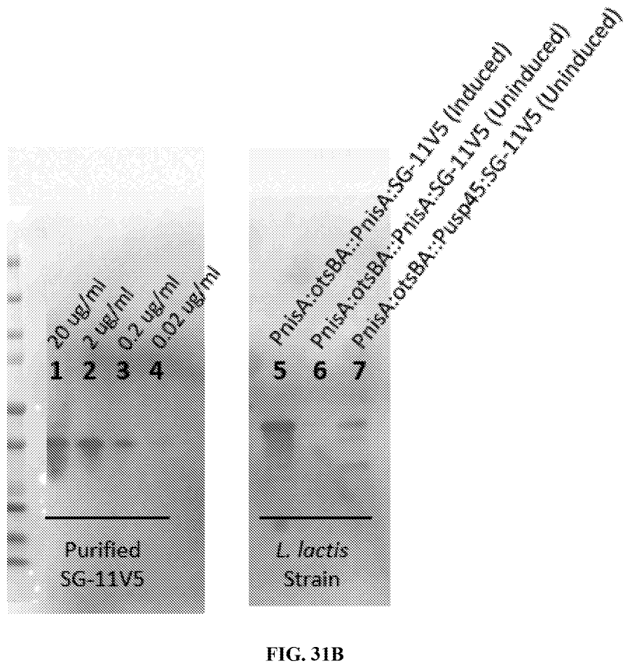

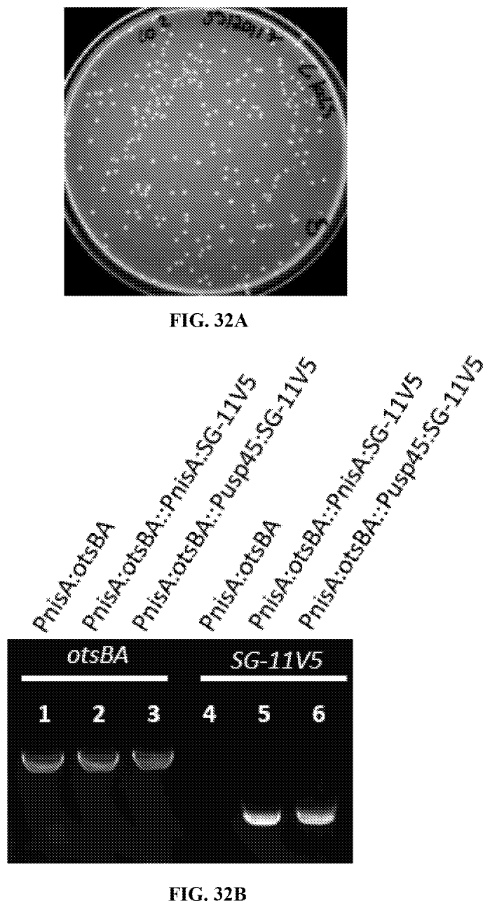

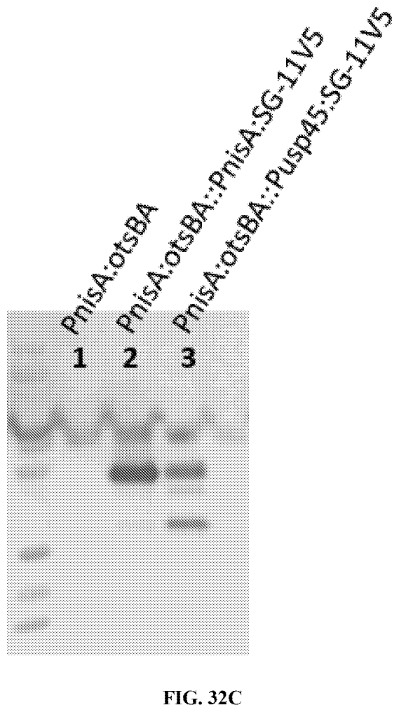

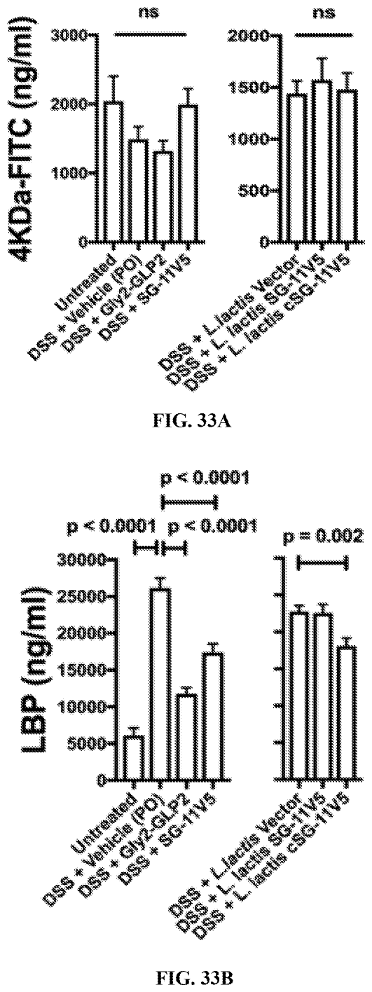

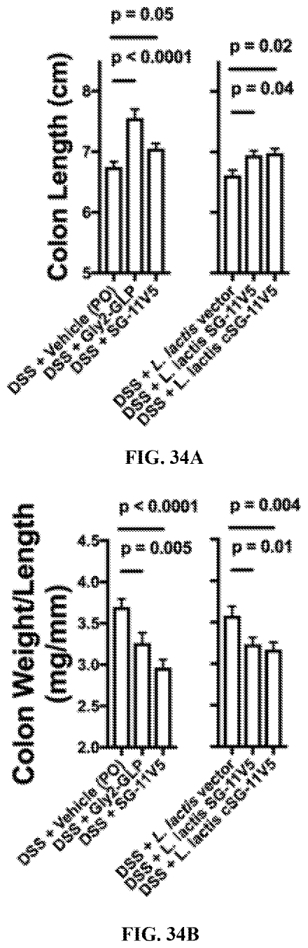

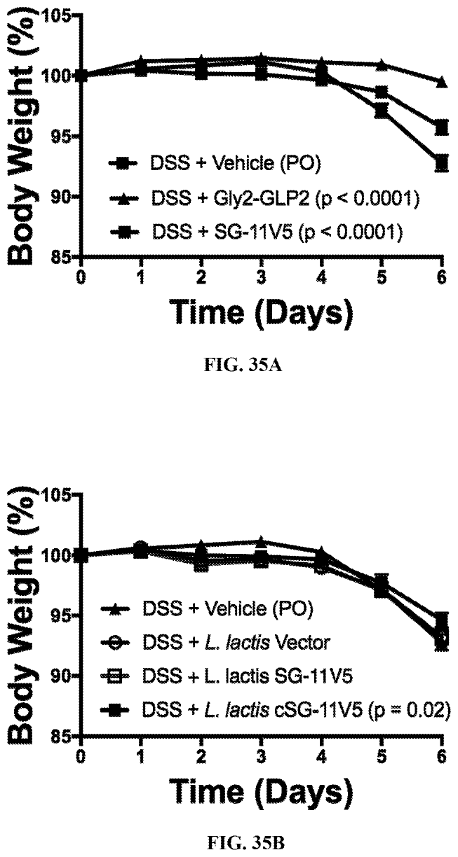

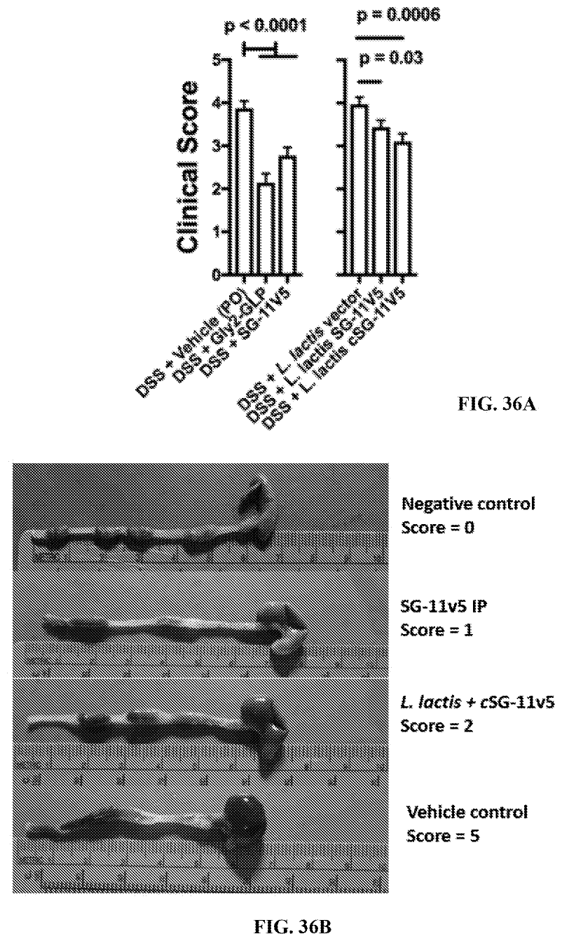

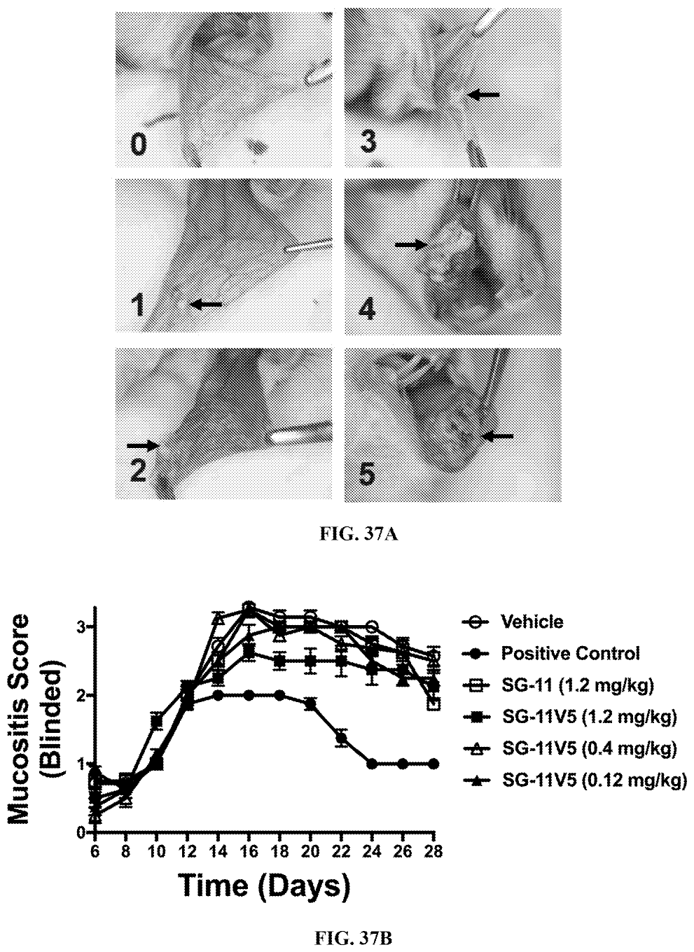

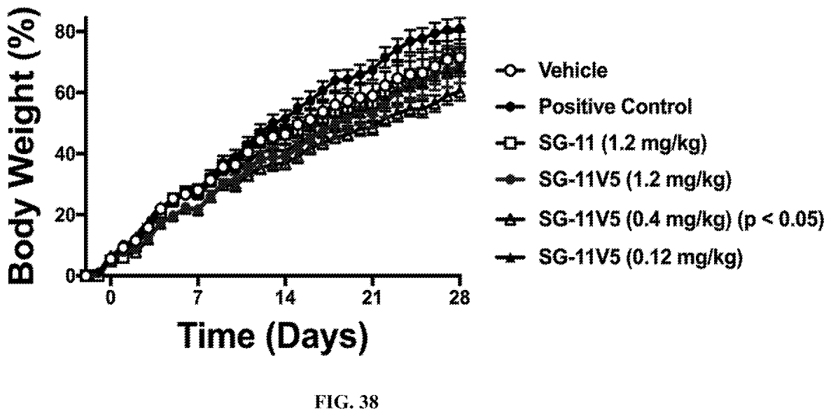

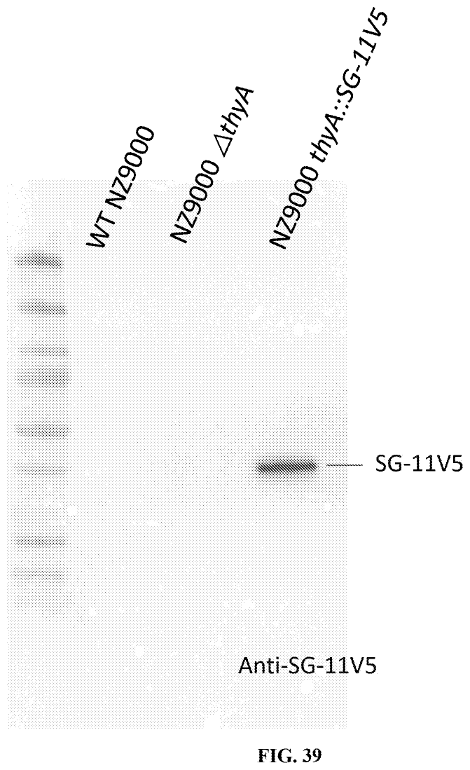

In addition, histopathology analysis was performed on proximal and distal colon tissues from the DSS model animals. Proximal (FIG. 7A) and distal (FIG. 7B) colon scores (range 0-4) are presented as well as the total score (FIG. 7C) for the colon which represents the sum of proximal and distal colon scores (scored on a scale of 0-8), SG-11 treatment reduced edema to a similar level as Gly2-GLPs, though the difference did not reach statistical significance. LMA Loss of mucosal architecture, Edema=Edema, INF=Inflammation. TMI=Transmural inflammation, MH=Mucosal hyperplasia, DYS=Dysplasia. Graphs represent data pooled from two independent experiments, and are plotted as mean±SEM. Statistical analysis was performed by a one-way ANOVA compared to DSS+vehicle followed by a Fisher's LSD test for multiple comparisons.

### SG-11 Minimizes the Colon Shortening Effect in Response to DSS Treatment

The following experiment demonstrates the therapeutic ability of a protein as disclosed herein to treat inflammatory bowel disease in an m vivo model, by showing an ability to prevent or minimum colon shortening.

Colon length was measured in mice included in the DSS model described above. SG-11 administration to DSS treated mice prevented colon shortening elicited by DSS. A significant improvement in colon length was observed with Gly2-GLP2 and Gly2-GLP2 treatment had a significant improvement over SG-11 treatment. Results am shown in FIG. 8A. Additionally, treatment of mice exposed to DSS with either Gly-2-GLP2 or SG-11 resulted in a significant improvement in colon weight to length ratios (FIG. 8B). The graphs in FIGS. 8A and 8B represent data pooled from 3 independent experiments (n=30). Data are graphed as mean SEM and are pooled from three independent experiments (n=30; each experiment using 10 mice). Statistical analysis was performed by a one-way ANOVA followed by a Fisher's LSD multiple comparisons test.

### Example 5

### SG-11 Demonstrates Therapeutic Activity in a DSS Model of Inflammatory Disease

In this example, experiments were performed to study the effects of SG-11 in the DSS mouse model when the SG-11 protein is administered to the mice after DSS treatment for 7 days. This differs from the treatment regimen of Example 4 in which mice were administered SG-11 protein shortly before treatment with DSS. This example further demonstrates the therapeutic ability of a protein as disclosed herein to treat inflammatory bowel disease in an in vivo model and is therefore a demonstration that the aforementioned in vitro models, which described important functional and possible mechanistic modes of action, will translate into an in vivo model system of inflammatory bowel disease.

Eight-week-old male C57BL/6 mice were housed 5 animals per cage and given food and water ad libitum for seven days. Following a 7-day acclimation period, the mice were provided with drinking water containing 2.5% DSS for 7 days. Fresh 2.5% DSS water was prepared every 2 days during the 7 day DSS administration. For this therapeutic DSS study, SG-11 used to treat the animals was fused at its N-terminus to a FLAG Tag (DYKDDDDK; SEQ ID NO:32).

On day 7, normal drinking water was restored and i.p. treatments of 50 nmole/kg of SG-11 (13 m/kg) or Gly2-GLP2 (0.2 mg/kg) were initiated. Treatments were administered twice a day (b.i.d.), with a morning and evening dose (every 8 and 16 hours) for six days.

As detailed below, results of the treatments were Analyzed with respect to animal health including body weight and gross pathology, histopathology of colon tissue, assessment of barrier disruption, and levels of LPS binding protein.

Body weight was measured daily during the morning treatment. The colon tissue was then harvested and length was measured in centimeters and the tissue was weighed. Fecal material was flushed from the colon and residual PBS removed by gently running the colon tissue through a pair of forceps. The colon tissue was then weighed and colon weight to length ratio in mg/mm was determined. Following weight measurements proximal and distal colon tissue was banked for RNA and protein analysis and the remaining tissues was fixed in 10% neutral buffered formalin for histopathology. Statistical analysis was performed by a one-way ANOVA compared to DSS+vehicle for serum 4KDa-FITC translocation, scrum LBP concentrations, colon length, and colon weight to length ratio, while a two-way ANOVA was performed for analysis of body weight. In all analysis, a Fisher's LSD test for multiple comparisons was used. Graphs represent data pooled from two experiments, and are plotted as mean±SEM.

This therapeutic model measured recovery of an established DSS insult. Because untreated mice also recover following removal of DSS from the drinking water, no increase in 4KDa-TC signal was observed following 6 days of DSS treatment (FIG. 9). Furthermore, no reduction in LBP was observed following Gly2-GLP2 or SG-11 treatment (FIG. 10). Therefore, no changes in barrier action readouts were observed in the therapeutic model of DSS.

Although no changes in barrier function readouts were observed in the therapeutic DSS model, significant improvements in clinical parameters such as body weight (FIG. 11), colon length (FIG. 12A), and colon weight to length (FIG. 12B) were observed. Similar to barrier readouts, the gross pathology scoring system based on bloody feces was no longer relevant as even DSS mice had recovered following 6 days of treatment. However, while there was no visible blood remaining in the colon, a thickened colon was still observed. From gross pathology observations, a reduction in the frequency of thick colons was observed with SG-11 treatment (88% in DSS+vehicle and 25% In DSS+SG-11, p<0.0001 by Fisher's Exact test data not shown).

Histopathology analysis was performed on proximal and distal colon tissues from the therapeutic DSS model described above. Proximal (FIG. 13A) and distal (FIG. 13B) colon scores (range 0-4) are presented as well as the total score for the colon which represents the sum of proximal and distal colon scores (Range 0-8) (FIG. 13C). LMA=Loss of mucosal architecture, Edema=Edema, INF=Inflammation, TMI=Transmural inflammation, MH=Mucosal hyperplasia, DYS=Dysplasia. Graphs represent data pooled from two independent experiments, and are plotted as mean±SEM. Statistical analysis was performed by a one-way ANOVA compared to DSS+vehicle followed by a Fisher's LSD test for multiple comparisons.

SG-11 and Gly2-GLP2 treatment resulted in a modest, but significant reduction in the loss of mucosal architecture score, with no change in inflammation and transmural inflammation scores. Similar to the results provided in Example 4, similar patterns of histopathology changes were observed with SG-11 and Gly2-GLP2, providing additional evidence that SG-11 may target epithelial cells.

### Example 6

### Design of Stable and Therapeutically Active SG-11 Variants

SG-11 Is a therapeutic protein derived from the commensal bacterium Roseburia hominis. Administration of R. hominis as a probiotic in the DSS model demonstrated efficacy with improvements in intestinal barrier function (4KDa-FITC and LBP), body weight, and clinical score (data not shown).

Recombinant production of a therapeutic protein can also be affected by post-translational modifications (PTMs) which may occur during large-scale expression and purification as well as during long-term storage. Such PTMs include but are not limited to oxidation of methionine, deamidation of asparagine and inter- and/or intra-molecular disulfide bonds between two cysteines. Accordingly, studies were performed to replace residues which may affect protein stability. These studies are described in Examples 6-11.

As a first step, the SG-11 amino acid sequence (SEQ ID NO:7) was aligned to similar prokaryotic proteins. The identified residues based on the search results can be used for the amino acid substitution for enhancing the stability of the therapeutic protein(s).

At first, a Blast search of the GenBank non-redundant protein database (NCBI BLAST/default parameters/BLOSUM62 matrix) was performed to identify other prokaryotic proteins that may be homologous to SG-11. The identified protein sequences are shown in FIG. 17. SEQ ID NO:21 is a hypothetical protein from Roseburia intestinalis (GenBank: WP_006857001.1; BLAST E value: 3e-90); SEQ ID NO:22 is a hypothetical protein from Roseburia sp. 831b (GenBank: WP_073679733.1; BLAST E value: 4e-58); and SEQ ID NO:23 is a hypothetical protein from Roseburia inulinivorans (GenBank: WP_055301040.1; BLAST E value: 1e-83).

Each of SEQ ID NO:21, SEQ ID NO22 and SEQ ID NO:23 is a predicted mature form of the indicated protein (lacks a signal peptide) and contains an N-terminal methionine. A multiple sequence alignment of these sequences with SG-11 (SEQ ID NO:7) was performed to identify regions conserved among the proteins. The alignment is shown in FIG. 14. The alignment was used to identify residues which were most conserved among the different proteins in order to assess the potential impact of substituting a particular amino acid(s). Portions of the SG-11 are somewhat or highly conserved in which an amino acid at a particular position in the protein is identical in all 4 of the aligned proteins or at least in 2 (positions) or 3 (positions) of the 4 proteins. The high sequence conservation among these homologs of SG-11 suggests that SEQ ID NO:21, SEQ ID NO:22 and SEQ ID NO:23 may also possess a function important in maintaining a healthy epithelial barrier.

### Pot-Translational Modification (PTM) Analysis of SG-11

Studies were performed to identify residues of SG-11 particularly susceptible to PTMs using LC/MS/MS. The analysis was performed by LakePharma (Belmont, Calif.) to 1) confirm the amino acid sequence of SG11 (SEQ ID NO-9), and 2) determine any post-translational modification which could lead to reduced biological activity and immunogenicity, particularly deamidation and oxidation.

For peptide mapping and PTM analysis, samples were treated with DTT and IAA, followed by trypsin digestion. The digested sample was then analyzed by Waters ACQUITY UPLC coupled to Xevo G2-XS QTOF mass spectrometer using a Protein BEH C18 column.

Peptide mapping and sequencing confirmed the predicted amino acid sequence and also indicated multiple deamidation sites and one oxidation site. Among them, 7.84% of N53 and 3.77% N83 is deamidated. These results presented in Table 7 indicate that N53 and N83 are primary sites of deamidation under non-stress conditions. N53 indicates Asparaginic (Asn; N) located at the 53th position in mature SG-11 with a methionine at the first position (SEQ ID NO:7).

TABLE 7
Post-Translation Modification of SG-11

| | SEQ | | | % | |
|---|---|---|---|---|---|
| Amino | ID | | | total | % |
| Acid¹ | NO | Peptide | Modifiers | ion² | peptide³ |
| N53 | 30 | NAVK | Deamidation | 0.01 | 7.84 |
| N83 | 26 | TPEDYTAFNG | Deamidation | 0.25 | 3.77 |
| | | IELYQGK | | | |
| N137 | 27 | ANTDVK | Deamidation | 0.25 | 1.03 |
| N153 | 28 | VDGEICYVSC | Deamidation | 0.01 | 0.2 |
| | | QNVK | | | |
| M1 | 24 | MLEGEESVVY | Oxidation | <0.01 | NA |
| | | VGK | | | |

¹Amino acid position in SG-11 (SEQ ID NO: 7)
²Normalized to total peptide ion intensity
³Normalized to the total intensity of corresponding precursor with or without modification

### Example 8

### Forced Degradation of SG-11

SG-11 (SEQ ID NO:9) was also tested under a series of stress conditions shown in Table 8 below to further characterized the stability of recombinant, purified SG-11. Stressed samples were analyzed either by SEC-HPLC for the presence of aggregates and/or degradants. LC/MS/MS was performed for determination of levels of deamidation and oxidation.

TABLE 8

| | | Analytic | | Observa- |
|---|---|---|---|---|
| Stress factor | | method | Criteria | tion |
| Temperature | 4° C. | HPLC | % monomer | Solution |
| (2 weeks) | | | (>90% pass) | clear |
| | 25° C. | HPLC | % monomer | Solution |
| | | | (>90% pass) | clear |
| | 37° C. | HPLC | % monomer | Solution |
| | | | | clear |
| | 40° C. | uPLC | % monomer | Solution |
| | | LC/MS/MS | | clear |
| Oxidation | Hydroperoxide | uPLC | % oxidation | Solution |
| | (0.005%) | LC/MS/MS | and sites | clear |
| | 40° C., 16 hr | | | |
| Mechanical | 350 rpm | HPLC | % monomer | Solution |
| stress | shake, 4° C., | | (>90% pass) | clear |
| | 24 hr | | | |
| pH | pH 4 and pH 9 | uPLC | % deamidation | Solution |
| | | LC/MS/MS | sites | clear |
| Freeze and | −80° C. | HPLC | % monomer | Solution |
| thaw (6 | to room | | | clear |
| cycles) | temperature | | | |

For this analysis, SG-11 (SEQ ID NO:9) was present at a concentration of 1 mg/ml in PBS (50 mM sodium phosphate, 150 mM NaCl, 10% glycerol, pH 8.0), with the exception of tests under pH 4 and pH 9. For pH 4, SG-11 (SEQ ID NO:9) was prepared at a concentration of 1 mg/ml in sodium acetate buffer (50 mM sodium acetate, 150 mM NaCl, pH 4). For pH 9, SG-11 (SEQ ID NO:9) was prepared at a concentration of 1 mg/ml in CAPSO) (3-cyclohexylamino-2-hydroxy-1-propanesulfonic acid) buffer (50 mM CAPSO, 150 mM NaCl, pH9).

The analysis shows that the SG-11 (SEQ ID NO:9) sample treated at 4° C. has a low level of aggregates. With increasing temperature, aggregation increased. At 37° C., major aggregation occurred. In contrast, mechanical stress and repeated freeze and thaw did not cause either protein aggregation or degradation.

Three samples treated by incubation at 40° C. for two weeks, oxidation (H₂O₂), or high pH 9, respectively, were analyzed by LC/MS/MS for PTMs. As shown in Table 9, significant deamidation of N83 occurred after sample treatment at 40° C. with almost 100% deamidation. Significant deamidation of N83 (37%) and oxidation of M200 (63.9%) were observed in samples treated with hydrogen Peroxide. 7.84% of N53 was deamidated without any treatment.

TABLE 9

| | | % | | | |
|---|---|---|---|---|---|
| | | | | | No |
| Peptides | Modification¹ | 40° C. | Oxidation | pH 9 | treatment |
| MLEGEESVVYVGK | No modification | 99.82 | 99.88 | 99.94 | 99.78 |
| (SEQ ID NO: 24) | Oxidation of M | 0.18 | 0.12 | 0.61 | 0.22 |
| GVIASLDVETLDQSYYDETELK | No modification | 99.91 | 99.93 | 99.95 | 100 |
| (SEQ ID NO: 25) | Deamidation Q30 | 0.09 | 0.07 | 0.05 | 0 |
| TPEDYTAFNGIELYQGX | No modification | 0 | 63 | 80.9 | 82.57 |
| (SEQ ID NO: 26) | Deamidation N83 | 99.88 | 37 | 19.1 | 17.43 |
| | Deamidation N83 | 0.12 | 0 | 0 | 0 |
| | Deamidation Q89 | | | | |
| ANTDVK | No modification | 85.39 | 83.56 | 76.05 | 98.96 |
| (SEQ ID NO: 27) | Deamidation N137 | 14.61 | 16.44 | 23.95 | 1.04 |
| VDGEICYVSCQNVK | No modification | 44.85 | 63.23 | 8.76 | 99.98 |
| (SEQ ID NO: 18) | Carbamidomethy | 33.59 | 34.52 | 71.71 | NA |
| | C147 | | | | |
| | Carbamidomethy | 18.45 | 1.93 | 12.45 | NA |
| | C151 | | | | |
| | Deamidation | 0 | 0 | 0.35 | 0.02 |
| | N153 | | | | |
| | Deamidation | | | | |
| | Q152 | 3.1 | 0.31 | 28 | 0 |
| | Deamidation | | | | |
| | N153 | | | | |
| | Carbamidomethy | | | | |
| | C151 | | | | |
| GYYLETGSVTASVDVTGQESVGTE | No modification | 96.38 | 19.17 | 88.16 | 100 |
| QLSGTEQMEMTGEPVNADDTEQT | Deamdation | 3.62 | 0 | 11.84 | 0 |
| EAAAGDGSFETDVYTFIVYK | N206 | | | | |
| (SEQ ID NO: 29) | Deamidation | | | | |
| | Q152 | | | | |
| | Oxidation M | | | | |
| | Oxidation M198 | 0 | 16.92 | 0 | 0 |
| | Oxidation M2.00 | 0 | 63.9 | 0 | 0 |
| NAVK | Deamidation N53 | NA | NA | NA | 7.84 |
| (SEQ ID NO 30) | | | | | |

¹Amino acid position in SG-11 (SEQ ID NO: 7)

After reduction, free cysteines were artificially carbamidomethylated by iodoacetamide to block cysteine residues from oxidation in the assays.

### Example 9

### Cysteine Residues and the Stability of SG-11

The stability of SG-11 (SEQ ID NO:9) was evaluated following the incubation at 37° C. for one week and at 4° C. for 3 weeks in Buffer C (100 mM sodium phosphate, pH 7.0, 0.5 M sorbitol). The stability was assessed by monitoring aggregation formation with analytical size exclusion chromatography (SEC) equilibrated with Buffer D (100 mM sodium phosphate, pH 7.0, 10% glycerol). No noticeable change was observed after 3 weeks of storage at 4° C. compared with the freshly thawed protein, as both samples showed a single peak at 1.57 mL. However, after a one-week incubation at 37° C., the sample clearly showed aggregation peaks at 1.29 and 1.41 ml in addition to a monomer peak at 1.57 mL, which was the smallest peak. The cause of the aggregation was investigated as follows. There are two cysteine residues found in SG-11 at positions 147 and 151 (relative to SEQ ID NO:7). Ellman's reagent assay revealed the presence of free sulfhydryl groups in SG-11 (SEQ ID NO:9), which indicated Cys¹⁴⁷ and/or Cys¹⁵¹ does not form stable disulfide bonds. As free sulphydryl groups could cause aggregation by forming unpreferable intermolecular disulfide bonds, it was examined whether the presence of reducing agent, such as β-mercaptoethanol, could prevent the aggregation. Aggregation was greatly suppressed in the presence of 2.5% (v/v) β-mercaptoethanol in a buffer (50 mM sodium phosphate, 150 mM NaCl and 10% glycerol) following the 4-days incubation at 37° C., in contrast to the aggregations that were formed without β-mercaptoethanol. The results suggested that Cys¹⁴⁷ and/or Cys¹⁵¹ provides free sulfhydryl groups that caused aggregation.

### Example 10

### Post-Translational Modification of an SG-11 Variant

Although SG-11 protein is stable at high temperature, forming aggregations at 37° C. in a week could be the problem at the downstream processing stage. Deamidation of asparagine residues found by LC/MS/MS are also a risk factor. In order to improve the manufacturability of a protein comprising SEQ ID NO-3 or variants thereof, the results of Examples 10 to 12 were considered in the design of SG-11 variants (e.g., SG-11V1 (SEQ ID NO:11). SG-11V2 (SEQ ID NO:13), SG-11V3 (SEQ ID NO:15), SG-11V4 (SEQ ID NO:17) and SG-11V5 (SEQ ID NO:19)) to reduce incidence of detrimental PTMs.

Examples 13-16 describe experiments performed to characterize the effects of amino acid substitutions on stability and function of the SG-11 variant SG-11V5 (SEQ ID NO:19, comprising N53S, N83S, C147V, C151S with respect to SEQ ID NO:7). SG-11V5 (expressed and purified as described in Example 1).

In accordance with PTMs observed when SG-11 (SEQ ID NO:9) was subjected to stress conditions (Example 11), SG-11V5 (SEQ ID NO:19) was analyzed by LC-MS/MS for post translational modifications using the methods described in Example 11, and compared with PTMs for SG-11 (SEQ ID NO:7)

For this analysis, PTMs of wildtype SG-11 (SEQ ID NO:7) and SG-11V5 (SEQ ID NO:19) were compared. In the first analysis (results provided in Table 10 below), the proteins were stored at a concentration of 1 mg/ml in Buffer 1 (50 mM NaPO₄, pH 8, 150 mM NaCl, 10% glycerol) and stored for 2 weeks at either 4° C. or 40° C. The proteins were then treated with DTT and Iodoacetamide (IAA), followed by trypsin digestion. The digested samples were then analyzed by Waters ACQUITY UPLC couples to Xevo G2-XS QTOF mass spectrometer using a Protein BEH C18 column. Analysis of the proteins by LC-MS/MS showed that the SG11V5 protein had significantly lower percentages of oxidation of the start methionine and deamidation of N137 as compared to SG-11 at both 4° C. and 40° C.

TABLE 10

| Protein | PTM site | Mod | 4° C. | 40° C. |
|---|---|---|---|---|
| SG-11 | MLEGEESVVYVGK | Oxidation | 8.9% | 12.5% |
| (SEQ ID NO: 7) | (SEQ ID NO: 26) | of M1 | | |
| SG-11V5 | MLEGEESVVYVGK | Oxidation | 2.5% | 4.7% |
| (SEQ ID NO: 19) | (SEQ ID NO: 26) | of M1 | | |
| SG-11 | TPEDYTAENGIELYQGK | Deamidation | 19.4% | 98.1% |
| (SEQ ID NO: 7) | (SEQ ID NO: 26) | of N83 | | |
| SG-11V5 | TPEDYTAESGIELYQGK | Deamidation | — | — |
| (SEQ ID NO: 19) | (SEQ ID NO: 28) | of N83 | | |
| SG-11 | ANTDVK | Deamidation | 1.0% | 0.9% |
| (SEQ ID NO: 7) | (SEQ ID NO: 24) | of N137 | | |
| SG-11V5 | ANTOVK | Deamidation | 0.1% | 0.3% |
| (SEQ ID NO: 9) | (SEQ ID NO: 24) | of N137 | | |

In a second analysis, the SG-11 (SEQ ID NO:7) and SG-11V5 (SEQ ID NO:19) proteins were each stored at 40° C. in a variety of buffers. The results are provided in Table 11 below. The storage buffer used in this experiment was 100 mM NaPO₄, pH7, with 10% sorbitol (+Sor) or without 10% sorbitol (−Sor) and with 10% glycerol (+Gly) or without 10% glycerol (−Gly) as indicated in Table 11. As the data in Table 11 demonstrate, there was a large decrease in oxidation of the methionine in the first position for the SG-11V5 (SEQ ID NO:19) protein as compared to the SG-11 (SEQ ID NO:7) protein in all buffer conditions. There were also differences in levels of N137 deamidation for the two proteins with the presence of at least glycerol and also the presence of both sorbitol and glycerol resulting in large decreases in N137 deamidation. These data show that substitution of amino acids in the SG-11 protein can have significant beneficial effects on PTMs of the protein in a solution.

TABLE 11

| | | | − Sor | + Sor | − Sor | + Sor |
|---|---|---|---|---|---|---|
| Protein | PTM site | Modification | − Gly | − Gly | − Gly | − Sly |
| SG-11 | MLEGEESVVYVGX | Oxidation of | 20% | 12.1% | 38.6% | 27.8% |
| (SEQ ID | (SEQ ID NO: 26) | M1 | | | | |
| NO: 7) | | | | | | |
| SG-11V5 | MLEGEESVVYVGK | Oxidation of | 2.7% | 3.3% | 5.6% | 5.9% |
| (SEQ ID | (SEQ ID NO: 26) | M1 | | | | |
| No: 19) | | | | | | |
| SG-11 | TPEDYTAFNGIELYQGK | Deamidation | 98.1% | 93.9% | 96.2% | 87.4% |
| (SEQ ID | (SEQ ID NO: 26) | of N83 | | | | |
| NO: 7) | | | | | | |
| SG-11V5 | TPEDYTAFSGIELYQGK | Deamidation | — | — | — | — |
| (SEQ ID | (SEQ ID NO: 28) | of N83 | | | | |
| NO: 19) | | | | | | |
| SG-11 | ANTDVK | Deamidation | 0.9% | 2.6% | 3.0% | 2.5% |
| (SEQ ID | (SEQ ID NO: 24) | of N137 | | | | |
| NO: 7) | | | | | | |
| SG-11V5 | ANTDVK | Deamidation | 1.5% | 3.3% | 0.4% | 0.1% |
| (SEQ ID | (SEQ ID NO: 24) | of N137 | | | | |
| NO: 19) | | | | | | |

### Example 11

### SG-11 Variant Construction and Stability Analysis

Although SG-11 protein is very stable at high temperature, forming aggregations at 37° C. in a week could be a problem at the downstream processing stage. Deamidation of asparagine residues found by LC/MS/MS we also a risk factor. In order to improve the manufacturability of a protein comprising SEQ ID NO:3 or variants thereof, the protein depicted as SG-11 (SEQ ID NO:7) was mutated to contain the following 4 substitutions: N53S, N83S, C147V and C151S. This variant with 4 substitutions is designated as SG-11V5, provided herein as SEQ ID NO:19. The stability of purified SG-11 and SG-11V5 was tested in different storage buffer formulations. SG-11V5 (SEQ ID NO:19) has about 98.3% sequence identity to SEQ ID NO:7.

### Stability Analysis of SG-11

FIG. 15A-15I shows effects of conditions on SG-11 (SEQ ID NO:7) stability. Specifically, purified SG-11 (SEQ ID NO:7) was incubated in pH 5.2 (FIGS. 15A, 15B and 15C), pH 7.0 (FIGS. 15D, 15E and 15F) and pH 8.0 (FIGS. 15G, 15H and 15I). Effect of additives was also tested at the 3 different pH conditions: 150 mM NaCl (FIGS. 15A, 15D and 15G); 150 mM NaCl and 100 mM arginine (FIGS. 15B, 15E and 15H): and 150 mM NaCl and 0.5 M sorbitol (FIGS. 15C, 15F and 15I). Stability was analyzed by analytical SEC. Arrow heads indicate the retention time of the monomeric form.

### Stability Analysis of SG-11V5

FIG. 16A-16I shows effects of conditions on SG-11V5 (SEQ ID NO-19) stability. SG-11V5 (SEQ ID NO:16) was incubated in pH 5.2 (FIGS. 16A, 16B and 16C), pH 7.0 (FIGS. 16D, 16E and 16F) and pH 8.0 (FIGS. 16G, 16H and 16I). Effect of additives was also tested at the 3 different pH conditions: 150 mM NaCl (FIGS. 16A, 16D and 16G); 150 mM NaCl and 100 mM Arg (FIGS. 16B, 16E and 16H); and 150 mM NaCl and 0.5 M sorbitol (FIGS. 16C, 16F and 16I). Stability was analyzed by analytical SEC. Arrow heads indicate the retention time of the monomeric form.

In the presence of 100 mM arginine at pH 7.0, aggregate formation of the purified SG-11 (SEQ ID NO:7) protein was greatly suppressed. However, some small peaks were observed at an earlier retention time, which indicated there were different forms other than the monomeric form. SG-11V5 (SEQ ID NO:19) did not show a large amount of aggregation under all conditions tested in this example. Even without any additives, the discrete monomeric peak was observed. The small aggregation peak at 1.34 mL were suppressed by 100 mM arginine or 0.5 M sorbitol. The purified SG-11 (SEQ ID NO:7) and SG-11V5 (SEQ ID NO:19) were precipitated at pH 5.2.

Elevated temperature can increase protein degradation and aggregation, while also enhancing susceptibility to deamidation. To minimize potential liabilities associated with deamidation and aggregation, the mutations N53S, N83S C147V and C151S were introduced into in SG-11. Thus, SG-11V5 showed improved stability at the pH 7.0 and pH 8.0.

### Example 12

In vitro functional analysis of SG-11V5

An in vitro TEER assay was performed to demonstrate that SG-11 variants, e.g., SG-11V5, maintain functionality related to maintenance of epithelial barrier function as shown for SG-11 proteins (see. e.g., Example 2).

Cell culture was performed as described in Example 2. Briefly, following 8-10 days of culture, the transwell plate containing enterocytes wore treated with 10 ng/ml IFN-© added to the basolateral chamber of the transwell plate for 24 hours at 37° C.+5% CO₂. After 24 hours, fresh cRPMI was added to the epithelial cell culture plate. TEER readings were measured after the IFN-© treatment and were used as the pre-treatment TEER values. SG-11 (SEQ ID NO:9) or SG-11V5 (SEQ ID NO:19) was then added to the apical chamber of the transwell plate at a final concentration of 1 [symbol] g/ml (40 nM). The MLCK inhibitor peptide 18 (BioTechne, Minneapolis, Minn.) was used at 50 nM as a positive control to prevent inflammation induced barrier disruption (Zolotarevskky et al., 2002. Gastroenterology, 123:163-172). Compounds were incubated on enterocytes for 6 hours. Following pre-incubation with test compounds, the transwell insert containing the enterocytes was transferred on top of the receiver plate containing U937 monocytes. Heat killed E. coli (HK E. coli) (bacteria heated to 80° C. for 40 minutes) was then added to both the apical and basolateral chambers and a multiplicity of infection (MOI) of 10. Transwell plates were incubated at 37° C.+5% CO₂ for 24 hours and a post treatment TEER measurement was made. SG-11 (SEQ ID NO:9) increased TEER from 78.6% disruption by HK E. coli to 89.5% (p<0.0001), while SG-11V5 (SEQ ID NO:19) increased to 89.1% (p<0.0001) (FIG. 17). Statistical analysis was performed using a one-way ANOVA compared to HK E. coli followed by a Fisher's LSD multiple comparison test. The graphs in FIG. 17 represent data pooled from four plates performed in two individual experiments (n=12).

### Example 13

### In Vivo Functional Analysis of SG-11V5

Next, the DSS animal model experiments performed as described above in Examples 4 and 5 were repeated to test SG-11 or SG-11V5 (SEQ ID NO-19) in parallel. In these experiments, SG-11 or SG-11V5 was administered to a mouse concurrent with the initiation of treatment with DSS (as in Example 4) or after prior DSS administration. The only difference is that mice in Example 5 were treated with SG-11 or SG-11V5 (SEQ ID NO:19) for 4 days rather than 6 days.

Briefly, in the first DSS mouse model (Example 13A), mice were treated on day zero with test compound intraperitoneally (i.p.) and 6 hours later DSS treatment was initiated. Doses administered included 50 nmoles/kg for SG-11 (SEQ ID NO:9) (1.3 mg/ml), and Gly2-GLP2 (0.2 mg/kg), and a dose response for SG-11V5 (SEQ ID NO:19) including 16 nmoles/kg (0.4 mg/m), 50 nmoles/kg (1.3 mg/ml) and 158 nmoles/kg (4.0 mg/kg). The mice were treated with 2.5% DSS in their drinking water for 6 days (day zero through day 6). Therapeutic protein treatments were administered twice a day for the duration of the DSS exposure.

In the second experiment (Example 13B), mice were provided with drinking water containing 2.5% DSS for 7 days. On day 7, normal drinking water was restored and i.p. treatments of 50 mmole/kg of SG-11 (SEQ ID NO:9)(1.3 mg/kg), SG-11V5 (SEQ ID NO:19) (1.3 mg/kg), or Gly2-GLP2 (0.2 mg/kg) were initiated. Treatments were administered twice a day (b.i.d.), with a morning and evening dose (every 8 and 16 hours) for 4 days. For both the prophylactic and therapeutic models, fresh 2.5% DSS water was prepared every 2 days during the DSS administration.

At the end of each DSS model study, mice were fasted for 4 hours and then orally gavaged with 600 mg/kg 4KDa dextran labeled with FITC [4KDa-FITC]. One hour after the 4KDa-FITC gavage, mice were euthanized, blood was collected, and FITC signal was measured in serum. For the first model, a significant increase in 4KDa-FITC dextran translocation across the epithelial barrier was observed in vehicle treated DSS mice as compared to untreated mice. The results are illustrated in FIG. 18A: SG-11 (SEQ ID NO:9) significantly reduced the 4KDa-FITC signal (p=0.04), and in FIG. 18B: SG-11V5 (SEQ ID NO:19) also reduced the 4KDa-FITC signal, although the difference did not reach statistical significance (p=0.21). Data in both graphs are plotted as mean±SEM and each figure represent data from an individual experiment (n=10 per group).

### Effects of SG-11V5 on Inflammation Centric Readouts of Barrier Function in a DSS Model of Inflammatory Bowel Disease

Upon completion of the OSS models above. LBP levels were measured as an inflammation centric readout of barrier function following the protocol detailed in Example 5. Upon completion of both DSS models (Examples 13A and 13B), blood was collected and serum was isolated. LPS binding protein (LBP) levels were measured in serum using a commercially available ELISA Kit (Enzo Lift Sciences). Results are provided in FIG. 19A and FIG. 195. A significant increase in LBP was observed in the Example 13A DSS model in response to DSS exposure. At the 50 nmoles/g dose of SG-11 (SEQ ID NO:9) and SG-11V5 (SEQ ID NO:19), similar reductions in LBP were observed although neither were statistically significant. However, SG-11V5 (SEQ ID NO:19) treatment at a higher dose of 158 nmoles/kg resulted in a significant reduction in LBP production (p=0.003) (FIG. 19A). In the Example 13B DSS model, exposure to DSS resulted in a significant increase in LBP production (FIG. 19B). However, no reduction in LBP was observed for any of the treatments and similar effects were observed for both SG-11 (SEQ ID NO:9) and SG-11V5 (SEQ ID NO:19).

### Effects of SG-11 and SG-11V5 on Body Weight in a DSS Model of Inflammatory Bowel Disease

Body weight was measured throughout the experimental models in both Example 13A and Example 13B. In the Example 13A DSS model (FIG. 20A) similar trends in body weight were observed for SG-11 (SEQ ID NO:9) and SG-11V5 (SEQ ID NO:19) treatments at 50 nmoles/kg, and a significant improvement in body weight was observed at day 6 for SG-11V5 (SEQ ID NO:19) at 158 nmoles/kg. Similar patterns were observed in the therapeutic DSS model where SG-11 (SEQ ID NO-9) and SG-11V5 (SEQ ID NO:19) at the 50 nmoles/kg dose had similar changes in body weight with both having statistically improved body weight changes at day 11 (p<0.05). For FIG. 20A and FIG. 20B, data are graphed as mean±SEM and each graph represent data from an individual experiment. Statistical analysis was performed using a two-way ANOVA as compared to the DSS+vehicle group with a Fisher's LSD multiple comparison test.

### Effects of SG-11 and SG-11V5 on Gross Pathology in a DSS Model of Inflammatory Bowel Disease

Gross pathology observations of colon tissue were made as described in Example 7. Briefly, a scoring system based on the level of visible blood and fecal pellet consistency was used. The scoring system used was: (0)=no gross pathology, (1)=streaks of blood visible in feces, (2)=completely bloody fecal pellets, (3) bloody fecal material visible in cecum, (4) bloody fecal material in cecum and loose stool, (5)=rectal bleeding. Similar results were obtained for SG-11 (SEQ ID NO:9) and SG-11V5 (SEQ ID NO:19) at the dose of 50 moles/kg and a dose dependent effect was observed for SG-11V5 (SEQ ID NO:19) with the 160 nmoles/kg dose resulting in a significant improvement (p<0.002). Data, illustrated in FIG. 21, am presented as mean±SEM and include data from an individual experiment. Statistical analysis was performed using a one-way ANOVA followed by a Fisher's LSD multiple comparison test.

### Effects of SG-11 and SG-11V5 on Colon Length in a DSS Model of Inflammatory Bowel Disease

DSS models from Example 13 were also analyzed for the effect of SG-11 and SG-11 variant proteins on the colon length. Colon length measurements were made for the Example 13A (FIG. 22A) or Example 13B (FIG. 22B) DSS models. Similar results were obtained with SG-11 (SEQ ID NO:9) and SG-11V5 (SEQ ID NO:19) in both DSS models, where both treatment regimens resulted in a significant increase in the colon length. However, no dose-dependent effect on colon length was observed with SG-11V5 (SEQ ID NO:19) in the prophylactic DSS model. Data in both graphs are presented as mean±SEM and represent data from an individual experiment. Statistical analysis was performed using a one-way ANOVA compared to DSS+ vehicle followed by a Fishers LSD multiple comparison test.

### Effects of SG-11 and SG-11V5 on Colon Weight-to-Length Ratios in a DSS Model of Inflammatory Bowel Disease

DSS models from Example 13 were also analyzed for the effect of SG-11 and SG-11 variant proteins on the colon weight-to-length ratio. Colon weight to length ratios were similar between SG-11 (SEQ ID NO:9) and SG-11V5 (SEQ ID NO:19) in the Example 13A (FIG. 23A) and Example 13B (FIG. 23B) DSS model treatment regimens. In the Example 13A treatment, all treatments and doses significantly improved colon weight to length ratios (p<0.05). In the Example 13B treatment regiment, SG-11 (SEQ ID NO:9) and SG-11V5 (SEQ ID NO:19) both significantly improved colon weight to length ratios (p<0.01), while the positive control Gly2-GLP2 did not. Statistical analysis was performed by a one-way ANOVA as compared to DSS+vehicle using a Fisher's LSD multiple comparisons test. Data are graphed as mean±SEM and each figure represent data from a single experiment.

### Example 14

Identification of a SG-11 Variant with Lower Apparent Molecular Weight

Studies were done in order to assess stability of the SG-11 protein in the intestinal environment; specifically, in the large intestine where fecal matter is present. These studies are an important aspect of designing a product which can be successfully delivered via rectal administration. These studies also help to identify functional domains of the protein. Initial studies showed that incubation of purified recombinantly expressed SG-11 (these experiments were repeated with proteins depicted by SEQ ID NO:9, SEQ ID NO:7, and SEQ ID NO:19) in a fecal slurry at room temperature degraded to form a predominant form with an apparent molecular weight of about 25 kDa when analyzed by SDS-PAGE gel (4-20% Mini-PROTEAS® TGX™ precast protein gel; BioRad) and Coomassie blue staining. FIG. 25 shows results of an experiment in which purified SG-11 (SEQ ID NO:9) was incubated in the presence or absence of fecal slurry or incubated in fecal slurry for different periods of time at 37° C. Fecal slurry is prepared by dissolving 2 g fecal pellets (human) in 1 ml PBS buffer, in which the SG-11 protein was incubated (Lane 3: 20 μg in 20 μl reaction mix; Lanes 6-9: 60 μg in 20 μl reaction mix). Reactions were terminated by immediate transfer to sample buffer and boiling at 95° C. for 5 min. FIG. 25, Lane 1: Molecular weight markers (Precision Plus Protein™ Dual Color Standards (BioRad, Hercules, Calif.); Lane 2: purified SG-11 (SEQ ID NO:9); Lane 3: fecal slurry only; Lane 4: SG-11 in focal slurry, 10 min at 37° C.; Lane 5: fecal slurry only, 10 min at 37° C.; Lanes 6-9: SG-11 in fecal slurry for 10 min, 30 min, 1 hr, 2 hr, respectively. The results show the generation of a predominant band with an apparent molecular weight of about 25 kDa with minor bands apparent by Coomassie Blue staining at 18 kDa and 10 kDa.

An experiment was performed to assess generation of the fragment upon incubation in the presence of trypsin. Columns were prepared to contain 100 μl immobilized Trypsin slurry, washed twice with PBS, loaded with SG-11 (SEQ ID NO9) diluted in PBS, pH 7.4, then incubated at room temperature for varied times. To stop the reaction, each column was centrifuged to remove protein from the column, then analyzed on an SDS-PAGE gel using Coomassie Blue visualization. The gel analysis is shown in FIG. 26. Lane 1: Molecular weight markers (kDa) (Precision Plus Protein™ Dual Color Standards, BioRad, Hercules, Calif.); Lane 2: SG-11 (SEQ ID NO:9) only; Lanes 3-6: incubation of SG-11 with trypsin at room temperature for 10 min, 30 min, 1 hr, or 2 hr, respectively. These data show that a predominant band is generated in the presence of trypsin which migrates to a position which appears to be the same as that of the product generated when SG-11 is incubated in fecal slurry, supporting the assertion that the predominant band which migrates to an apparent molecular weight of about 25 kDa results from cleavage of the mature SG-11 protein.

Next, SG-11 protein was incubated in fecal slurry in the absence or presence of a trypsin inhibitor (soybean trypsin inhibitor (SBTI), Millipore Sigma, St. Louis, Mo.). SG-11 (SEQ ID NO:7) was mixed with fecal slurry as described above. The SG-11 samples were then incubated at 37° C. for about 1 hr prior to mixing the sample with SDS sample buffer to terminate any further enzyme activity. Samples were then analyzed using SDS-PAGE (4-20% Mini-PROTEAS® TGX™ precast protein gel; BioRad) and stained with Coomasie Blue. As shown in FIG. 27, in the presence of fecal slurry, a band appears with an apparent molecular weight of about 25 kDa. In the presence of both fecal slurry and trypsin inhibitor, most of the SG-11 protein remains intact. (FIG. 27: Lane 1: Molecular weight markers (kDa) (Precision Plus Protein™ Dual Color Standards, BioRad, Hercules, Calif.); Lane 2: SG-11 (SEQ ID NO:7) in PBS; Lane 3: fecal slurry only; Lane 4: SG-11 with in fecal slurry; Lane 5: SG-11 with fecal slurry and 1 μg SBTI; Lane 6: 1 μg SBTI inhibitor only. These data show that the generation of the predominant band (which migrates to about 25 kDa) in fecal slurry is almost completely inhibited in the presence of the trypsin inhibitor, supporting the assertion that the predominant band which migrates to an apparent molecular weight of about 25 kDa results rom cleavage of the mature SG-11 protein.

Additional studies showed that addition of EDTA to an incubation mixture containing 3 μg SG-11, fecal slurry, and 1 μg SBTI resulted in the generation of the apparent ˜25 kDa band (data not shown).

Accordingly, it is concluded that the SG-11 protein can be processed in fecal slurry in vitro and likely in vivo if exposed to intestinal fecal matter to generate a fragment of the SG-11 protein, referred to herein as SG-21.

### Example 15

### SG-21 Activity in an In Vitro Barrier Function Assay

The next study was performed to confirm that the SG-11 variant SG-21 maintains functional activity equivalent to at of SG-11. Specifically, a TEER assay as described in Example 1 above, was done using a test agent comprised of fecal slurry and SG-11 protein (SEQ ID NO:9).

Mouse fecal pellets were collected from C57BL6 mice and a fecal suspension was prepared as described in Example 14. Tissue culture was performed as described in Example 1 above. Briefly, following 9-10 days of culture, the transwell plate containing enterocytes were treated with 10 ng/ml IFN-γ added to the basolateral chamber of the transwell plate for 24 hours at 37° C.+5% CO₂. After 24 hours, fresh cRPMI was added to the epithelial cell culture plate. TEER readings were measured after the IFN-γ treatment and were used as the pre-treatment TEER values. Test samples included: 1 μg/ml of SG-11 (SEQ ID NO9), 1 μg/m of SG-11 digested in the fecal slurry as described in Example 14, or an equivalent volume of fecal slurry. Treatments were added to the apical chamber of the transwell plate. The MLCK inhibitor peptide 18 (BioTechne, Minneapolis, Minn.) was used at 50 nM as a positive control to prevent inflammation induced barrier disruption (Zolotarevskky et al., 2002, Gastroenterology, 123:163-172). Test and control agents were incubated on enterocytes for 6 hours. Following pre-incubation with test and control agents, the transwell insert containing the enterocytes was transferred on top of the receiver plate containing U937 monocytes. Heat killed E. coli (HK E. coli) (bacteria heated to 80° C. for 40 minutes) was then added to both the apical and basolateral chambers and a multiplicity of infection (MOI) of 10. Transwell plates were incubated at 37° C.+5% CO₂ for 24 hours and a post treatment TEER measurement was made. SG-11 increased TEER from 78.6% disruption by HK E. coli to 89.5% (p<0.0001), while fecal slurry-digested SG-11 increased TEER to 90.2% (p<0.0001) (FIG. 28). Statistical analysis was performed using a one-way ANOVA compared to HK E. coli followed by a Fisher's LSD multiple comparison test. The graph in FIG. 28 represent data pooled Rom four plates performed in two individual experiments (n=12). Notably, similar results were observed when the TEER assay was performed using SG-11 (SEQ ID NO:9) digested with trypsin as described in Example 14 rather than incubated with fecal slurry (data not shown).

### Example 16

### Determining the SG-21 N-Terminus

The results obtained in Example 14 above indicate that SG-11 is processed in the intestine to a smaller fragment such as the apparent ˜25 kDa fragment observed in the experiments described here. Accordingly, it was of interest to identify the portion of SG-11 contained within this fragment and whether or not this fragment possesses functional activity comparable to the functional activity of full-length SG-11.

First, SG-11 (SEQ ID NO:9) was incubated in a fecal slurry mix or with trypsin as above at 37° C. for about 2 hours. The reaction mixtures were run on an SDS-PAGE and stained with Coomasie Blue as above. Individual gel slices containing the ˜25 kDa band and 2 much fainter, additional bands (at about 18 kDa and 10 kDa) were excised and sent for peptide mapping analysis (Alphalyse Inc., Palo Alto, Calif.)

Each sample was reduced with DTT, alkylated with IAA and in-gel digested with trypsin. Each sample was then analyzed on a Bruker Maxis instrument connected with a Dionex nanoLC instrument vi an ESI-source. Equal amounts of the samples were separated by on a reversed phase using a 60 min gradient program with a flow of 300 nL/min. The data were acquired in data-dependent mode where a survey spectrum of m/z range 350-2000 is followed by MS/MS [m/z range 80-2000] of the most intense multiply charged ions using collision induced dissociation. The data were processed using a combination of software tools including Mascot 2.4.0, and Skyline 3.7.0.11317 to extract and match the experimental data with the theoretical parent masses and fragmentation spectra. The data were searched with semi-tryptic constraints and oxidation (M), pyro-glutamine (N-term Q), pyro-glutamate (N-term E) and acetylation of lysine.

Normalized peak intensities for each of 513 peptides identified by Alphalyse. From these data, total amounts of peptides having the same amino acid start were quantified (in terms of peak height and total area) and mapped along the amino acid sequence. These data showed an increased number of peptides identified starting at amino acid 73 of SEQ ID NO:7 (40 peptides identified) and 75 of SEQ ID NO:7 (44 peptides identified) for both the trypsin and the fecal digests. 28 peptides were identified with an N-terminus as position 71 of SEQ ID NO:7. A total of 68 peptides were identified having N-termini before position 71 (having N-termini at positions 14, IS, 36, 38, 40, 52 and 56 of SEQ ID NO:7) but the sum of the total area and the maximum height for these peptides were significantly less than those of the peptides having N-termini at positions 70 to % of SEQ ID NO:7. From these data, its concluded that the region (between about positions 70 to 96) represent the N-terminus of the fragment which migrates to about the 25 kD position in SDS-PAGE analysis. The C-terminal residue was not definitively identified because it does not contain any trypsin cleavage sites, and is therefore not detectable by mass spectroscopy analysis.

The analysis of the peptides identified by the process above strongly suggests that the predominant fragment observed in the SDS-PAGE analysis of the fecal-treated SG-11 protein is a C-terminal fragment of SG-2-11, e.g., comprising at least amino acids 100 of SG-11 and possibly having an N-terminus beginning at residue 71, 72, 73, 74, 75, 76, 77, 78, 79, 80, 81, 83, 83, 84 or 85 of SG-11 (SEQ ID NO7).

### Example 17

### Expression of SG21 and SI-21V5

To confirm that the functional activity of SG-11 resides in the C-terminal portion of the protein, expression constructs were designed and used to express a protein comprising amino acids 96-256 of SG-11 and SG-11V5.

For expression of the C-terminal fragment with an N-terminal His tag, a polynucleotide encoding amino acids 73 to 233 of SG-11 (where the protein is SEQ ID NO:34) and of SG-11V5 (SEQ ID NO:19) was PCR-amplified and sub-cloned into the pET-28a vector (Novagen) using standard methods as described in Example 1 to generate protein having the sequence disclosed herein as SEQ ID NO:4 and SEQ ID NO:45, respectively. Also expressed were SG-21 and SG-21V5 proteins without N-terminal tags (SEQ ID NO:36 and SEQ ID NO:43, respectively) using standard protein expression and purification protocols.

### Example 18

### Functional Activity of SG-21 and Variants Thereof to Restore Epithelial Barer Integrity In Vitro

To further show that SG-21 or variants thereof possess activity that is equivalent to that of SG-11 or variants thereof, any one of the proteins prepared as described, for example, in Example 17, with or without N-terminal tags, can be tested in in vivo TEER assays as described in Example 2 above. For example, a test protein comprising amino acids 72 to 233 of SEQ ID NO:7 and having a total length of no more than 170 amino acids can be used in the TEER assays. The TEER assays can be performed to compare activity of the test proteins, e.g., SG-21 protein comprising SEQ ID NO:3 with, e.g., SG-11 (SEQ ID NO:7), or to compare activity of SG-21 protein comprising SEQ ID NO:3 with, e.g., SG-21V5 comprising SEQ CD NO:19 (see, e.g., Example 12 above). Additionally, an in vitro assay to measure effects of a SG-11 protein or fragment or variant thereof on epithelial barrier function, such as a TEER assay, can be used to test the effects of SG-11 fragments such as those described herein as SEQ ID NO:46, SEQ ID NO:47, SEQ ID NO:48 or SEQ ID NO:49 (see Table 12 below).

TABLE 12

| | | Residues |
|---|---|---|
| | | with |
| SEQ | | respect |
| ID | | to SG 11 |
| NO: | Sequence | SEQ ID NO: 7 |
| 46 | YYLETGSVTASVDVTGQESVGTEQLSGTEQMEM | 97-148 |
| | TGEPVNADDIEQTEAAAGD | |
| 47 | TPEDYTAFNGIELYQGKVVASLAAGYVYDGEFA | 4-49 |
| | RVEEGKVVGAATK | |
| 48 | QDIYSEDDLKVAIIRANTDVKVDGEICYVSCQN | 50-96 |
| | VKLTGKDSVSIRDG | |
| 49 | LAAGYVYDGEFARVEEGKVVGAATKQDIYSEDD | 25-74 |
| | LKVAIIRANTDVKVDGE | |

The HCT8 human enterocyte col line (ATCC Cat. No. CCL-244) is maintained in RPMI-1640 medium supplemented with 10% fetal bovine serum, 100 IU/ml penicillin, 100 [symbol] g/ml streptomycin, 10 [symbol] g/ml gentamicin and 0.25 μg/ml amphotericin (cRPMI). HT29-MTX human goblet cells (Sigma-Aldrich (St. Louis, Mo.; Cat. No. 12040401) are maintained in DMEM medium with 10% fetal bovine serum, 100 IU/ml penicillin, 100 [symbol] g/ml streptomycin, 10 [symbol] g/ml gentamicin and 0.25 μm amphotericin (cDMEM). Epithelial cells are passaged by trypsinization and were used between 5 and 15 passages following thawing from liquid nitrogen stocks. U937 monocytes (ATCC Cat. No. 700928) are maintained in cRPMI medium as a suspension culture, and split by dilution as needed to maintain cells between 5×10⁵ and 2×10⁶ cells/ml. U937 cells are used up to passage 18 following thawing from liquid nitrogen stocks.

Epithelial cell culture A mixture of HCT8 enterocytes and HT29-MTX goblet cells are plated at about a 9:1 ratio, respectively, in the apical chamber of the transwell plate as described previously (Berget et al., 2017, Int J Mol Sci, 18:1573; Beduneau et al., 2014, Eur J Pharm Biopharm, 87:290-298). A total of 10⁵ cells are plated in each well (9×10⁴ HCT8 cells and 1×10⁴ HT29-MTX cells per well), Epithelial cells are trypsinized from culture flasks and viable cells determined by trypan blue counting. The correct volumes of each cell type are combined in a single tube and centrifuged. The cell pellet is resuspended in cRPMI and added to the apical chamber of the transwell plate. Cells are cultured for 5 to 10 days at 37° C.+5% CO₂, and media is changed every 2 days.

Monocyte culture. On day 6 of epithelial cell culture 2×10⁵ cells/well U937 monocytes are plated into a 96 well receiver plate. Cells are cultured at 37° C.+5% CO₂ and media is changed every 24 hours for four days.

Co-culture assay. Following 8-10 days of culture the transwell plate containing enterocytes is treated with 10 ng/ml IFN-© added to the basolateral chamber of the transwell plate for 24 hours at 37° C.+5% CO₂. After 24 on fresh cRPMI is added to the epithelial cell culture plate. TEER readings are measured after the IFN-© treatment and are used as the pre-treatment TEER values. SG-21 protein or variant thereof is then added to the apical chamber of the transwell plate at a final concentration of about 1 [symbol] g/ml (40 nM). The MLCK inhibitor peptide 18 (BioTechne, Minneapolis, Minn.) is used at 50 nM as a positive control to prevent inflammation induced barrier disruption (Zolotarevskky et al., 2002, Gastroenterology, 123:163-172). The bacterially derived molecule staurosporine is used at 100 nM as a negative control to induce apoptosis and exacerbate barrier disruption (Antonsson and Persson, 2009, Anticancer Res, 29:2893-2898). Compounds are incubated on enterocytes for 1 hour or 6 hours. Following pre-incubation with test compounds the transwell insert containing the enterocytes is transferred on top of the receiver plate containing U937 monocytes. Heat killed E. coli (HK E. coli) (bacteria heated to 80° C. for 40 minutes) is then added to both the apical and basolateral chambers and a multiplicity of infection (MOI) of 10. Transwell plates are incubated at 37° C.+5% CO₂ for 24 hours and a post treatment TEER measurement is made.

Data analysis. Raw electrical resistance values in ohms (\hat{ }) can be converted to ohms per square centimeter (\hat{ }cm²) based on the surface area of the transwell insert (0.143 cm²). To adjust for differential resistances developing over 10 days of culture, individual well post treatment \hat{ }cm² readings can be normalized to pro-treatment \hat{ }cm² readings. Normalized \hat{ }cm² values am then expressed as a percent change from the mean \hat{ }cm² values of untreated samples.

Test protein is added 1 hour or 6 hours prior to exposure of both epithelial cells and monocytes to beat killed Escherichia coli (HK E. coli), inducing monocytes to produce inflammatory mediators resulting in disruption of the epithelial monolayer as indicated by a reduction in TEER. A myosin light chain kinase (MLCK) inhibitor is utilized as a control compound, which ha been shown to prevent barrier disruption and/or reverse barrier loss triggered by the antibacterial immune response. Staurosporine is used as a control compound that caused epithelial cell apoptosis and/or death, thus resulting in a drastic decrease in TEER, which indicates disruption and/or loss of epithelial cell barrier integrity/function.

### Example 19

### Functional Activity of SG-21 and Variants Thereof in an In Vivo Model of Colitis

To further show that SG-21 or variants thereof possess activity which is equivalent to that of SG-11 or variants thereof any one of the proteins prepared as described, for example, in Example 17 above, with or without N-terminal tags, can be administered to an animal model of colitis as described, for example, in Example 13 above. Again, a test protein comprising amino acids 72 to 233 of SEQ ID NO:7 and having a total length of no more than 170 amino acids can be used in the in vivo assays. The in vivo assays can be performed to compare activity of the test proteins, e.g., SG-21 protein comprising SEQ ID NO:36 with, e.g., SG-11 (SEQ ID NO:7), or to compare activity of SG-21 protein comprising SEQ ID NO:36 with, e.g., SG-21V5 comprising SEQ ID NO:42 (see, e.g., Examples 4, 5, and 13 above).

In these experiments, for example SG-21 or SG-21V5 am administered to a mouse concurrent with the initiation of treatment with DSS (as in Example 4) or after prior DSS administration. The only difference is that mice in Example 5 were treated with SG-11 or SG-11V5 (SEQ ID NO:19) for 4 days rather than 6 days.

Briefly, in the first DSS mouse model (as described in Example 13A), mice are treated on day zero with test compound intraperitoneally (i.p.) and 6 hours later DSS treatment is initiated. Does administered included 50 nmoles/kg for SG-21 (1.3 mg/ml), and Gly2-GLP2 (0.2 mg/kg), and a dose response for SG-21V5 (SEQ ID NO:19) including 16 nmoles/kg (0.4 mg/ml), 50 nmoles/kg (1.3 mg/ml) and 158 nmoles/kg (4.0 mg/kg). The mice were treated with 2.5% DSS in their drinking water for 6 days (day zero through day 6). Therapeutic protein treatments were administered twice a day for the duration of the DSS exposure.

In the second experiment (Example 13B), mice arm provided with drinking water containing 2.5% DSS for 7 days. On day 7 normal drinking water is restored and i.p. treatments of 50 nmole/kg of SG-21 (1.3 mg/kg), SG-21V5 (13 mg/kg), or Gly2-GLP2 (0.2 mg/kg) are initiated. Treatments are administered twice a day (b.i.d.), with a morning and evening dose (every 8 and 16 hours) for 4 days. For both the prophylactic and therapeutic models fresh 2.5% DSS water was prepared every 2 days during the DSS administration.

At the end of each DSS model study, mice are fasted for 4 hours and then orally gavaged with 600 mg/kg 4KDa dextran labeled with fluorescein isothiocyanate (FITC) [4KDa-FITC]. One hour after the 4KDa-FITC gavage mice are euthanized, blood is collected and FITC signal is measured in scrum.

### Effects of SG-21 and SG-21V5 on Inflammation Centric Readouts of Barrier Function in a DSS Model of Inflammatory Bowel Disease

Upon completion of the DSS models above, LBP levels are measured as an inflammation centric readout of barrier function following the protocol detailed in Example 4. Upon completion of both DSS models (Examples 13A and 13B) blood is collected and serum is isolated. LBP levels are measured in scrum using a commercially available ELISA Kit (Enzo Life Sciences).

### Effects of SG-21 and SG-21V5 on Body Weight in a DSS Model of Inflammatory Bowel Disease

Body weight is measured throughout the experimental models as in both Example 13A and Example 13B.

### Effects of SG-21 and SG-21V5 on Gross Pathology in a DSS Model of Inflammatory Bowel Disease

Gross pathology observations of colon tissue are made as described in Example 4 above. Briefly, a scoring system based on the level of visible blood and fecal pellet consistency is used. The scoring system is: (0)=no grow pathology, (1)=streaks of blood visible in feces, (2)=completely bloody fecal pellets, (3) bloody fecal material visible in cecum, (4) bloody fecal material in cecum and loose stool, (5)=rectal bleeding.

### Effects of SG-21 and SG-21V5 on Colon Length in a DSS Model of Inflammatory Bowel Disease

DSS models from Example 19 are also analyzed for the effect of SG-21 and SG-21 variant proteins on the colon length and colon weight-to-length ratios as described in Example 13 above.

### Example 20

Expression of SG-11V5 in Lactococcus Lactis (L. lactis) Strain

Studies were performed to test the ability to administer a therapeutic protein to a subject by administering a bacterium engineered to express the therapeutic protein in vo. For this specific example, the bacterium Lactococcus lactis was used. L. lactis is extensively used in the production of dairy products.

A polynucleotide (SEQ ID NO: 20) encoding SG-11V5 (residues 2-233 of SEQ ID NO:19) was cloned into an expression vector and used to transform bacterial cells for expression of SG-11V5 as detailed below, using culturing and purification methods which are routine in the art. The vector constructions and protein expression in bacterial cells can be performed to test polynucleotides encoding SG-11 and variants thereof (SEQ ID NOs:1, 3, 5, 7, 9, 11, 13, 15, 17, and 19) proteins and SG-21 protein and variants thereof (SEQ ID NOs 34, 36, 38, 39, 40, 42, 44, 45, 46, 47, 48, 49, and 50) according to methods and protocols described below.

Construction of recombinant vectors for expressing SG-11 or variants thereof was achieved using a pNZ8124 vector system (see NICE® Expression System for Lactococcus lactis, MoBitec GmbH) which is designed for inducible, high-level expression of genes or gene fragments. The vector has a strictly Nisin controlled gene expression system using an inducible nisin A promoter (PnisA) for chemically inducible, high-level expression in L. lactis. This expression system may be applied to other bacterial strains such as Lactobacillus brevis, Lactobacillus helveticus, Lactobacillus plantarum, Streptococcus pyogenes, Streptococcus agalactiae, Streptococcus pneumoniae, Streptococcus zooepidemicus, Enterococcus faecalis, and Bacillus subtilis. The pNZ8124 vector also contains a sequence downstream of the nisA promoter which encodes for the signal peptide of the USP45 protein. In order to evaluate overall production of the protein of interest, various expression constructs were tested that included a constitutive active promoter and/or an inducible promoter. Furthermore, an expression cassette for trehalose accumulation was subcloned into the pNZ8124 expression vector system.

TABLE 13
L. lactis expression vector (pNZ8124) (SEQ ID NO: 51)

| Feature | | | | |
|---|---|---|---|---|
| name | Type | Nucleotides | Length | Direction |
| PnisA | misc_feature | 5-201 | 197 | forward |
| promoter | | | | |
| Usp45 | misc_feature | 204-284 | 81 | forward |
| signal | | | | |
| peptide | | | | |
| EcoRV | misc_feature | 285-290 | 6 | forward |
| cloning | | | | |
| site | | | | |
| 246 F | primer_bind | 246-265 | 20 | forward |
| 454 R | primer_bind_reverse | 435-454 | 20 | reverse |
| ds origin | Origin of Replication | 905-925 | 21 | forward |
| T7 term | Terminator | 958-1001 | 44 | forward |
| repC | Regulatory | 1146-1285 | 140 | forward |
| | Sequence | | | |
| repA | Regulatory | 1392-2075 | 684 | forward |
| | Sequence | | | |
| SV40_int | Gene | 3044-3061 | 18 | reverse |
| Cm | misc_feature | 2517-3167 | 651 | forward |
| SV40_int | Gene | 3044-3061 | 18 | reverse |

Bacterial Strains and Media

The present study was performed using the Lactococcus lactis strain NZ9000 (NICE Expression System, MoBiTec GmbH). This strain is a derivative of L. lactis subsp. cremoris M01363. To construct this strain, the genes for nisK and nisR were integrated into the pepN gene (broad range amino acid peptidase) of MG1363. These two genes are transcribed from their own constitutive promoter and function to activate transcription nom a nisA promoter in the presence of nisin. Bacterial strains used herein were routinely grown as standing cultures at 30° C. in M17 broth with 0.5% Lactose (Sigma-Aldrich) supplemented with 0.3% glucose and 10 μg/ml chloramphenicol when appropriate (GM17C). Stock suspensions of L. lactis strains were stored at ˜80° C. with 10% Glycerol in GM17C.

Plasmid Constructions

FIG. 29 shows expression cassettes in a L. lactis expression plasmid, pNZ8124. The pMZ8124 plasmid is designed for expressing a gene of interest (e.g. SG-11V5) under control of an inducible nisin A promoter (PnisA) and the lactococcus usp45 secretion leader (aka signal peptide) sequence. Alternatively, for the constitutive expression of a gene of interest (e.g. SG-11V5), the PnisA promoter can be replaced with a strong constitutive promoter (Usp4) in the L. lactis expression plasmids. To induce trehalose accumulation in the L. lactis strain, an additional expression cassette (PnisA-otsBA operon) comprising trehalose-6-phosphate phosphatase (otsB) and trehalose-6-phosphate synthase (otsA) genes placed downstream of an inducible nisin A promoter (PnisA) was cloned into a pNZ8124 plasmid.

Expression vectors were constructed using the pNZ8124 vector described above to contain protein-coding sequences under the control of the inducible nisA promoter (PnisA; SEQ ID NO:52). Specifically, 4 different expression cassettes were constructed and inserted into the pNZ8124 for further studies as described below:
a. PnisA:SPusp45SG-11V5:Flag (SEQ ID NO:53)
b. PnisA:otsBA (negative control without SG-11V5)(SEQ ID NO:56)
c. PnisA:otsBA::PnisA:SPusp45:SG-11V5
d. PnisA:otsBA::Pusp45:SPusp45; SG-11V5

PnisA refers to the inducible nisinA promoter which is induced by low concentrations of nisin. Pusp45 is the natural constitutive promoter for the usp45 gene. Accordingly, references to Pusp45:SG-11V5 in the present disclosure means that there is a USP45 signal peptide at the N-terminus of the SG-11V5 protein, i.e., Pusp45:SG-11V5 is the same as Pusp45:SPusp45:SG-11V5. Thus, Pusp45:SG-11V5 is interchangeably used with Pusp45:SPusp45:SG-11V5 in the present disclosure. The construct comprising Pusp45:SPusp45:SG-11V5 sequence is set forth in SEQ ID NO:61. The construct comprising PnisA:SPusp45:SG-11V5 is set forth in SEQ ID NO:66.

In each case, the SG-11 variant (residues 2-233 of SEQ ID N:19) was expressed with an N-terminal signal peptide derived from the usp45 protein (MKKKIISAILMSTVILSAAA PLSGVYA; SEQ ID NO:67; see GenBank accession no. AAA25230).

PnisA:SPusp45SG-11V5:Flag construction. The DNA sequence encoding SG-11V5 with an C-terminal Flag Tag was PCR-amplified with AGGTGTTTACGCTGATATC TTOGAOG (TGAAGAGTCrGT (SG11fW: SEQ ID NO:68) and AAAGCTTGAGCTCTCTAGATTACTTGTCGTCATCGTCTTTGTAGTCCTTGTACACGAT AAAGGTGT (SG11rv: SEQ ID NO:69) and inserted downstream of, and in-frame with, the sequence encoding the USP45 signal peptide (the PnisA:SPusp45SG-11V5:Flag operon is provided herein as SEQ ID NO:53). SPusp45:SG-11V5:Flag operon sequence without a TGA stop codon is provided in SEQ ID NO:54. SPusp45-SG-11V5-Flag fusion protein sequence is set forth in SEQ ID NO:55. Accordingly, SG-11V5 gene expression was placed under control of the nisA inducible promoter and translated SG-11V5 protein should be secreted by the cell.

PnisA:otsBA construction. An expression vector (which does not contain a SG-11 sequence) was generated to include the trehalose biosynthesis operon otsBA (see, e.g., GenBank accession no. X69160; see also Termont et al., App, and Envir. Microb. 2006, 72(12): 7694-7700). The operon includes the trehalose biosynthesis genes otsA and otsB, encoding trehalose-6-phosphate synthase and trehalose-6-phosphate phosphatase, respectively.

To obtain the otsBA DNA for insertion into the pNZ8124 parent vector, genomic DNA was purified from E. coli strain DHS with a QIAGEN DNeasy kit (Hilden, Germany). The DNA sequence encompassing the otsBA genes, together with primer sequences containing suitable restriction sites for insertion into pNZ8124 downstream of the nisA promoter, were PCR amplified using an otsBA forward primer (otsBAfw: TTATAAGGAGGCACTCAAAATGACAG AACCGTTAACC; SEQ ID NO:57) and mvBA reverse primer (oatBArw: CTGAGATAATCT TTTTTCATCTACGCAAGCTTTGGAAAGOTA; SEQ ID NO:58) containing flanking regions for Gibson overlap from promoter pTreI. pTreI vector is described in Termont et al., App. and Envir. Microb. 2006, 72(12): 7694-7700.

To construct a pNZ8124-based expression vector comprising the otsBA operon downstream of the nisA promoter, the pNZ8124 plasmid was linearized by amplification with pNZ8124 forward primer (pNZ8124fw: TTGAGTGCCTCCTTATAA; SEQ ID NO:59) and pNZ8124 reverse primer (pNZ8124rv: ATGAAAAAAAAGATTATCTC; SEQ ID NO:60). The linearized plasmid and amplified otsBA gene focus were fused using the Gibson Assembley® Cloning Kit (New England Biolabs). The coding sequence of otsBA was fused downstream of, and in frame with, the initiator ATG of the nisA ribosome binding site to create the operon provided herein as SEQ ID NO:66. The region encompassing the nisA promoter, the nisA ribosome binding site, and the junction of the initiator ATG with the otsB cistron, was verified by ELIM Biopharm and analyzed using Geneious®.

PnisA:otsBA::Pusp45:SPusp45:SG-11V5 construction. Also constructed was the otsBA-containing L. lactis pNZ8124 plasmid, which further contains an expression cassette comprising the usp45 secretion leader and SG-11V5 gene driven by the constitutive usp45 promoter (Pusp45). This operon includes the promoter, ribosomal binding site and the usp45 signal peptide sequence, which was amplified using a usp45 forward primer (usp45fW: (atcggGATATCTOTTTTOTAATCATAAAGAAATATTAAGGT; SEQ ID NO-62) containing an EcoRV restriction site, and usp45rv (atcggCCATGGAGCGTAAACACCTGACAACG GGGCTGCAG; SEQ ID NO: 63) containing a NcoI restriction site. The nucleotide sequence of SEQ ID NO:20, which encodes SG-11V5, was amplified using SG-11V5NcoI forward primer (SG-11V5NcoIfw: atcggCCATGGTT GGAGGGTGAAGAGTCTGT; SEQ 11) NO:64) and SG-11V5XbaI reverse primer (SG-11V5XbaIrv: atcggTCTAGATTACTTGTACACGATAAAGGTGT; SEQ ID NO:65) containing a NcoI and a XbaI restriction site, respectively. Thus, the usp45 promoter and SG-11V5 nucleotide sequences were inserted into a PnisA:otsBA-containing pNZ8124 construct using the respective restriction enzymes (NEB) and ligated using the T4 ligase (NEB). The orientation of the insert was verified by DNA sequencing. Final plasmids were sequenced by ELIM Biopharm and analyzed using Geneious®. The insert comprising the Pusp45:SPusp45:SG-11V5 construct is presented herein as SEQ ID NO:61.

PnisA:otsBA::PnisA:SPusp45:SG-11V5 construction. Also constructed was the otsBA-containing L. lactis pNZ8124 plasmid in which the expression of both the otsBA operon and SG-11V5 is under control of the sin-inducible promoter (PnisA). Again, the construct encodes the usp45 signal peptide (SPusp45) at the N-terminus of the SG-11V5 sequence. Specifically, a polynucleotide comprising a nucleotide sequence encoding SG-11V5 (residues 2-233 of SEQ ID NO:19) was fused downstream of, and in frame with, the nisA promoter sequence and usp45 signal peptide then inserted downstream of the PnisA-otsBA operon, which had already been inserted into the pNZ8124 plasmid as described above, to express SG-11V5 having an N-terminal signal peptide by a nisin induction system. The construct comprising PnisA:SPusp45:SG-11V5 is provided as SEQ ID NO:66.

### In Vitro SG-11V5 Protein Detection

In vitro expression of the SG-11 variant by L. lactis strains transformed with the vectors described above was tested. The transformed cells were grown overnight in M17 broth with 0.5% Lactose (Sigma-Aldrich). OD600 was measured, and cells were centrifuged at 3500 rpm, 5 min at RT, and normalized to an OD of 3 (the equivalent of 10⁹ cells) in fresh in M17 broth with 0.5% lactose and incubated at 37° C. for 1 h to 2 h. Ten μl of supernatant was boiled in SDS Laemmli buffer and separated via SDS page (Biorad). Gels were blotted via Turbo-Blot, and SG-11V5 protein was detected via anti-SG-11 antibodies (1:5000 dilution) for 2 hours incubation and goat-anti-rabbit-Hrp (1:25000, Fisher Sci) as a secondary antibody. Polyclonal antibody generation used a 77-day protocol in rabbits and SG-11V5 as an antigen.

FIG. 30 shows results of a western blot analysis in which the proteins extracted from different L. lactis strains transformed by the 4 recombinant plasmids described above. Five transformed L. lactis strains were tested in the absence or presence of nisin induction (0.1.5 ng/ml). Protein samples for Lanes 1-5 were obtained from the L. lactis strains that were not treated with nisin, while protein samples for lanes 6-10 were obtained from nisin-treated L. lactis strains. Lane 1: protein extracted from the L. lactis strain transformed with PnisA:otsBA (negative control without SG-11V5); Lane 2: protein extracted from the L. lactis strain transformed with PnisA:SG-11V5:Flag; Lane 3: protein extracted from the L. lactis strain transformed with PnisA:otsBA::PnisA:SPusp45:SG-11V5; Lane 4: protein extracted from the L. lactis strain transformed with PnisA-otsBA:Pusp45:SPusp45:SG-11V5; Lane 5: protein extracted from the L. lactis strain transformed with PnisA:otsBA::Pusp45:SPusp45:SG-11V5; Lane 6: same as Lane 1 but nisin-treated; Lane 7: same as Lane 2 but nisin-treated; Lane 8: same as Lane 3 but nisin-treated: Lane 9: same as Lane 4 but nisin-treated; Lane 10: same as Lane 5 but nisin-treated. As shown in FIG. 30, the nisin-treated L. lactis strains expressing SG-11V5 under control of the nisin inducible promoter (Lanes 7-g) produced more SG-11V5 protein production than the L. lactis strains expressing the protein driven by the constitutive promoter (Lanes 4-4 and 9-10).

### Example 21

Expression of SG-11V5 from Lactococcus lactis (L. lactis) strains in a mouse model

An experiment was performed to assess survival of L. lactis strains producing SG-11V5 protein in a mouse model in vivo. The L. lactis strains were administered into C57BL/6 mice topically by oral gavage (p.o.), and mouse fecal samples were collected from C57BL/6 mice from the mice 5 hours after the bacterial infection. A fecal suspension was prepared as described in Example 15 and protein samples were prepared for the western blot analysis according to standard extraction and purification protocols. Also, multiple doses of purified SG-11V5 proteins were administered to mice by intraperitoneal injections as a control and the proteins were prepared as the procedure described above.

The western blot analysis using anti-SG-115V antibody is shown in FIG. 31A. Ten μl of the noted samples was loaded onto each of lanes 1-8. Lane 1: 10 μg/ml purified SG-11V5; Lane2: 1 μg/ml purified SG-11V5; Lane 3:0.1 μg/ml purified SG-11V5; Lane 4:0.01 μg/ml SG-11V5; Las 5-6 protein extracted from fecal sample of mice administered with the L. lactis strain transformed with PnisA:SG-11V5:Flag; Lanes 7-8: protein extracted from the L. lactis strain transformed with PnisA:otsBA::Pusp45:SPusp45:SG-11V5. As shown in FIG. 31A, the L. lactis strains survive in the mice after administration and SG-11V5 proteins are expressed and secreted in vivo from the L. lactis strains administered to the test mice.

Another western blot analysis using anti-SG-115V antibody is shown in FIG. 31B. Ten μl of the noted samples was loaded onto each of lanes 1-7. Lane 1: 20 μg/ml purified SG-11V5; Lane 2: 2 μg/ml the purified SG-11V5; Lane 3: 0.2 μg/ml purified SG-11V5 protein administration; Lane 4:0.02 μg/ml purified SG-11V5; Lane 5: protein extracted tom fecal sample of mice administered with the L. lactis strain transformed with PnisA:otsBA::PnisA:SPusp45:SG-11V5 (nisin-induced); Lane 6: protein extracted from fecal sample of mice administered with the L. lactis strain transformed with PnisA:otsBA::PnisA:SPusp45:SG-11V5 (no nisin induction); Lane 7: protein extracted from the L. lactis strain transformed with PnisA:otsBA::Pusp45:SPusp45:SG-11V5 (no nisin induction). As shown in FIG. 31B, the L. lactis strains survive in the mice after administration and SG-11V5 proteins are expressed and secreted in vivo from the L. lactis strains administered to the test mice. Especially, amounts of the secreted SG-11V5 protein are higher under the control of the inducible nisinA promoter (nisin-induced) than under the control of the constitutive promoter, as evidenced by the comparison between lanes 5 and 7. On the other hand, western blot results indicate that SG-11V5 proteins are expressed independent of pre-induction of nisin. Based on the inputs of bacterial strain administration and protein expression level, it is estimated that up to 5 μg of nisin-induced SG-11V5 protein per 10⁹ cells per hour may be present in colon within 24 hours of administration, and up to 0.5 μg of SG-11V5 protein expressed under control of the constitutive promoter can be detected in colon.

### Example 22

Therapeutic Activity of SG-11V5 and SG-11V5-Expressing L. lactis in an In Vivo Model of Colitis

An in vivo study was performed to assess the therapeutic activity of L. lactis strains expressing SG-11V5 using a constitutive and inducible expression system. Before administering the L. lactis strains expressing SG-11V5 protein into an in vivo model of colitis, quality control test for the strains was performed. FIG. 32A shows colonies of the L. lactis attains for functional analysis described below. Colonies were counted to calculate a colony-forming unit (CFU) and estimate the number of viable L. lactis bacterial cells. (OD100=10¹¹ cells/ml). FIG. 32B shows PCR amplification to confirm target genes, otsBA and SG-11V5-coding sequence, cloned into the SG-11V5 expression plasmids. Lanes 1 and 4: the L. lactis strain transformed with PnisA:otsBA (negative control: without SG-11V5); Lanes 2 and 5: the L. lactis strain transformed with PnisA:otsBA::PnisA:SPusp45:SG11V5 (inducible expression of SG-11V5); Lanes 3 and 6: protein extracted from the L. lactis strain transformed with PnisA:otsBA::Pusp45:SPusp45:SG-11V3 (constitutive expression of SG-11V5). All the L. lactis strains have the otsBA expression cassette (Lanes 1-3) and two L. lactis strains possess the SG-11V5 expression cassette (Lanes 5-6) as Lane 4 is a negative control without SG-11V5-coding sequence. All the constructs tested were confirmed as expected. FIG. 32C shows western blot analysis of in vitro SG-11V5 protein expressed from the L. lactis expression plasmids with the constitutive promoter and the inducible promoter, respectively for SG-11V5 expression Thus, these strains are suitable for functional analysis of probiotic therapeutics comprising SG-11V5 to treat a gastrointestinal disorder or disease including colitis and mucositis.

Effects of SG-11 Administration and SG-11V3-Expressing L. lactis Administration on Epithelial Centric Barrier Function Readouts of Barrier Function in a DSS Model of Inflammatory Bowel Disease

To show that L. lactis strains expressing SG-11V5 or variants thereof possess functional activity which is equivalent to that of the purified SG-11V5 protein or variants thereof, the L. lactis generated as described, for example, in Examples 20 and 21 were administered to the DSS animal model of colitis as described, for example, in Examples 13 and 19. The in vivo assays were performed to compare activity of the test strains, e.g., L. lactis strain expressing SG-11V5 under the control of the inducible nisA promoter with nisin induction (PnisA:otsBA::PnisA:SPusp45:SG-11V5), and L. lactis strain expressing SG-11V5 under the control of the constitutive usp45 promoter (PnisA:otsBA::Pusp45:SPusp45:SG-11V5), with that of L. lactis strain not expressing SG-11V5 (parent pNZ8124 vector) as a negative control.

Specifically, the mice in this Example 22 were treated with DSS, a chemical known to induce intestinal epithelial damage and thereby reduce intestinal barrier integrity and function. These DSS mice were then administered an L. lactis strain expressing SG-11V5 as described above, or a positive or negative control treatment.

In this study, 3 independent groups of mice (10 mice per group) were used to test the 3 different L. lactis strains: Group 1: L. lactis harboring parent pNZ8124 vector, Group 2: L. lactis harboring inducible SG-11V5 vector (PnisA:otsBA::PnisA:SPusp45:SG-11V5), and Group 3: L. lactis harboring constitutive SG-11V5 vector (PnisA:otsBA::Pusp45:SPusp45:SG-11V5). Each of the strains in Groups 1-3 had been grown until the cultures reached an OD₆₀₀ of about 0.5, induced with nisin for 2 hours, concentrated to about 10¹¹ cells/mL in PBS with glycerol, and stored at −80° C. Cells were analyzed by Western blot to confirm protein expression. An additional 4 groups of mice (n=10 per group) were included as controls: Group 4: untreated; Group 5: treated p.o. with vehicle only; Group 6: intraperitoneal (i.p.) administration of Gly2-GLP2 (50 nmoles/kg); and Group 7: i.p. administration of SG-11V5 protein (160 nmoles/kg (4.0 mg/kg).

I.p. administration of Gly2-GLP2 and SG-11V5 was done twice per day, with i.p. administration to the right abdomen in the morning and to the left abdomen in the evening for 6 consecutive days (Day 0-Day 5) and then to the right abdomen on Day 6 prior to euthanasia and tissue recovery. Gly2-GLP2 (CPC Scientific Peptide Company) was prepared by dissolving in PBS(Corning 21-040-CV) with 5 mM NaOH to a concentration of 5 mg/mL Aliquots were stored at −80C prior to use.

Mice were housed 5 animals per cage and given food and water ad libitum. Following a 7-day acclimation period, treatments wore initiated in the morning (AM) of Day 0 with i.p. administration of Gly2-GLP2 or purified SG-11V5 protein as a positive control, oral gavage of strain vehicle only (phosphate buffered saline (PBS; Corning 21-040-CV)), or with oral gavage administration of the appropriate L. lactis expression strain.

Six hours after the initial treatment, the drinking water was changed to water containing 2.5% DSS. Fresh drinking water treated with 2.5% DSS was prepared and provided to all the mice on Days 0, 2, and 4 about 6 hours after AM dosing. Treatments were continued with SG-11V5 or Gly2-GLP2 twice a day (b.i.d.) in the morning and evening with i.p. injections at 50 nmoles/kg of Gly2-GLP2 and 160 nmoles/kg of SG-11V5. Also, treatments were continued with the L. lactis strains described above once a day (q.d) in the morning with p.o. administration at 10¹⁰ CFU of the strains comprising i) pNZ8124 vector, ii) inducible SG-11V5 vector, or iii) constitutive SG-11V5 vector.

On Day 6, only AM dosing was performed for intraperitoneal (i.p.) injection of protein and oral gavage (p.o) administration of L. lactis strains expressing SG-11V5 protein. Mice were fasted for 4 hours and then orally gavaged with 600 mg/kg 4KDa dextran labeled with fluorescin isothiocyanate (FITC) [4KDa-FITC]. About 50 minutes after the 4KDa-FITC gavage, mice were anesthetic with ketamine (ket)/xylazine (xyl) drug. Mice were injected with 10 ml/kg i.p of 10 mg/ml ketamine and 1 mg/ml xylazine (100 ul per 10 g body weight). One hour after administering 4KDa-FITC and about 10 minutes after ketamine/xylazine anesthesia, mice were euthanized, and blood and tissues were collected to assess barrier function and DSS model readouts. Table 14 summarizes the dosing schedule for an barrier functional study of protein therapeutics (i.p. dosing) and probiotic therapeutics (p.o. dosing).

TABLE 14
Dosing schedule for in vivo functional study

| Study | |
|---|---|
| Day | Activity |
| 0 | AM: body weights, oral gavage dosing, intraperitoneal |
| | dosing (right side) |
| | 6 hours post-AM dosing: DSS added to water |
| | PM: intraperitoneal dosing (left side) |
| 1 | AM: body weights, oral gavage dosing, intraperitoneal |
| | dosing (right side) |
| | PM: intraperitoneal dosing (left side) |
| 2 | AM: body weights, oral gavage dosing, intraperitoneal |
| | dosing (right side) |
| | PM: intraperitoneal dosing (left side), DSS water |
| | change |
| 3 | AM: body weights, oral gavage dosing, intraperitoneal |
| | dosing (right side) |
| | PM: intraperitoneal dosing (left side) |
| 4 | AM: body weights, oral gavage dosing, intraperitoneal |
| | dosing (right side) |
| | PM: intraperitoneal dosing (left side), DSS water |
| | change |
| 5 | AM: body weights, oral gavage dosing, intraperitoneal |
| | dosing (right side) |
| | PM: intraperitoneal dosing (left side) |
| 6 | AM: body weights, oral gavage dosing, intraperitoneal |
| | dosing (right side), change animals to new cage and fast |
| | 4 hours post-fasting: FITC-dextran dosing (oral gavage) |
| | 4 hours 50 minutes post-fasting: ket/xyl drug |
| | administration followed by euthanasia and tissue |
| | recovery |

A non-significant increase in 4KDa-FITC dextran translocation across the epithelial barrier was observed in mice receiving DSS and treated with SG-11V5 protein, as compared to DSS mice treated with vehicle mice in comparison to vehicle treated DSS mice. The magnitude of 4KDa-FITC dextran translocation observed for SG-11 seems higher than the positive control of Gly2-GLP2, but these values are not significant. Additionally, no significant change in 4KDa-FITC dextran was observed in mice receiving DSS and treated with either L. lactis expressing SG-11V5 protein inducibly or L. lactis expressing SG-11V5 protein constitutively, as compared to DSS mice rated with L. lactis expressing vehicle vector. Results are shown in FIG. 33A, and are presented as mean±SEM. The graph in FIG. 33A represents data pooled from one independent experiment (n=10 per group).
Effects of SG-11V5 Administration and SG-V5-Expressing L. lactis Administration on Inflammation Centric Readouts of Barrier Function in a DSS Model of Inflammatory Bowel Disease

Upon completion of the DSS models above, LBP levels were measured as an inflammation centric readout of barrier function following the protocol detailed above in Examples 7 and 13. From DSS models treated with protein and L. lactis strains described above, blood was collected and serum was isolated. LBP levels were measured in serum using a commercially available ELISA Kit (Enzo Life Sciences). Results are provided in FIG. 33B. A significant increase in LBP was observed in the model in response to DSS exposure. The positive control Gly2-GLp2 and SG-11V5 (SEQ ID NO:19) at a dose of 160 nmoles/kg were observed to show similar reductions in LBP with statistical significance (P<0.00001). On the other hand, no significant reduction in LBP was observed for the L. lactis strain which was induced to express SG-11V5, while the L. lactis strain constitutively expressing SG-11V5 showed significant reduction in LBP production (p=0.002)(FIG. 33B).

Effects of SG-11V5 Administration and SG-11V5-Expressing L. lactis Administration on Colon Length in a DSS Model of Inflammatory Bowel Disease

DSS models from Example 22 were also analyzed for the effect of SG-11V5 and SG-11V5-expressing L. lactis on the colon length. Colon length measurements were made and the results are shown in FIG. 34A. Similar results were obtained with SG-11V5 protein and SG-11V5-expressing L. lactis strains in both groups of DSS models where both treatment regimens resulted in a significant increase in the colon length. Especially, both L. lactis strains expressing SG-11V5 inducibly and constitutively show a significant improvement in colon length compared to a control strain (p=0.02 and p=0.04, respectively). Data in both graphs are presented as mean±SEM and represent data from an individual experiment. Statistical analysis was performed using a one-way ANOVA compared to DSS+vehicle followed by a Fishers LSD multiple comparison test.

Effects of SG-11V5 Administration and SG-11V5-Expressing L. lactis Administration on Colon Weight-to-Length Ratios in a DSS Model of Inflammatory Bowel Disease

DSS models from Example 22 were also analyzed for the effect of SG-11V5 protein and SG-11V5-expressing L. lactis on the colon weight-to-length ratio. Colon weight to length ratios were made and the results are shown in FIG. 34B. Similar results were obtained with SG-11V5 protein and SG-11V5-expressing L. lactis strains in both groups of DSS models where both treatment regimens resulted in a significant decrease in the colon weight-to-length ratio. All treatments of both L. lactis strains expressing SG-11V5 inducibly and constitutively improved colon weight to length ratios (p=0.01 and p=0.004, respectively). Statistical analysis was performed by a one-way ANOVA as compared to DSS+vehicle using a Fishers LSD multiple comparisons test Data are graphed as mean±SEM and each figure represent data from a single experiment.

Effects of SG-11V5 Administration and SG-11V5-Expressing L. lactis Administration on Body Weight in a DSS Model of Inflammatory Bowel Disease

Body weight was measured throughout the experimental models in this Example. In these models (FIG. 35A and FIG. 35B), similar trends in body weight were observed for SG-11V5 and L. lactis strains expressing SG-11V5 inducibly and constitutively. A significant improvement in body weight was observed at day 6 for SG-11V5 (SEQ ID NO:19) at 160 nmoles/kg. Similar patterns were observed in the DSS models where L. lactis strains expressing SG-11V5 protein inducibly and constitutively were administered. A group of the DSS models administrated with the L. lactis strains constitutively expressing SG-11V5 shows a statistically improved body weight changes at day 6 (p=0.02). For FIG. 35A and FIG. 35B, data are graphed as mean SEM and each graph represent data from an individual experiment. Statistical analysis was performed using a two-way ANOVA as compared to the DSS+vehicle group with a Fisher's LSD multiple comparison test.

Effects of SG-11V5 Administration and SG-11V5-Expressing L. lactis Administration on Gross Pathology in a DSS Model of Inflamatory Bowel Disease

Gross pathology observations of colon tissue are made for this Example as described in Examples 7 and 13 above. Briefly, a scoring system based on the level of visible blood and fecal pellet consistency was used. The scoring system used was: (0)=no gross pathology, (1)=streaks of blood visible in feces, (2)=completely bloody fecal pellets, (3) bloody fecal material visible in cecum, (4) bloody fecal material in cecum and loose stool. (5)=rectal bleeding. Similar results were obtained for SG-11V5 protein and L. lactis strains expressing SG-11V5 inducibly and constitutively. A significant improvement in gross pathology was observed for SG-11V5 (p<0.0001) and for the L. lactis strains expressing SG-11V5 inducibly and constitutively (p=0.03 and 0=0.0006, respectively). Data, illustrated in FIG. 36A, we presented as mean±SEM and include data from an individual experiment. Statistical analysis was performed using a one-way ANOVA followed by a Fisher's LSD multiple comparison test. FIG. 36B shows images of the entire colon from cecum to rectum from mice tested with clinical scores, as described above.

### Example 23

### Functionality of SG-11 and Variants Thereof in an In Vivo Model of Mucositis

Example 23 demonstrates the ability of SG-11 protein and variants thereof as disclosed herein to treat mucositis, such as oral mucositis, in an in vivo model. The experiment is therefore a demonstration that the aforementioned in vitro models, which described important functional and possible mechanistic modes of action, will translate into an in vivo model system of mucositis.

Forty-eight (48) male Syrian Golden Hamsters were used in the study.

Mucositis was induced by administering a single dose of radiation (40 Gy) directed to the left buccal cheek pouch at a rate of 2-2.5 Gy/min administered on Day 0. Radiation was generated with a 160 kilovolt potential (18.75-ma) source at a fecal distance of 10 cm, hardened with a 3.0 mm Al filtration system. Prior to irradiation, animals were anesthetized with an intraperitoneal injection of ketamine (160 mg/kg) and xylazine (8 mg/kg). The left buccal pouch was everted, fixed and isolated using a lead shield. Mucositis was evaluated clinically starting on Day 6 and continuing on alternate days until Day 28. The acute model has little systemic toxicity, resulting in few hamster deaths, thus permitting the use of smaller groups (n=7-8) for initial efficacy studies. It has abo been used to study specific mechanistic elements in the pathogenesis of mucositis.

The animals were divided into 6 treatment groups in which they were administered: Test agents (SG-11 or SG-11V5), a positive control (proprietary to Biomodels, LLC, Watertown, Mass.) or vehicle only were given by topical application to the left cheek pouch as detailed in Table 15 below.

TABLE 15
Detailed information of study design

| Group | Number of | | | Concentrations/ | | Dose | | Mucostitis |
|---|---|---|---|---|---|---|---|---|
| no. | Animals | Radiation | Treatment | dose (0.2 ml) | | schedule | Route | Evaluation |
| 1 | 8 males | Day 0 | Vehicle | — | | b.i.d. | Topical | Day 6-28 |
| 2 | 8 males | 40 Gy | Internal | N/A | | Day −2 | | |
| | | | positive | | | to 28 | | |
| | | | control | | | | | |
| 3 | 8 males | | SG-11 | 0.75 | mg/ml | b.i.d. | | |
| | | | | | | Day 0 | | |
| | | | | | | to 14 | | |
| 4 | 8 males | | SG-11V5 | 0.75 | mg/ml | b.i.d. | | |
| 5 | 8 males | | SG-11V5 | 0.24 | mg/ml | Day −2 | | |
| 6 | 8 males | | SG-11V5 | 0.075 | mg/ml | to 28 | | |

On Day 0, morning dosing was performed at least 1 hour prior to irradiation and at least 1 hour post-irradiation (for PM dose). On Day 28, animals were euthanized and the left cheek pouch rom animals in Groups 1, and Groups 3-6 were excised, placed in a cryovial, snap frozen, and stored at −80° C. until shipment.

Starting on Day 6 and continuing every second day thereafter (Days 8, 10, 12, 14, 16, 18, 20, 22, 24, 26, 28) each animal was photographed and evaluated for mucositis scoring. For the evaluation of mucositis, the animals were anesthetized with an inhalation anesthetic and the left pouch everted. Mucositis was scored visually by comparison to a validated photographic scale (FIG. 37A), ranging from 0 for normal, to 5 for severe ulceration (clinical scoring). In descriptive terms, this scale is defined in Table 16.

TABLE 16
Mucositis Scoring

| Score | Description |
|---|---|
| 0 | Pouch completely healthy. No erythema or vasodilation. |
| 1 | Light to severe erythema and vasodilation. No erosion |
| | of mucosa. |
| 2 | Severe erythema and vasodilation. Erosion of |
| | superficial aspects of mucosa leaving denuded areas. |
| | Decreased stippling of mucosa. |
| 3 | Formation of off-white ulcers in one or more places. |
| | Ulcers may have a yellow/gray color due to pseudomembrane. |
| | Cumulative size of ulcers should equal less than or equal |
| | to ¼ of the pouch. Severe erythema and vasodilation. |
| 4 | Cumulative seize of ulcers should equal about ½ of the |
| | pouch. Loss of pliability. Severe erythema and vasodilation. |
| 5 | Virtually all of pouch is ulcerated. Loss of pliability |
| | (pouch can only partially be extracted from mouth). |

A score of 1-2 is considered to represent a mild stage of the disease, whereas a score of 3.5 is considered to indicate moderate to severe ulcerative mucositis. At the conclusion of the experiment, the photographs were randomly numbered and scored by two independent, trained observers who graded the images in blinded fashion using the above-described scale (blinded scoring). Hamsters reaching a mucositis severity score of 4 or higher received buprenorphine (0.5 mg/kg) SC twice a day for 48 hours or until score dropped below 4.

Mean daily mucositis scores are shown in FIG. 37B. The maximum mean mucositis score observed in the Vehicle (Group 1) was 3.29±0.13 and was observed on Day 16. Animals dosed with the internal positive control (Group 2) exhibited peak mean mucositis scores of 2.00 on Day 14. Animals dosed with SG-11 (Group 3) experienced peak mean mucositis scores of 3.25 on Day 16. Animals dosed with SG-11V5 (Groups 4-6) at decreasing concentrations exhibited peak mucositis scores of 2.63, 3.13, and 3.00, respectively, on Days 16 and 18. The internal positive control group demonstrated the most robust decrease in mean mucositis scores out of any of the treatment groups, with the group dosed with 0.75 mg/mL (1.2 mg/kg) SG-11V5 (Group 4) showing the next best response. The other treatment groups showed some days with mean scores higher and some days with mean scores lower than vehicle, but were generally in-line with the mean scores of the vehicle group.

The mean daily percent bodyweight change data are shown in FIG. 38 for animals in all groups. Animals steadily pined weight throughout the duration of the study. In comparison to the vehicle group, animals in the treatment groups did not exhibit statistically significant weight change determined by using Area-Under-the-Curve (AUC) analysis followed by evaluation with one-way ANOVA with Holm-Sidak's multiple comparisons post-hoc test.

To examine the levels of clinically significant mucositis, as defined by presentation with open ulcers (score ≥3), the total number of days in which an animal exhibited an elevated score was summed and expressed as a percentage of the total number of days scored for each group. Statistical significance of observed differences was calculated using chi-squared analysis. The significance of differences observed between the control and treatment groups was evaluated by comparing the days with mucositis scores ≥3 and <3 between groups using chi-square analysis. The results of this analysis are shown in Table 17 far the entire study duration (through Day 28). Over the course of the study (Table 17), the percentage of animal days with a score of ≥3 in the Vehicle Group was 59.52%. The percentage of days with a score of ≥23 was statistically lower for animals dosed with the internal positive control (p<0.001), and with the 0.75 mg/mL and 0.075 mg/mL concentrations of SG-11V5 (p<0.001 and p=0.007, respectively) in comparison to the Vehicle Group.

TABLE 17
Chi-Square Analysis of Percent of Animal Days with a Mucositis Score ≥3

| | | | Total | | Chi Sq vs. | |
|---|---|---|---|---|---|---|
| Treatment | Days ≥3 | Days <3 | Animal Days | % Days ≥3 | Vehicle | P Value |
| Group 1: Vehicle | 100 | 68 | 168 | 59.52% | — | — |
| Group 2: Internal | 0 | 192 | 192 | 0.00% | 155.289 | <0.001 |
| Positive Control | | | | | | |
| Group 3: SG-11 | 96 | 96 | 192 | 50.00% | 2.904 | 0.088 |
| (0.75 mg/mL) | | | | | | |
| Group 4: SG-11V5 | 72 | 120 | 192 | 37.50% | 16.547 | <0.001 |
| (0.75 mg/mL) | | | | | | |
| Group 5: SG-11V5 | 103 | 89 | 192 | 53.65% | 1.031 | 0.310 |
| (0.24 mg/mL) | | | | | | |
| Group 6: SG-11V5 | 86 | 106 | 192 | 44.79% | 7.208 | 0.007 |
| (0.075 mg/mL) | | | | | | |

For these experiments, animals dosed with the positive control displayed multiple days of significant improvement in mucositis scores compared to the Vehicle control group. Aminals dosed with SG-11 showed one day of improvement, towards the aid of the study, while animals dosed with SG-11V5 showed multiple days, particularly the highest and lowest dose administered.

### Example 24

L. lactis strain NZ9000 wild type, and L. lactis strain NZ9000 with the thyA gene is deleted, and L. lactis strain NZ9000 with the thyA gene is replaced by SG-11V5, preceded by a usp45 signal peptide were started as overnight culture from −80 C. Then OD600 was measured for all strains and bacteria were resuspended into fresh media to OD-10 (˜*10\hat{ }10 bacteria/ml). Bacteria were incubated for 1 h at 30 C to express and secrete proteins and then supernatants were collected by spinning cultures down at 10000 g for 2 min and 5 ul were loaded on an SDS-page for protein detection with Western Blot, Gels were blotted using TurboBlot, and blocked with SuperBlock (Thermo Fisher) for 1 h. Rabbit-Anti-776 was added 1:3000 in SuperBlock overnight at 4 C followed by Anti-rabbit HRP was added 1:25000 for 30 min at RT in SuperBiock. Band were visualized with ChemiDoc Gel Imaging System using Luminata™ Forte Western HRP Substrate.

The gene sequence for SG-11V5, preceded by a usp45 signal peptide, was inserted into the native thyA site of L. lactis strain NZ9000, resulting in deletion of the native thyA gene. A negative control ti was also produced that only deleted the native thyA gene without inserting any additional sequence. FIG. 39 shows the ability of the chromosomally-inserted SG-11V5 gene to be expressed and secreted, as detected by Western blot of culture supernatants using an anti-. SG-11V5 polyclonal antibody. The negative control strain, as expected, does not show any evidence of SG-11V5 in culture supernatants.

Table 18 demonstrates SEQ ID NOs of the present disclosure with detailed information.

TABLE 18

| SEQ ID NO | Type | Description | Name |
|---|---|---|---|
| 1 | PRT | Full-length protein with signal sequence | SG-11 |
| 2 | DNA | coding sequence (cds) for SEQ ID NO: 1 | |
| 3 | PRT | SEQ ID NO: 1 without signal sequence and | SG-11 |
| | | without “start methionine” | |
| 4 | DNA | cds for SEQ ID NO: 3 | |
| 5 | PRT | SEQ ID NO: 3 expressed in pGEX6 vector and | SG-11 |
| | | cleaved by PreScission protease | |
| 6 | DNA | cds for SEQ ID NO: 5 | |
| 7 | PRT | SEQ ID NO: 3 with “start methionine” | SG-11 |
| 8 | DNA | cds for SEQ ID NO: 7 (codon optimized) | |
| 9 | PRT | SEQ ID NO: 3 With N-terminal FLAG tag | SG-11 |
| 10 | DNA | cds for SEQ ID NO: 9 (not codon optimized) | |
| 11 | PRT | Artificial variant of SEQ ID NO: 7 (C147V, C151S) | SG-11V1 |
| 12 | DNA | cds for SEQ ID NO: 11 (codon optimized) | |
| 13 | PRT | Artificial variant of SEQ ID NO: 7 (G84D, C147V, | SG-11V2 |
| | | C151S) | |
| 14 | DNA | cds for SEQ ID NO: 13 (codon optimized) | |
| 15 | PRT | Artificial variant of SEQ ID NO: 7 (N83S, C147V, | SG-11V3 |
| | | C151S) | |
| 16 | DNA | cds for SEQ ID NO: 15 (codon optimized) | |
| 17 | PRT | Artificial variant of SEQ ID NO: 7 (N53S, G84D, | SG-11V4 |
| | | C147V, C151S) | |
| 18 | DNA | cds for SEQ ID NO: 17 (codon optimized) | |
| 19 | PRT | Artificial variant of SEQ ID NO: 7 (N53S, N83S, | SG-11V5 |
| | | C147V, C151S) | |
| 20 | DNA | cds for SEQ ID NO: 19 (codon optimized) | |
| 21 | PRT | R. intestinalis hypothetical protein (GenBank | |
| | | WP_006857001.1) | |
| 22 | PRT | Roseburia sp. 831b hypothetical protein | |
| | | (GenBank WP_075679733.1) | |
| 23 | PRT | R. inulinivorans hypothetical protein (GenBank | |
| | | WP_055301040.1) | |
| 24 | PRT | Fragment of SEQ ID NO: 7 from Tables 6 and 8 | |
| 25 | PRT | Fragment of SG-11 from Table 6 | |
| 26 | PRT | Fragment of SG-11 from Table 6 | |
| 27 | PRT | Fragment of SG-11 from Table 6 | |
| 28 | PRT | Fragment of SG-11 Table 6 | |
| 29 | PRT | Fragment of SG-11 from Table 8 | |
| 30 | PRT | Fragment of SG-11 from Tables 6 and 8 | |
| 31 | PRT | Fragment of SG-11 from Table 8 | |
| 32 | PRT | FLAG tag | FLAG |
| 33 | PRT | Variant protein with X at positions 53, 83, 84, | |
| | | 147 and 151 | |
| 34 | PRT | Residues 72-232 of SEQ ID NO: 3 (73-233 of SEQ | SG-21 |
| | | ID NO: 7) | |
| 35 | DNA | cds for SEQ ID NO: 34 | |
| 36 | PRT | Met1-Res72-232 of SEQ ID NO: 3 | |
| 37 | DNA | cds for SEQ ID NO: NO: 36 | |
| 38 | PRT | Artificial variant of SEQ ID NO: 34 (C75V, C79S) | SG-21V1 |
| 39 | PRT | Artificial variant of SEQ ID NO: 34 (G12D, C75V, | SG-21V2 |
| | | C79S) | |
| 40 | PRT | Artificial variant of SEQ ID NO: 34 (N11S, C75V, | SG-21V5 |
| | | C79S) | |
| 41 | DNA | cds for Res72-232 of SEQ ID NO: 40 | |
| 42 | PRT | Artificial variant of SEQ ID NO: 36 (N12S, C76V, | |
| | | C80S) (with Met1) | |
| 43 | DNA | cds for SEQ ID NO: 42 | |
| 44 | PRT | Met-His-Clv-Res72-232 of NO: 3 | SG-21 |
| 45 | PRT | Artificial variant of SEQ ID NO: 44 (N18S, C82V, | SG-21V5 |
| | | C86S) | |
| 46 | PRT | C-Term Peptide of SEQ ID NO: 36 (res. 97-148) | |
| 47 | PRT | C-Term Peptide of SEQ ID NO: 36 (res. 4-49) | |
| 48 | PRT | C-Term Peptide of SEQ ID NO: 36 (res. 50-96) | |
| 49 | PRT | C-Term Peptide of SEQ ID NO: 36 (res. 25-74) | |
| 50 | PRT | Variant protein with X at positions 1, 12, 13, 76 | |
| | | and 80 | |
| 51 | DNA | pNZ8124 vector | |
| 52 | DNA | NisinA promoter (PnisA) | |
| 53 | DNA | PnisA:SPusp45:SG-11V5:Flag operon | |
| 54 | DNA | SPusp45:SG-11V5:Flag without TGA stop codon | |
| 55 | PRT | SPusp45-SG-11V5-Flag fusion protein | |
| 56 | DNA | PnisA:otsBA operon | |
| 57 | DNA | otsBA forward primer (otsBAfw) | |
| 58 | DNA | otsBA reverse primer (otsBArv) | |
| 59 | DNA | pNZ8124 forward primer (pNZ8124fw) | |
| 60 | DNA | pNZ8124 reverse primer (pNZ8124BArv) | |
| 61 | DNA | Pusp45:SPusp45:SG-11V5 operon | |
| 62 | DNA | usp45 forward primer (usp45fw) | |
| 63 | DNA | usp45 reverse primer (usp45rv) | |
| 64 | DNA | SG-11V5Ncol forward primer (SG-11V5Ncolfw) | |
| 65 | DNA | SG-11V5Xbal reverse primer (SG-11V5Xbalrv) | |
| 66 | DNA | PnisA:SPusp45:SG-11V5 operon | |
| 67 | PRT | Signal peptide of USP45 protein | |
| 68 | DNA | SG11 forward primer (SG11fw) | |
| 69 | DNA | SG11 reverse primer (SG11rv) | |
| 70 | DNA | L. lactis usp45 promoter | |
| 71 | DNA | L. lactis thyA promoter | |
| 72 | PRT | L. lactis thyA | |
| 73 | PRT | L. lactis dapA | |
| 74 | PRT | L. lactis sacA | |
| 75 | PRT | L. lactis mapA | |
| 76 | PRT | L. lactis lacZ | |
| 77 | PRT | L. lactis lacG | |
| 78 | PRT | L. lactis trePP | |
| 79 | PRT | L. lactis ptcC | |
| 80 | PRT | L. lactis sacB | |
| 81 | PRT | L. lactis malE | |
| 82 | PRT | L. lactis malF | |
| 83 | PRT | L. lactis malG | |
| 84 | PRT | L. lactis lacE | |
| 85 | PRT | L. lactis lacF | |
| 86 | PRT | L. lactis lacY | |
| 87 | PRT | L. lactis busAB | |
| 88 | PRT | E. coli otsA | |
| 89 | PRT | E. coli otsB | |
| 90 | DNA | L. lactis trehalose operon | |

Although the foregoing disclosure has been described in some detail by way of illustration and examples, which a for purposes of clarity of understanding, it will be apparent to those skilled in the art that certain changes and modifications may be practiced without departing from the spirit and scope of the disclosure, which is delineated in the appended claims. Therefore, the description should not be construed as limiting the scope of the disclosure.

### INCORPORATION BY REFERENCE

All references, articles, publications, patents, patent publications, and patent applications cited hemin am incorporated by reference in their entireties for all purposes.

However, mention of any reference, article, publication, patent, patent publication, and patent application cited herein is not, and should not be taken as, an acknowledgment or any form of suggestion that they constitute valid prior art or form part of the common general knowledge in any country in the world.

### REFERENCES

Molodecky et al. 2012, Increasing incidence and prevalence of the inflammatory bowel diseases with time, based on systematic review, Gastroenterology 142(1): 46-54.
Aroniadis et al, 2013, Fecal microbiota transplantation: past, present and future, Curr. Opin. Gastroenterol. 29(1): 79-84.
Maloy et al., 2011, Intestinal homeostasis and its breakdown in inflammatory bowel
IUPAC Commission on the Nomenclature of Organic Chemistry (CNOC) and IUPAC-IUB Commission on Biochemical Nomenclature (CBN), 1975, Nomenclature of α-Amino Acids, (Recommendations 1974), Biochemistry 14:449-462.
Remington's Pharmaceutical Sciences, 17th edition. Alfonso R, Gennaro (Ed.), Mark Publishing Company, Easton, Pa., USA, 1985.
Encyclopaedia of Pharmaceutical Technology, 3rd edition, James Swarbrick (Ed.), Informa Healthcare USA (Inc.), NY, USA, 2007.
Handbook of Pharmaceutical Salts: Properties, Selection, and Use, Stahl and Wermuth, Wiley-VCH, 2002,
Botoman et al., 1998, Management of Inflammatory Bowel Disease, Am. Fam. Physician, 57 (t): 57-68.
Neurath, 2014, Cytokines in Inflammatory Bowel Disease, Nature Reviews Immunology 14:329-342.
Zhang et al., 2007. Cytokines, Inflammation and Pain, Int. Anesthesiol. Clin. 45(2): 27-37.
Strober et al., Proinflammatory cytokines in the pathogenesis of inflammatory bowel diseases, Gastroenterology 140 (6): 1756-1767.
Boltin et al., 2013, Mucin Function in Inflammatory Bowel Disease: An Update, J. Clin. Gastroenterol. 47(2):106-111.
Kim et al., 2012, investigating Intestinal Inflammation in DSS-induced Model of IBD, Journal of Visualized Experiments, 60: 2-6.
Levesque et al., 2015 Converging goals of treatment of inflammatory bowel disease from clinical trials and practice, Gastroenterology 148:37-51.
Best et al., 1976, Development of a Crohns disease activity index. National Cooperative Crohns Disease Study, Gastroenterol. 70: 439-444.
Johansson, et al., 2014, Bacteria penetrate the normally impenetrable inner colon mucus layer in both marine colitis models and patients with ulcerative colitis, Gut 63:281-291.
Simmonds et al., 2014, Paneth cell metaplasia in newly diagnosed inflammatory bowel disease in children, BMC Gastroenterol. 14:93.
Gassler et al., 2001, Inflammatory bowel disease is associated with changes of enterocytic junctions, Am. J. Physiol. Gastrointest. Liver Physiol. 281:G216.0228.
Dewi, et al., 2004. In vitro assessment of human endothelial cell permeability: effects of inflammatory cytokines and dengue virus infection. J. Virol. Methods. 121: 171-180.
Mandic, et al., 2004, Evaluation of head and neck squamous cell carcinoma invasiveness by the electrical resistance breakdown assay, Clin. Exp. Metast. 21: 699-704.
Dewi, et al., 2008, Peripheral blood mononuclear cells increase the permeability of dengue virus-infected endothelial cells in association with down-regulation of vascular endothelial cadherin, J. Gen, Viral. 89642-652.
Sands, 2004, From symptom to diagnosis: clinical distinctions among various forms of intestinal inflammation, Gastroenterology 126: 1518-1532.
Danese et al., 2006, Etoopathogenesis of inflammatory bowel diseases, World J. Gastroenterol. 12:48074812.
Duncan, S. H., Aminov, R. L, Scott, K. P., Louis, P., Stanton, T. B., Flint, H. J. (2006). Proposal of Roseburia faecis sp. nov., Roseburia hominis sp. nov, and Roseburia inulinivorans sp. nov., based on isolates from human faeces. Int. J. Syst. Evol. Microbiol. Vol. 56, pgs. 2437-2441.
Bratt, et al., 2006 “A phase 1 trial with transgenic bacteria expressing interleukin-10 in Crohn's disease,” Clinical Gastroenterology and Hepatology, Vol. 4, pgs. 754-759.
Shigemori, et al., 2015, “Oral delivery of Lactococcus lactis that secretes bioactive heme oxygenase-1 alleviates development of acute colitis in mice,” Microbial Cell Factories, Vol. 14:189.
Steidler, et al., 2000 “Treatment of murine colitis by Lactococcus lactis secreting interleukin-10,” Science, Vol. 289, pgs. 1352-1355.
Hanniffy, et al., 2007. “Mucosal delivery of a pneumococcal vaccine using Lactococcus lactis affords protection against respiratory infection,” Journal of Infectious Diseases, Vol. 195, pgs. 185-193.
Vandenbroucke, et al., 2004, “Active delivery of trefoil factors by genetically modified Lactococcus lactis prevents and heals acute colitis in mice,” Gastroenterology, Vol. 127, pp. 502-513.
Sheth, et al., 2016, “Manipulating bacterial communities by in situ microbiome engineering,” Trends in Genetics, Vol. 32, Issue 4, pgs. 189-200.
Chan et al., 2016, “‘Deadman’ and ‘Passcode’ microbial kill switches for bacterial containment,” nature chemical biology, 12:82-86
Osorio J., 2016, “Synthetic biology: Genetic kill switches—a matter of fife or death,” Nat. Rev. Genet. 17(2):67
Stirling et al, 2017, “Rational Design of Evolutionarily Stable Microbial Kill Switches,” Molecular Cell 68:686-697
Termont et al., 2006, “Intracellular Accumulation of Trehalose Protects Lactococcus lactis from Freeze-Drying Damage and Bile Toxicity and Increases Gastric Acid Resistance,” App, and Envir. Microb. 72(12): 7694-7700.
Bolotin, Wincker et al. “The complete genome sequence of the lactic acid bacterium Lactococcus lactis ssp. lactis IL1403,” 2001, Genome Res, 11, 731-753
Steidler, Robinson et al. 1998, “Mucosal delivery of murine interleukin-2 (IL-2) and IL-6 by recombinant strains of Lactococcus lactis coexpressing antigen and cytokine,” Infect Immun, 66, 3183-3189
Bermudez-Humaran, Cortes-Perez et al. 2004, “Mucosal delivery of murine interleukin-2 (IL-2) and IL-6 by recombinant strains of Lactococcus lactis coexpressing antigen and cytokine,” J Med Microbiol, 53, 427-433
Hanniffy, Wiedermann et al. 2004, “Potential and opportunities for use of recombinant lactic acid bacteria in human health,” Adv Appl Microbiol. 56, 1-64
Wells and Mercenier 2008. “Mucosal delivery of therapeutic and prophylactic molecules using lactic acid bacteria,” Nat Rev Microbiol, 6, 349-362
Bermudez-Humaran, Kharrat et al. 2011, “Lactococci and lactobacilli as mucosal delivery vectors for therapeutic proteins and DNA vaccines,” Microb Cell Fact, 10 suppl 1, S4
Mielcarek, Alonso et al. 2001, “Nasal vaccination using live bacterial vectors,” Adv Drug Deliv Rev, 51, 55-69
Eriksson and Holmgren 2002, “Recent advances in mucosal vaccines and adjuvants,” Curr Opin Immunol, 14, 666-672

1. A recombinant host comprising:
a first nucleic acid comprising a promoter operably linked to a nucleic acid sequence encoding a signal peptide and a protein of interest;
wherein the signal peptide is N-terminal to the protein of interest;
wherein the promoter is selected from the group consisting of usp45 and thyA;
wherein the first nucleic acid is integrated into the genome of the host; and
wherein the host is a thymidylate synthase (thyA) auxotroph, a 4-hydroxy-tetrahydrodipicolinate synthase (dapA) auxotroph, or both.

2. The host of claim 1, wherein the host is a bacterium.

3. The host of claim 2, wherein the signal peptide is a usp45 signal peptide.

4. The host of claim 2, said host further comprising a viability enhancement.

5. The host of claim 4, wherein the viability enhancement comprises disruption of an endogenous gene encoding a protein involved in the catabolism or export of lactose, maltose, sucrose, trehalose, or glycine betaine.

6. The host of claim 5, wherein the protein involved in the catabolism of lactose, maltose, sucrose, trehalose, or glycine betaine is selected from the group consisting of a sucrose 6-phosphate, a maltose phosphorylase, a beta-galactosidase, a phospho-b-galactosidase, a trehalose 6-phosphate phosphorylase, permease IIC component, and combinations thereof.

7.-8. (canceled)

9. The host of claim 4, wherein the viability enhancement comprises an exogenous nucleic acid encoding a protein involved in the import or production of lactose, maltose, sucrose, trehalose, or glycine betaine.

10. The host of claim 9, wherein the protein involved in the import of lactose, maltose, sucrose, trehalose, or glycine betaine is selected from the group consisting of a sucrose phosphotransferase, a maltose ABC-transporter permease, a maltose binding protein, a lactose phosphotransferase, a lactose permease, a glycine betaine/proline ABC transporter permease component, a trehalose-6-phosphate synthase, a trehalose-6-phosphate phosphatase and combinations thereof.

11.-12. (canceled)

13. The host of claim 2, wherein the host is a non-pathogenic bacterium.

14. The host of claim 13, wherein the bacterium is a probiotic bacterium.

15. The host of claim 14, wherein the bacterium is selected from the group consisting of Bacteroides, Bifidobacterium, Clostridium, Escherichia, Eubacterium, Lactobacillus, Lactococcus, and Roseburia.

16. The host of claim 15, wherein the host is Lactococcus lactis.

17. The host of claim 16, wherein Lactococcus lactis is strain MG1363 or strain NZ9000.

18. The host of claim 15, wherein the protein of interest comprises an amino acid sequence with at least about 90%, at least about 95%, at least about 97%, at least about 98%, or at least about 99% sequence identity to SEQ ID NO: 19 and/or SEQ ID NO: 34.

19.-22. (canceled)

23. The host of claim 18, wherein the protein of interest comprises the amino acid sequence of SEQ ID NO: 19 or SEQ ID NO:34.

24. The host of claim 18, wherein the protein of interest comprises an amino acid sequence having at least about 90% sequence identity to SEQ ID NO: 19; and wherein
(i) the amino acid at position 147 of the protein of interest is valine, and/or
(ii) the amino acid at position 151 of the protein of interest is serine, and/or
(iii) the amino acid at position 84 of the protein of interest is aspartic acid, and/or
(iv) the amino acid at position 83 of the protein of interest is serine, and/or
(v) the amino acid at position 53 of the protein of interest is serine.

25. The host of claim 18, wherein the protein of interest comprises an amino acid sequence having at least about 90% sequence identity to SEQ ID NO: 19; and wherein
i. the amino acid at position 147 of the protein of interest is valine and the amino acid at position 151 of the protein of interest is serine; or
ii. the amino acid at position 84 of the protein of interest is aspartic acid, the amino acid at position 147 of the protein of interest is valine, and the amino acid at position 151 of the protein of interest is serine: or
iii. the amino acid at position 83 of the protein of interest is serine, the amino acid at position 147 of the protein of interest is valine, and the amino acid at position 151 of the protein of interest is serine; or
iv. the amino acid at position 53 of the protein of interest is serine, the amino acid at position 84 of the protein of interest is aspartic acid, the amino acid at position 147 of the protein of interest is valine, and the amino acid at position 151 of the protein of interest is serine; or
v. the amino acid at position 53 of the protein of interest is serine, the amino acid at position 83 of the protein of interest is serine, the amino acid at position 147 of the protein of interest is valine, and the amino acid at position 151 of the protein of interest is serine; or
vi. the amino acid at position 147 of the protein of interest is not cysteine, the amino acid at position 151 of the protein of interest is not cysteine, the amino acid at position 83 of the protein of interest is not asparagine, and/or the amino acid at position 53 of the protein of interest is not asparagine.

26.-30. (canceled)

31. The host of claim 18, wherein the protein of interest comprises an amino acid sequence having at least about 90% sequence identity to SEQ ID NO: 34; and wherein
(i) the amino acid at position 76 of the protein of interest is valine, and/or
(ii) the amino acid at position 80 of the protein of interest is serine; and/or
(iii) the amino acid at position 13 of the protein of interest is aspartic acid; and/or
(iv) the amino acid at position 12 of the protein of interest is serine.

32. The host of claim 18, wherein the protein of interest comprises an amino acid sequence having at least about 90/a sequence identity to SEQ ID NO: 34; and wherein
i. the amino acid at position 76 of the protein of interest is valine, and the amino acid at position 80 of the protein of interest is serine; or
ii. the amino acid at position 13 of the protein of interest is aspartic acid, the amino acid at position 76 of the protein of interest is valine, and the amino acid at position 80 of the protein of interest is serine; or
iii. the amino acid at position 12 of the protein of interest is serine, the amino acid at position 76 of the protein of interest is valine, and the amino acid at position 80 of the protein of interest is serine; or
iv. the amino acid at position 76 of the protein of interest is not cysteine, the amino acid at position 80 of the protein of interest is not cysteine, and the amino acid at position 12 of the protein of interest is not asparagine.

33.-35. (canceled)

36. The host of claim 15, wherein the protein of interest comprises an amino acid sequence having at least about 90% sequence identity to SEQ ID NO:46, SEQ ID NO: 47, SEQ ID NO: 48, or SEQ ID NO: 49.

37.-38. (canceled)

39. A method of treating a gastrointestinal epithelial cell barrier function disorder, comprising: administering to a subject in need thereof a pharmaceutical composition comprising:
i. a therapeutically effective amount of the recombinant host of claim 2;
ii. a pharmaceutically acceptable carrier.

40. The method of claim 39, wherein the composition comprises viable recombinant hosts.

41. The method of claim 39, wherein the composition comprises non-viable recombinant hosts.

42. The method of claim 39, wherein the gastrointestinal epithelial cell barrier function disorder is a disease associated with decreased gastrointestinal mucosal epithelium integrity.

43. The method of claim 39, wherein the disorder is selected from the group consisting of: inflammatory bowel disease, ulcerative colitis, Crohn's disease, short bowel syndrome, GI mucositis, oral mucositis, chemotherapy-induced mucositis, radiation-induced mucositis, necrotizing enterocolitis, pouchitis, a metabolic disease, celiac disease, inflammatory bowel syndrome, and chemotherapy associated steatohepatitis (CASH).

44. (canceled)

45. The method of claim 39, wherein the composition is formulated for oral ingestion.

46. The method of claim 39, wherein the composition is an edible product or the composition is formulated as a pill, a tablet, a capsule, a suppository, a liquid, or a liquid suspension.

47.-101. (canceled)


SEQUENCE LISTING (90 sequences)

SEQ ID NO: 1 - protein, 256 aa, Rosburia hominis
MKRLVCTVCSVLLCAGLLSGCGTSLEGEESVVYVGKKGVIASLDVETLDQSYYDETELKSYVDAEVEDYTAEHGKNAVKVESLKVEDGVAKLKMKYKTPEDYTAFNGIELYQGKVVASLAAGYVYDGEFARVEEGKVVGAATKQDIYSEDDLKVAIIRANTDVKVDGEICYVSCQNVKLTGKDSVSIRDGYYLETGSVTASVDVTGQESVGTEQLSGTEQMEMTGEPVNADDTEQTEAAAGDGSFETDVYTFIVYK

SEQ ID NO: 2 - DNA, 768 bp, Rosburia hominis
atgaagagattagtgtgcacggtctgcagtgtactgttgtgtgcgggacttctctccggatgcggtacctcgctggagggagaggaaagtgtcgtgtacgtgggaaagaaaggcgtgatagcgtcgctggatgtggagacgctcgatcagtcctactacgatgagacggaactgaagtcctatgtggatgcagaggtggaagattacaccgcggagcatggtaaaaatgcagtcaaggtggagagccttaaggtggaagacggtgtggcgaagcttaagatgaagtacaagacaccggaggattataccgcatttaatggaattgaactctatcaggggaaagtcgttgcttccctggcggcaggatacgtctacgacggggagttcgcccgcgtggaggaaggcaaggttgtgggagctgccacaaaacaggatatttactctgaggatgatttgaaagttgccatcatccgtgccaatacggatgtgaaggtggacggtgagatctgctatgtctcctgtcagaatgtgaagctgaccggaaaagacagtgtgtcgatccgtgacggatattatcttgagacgggaagcgtgacggcatccgtggatgtgaccggacaggagagcgtcgggaccgagcagctttcgggaaccgaacagatggagatgaccggggagccggtgaatgcggatgataccgagcagacagaggcggcggccggtgacggttcgttcgagacagacgtatatactttcattgtctacaaa

SEQ ID NO: 3 - protein, 232 aa, Rosburia hominis
LEGEESVVYVGKKGVIASLDVETLDQSYYDETELKSYVDAEVEDYTAEHGKNAVKVESLKVEDGVAKLKMKYKTPEDYTAFNGIELYQGKVVASLAAGYVYDGEFARVEEGKVVGAATKQDIYSEDDLKVAIIRANTDVKVDGEICYVSCQNVKLTGKDSVSIRDGYYLETGSVTASVDVTGQESVGTEQLSGTEQMEMTGEPVNADDTEQTEAAAGDGSFETDVYTFIVYK

SEQ ID NO: 4 - DNA, 696 bp, Rosburia hominis
ttggagggagaggaaagtgtcgtgtacgtgggaaagaaaggcgtgatagcgtcgctggatgtggagacgctcgatcagtcctactacgatgagacggaactgaagtcctatgtggatgcagaggtggaagattacaccgcggagcatggtaaaaatgcagtcaaggtggagagccttaaggtggaagacggtgtggcgaagcttaagatgaagtacaagacaccggaggattataccgcatttaatggaattgaactctatcaggggaaagtcgttgcttccctggcggcaggatacgtctacgacggggagttcgcccgcgtggaggaaggcaaggttgtgggagctgccacaaaacaggatatttactctgaggatgatttgaaagttgccatcatccgtgccaatacggatgtgaaggtggacggtgagatctgctatgtctcctgtcagaatgtgaagctgaccggaaaagacagtgtgtcgatccgtgacggatattatcttgagacgggaagcgtgacggcatccgtggatgtgaccggacaggagagcgtcgggaccgagcagctttcgggaaccgaacagatggagatgaccggggagccggtgaatgcggatgataccgagcagacagaggcggcggccggtgacggttcgttcgagacagacgtatatactttcattgtctacaaa

SEQ ID NO: 5 - protein, 241 aa, Artificial Sequence
  SG-11 expressed in pGEX6 vector and cleaved by Precision protease
GPLGSLEGEESVVYVGKKGVIASLDVETLDQSYYDETELKSYVDAEVEDYTAEHGKNAVKVESLKVEDGVAKLKMKYKTPEDYTAFNGIELYQGKVVASLAAGYVYDGEFARVEEGKVVGAATKQDIYSEDDLKVAIIRANTDVKVDGEICYVSCQNVKLTGKDSVSIRDGYYLETGSVTASVDVTGQESVGTEQLSGTEQMEMTGEPVNADDTEQTEAAAGDGSFETDVYTFIVYKAAAS

SEQ ID NO: 6 - DNA, 723 bp, Artificial Sequence
  SG-11 expressed in pGEX6 vector and cleaved by Precision protease
gggcccctgggatccctggagggagaggaaagtgtcgtgtacgtgggaaagaaaggcgtgatagcgtcgctggatgtggagacgctcgatcagtcctactacgatgagacggaactgaagtcctatgtggatgcagaggtggaagattacaccgcggagcatggtaaaaatgcagtcaaggtggagagccttaaggtggaagacggtgtggcgaagcttaagatgaagtacaagacaccggaggattataccgcatttaatggaattgaactctatcaggggaaagtcgttgcttccctggcggcaggatacgtctacgacggggagttcgcccgcgtggaggaaggcaaggttgtgggagctgccacaaaacaggatatttactctgaggatgatttgaaagttgccatcatccgtgccaatacggatgtgaaggtggacggtgagatctgctatgtctcctgtcagaatgtgaagctgaccggaaaagacagtgtgtcgatccgtgacggatattatcttgagacgggaagcgtgacggcatccgtggatgtgaccggacaggagagcgtcgggaccgagcagctttcgggaaccgaacagatggagatgaccggggagccggtgaatgcggatgataccgagcagacagaggcggcggccggtgacggttcgttcgagacagacgtatatactttcattgtctacaaagcggccgcatcg

SEQ ID NO: 7 - protein, 233 aa, Artificial Sequence
  SG-11 with start methionine
MLEGEESVVYVGKKGVIASLDVETLDQSYYDETELKSYVDAEVEDYTAEHGKNAVKVESLKVEDGVAKLKMKYKTPEDYTAFNGIELYQGKVVASLAAGYVYDGEFARVEEGKVVGAATKQDIYSEDDLKVAIIRANTDVKVDGEICYVSCQNVKLTGKDSVSIRDGYYLETGSVTASVDVTGQESVGTEQLSGTEQMEMTGEPVNADDTEQTEAAAGDGSFETDVYTFIVYK

SEQ ID NO: 8 - DNA, 699 bp, Artificial Sequence
  SG-11 with start methionine
atgttggagggtgaagagtctgttgtctatgtgggtaagaaaggtgtgatcgcgtccctggacgtcgagactctggaccagtcttactatgatgaaaccgagctgaagtcgtatgtggacgccgaagttgaggattacacggccgagcacggcaaaaatgccgtcaaagttgagagcttgaaagttgaggacggcgtggcaaagctgaagatgaaatacaagaccccagaggactacacggcgttcaatggtatcgagctgtatcagggcaaagtcgtcgcatccctggcagcgggctatgtgtacgacggtgagtttgcgcgcgtcgaagaaggcaaagttgtgggtgcggctacgaaacaagatatctacagcgaagatgacctgaaagtcgcgattattcgtgctaacaccgatgttaaagttgatggcgagatttgctacgttagctgtcaaaacgtaaagctgacgggtaaagatagcgtgagcattcgtgatggctattatctggaaaccggtagcgttacggcgagcgtcgatgttaccggtcaagagagcgtgggtaccgaacagctgagcggcaccgaacagatggaaatgaccggtgaaccggttaacgcagacgacacggaacaaaccgaagccgcggcaggcgacggtagcttcgagactgacgtgtacacctttatcgtgtacaag

SEQ ID NO: 9 - protein, 246 aa, Artificial Sequence
  SG-11 with N-terminal FLAG tag
MDYKDDDDKGSSHMLEGEESVVYVGKKGVIASLDVETLDQSYYDETELKSYVDAEVEDYTAEHGKNAVKVESLKVEDGVAKLKMKYKTPEDYTAFNGIELYQGKVVASLAAGYVYDGEFARVEEGKVVGAATKQDIYSEDDLKVAIIRANTDVKVDGEICYVSCQNVKLTGKDSVSIRDGYYLETGSVTASVDVTGQESVGTEQLSGTEQMEMTGEPVNADDTEQTEAAAGDGSFETDVYTFIVYK

SEQ ID NO: 10 - DNA, 738 bp, Artificial Sequence
  SG-11 with N-terminal FLAG tag (not codon optimized)
atggactacaaagacgatgacgacaagggcagcagccatatgctggagggagaggaaagtgtcgtgtacgtgggaaagaaaggcgtgatagcgtcgctggatgtggagacgctcgatcagtcctactacgatgagacggaactgaagtcctatgtggatgcagaggtggaagattacaccgcggagcatggtaaaaatgcagtcaaggtggagagccttaaggtggaagacggtgtggcgaagcttaagatgaagtacaagacaccggaggattataccgcatttaatggaattgaactctatcaggggaaagtcgttgcttccctggcggcaggatacgtctacgacggggagttcgcccgcgtggaggaaggcaaggttgtgggagctgccacaaaacaggatatttactctgaggatgatttgaaagttgccatcatccgtgccaatacggatgtgaaggtggacggtgagatctgctatgtctcctgtcagaatgtgaagctgaccggaaaagacagtgtgtcgatccgtgacggatattatcttgagacgggaagcgtgacggcatccgtggatgtgaccggacaggagagcgtcgggaccgagcagctttcgggaaccgaacagatggagatgaccggggagccggtgaatgcggatgataccgagcagacagaggcggcggccggtgacggttcgttcgagacagacgtatatactttcattgtctacaaa

SEQ ID NO: 11 - protein, 233 aa, Artificial Sequence
  Artificial variant of SG-11  (C147V, C151S)
MLEGEESVVYVGKKGVIASLDVETLDQSYYDETELKSYVDAEVEDYTAEHGKNAVKVESLKVEDGVAKLKMKYKTPEDYTAFNGIELYQGKVVASLAAGYVYDGEFARVEEGKVVGAATKQDIYSEDDLKVAIIRANTDVKVDGEIVYVSSQNVKLTGKDSVSIRDGYYLETGSVTASVDVTGQESVGTEQLSGTEQMEMTGEPVNADDTEQTEAAAGDGSFETDVYTFIVYK

SEQ ID NO: 12 - DNA, 699 bp, Artificial Sequence
  Artificial variant of SG-11  (C147V, C151S) (codon optimized)
atgttggagggtgaagagtctgttgtctatgtgggtaagaaaggtgtgatcgcgtccctggacgtcgagactctggaccagtcttactatgatgaaaccgagctgaagtcgtatgtggacgccgaagttgaggattacacggccgagcacggcaaaaatgccgtcaaagttgagagcttgaaagttgaggacggcgtggcaaagctgaagatgaaatacaagaccccagaggactacacggcgttcaatggtatcgagctgtatcagggcaaagtcgtcgcatccctggcagcgggctatgtgtacgacggtgagtttgcgcgcgtcgaagaaggcaaagttgtgggtgcggctacgaaacaagatatctacagcgaagatgacctgaaagtcgcgattattcgtgctaacaccgatgttaaagttgatggcgagattgtgtacgttagcagccaaaacgtaaagctgacgggtaaagatagcgtgagcattcgtgatggctattatctggaaaccggtagcgttacggcgagcgtcgatgttaccggtcaagagagcgtgggtaccgaacagctgagcggcaccgaacagatggaaatgaccggtgaaccggttaacgcagacgacacggaacaaaccgaagccgcggcaggcgacggtagcttcgagactgacgtgtacacctttatcgtgtacaag

SEQ ID NO: 13 - protein, 233 aa, Artificial Sequence
  Artificial variant of SG-11  (G84D, C147V, C151S)
MLEGEESVVYVGKKGVIASLDVETLDQSYYDETELKSYVDAEVEDYTAEHGKNAVKVESLKVEDGVAKLKMKYKTPEDYTAFNDIELYQGKVVASLAAGYVYDGEFARVEEGKVVGAATKQDIYSEDDLKVAIIRANTDVKVDGEIVYVSSQNVKLTGKDSVSIRDGYYLETGSVTASVDVTGQESVGTEQLSGTEQMEMTGEPVNADDTEQTEAAAGDGSFETDVYTFIVYK

SEQ ID NO: 14 - DNA, 699 bp, Artificial Sequence
  Artificial variant of SG-11  (G84D, C147V, C151S) (codon optimized)
atgttggagggtgaagagtctgttgtctatgtgggtaagaaaggtgtgatcgcgtccctggacgtcgagactctggaccagtcttactatgatgaaaccgagctgaagtcgtatgtggacgccgaagttgaggattacacggccgagcacggcaaaaatgccgtcaaagttgagagcttgaaagttgaggacggcgtggcaaagctgaagatgaaatacaagaccccagaggactacacggcgttcaatgacatcgagctgtatcagggcaaagtcgtcgcatccctggcagcgggctatgtgtacgacggtgagtttgcgcgcgtcgaagaaggcaaagttgtgggtgcggctacgaaacaagatatctacagcgaagatgacctgaaagtcgcgattattcgtgctaacaccgatgttaaagttgatggcgagattgtgtacgttagcagccaaaacgtaaagctgacgggtaaagatagcgtgagcattcgtgatggctattatctggaaaccggtagcgttacggcgagcgtcgatgttaccggtcaagagagcgtgggtaccgaacagctgagcggcaccgaacagatggaaatgaccggtgaaccggttaacgcagacgacacggaacaaaccgaagccgcggcaggcgacggtagcttcgagactgacgtgtacacctttatcgtgtacaag

SEQ ID NO: 15 - protein, 233 aa, Artificial Sequence
  Artificial variant of SG-11  (N83S, C147V, C151S)
MLEGEESVVYVGKKGVIASLDVETLDQSYYDETELKSYVDAEVEDYTAEHGKNAVKVESLKVEDGVAKLKMKYKTPEDYTAFSGIELYQGKVVASLAAGYVYDGEFARVEEGKVVGAATKQDIYSEDDLKVAIIRANTDVKVDGEIVYVSSQNVKLTGKDSVSIRDGYYLETGSVTASVDVTGQESVGTEQLSGTEQMEMTGEPVNADDTEQTEAAAGDGSFETDVYTFIVYK

SEQ ID NO: 16 - DNA, 699 bp, Artificial Sequence
  Artificial variant of SG-11  (N83S, C147V, C151S) (codon optimized)
atgttggagggtgaagagtctgttgtctatgtgggtaagaaaggtgtgatcgcgtccctggacgtcgagactctggaccagtcttactatgatgaaaccgagctgaagtcgtatgtggacgccgaagttgaggattacacggccgagcacggcaaaaatgccgtcaaagttgagagcttgaaagttgaggacggcgtggcaaagctgaagatgaaatacaagaccccagaggactacacggcgttcagcggtatcgagctgtatcagggcaaagtcgtcgcatccctggcagcgggctatgtgtacgacggtgagtttgcgcgcgtcgaagaaggcaaagttgtgggtgcggctacgaaacaagatatctacagcgaagatgacctgaaagtcgcgattattcgtgctaacaccgatgttaaagttgatggcgagattgtgtacgttagcagccaaaacgtaaagctgacgggtaaagatagcgtgagcattcgtgatggctattatctggaaaccggtagcgttacggcgagcgtcgatgttaccggtcaagagagcgtgggtaccgaacagctgagcggcaccgaacagatggaaatgaccggtgaaccggttaacgcagacgacacggaacaaaccgaagccgcggcaggcgacggtagcttcgagactgacgtgtacacctttatcgtgtacaag

SEQ ID NO: 17 - protein, 233 aa, Artificial Sequence
  Artificial variant of SG-11  (N53S, G84D, C147V, C151S)
MLEGEESVVYVGKKGVIASLDVETLDQSYYDETELKSYVDAEVEDYTAEHGKSAVKVESLKVEDGVAKLKMKYKTPEDYTAFNDIELYQGKVVASLAAGYVYDGEFARVEEGKVVGAATKQDIYSEDDLKVAIIRANTDVKVDGEIVYVSSQNVKLTGKDSVSIRDGYYLETGSVTASVDVTGQESVGTEQLSGTEQMEMTGEPVNADDTEQTEAAAGDGSFETDVYTFIVYK

SEQ ID NO: 18 - DNA, 699 bp, Artificial Sequence
  Artificial variant of SG-11  (N53S, G84D, C147V, C151S) (codon optimized)
atgttggagggtgaagagtctgttgtctatgtgggtaagaaaggtgtgatcgcgtccctggacgtcgagactctggaccagtcttactatgatgaaaccgagctgaagtcgtatgtggacgccgaagttgaggattacacggccgagcacggcaaatccgccgtcaaagttgagagcttgaaagttgaggacggcgtggcaaagctgaagatgaaatacaagaccccagaggactacacggcgttcaatgacatcgagctgtatcagggcaaagtcgtcgcatccctggcagcgggctatgtgtacgacggtgagtttgcgcgcgtcgaagaaggcaaagttgtgggtgcggctacgaaacaagatatctacagcgaagatgacctgaaagtcgcgattattcgtgctaacaccgatgttaaagttgatggcgagattgtgtacgttagcagccaaaacgtaaagctgacgggtaaagatagcgtgagcattcgtgatggctattatctggaaaccggtagcgttacggcgagcgtcgatgttaccggtcaagagagcgtgggtaccgaacagctgagcggcaccgaacagatggaaatgaccggtgaaccggttaacgcagacgacacggaacaaaccgaagccgcggcaggcgacggtagcttcgagactgacgtgtacacctttatcgtgtacaag

SEQ ID NO: 19 - protein, 233 aa, Artificial Sequence
  Artificial variant of SG-11  (N53S, N83S, C147V, C151S)
MLEGEESVVYVGKKGVIASLDVETLDQSYYDETELKSYVDAEVEDYTAEHGKSAVKVESLKVEDGVAKLKMKYKTPEDYTAFSGIELYQGKVVASLAAGYVYDGEFARVEEGKVVGAATKQDIYSEDDLKVAIIRANTDVKVDGEIVYVSSQNVKLTGKDSVSIRDGYYLETGSVTASVDVTGQESVGTEQLSGTEQMEMTGEPVNADDTEQTEAAAGDGSFETDVYTFIVYK

SEQ ID NO: 20 - DNA, 699 bp, Artificial Sequence
  Artificial variant of SG-11  (N53S, N83S, C147V, C151S) (codon optimized)
atgttggagggtgaagagtctgttgtctatgtgggtaagaaaggtgtgatcgcgtccctggacgtcgagactctggaccagtcttactatgatgaaaccgagctgaagtcgtatgtggacgccgaagttgaggattacacggccgagcacggcaaatccgccgtcaaagttgagagcttgaaagttgaggacggcgtggcaaagctgaagatgaaatacaagaccccagaggactacacggcgttcagcggtatcgagctgtatcagggcaaagtcgtcgcatccctggcagcgggctatgtgtacgacggtgagtttgcgcgcgtcgaagaaggcaaagttgtgggtgcggctacgaaacaagatatctacagcgaagatgacctgaaagtcgcgattattcgtgctaacaccgatgttaaagttgatggcgagattgtgtacgttagcagccaaaacgtaaagctgacgggtaaagatagcgtgagcattcgtgatggctattatctggaaaccggtagcgttacggcgagcgtcgatgttaccggtcaagagagcgtgggtaccgaacagctgagcggcaccgaacagatggaaatgaccggtgaaccggttaacgcagacgacacggaacaaaccgaagccgcggcaggcgacggtagcttcgagactgacgtgtacacctttatcgtgtacaag

SEQ ID NO: 21 - protein, 227 aa, Roseburia intestinalis
MLDADTDTVYVQKNGTVLSVDVETLDKDYYDETELKDYVTDAVSTYTGEHGKSAVKLENLSVKDGTATLKMKYKTPEDYTGFNGIELYEGKVVKALAAGYDFKTDFVSVEDGKVTGTATKEEIYSGEDLKVVIIKANRDVKVDGTICYVSSENVKLTGTDSVSIRDGYSLNSGSTADESDSDENIADGTESIGGSTEVSDTDVNDDTTYVKDDGAFETDVYTYIIYK

SEQ ID NO: 22 - protein, 215 aa, Roseburia sp. 831b
MLDVEESTVYVQKNGSVISTDIEDFSADYYDEDELKDYIGDEISSYTSENGKKSVSLESVSVKDSVAKLTMKYKTAEDYTNFNGVELYTGTIVKAMAAGYDFGVDFVSVKDGAVTGTATKDEIVDHDDYKVAVIKANTDVKVDGTIVYVSSQNVKVTGKNTVSIREGYLAADTTNVVGSTETVAETEAEEANQTEAVLEDEFASESDVYTYVIFK

SEQ ID NO: 23 - protein, 236 aa, Roseburia inulinivorans
MLEADTNTVYVSKHGKVVSMDVEQLDQSYYDETELKEFVDSAVDEYNTENGKNSVKVDDLTVEDGTAKLRMDYETVDDYTAFNGVELYEGKIVQALAAGYDFDTDFAGVDKDGCVTGVTRGDILAQEDLKVVIIKANTDVKIDGKILYVSCDNVTVTGKDSVSIKEGTGIEKTWITEAEEVPSTEAVLETESTEDAGDVIEGEVIIGTEEASGNDVVTNLSGGSSGTDVYTYIIYK

SEQ ID NO: 24 - protein, 13 aa, Artificial Sequence
  Fragment of SG-11 with start methionine
MLEGEESVVYVGK

SEQ ID NO: 25 - protein, 22 aa, Artificial Sequence
  Fragment of SG-11
GVIASLDVETLDQSYYDETELK

SEQ ID NO: 26 - protein, 17 aa, Artificial Sequence
  Fragment of SG-11
TPEDYTAFNGIELYQGK

SEQ ID NO: 27 - protein, 6 aa, Artificial Sequence
  Fragment of SG-11
ANTDVK

SEQ ID NO: 28 - protein, 14 aa, Artificial Sequence
  Fragment of SG-11
VDGEICYVSCQNVK

SEQ ID NO: 29 - protein, 67 aa, Artificial Sequence
  Fragment of SG-11
GYYLETGSVTASVDVTGQESVGTEQLSGTEQMEMTGEPVNADDTEQTEAAAGDGSFETDVYTFIVYK

SEQ ID NO: 30 - protein, 4 aa, Artificial Sequence
  Fragment of SG-11
NAVK

SEQ ID NO: 31 - protein, 17 aa, Artificial Sequence
  Fragment of SG-11
TPEDYTAFSGIELYQGK

SEQ ID NO: 32 - protein, 8 aa, Artificial Sequence
  FLAG tag
DYKDDDDK

SEQ ID NO: 33 - protein, 233 aa, Artificial Sequence
  Artificial variant of SG-11
  misc_feature (53)..(53): X is any amino acid as defined in the specification
  misc_feature (83)..(84): X is any amino acid as defined in the specification
  misc_feature (147)..(147): X is any amino acid as defined in the specification
  misc_feature (151)..(151): X is any amino acid as defined in the specification
MLEGEESVVYVGKKGVIASLDVETLDQSYYDETELKSYVDAEVEDYTAEHGKXAVKVESLKVEDGVAKLKMKYKTPEDYTAFXXIELYQGKVVASLAAGYVYDGEFARVEEGKVVGAATKQDIYSEDDLKVAIIRANTDVKVDGEIXYVSXQNVKLTGKDSVSIRDGYYLETGSVTASVDVTGQESVGTEQLSGTEQMEMTGEPVNADDTEQTEAAAGDGSFETDVYTFIVYK

SEQ ID NO: 34 - protein, 161 aa, Artificial Sequence
  SG-21
YKTPEDYTAFNGIELYQGKVVASLAAGYVYDGEFARVEEGKVVGAATKQDIYSEDDLKVAIIRANTDVKVDGEICYVSCQNVKLTGKDSVSIRDGYYLETGSVTASVDVTGQESVGTEQLSGTEQMEMTGEPVNADDTEQTEAAAGDGSFETDVYTFIVYK

SEQ ID NO: 35 - DNA, 483 bp, Artificial Sequence
  SG-21
tacaagacaccggaggattataccgcatttaatggaattgaactctatcaggggaaagtcgttgcttccctggcggcaggatacgtctacgacggggagttcgcccgcgtggaggaaggcaaggttgtgggagctgccacaaaacaggatatttactctgaggatgatttgaaagttgccatcatccgtgccaatacggatgtgaaggtggacggtgagatctgctatgtctcctgtcagaatgtgaagctgaccggaaaagacagtgtgtcgatccgtgacggatattatcttgagacgggaagcgtgacggcatccgtggatgtgaccggacaggagagcgtcgggaccgagcagctttcgggaaccgaacagatggagatgaccggggagccggtgaatgcggatgataccgagcagacagaggcggcggccggtgacggttcgttcgagacagacgtatatactttcattgtctacaaa

SEQ ID NO: 36 - protein, 162 aa, Artificial Sequence
  SG-21 with start methionine
MYKTPEDYTAFNGIELYQGKVVASLAAGYVYDGEFARVEEGKVVGAATKQDIYSEDDLKVAIIRANTDVKVDGEICYVSCQNVKLTGKDSVSIRDGYYLETGSVTASVDVTGQESVGTEQLSGTEQMEMTGEPVNADDTEQTEAAAGDGSFETDVYTFIVYK

SEQ ID NO: 37 - DNA, 486 bp, Artificial Sequence
  SG-21 with start methionine
atgtacaagacaccggaggattataccgcatttaatggaattgaactctatcaggggaaagtcgttgcttccctggcggcaggatacgtctacgacggggagttcgcccgcgtggaggaaggcaaggttgtgggagctgccacaaaacaggatatttactctgaggatgatttgaaagttgccatcatccgtgccaatacggatgtgaaggtggacggtgagatctgctatgtctcctgtcagaatgtgaagctgaccggaaaagacagtgtgtcgatccgtgacggatattatcttgagacgggaagcgtgacggcatccgtggatgtgaccggacaggagagcgtcgggaccgagcagctttcgggaaccgaacagatggagatgaccggggagccggtgaatgcggatgataccgagcagacagaggcggcggccggtgacggttcgttcgagacagacgtatatactttcattgtctacaaa

SEQ ID NO: 38 - protein, 161 aa, Artificial Sequence
  Artificial variant of SG-21
YKTPEDYTAFNGIELYQGKVVASLAAGYVYDGEFARVEEGKVVGAATKQDIYSEDDLKVAIIRANTDVKVDGEIVYVSSQNVKLTGKDSVSIRDGYYLETGSVTASVDVTGQESVGTEQLSGTEQMEMTGEPVNADDTEQTEAAAGDGSFETDVYTFIVYK

SEQ ID NO: 39 - protein, 161 aa, Artificial Sequence
  Artificial variant of SG-21
YKTPEDYTAFNDIELYQGKVVASLAAGYVYDGEFARVEEGKVVGAATKQDIYSEDDLKVAIIRANTDVKVDGEIVYVSSQNVKLTGKDSVSIRDGYYLETGSVTASVDVTGQESVGTEQLSGTEQMEMTGEPVNADDTEQTEAAAGDGSFETDVYTFIVYK

SEQ ID NO: 40 - protein, 161 aa, Artificial Sequence
  Artificial variant of SG-21
YKTPEDYTAFSGIELYQGKVVASLAAGYVYDGEFARVEEGKVVGAATKQDIYSEDDLKVAIIRANTDVKVDGEIVYVSSQNVKLTGKDSVSIRDGYYLETGSVTASVDVTGQESVGTEQLSGTEQMEMTGEPVNADDTEQTEAAAGDGSFETDVYTFIVYK

SEQ ID NO: 41 - DNA, 696 bp, Artificial Sequence
  Artificial variant of SG-21
ttggagggtgaagagtctgttgtctatgtgggtaagaaaggtgtgatcgcgtccctggacgtcgagactctggaccagtcttactatgatgaaaccgagctgaagtcgtatgtggacgccgaagttgaggattacacggccgagcacggcaaatccgccgtcaaagttgagagcttgaaagttgaggacggcgtggcaaagctgaagatgaaatacaagaccccagaggactacacggcgttcagcggtatcgagctgtatcagggcaaagtcgtcgcatccctggcagcgggctatgtgtacgacggtgagtttgcgcgcgtcgaagaaggcaaagttgtgggtgcggctacgaaacaagatatctacagcgaagatgacctgaaagtcgcgattattcgtgctaacaccgatgttaaagttgatggcgagattgtgtacgttagcagccaaaacgtaaagctgacgggtaaagatagcgtgagcattcgtgatggctattatctggaaaccggtagcgttacggcgagcgtcgatgttaccggtcaagagagcgtgggtaccgaacagctgagcggcaccgaacagatggaaatgaccggtgaaccggttaacgcagacgacacggaacaaaccgaagccgcggcaggcgacggtagcttcgagactgacgtgtacacctttatcgtgtacaag

SEQ ID NO: 42 - protein, 162 aa, Artificial Sequence
  Artificial variant of SG-21
MYKTPEDYTAFSGIELYQGKVVASLAAGYVYDGEFARVEEGKVVGAATKQDIYSEDDLKVAIIRANTDVKVDGEIVYVSSQNVKLTGKDSVSIRDGYYLETGSVTASVDVTGQESVGTEQLSGTEQMEMTGEPVNADDTEQTEAAAGDGSFETDVYTFIVYK

SEQ ID NO: 43 - DNA, 699 bp, Artificial Sequence; identical to SEQ ID NO: 20
  Artificial variant of SG-21

SEQ ID NO: 44 - protein, 168 aa, Artificial Sequence
  SG-21 with N-terminal addition
QQMGRGSYKTPEDYTAFNGIELYQGKVVASLAAGYVYDGEFARVEEGKVVGAATKQDIYSEDDLKVAIIRANTDVKVDGEICYVSCQNVKLTGKDSVSIRDGYYLETGSVTASVDVTGQESVGTEQLSGTEQMEMTGEPVNADDTEQTEAAAGDGSFETDVYTFIVYK

SEQ ID NO: 45 - protein, 168 aa, Artificial Sequence
  SG-21 with N-terminal addition
QQMGRGSYKTPEDYTAFSGIELYQGKVVASLAAGYVYDGEFARVEEGKVVGAATKQDIYSEDDLKVAIIRANTDVKVDGEIVYVSSQNVKLTGKDSVSIRDGYYLETGSVTASVDVTGQESVGTEQLSGTEQMEMTGEPVNADDTEQTEAAAGDGSFETDVYTFIVYK

SEQ ID NO: 46 - protein, 52 aa, Artificial Sequence
  C-terminal peptide of SG-11
YYLETGSVTASVDVTGQESVGTEQLSGTEQMEMTGEPVNADDTEQTEAAAGD

SEQ ID NO: 47 - protein, 46 aa, Artificial Sequence
  C-terminal peptide of SG-11
TPEDYTAFNGIELYQGKVVASLAAGYVYDGEFARVEEGKVVGAATK

SEQ ID NO: 48 - protein, 47 aa, Artificial Sequence
  C-terminal peptide of SG-11
QDIYSEDDLKVAIIRANTDVKVDGEICYVSCQNVKLTGKDSVSIRDG

SEQ ID NO: 49 - protein, 50 aa, Artificial Sequence
  C-terminal peptide of SG-11
LAAGYVYDGEFARVEEGKVVGAATKQDIYSEDDLKVAIIRANTDVKVDGE

SEQ ID NO: 50 - protein, 162 aa, Artificial Sequence
  Artificial variant of SG-21
  misc_feature (1)..(1): X is optional and/or is any amino acid as defined in the specification
  misc_feature (12)..(13): X is any amino acid as defined in the specification
  misc_feature (76)..(76): X is any amino acid as defined in the specification
  misc_feature (80)..(80): X is any amino acid as defined in the specification
XYKTPEDYTAFXXIELYQGKVVASLAAGYVYDGEFARVEEGKVVGAATKQDIYSEDDLKVAIIRANTDVKVDGEIXYVSXQNVKLTGKDSVSIRDGYYLETGSVTASVDVTGQESVGTEQLSGTEQMEMTGEPVNADDTEQTEAAAGDGSFETDVYTFIVYK

SEQ ID NO: 51 - DNA, 3260 bp, Artificial Sequence
  pNZ8124 full construct
agatctagtcttataactatactgacaatagaaacattaacaaatctaaaacagtcttaattctatcttgagaaagtattggtaataatattattgtcgataacgcgagcataataaacggctctgattaaattctgaagtttgttagatacaatgatttcgttcgaaggaactacaaaataaattataaggaggcactcaaaatgaaaaaaaagattatctcagctattttaatgtctacagtgatcttaagtgctgcagccccgttgtcaggtgtttacgctgatatcacggccatgggtactgcaggcatgcggtaccactagttctagagagctcaagctttctttgaaccaaaattagaaaaccaaggcttgaaacgttcaattgaaatggcaattaaacaaattacagcacgtgttgctttgattgatagccaaaaagcagcagttgataaagcaattactgatattgctgaaaaattgtaatttataaataaaaatcaccttttagaggtggtttttttatttataaattattcgtttgatttcgctttcgatagaacaatcaaatcgtttctgagacgttttagcgtttatttcgtttagttatcggcataatcgttaaaacaggcgttatcgtagcgtaaaagcccttgagcgtagcgtggctttgcagcgaagatgttgtctgttagattatgaaagccgatgactgaatgaaataataagcgcagcgtccttctatttcggttggaggaggctcaagggagtttgagggaatgaaattccctcatgggtttgattttaaaaattgcttgcaattttgccgagcggtagcgctggaaaatttttgaaaaaaatttggaatttggaaaaaaatggggggaaaggaagcgaattttgcttccgtactacgaccccccattaagtgccgagtgccaatttttgtgccaaaaacgctctatcccaactggctcaagggtttgaggggtttttcaatcgccaacgaatcgccaacgttttcgccaacgttttttataaatctatatttaagtagctttatttttgtttttatgattacaaagtgatacactaattttataaaattatttgattggagttttttaaatggtgatttcagaatcgaaaaaaagagttatgatttctctgacaaaagagcaagataaaaaattaacagatatggcgaaacaaaaagatttttcaaaatctgcggttgcggcgttagctatagaagaatatgcaagaaaggaatcagaacaaaaaaaataagcgaaagctcgcgtttttagaaggatacgagttttcgctacttgtttttgataaggtaattatatcatggctattaaaaatactaaagctagaaattttggatttttattatatcctgactcaattcctaatgattggaaagaaaaattagagagtttgggcgtatctatggctgtcagtcctttacacgatatggacgaaaaaaaagataaagatacatggaatagtagtgatgttatacgaaatggaaagcactataaaaaaccacactatcacgttatatatattgcacgaaatcctgtaacaatagaaagcgttaggaacaagattaagcgaaaattggggaatagttcagttgctcatgttgagatacttgattatatcaaaggttcatatgaatatttgactcatgaatcaaaggacgctattgctaagaataaacatatatacgacaaaaaagatattttgaacattaatgattttgatattgaccgctatataacacttgatgaaagccaaaaaagagaattgaagaatttacttttagatatagtggatgactataatttggtaaatacaaaagatttaatggcttttattcgccttaggggagcggagtttggaattttaaatacgaatgatgtaaaagatattgtttcaacaaactctagcgcctttagattatggtttgagggcaattatcagtgtggatatagagcaagttatgcaaaggttcttgatgctgaaacgggggaaataaaatgacaaacaaagaaaaagagttatttgctgaaaatgaggaattaaaaaaagaaattaaggacttaaaagagcgtattgaaagatacagagaaatggaagttgaattaagtacaacaatagatttattgagaggagggattattgaataaataaaagcccccctgacgaaagtcgacggcaatagttacccttattatcaagataagaaagaaaaggatttttcgctacgctcaaatcctttaaaaaaacacaaaagaccacattttttaatgtggtcttttattcttcaactaaagcacccattagttcaacaaacgaaaattggataaagtgggatatttttaaaatatatatttatgttacagtaatattgacttttaaaaaaggattgattctaatgaagaaagcagacaagtaagcctcctaaattcactttagataaaaatttaggaggcatatcaaatgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccttggacttcatttactgggtttaacttaaatatcaataataatagtaattaccttctacccattattacagcaggaaaattcattaataaaggtaattcaatatatttaccgctatctttacaggtacatcattctgtttgtgatggttatcatgctggattgtttatgaactctattcaggaattgtcagataggcctaatgactggcttttataatatgagataatgccgactgtactttttacagtcggttttctaatgtcactaacctgccccgttagttgaagaaggtttttatattacagctcc

SEQ ID NO: 52 - DNA, 197 bp, Artificial Sequence
  nisA promoter
ctagtcttataactatactgacaatagaaacattaacaaatctaaaacagtcttaattctatcttgagaaagtattggtaataatattattgtcgataacgcgagcataataaacggctctgattaaattctgaagtttgttagatacaatgatttcgttcgaaggaactacaaaataaattataaggaggcactca

SEQ ID NO: 53 - DNA, 1031 bp, Artificial Sequence
  PnisASPusp45SG-11V5Flag operon
ctagtcttataactatactgacaatagaaacattaacaaatctaaaacagtcttaattctatcttgagaaagtattggtaataatattattgtcgataacgcgagcataataaacggctctgattaaattctgaagtttgttagatacaatgatttcgttcgaaggaactacaaaataaattataaggaggcactcaaaatgaaaaaaaagattatctcagctattttaatgtctacagtgatcttaagtgctgcagccccgttgtcaggtgtttacgctgatatcttggagggtgaagagtctgttgtctatgtgggtaagaaaggtgtgatcgcgtccctggacgtcgagactctggaccagtcttactatgatgaaaccgagctgaagtcgtatgtggacgccgaagttgaggattacacggccgagcacggcaaatccgccgtcaaagttgagagcttgaaagttgaggacggcgtggcaaagctgaagatgaaatacaagaccccagaggactacacggcgttcagcggtatcgagctgtatcagggcaaagtcgtcgcatccctggcagcgggctatgtgtacgacggtgagtttgcgcgcgtcgaagaaggcaaagttgtgggtgcggctacgaaacaagatatctacagcgaagatgacctgaaagtcgcgattattcgtgctaacaccgatgttaaagttgatggcgagattgtgtacgttagcagccaaaacgtaaagctgacgggtaaagatagcgtgagcattcgtgatggctattatctggaaaccggtagcgttacggcgagcgtcgatgttaccggtcaagagagcgtgggtaccgaacagctgagcggcaccgaacagatggaaatgaccggtgaaccggttaacgcagacgacacggaacaaaccgaagccgcggcaggcgacggtagcttcgagactgacgtgtacacctttatcgtgtacaaggactacaaagacgatgacgacaagtaatctagagagctcaagcttttga

SEQ ID NO: 54 - DNA, 829 bp, Artificial Sequence
  SPusp45SG-11V5Flag without TGA stop codon operon
atgaaaaaaaagattatctcagctattttaatgtctacagtgatcttaagtgctgcagccccgttgtcaggtgtttacgctgatatcttggagggtgaagagtctgttgtctatgtgggtaagaaaggtgtgatcgcgtccctggacgtcgagactctggaccagtcttactatgatgaaaccgagctgaagtcgtatgtggacgccgaagttgaggattacacggccgagcacggcaaatccgccgtcaaagttgagagcttgaaagttgaggacggcgtggcaaagctgaagatgaaatacaagaccccagaggactacacggcgttcagcggtatcgagctgtatcagggcaaagtcgtcgcatccctggcagcgggctatgtgtacgacggtgagtttgcgcgcgtcgaagaaggcaaagttgtgggtgcggctacgaaacaagatatctacagcgaagatgacctgaaagtcgcgattattcgtgctaacaccgatgttaaagttgatggcgagattgtgtacgttagcagccaaaacgtaaagctgacgggtaaagatagcgtgagcattcgtgatggctattatctggaaaccggtagcgttacggcgagcgtcgatgttaccggtcaagagagcgtgggtaccgaacagctgagcggcaccgaacagatggaaatgaccggtgaaccggttaacgcagacgacacggaacaaaccgaagccgcggcaggcgacggtagcttcgagactgacgtgtacacctttatcgtgtacaaggactacaaagacgatgacgacaagtaatctagagagctcaagcttt

SEQ ID NO: 55 - protein, 269 aa, Artificial Sequence
  SPusp45-SG-11V5-Flag fusion protein
MKKKIISAILMSTVILSAAAPLSGVYADILEGEESVVYVGKKGVIASLDVETLDQSYYDETELKSYVDAEVEDYTAEHGKSAVKVESLKVEDGVAKLKMKYKTPEDYTAFSGIELYQGKVVASLAAGYVYDGEFARVEEGKVVGAATKQDIYSEDDLKVAIIRANTDVKVDGEIVYVSSQNVKLTGKDSVSIRDGYYLETGSVTASVDVTGQESVGTEQLSGTEQMEMTGEPVNADDTEQTEAAAGDGSFETDVYTFIVYKDYKDDDDK

SEQ ID NO: 56 - DNA, 2421 bp, Artificial Sequence
  PnisAotsBA operon
ctagtcttataactatactgacaatagaaacattaacaaatctaaaacagtcttaattctatcttgagaaagtattggtaataatattattgtcgataacgcgagcataataaacggctctgattaaattctgaagtttgttagatacaatgatttcgttcgaaggaactacaaaataaattataaggaggcactcaaaatgacagaaccgttaaccgaaacccctgaactatccgcgaaatatgcctggttttttgatcttgatggaacgctggcggaaatcaaaccgcatcccgatcaggtcgtcgtgcctgacaatattctgcaaggactacagctactggcaaccgcaagtgatggtgcattggcattgatatcagggcgctcaatggtggagcttgacgcactggcaaaaccttatcgcttcccgttagcgggcgtgcatggggcggagcgccgtgacatcaatggtaaaacacatatcgttcatctgccggatgcgattgcgcgtgatattagcgtgcaactgcatacagtcatcgctcagtatcccggcgcggagctggaggcgaaagggatggcttttgcgctgcattatcgtcaggctccgcagcatgaagacgcattaatgacattagcgcaacgtattactcagatctggccacaaatggcgttacagcagggaaagtgtgttgtcgagatcaaaccgagaggtaccagtaaaggtgaggcaattgcagcttttatgcaggaagctccctttatcgggcgaacgcccgtatttctgggcgatgatttaaccgatgaatctggcttcgcagtcgttaaccgactgggcggaatgtcagtaaaaattggcacaggtgcaactcaggcatcatggcgactggcgggtgtgccggatgtctggagctggcttgaaatgataaccaccgcattacaacaaaaaagagaaaataacaggagtgatgactatgagtcgtttagtcgtagtatctaaccggattgcaccaccagacgagcacgccgccagtgccggtggccttgccgttggcatactgggggcactgaaagccgcaggcggactgtggtttggctggagtggtgaaacagggaatgaggatcagccgctaaaaaaggtgaaaaaaggtaacattacgtgggcctcttttaacctcagcgaacaggaccttgacgaatactacaaccaattctccaatgccgttctctggcccgcttttcattatcggctcgatctggtgcaatttcagcgtcctgcctgggacggctatctacgcgtaaatgcgttgctggcagataaattactgccgctgttgcaagacgatgacattatctggatccacgattatcacctgttgccatttgcgcatgaattacgcaaacggggagtgaataatcgcattggtttctttctgcatattcctttcccgacaccggaaatcttcaacgcgctgccgacatatgacaccttgcttgaacagctttgtgattatgatttgctgggtttccagacagaaaacgatcgtctggcgttcctggattgtctttctaacctgacccgcgtcacgacacgtagcgcaaaaagccatacagcctggggcaaagcatttcgaacagaagtctacccgatcggcattgaaccgaaagaaatagccaaacaggctgccgggccactgccgccaaaactggcgcaacttaaagcggaactgaaaaacgtacaaaatatcttttctgtcgaacggctggattattccaaaggtttgccagagcgttttctcgcctatgaagcgttgctggaaaaatatccgcagcatcatggtaaaattcgttatacccagattgcaccaacgtcgcgtggtgatgtgcaagcctatcaggatattcgtcatcagctcgaaaatgaagctggacgaattaatggtaaatacgggcaattaggctggacgccgctttattatttgaatcagcattttgaccgtaaattactgatgaaaatattccgctactctgacgtgggcttagtgacgccactgcgtgacgggatgaacctggtagcaaaagagtatgttgctgctcaggacccagccaatccgggcgttcttgttctttcgcaatttgcgggagcggcaaacgagttaacgtcggcgttaattgttaacccctacgatcgtgacgaagttgcagctgcgctggatcgtgcattgactatgtcgctggcggaacgtatttcccgtcatgcagaaatgctggacgttatcgtgaaaaacgatattaaccactggcaggagtgcttcattagcgacctaaagcagatagttccgcgaagcgcggaaagccagcagcgcgataaagttgctacctttccaaagcttgcgtagatgaaaaaaaagattatctcag

SEQ ID NO: 57 - DNA, 37 bp, Artificial Sequence
  otsBAfw oligonucleotide primer
ttataaggaggcactcaaaatgacagaaccgttaacc

SEQ ID NO: 58 - DNA, 44 bp, Artificial Sequence
  ostBArw oligonucleotide primer
ctgagataatcttttttttcatctacgcaagctttggaaaggta

SEQ ID NO: 59 - DNA, 19 bp, Artificial Sequence
  pNZ8124fw oligonucleotide primer
tttgagtgcctccttataa

SEQ ID NO: 60 - DNA, 20 bp, Artificial Sequence
  pNZ8124rv oligonucleotide primer
atgaaaaaaaagattatctc

SEQ ID NO: 61 - DNA, 897 bp, Artificial Sequence
  Pusp45SPusp45SG-11V5 operon
gatatctgttttgtaatcataaagaaatattaaggtggggtaggaatagtataatatgtttattcaaccgaacttaatgggaggaaaaattaaaaaagaacagttatgaaaaaaaagattatctcagctattttaatgtctacagtgatactttctgctgcagccccgttgtcaggtgtttacgctccatggttggagggtgaagagtctgttgtctatgtgggtaagaaaggtgtgatcgcgtccctggacgtcgagactctggaccagtcttactatgatgaaaccgagctgaagtcgtatgtggacgccgaagttgaggattacacggccgagcacggcaaatccgccgtcaaagttgagagcttgaaagttgaggacggcgtggcaaagctgaagatgaaatacaagaccccagaggactacacggcgttcagcggtatcgagctgtatcagggcaaagtcgtcgcatccctggcagcgggctatgtgtacgacggtgagtttgcgcgcgtcgaagaaggcaaagttgtgggtgcggctacgaaacaagatatctacagcgaagatgacctgaaagtcgcgattattcgtgctaacaccgatgttaaagttgatggcgagattgtgtacgttagcagccaaaacgtaaagctgacgggtaaagatagcgtgagcattcgtgatggctattatctggaaaccggtagcgttacggcgagcgtcgatgttaccggtcaagagagcgtgggtaccgaacagctgagcggcaccgaacagatggaaatgaccggtgaaccggttaacgcagacgacacggaacaaaccgaagccgcggcaggcgacggtagcttcgagactgacgtgtacacctttatcgtgtacaagtgatctaga

SEQ ID NO: 62 - DNA, 41 bp, Artificial Sequence
  usp45fw oligonucleotide primer
atcgggatatctgttttgtaatcataaagaaatattaaggt

SEQ ID NO: 63 - DNA, 40 bp, Artificial Sequence
  usp45rv oligonucleotide primer
atcggccatggagcgtaaacacctgacaacggggctgcag

SEQ ID NO: 64 - DNA, 31 bp, Artificial Sequence
  SG-11V5NcoIfw oligonucleotide primer
atcggccatggttggagggtgaagagtctgt

SEQ ID NO: 65 - DNA, 34 bp, Artificial Sequence
  SG-11V5XbaIrv oligonucleotide primer
atcggtctagattacttgtacacgataaaggtgt

SEQ ID NO: 66 - DNA, 982 bp, Artificial Sequence
  PnisASPusp45SG-11V5 operon
ctagtcttataactatactgacaatagaaacattaacaaatctaaaacagtcttaattctatcttgagaaagtattggtaataatattattgtcgataacgcgagcataataaacggctctgattaaattctgaagtttgttagatacaatgatttcgttcgaaggaactacaaaataaattataaggaggcactcaaaatgaaaaaaaagattatctcagctattttaatgtctacagtgatcttaagtgctgcagccccgttgtcaggtgtttacgctgatatcttggagggtgaagagtctgttgtctatgtgggtaagaaaggtgtgatcgcgtccctggacgtcgagactctggaccagtcttactatgatgaaaccgagctgaagtcgtatgtggacgccgaagttgaggattacacggccgagcacggcaaatccgccgtcaaagttgagagcttgaaagttgaggacggcgtggcaaagctgaagatgaaatacaagaccccagaggactacacggcgttcagcggtatcgagctgtatcagggcaaagtcgtcgcatccctggcagcgggctatgtgtacgacggtgagtttgcgcgcgtcgaagaaggcaaagttgtgggtgcggctacgaaacaagatatctacagcgaagatgacctgaaagtcgcgattattcgtgctaacaccgatgttaaagttgatggcgagattgtgtacgttagcagccaaaacgtaaagctgacgggtaaagatagcgtgagcattcgtgatggctattatctggaaaccggtagcgttacggcgagcgtcgatgttaccggtcaagagagcgtgggtaccgaacagctgagcggcaccgaacagatggaaatgaccggtgaaccggttaacgcagacgacacggaacaaaccgaagccgcggcaggcgacggtagcttcgagactgacgtgtacacctttatcgtgtacaag

SEQ ID NO: 67 - protein, 27 aa, Artificial Sequence
  N-terminal signal peptide derived from the usp45 protein
MKKKIISAILMSTVILSAAAPLSGVYA

SEQ ID NO: 68 - DNA, 39 bp, Artificial Sequence
  SG11fw oligonucleotide primer
aggtgtttacgctgatatcttggagggtgaagagtctgt

SEQ ID NO: 69 - DNA, 66 bp, Artificial Sequence
  SG11rv oligonucleotide primer
aaagcttgagctctctagattacttgtcgtcatcgtctttgtagtccttgtacacgataaaggtgt

SEQ ID NO: 70 - DNA, 99 bp, Lactococcus lactis
tgttttgtaatcataaagaaatattaaggtggggtaggaatagtataatatgtttattcaaccgaacttaatgggaggaaaaattaaaaaagaacagtt

SEQ ID NO: 71 - DNA, 152 bp, Lactococcus lactis
tggatattttttataaatctggtttgaacaaattatattgacatctctttttctatcctgataattctgagaggttattttgggaaatactattgaaccatatcgaggtggtgtggtataatgaagggaattaaaaaagataggaaaatttc

SEQ ID NO: 72 - protein, 279 aa, Lactococcus lactis
MTYADKIFKQNIQNILDNGVFSENARPKYKDGQTANSKYVTGSFVTYDLQKGEFPITTLRPIPIKSAIKELMWIYQDQTSELAILEEKYGVKYWGEWGIGDGTIGQRYGATVKKYNIIGKLLDGLAKNPWNRRNIINLWQYEDFEETEGLLPCAFQTMFDVRREQDGQIYLDATLIQRSNDMLVAHHINAMQYVALQMMIAKHFSWKVGKFFYFVNNLHIYDNQFEQANELVKRTASDKEPRLVLNVPDGTNFFDIKPEDFELVDYEPVKPQLKFDLAI

SEQ ID NO: 73 - protein, 297 aa, Lactococcus lactis
MSAKETIEKLQNARIITALVTPFKENGQINFGAFPKLIEDLLANHTEGLILAGTTAESPTLTHDEELAIFAAVNKIVDGRIPLIAGVGTNDTRDSVEFVKEVAELGYIDAGLAVTPYYNKPSQEGIYQHFKAIATASDLPIILYNIPGRVVTEIQVETILRLAELENVIAIKECTNTDNLAYLIEKLPKDFLVYTGEDGLAFHTKALGGQGVISVASHILGQEFFEMFAEIDQGSIQKAAAIQRKILPKINALFSVTSPAPIKTVLNAKGYEVGGLRLPLVACTTEESKIILEKIGN

SEQ ID NO: 74 - protein, 473 aa, Lactococcus lactis
MKWSTKQRYRTYDSYSESDLESLRKLALKSPWKSNFHIEPETGLLNDPNGFSYFNEKWHLFYQHFPFGPVHGLKSWVHLVSDDLVHFEKTGLVLYPDTKYDNAGVYSGSALAFENFLFLIYTGNHRGEDWVRTPYQLGAKIDKNNQLVKFTEPLIYPDFSQTTDHFRDPQIFSFQGQIYCLIGAQSSQKNGIIKLYKAIENNLTDWKDLGNLDFSKEKMGYMIECPNLIFINGRSVLVFCPQGLDKSIVKYDNIYPNVYVIADDFTTGSKNQLKNAGQLINLDEGFDCYATQSFNAPDGSAYAISWLGLPETSYPTDKYNVQGVLSMVKKLSIKDNKLYQYPVEKMKELRQMEQDLLLADNNIITSNSYELEVDFRQQTSTLLSLMTNEKGDSALKVEIDKENNTITLIRNYEKRLAHVKIEKMNVFIDQSIFEIFINDGEKVLSDCRVFPNKNQYSIRSQNPIKIKLWELKK

SEQ ID NO: 75 - protein, 751 aa, Lactococcus lactis
MKQIKRIMGIDPWKITSNQIEKEDRRLQESLTSIGNGYMGMRGNFSETYSGDSHQGTYIAGVWFPDKTRVGWWKNGYPEYFGKAINALNFASVRVFIDDKEVDLAASHVTDFNLSLDMQKGVLTYTYVAYGVRVTAERFFSIAQQELAVFAFMFESLDGEIHQIRTVSVIDANVRNEDSNYDEKFWTVKNLDNTATGSFIVTETIPNPFGVEQFTVAAKQSFAGDFARVKQETRETSVLDVYEAKLVENAPLTFIKNVLVVTSRDIKPSNLTKVLSNLTLEISKKTYNKFYKEQEEAWAKRWEIADVQIDGSAEAQQGIRFNLFQLFSTYYGEDERLNIGPKGFTGEKYGGATYWDTEAYAVPLYLALSDEKVAKNLLKYRHNQLPQAQHNARQQGLKGALYPMVTFTGVECHNEWEITFEEIHRNGAMAYAIYNYTNYTGDETYLAQEGLEVLVEIARFWADRVHYSQRNDKYMIHGVTGPNEYENNINNNWYTNKLAAWVLTYTAESLEKYPRTDLISSEEVAHWGEIVDKMYYPEDKELGIFVQHDGYLDKDLTPVAQLDPKNLPLNQNWSWDKVLRSPYIKQADVLQGIYFFGNQFSMAEKQRNFDFYEPLTVHESSLSPSIHAILAAELGMEDKAVEMYERTARLDLDNYNNDTEDGLHITSMTGSWLAIVHGFAQMKTWEAQLSFAPFLPQAWIGYAFHINYRGCLLKISVGQEVKIELLRGQALSLKIYDETVELSDSYITKTR

SEQ ID NO: 76 - protein, 998 aa, Lactococcus lactis
MAMMTMIDVLERKDWENPVVSNWNRLPMHTPMDLLEKQSLNGLWNFDHFSRISDVPKNWLELTESKTEIIVPSNWQIEFKDKSDVPIYTNVTYPIPIQPPYVPEANPVGAYSRYFDITKEWLESGHVHLTFEGVGSAFHFWLNGEYGGYSEDSRLPAEFDISNLAKEGQNCLKVLVFRWSKVTYFEDQDMWRMSGIFRSVNLQWLPDNYLLDFSIKTDLDEDLDFANVKLQAYAKNIDDACLEFKLYDDEQLIGECHGFDAEIGVVNPKLWSDEIPYLYRLELTLMDRSGAVFHKETKKIGIRKIAIEKGQLKINGKALLVRGVNKHEFTPEHGYVVSEEVMIKDIKLMKEHNFNAVRCSHYPNDSRWYELCDEYGLYVMDEANIETHGMTPMNRLTNDPTYLPLMSERVTRMVMRDRNHPSIIIWSLGNESGYGSNHQALYDWCKSFDSSRPVHYEGGDDASRGATDATDIICPMYARVDSPSINAPYSLKTWMGVAGENRPLILCEYAHDMGNSLGGFGKYWQAFREIDRLQGGFIWDWVDQGLLKDGNYAYGGDFGDKPNDRQFSLNGLVFPNRQAKPALREAKYWQQYYQFELEKTPLGQVFAFTVTNEYLFRSTDNEKLCYQLINGLEVLWENELILNMPAGGSMRIDLSELPIDGTDNLFLNIQVKTIEKCNLLESDFEVAHQQFVLQEKINFTDRIDSNEEITLFEDEELLTVRSAKQKFIFNKSNGNLSRWLDEKGNEKLLHELSEQFTRAPLDNDIGVSEVEHIDPNAWLERWKGIGFYELKTLLKTMIIQATENEVIISVQTDYEAKGKIAFSTIREYHIFRNGELLLKVDFKRNIEFPEPARIGLSLQLAEKAENVTYFGLGPDENYPDRRGASLFGQWNLRITDMTTPYIFPSENGLRMETRELNYDRLKVRAMGQSFAFNLSPYSQNQLAKKGHWHLLEEEAGTWLNIDGFHMGVGGDDSWSPSVAQEYLLTKGNYHYEVSFKLT

SEQ ID NO: 77 - protein, 314 aa, Lactococcus lactis
MKSTFKQDLYYMFHSKIIIIFLSISTLLVFLGALQAINLQKSSIIQFEQTKLIYKNKNDFLKDLNKNYTENNITDDESNINTEVNNSARYSYDYVKKSNYQLTSLGFPLFILKYLGLIFLPIVMGILGILLSTTDYKYGTYKRRLSTNSWKEIITGKIVGLSSVIFGLYFYILILSMAVGLFLPKFSKFIDLKQYNIDSPNPSLFSAFTLVLCVLVLALITGILSFLISLSIKNLFVSLIGFLLYYLALPNLGKFDYKNVVMNIFSNAGKDVLGQPIPYIPLDIKVSLILFAIYVALISTGVLIIFNKYTKYSM

SEQ ID NO: 78 - protein, 769 aa, Lactococcus lactis
MTEKDWIIQYDKKEVGKRSYGQESLMSLGNGYLGLRGAPLWSTCSDNHYPGLYVAGVFNRTSTEVAGHDVINEDMVNWPNPQLIKVYIDGELVDFEASVEKQATIDFKNALQIERYQVKLAKGNLTLVTTKFVDPINFHDFGFVGEIIADFSCKLRIETFTDGSVLNQNVERYRAFDSKEFEVTKISKGLLVAKTRTSEIELAIASKSFLNGLAFPKIDSENDEILAEAIEIDLQKNQEVQFDKTIVIASSYESKNPVEFVLTELSATSVSKIQENNTNYWEKVWSDADIVIESDHEDLQRMVRMNIFHIRQAAQHGANQFLDASVGSRGLTGEGYRGHIFWDEIFVLPYYAANEPETARDLLLYRINRLTAAQENAKVDGEKGAMFPWQSGLIGDEQSQFVHLNTVNNEWEPDNSRRQRHVSLAIVYNLWIYSQLTEDESILTDGGLDLIIETTKFWLNKAELGDDGRYHIDGVMGPDEYHEAYPGQEGGICDNAYTNLMLTWQLNWLTELSEKGFEIPKELLEKAQKVRKKLYLDIDENGVIAQYAKYFELKEVDFAAYEAKYGDIHRIDRLMKAEGISPDEYQVAKQADTLMLIYNLGQEHVTKLVKQLAYELPENWLKVNRDYYLARTVHGSTTSRPVFAGIDVKLGDFDEALDFLITAIGSDYYDIQGGTTAEGVHIGVMGETLEVIQNEFAGLSLREGQFAIAPYLPKSWTKLKFNQIFRGTKVEILIENGQLLLTASADLLTKVYDDEVQLKAGVQTKFDLK

SEQ ID NO: 79 - protein, 445 aa, Lactococcus lactis
MNNFIQNKIMPPMMKFLNTRAVTAIKNGMIYPIPFIIIGSVFLILGQLPFQAGQDFMNKIKLGPLFLQINNASFGIMALLAVFGIAYAWVRDAGYEGVPAGLTGVIVHILLQPDTIHQVTSVTDPTKTSTAFQVGGVIDRAWLGGKGMVLSIIVGLLVGWIYTGFMRRNITIKMPEQVPENVAASFTSLVPAGAIITMAGVVHGITTIGFNTTFIELVYKWIQTPLQHVTDGPVGVFVIAFMPVFIWWFGVHGATIIGGIMGPLLQANSADNAALYKAGHLSLSNGAHIVTQSFMDQYLTVTGSGLTIGLVIFLLVSAKSVQGKTLGRMEIGPAVFNINEPILFGLPIVLNPILAIPFILAPLISGILTYLVIYLGIIPPFNGAYVPWTTPAVLSGYLVGGWQGMVWQIIILALTTVLYWPFAKAYDNILLKEEAETEAGINAAE

SEQ ID NO: 80 - protein, 650 aa, Lactococcus lactis
MNHKQVAERILNAVGRDNIQGARHCATRLRLVLKDTGVIDQEALDNDPDLKGTFEAAGQYQIIVGPGDVNTVYEEFIKLTGISEASTADLKEIAGSQKKQNPVMALVKLLSDIFVPLIPALVAGGLLMALNNALTAEHLFATKSLVEMFPMWKGFADIVNTMSAAPFTFMPILIGYSATKRFGGNPYLGAVVGMIMVMPGLINGYNVAEAISNHTMTYWDIFGFKVAQAGYQGQVLPVIGVAFILAKLERFFHKYLNDAIDFTFTPLLSVIITGFLTFTIVGPALRFVSNGLTDGLVGLYNTLGALGMLVFGGFYSAIVVTGLHQSFPAIETMLITNYQHSGIGGDFIFPVAACANMAQAGATFAILFVTKNIKTKALAAPAGVSAILGITEPALFGINLKLKYPFFIALGASAIGSLFMGLFHVLAVSLGSAGLIGFISIKAGYNLQFMISIFISFLIAFVVTSIYGRRMEAKSITKEKNKQNATTQYQPEKVIIDPVKSGELLAPINGFVIPLSDVSDPVFSKEIMGKGIAIKPKSGELFSPADGEIIIAYETGHAYGIKTKNGGEVLLHIGIDTVSMNGNGFIQNVKVGQKVKAGDLLGSFDKEEIKKSGLDDTVIIVITNSASYNEILPLSENVDIKVGEKILLLN

SEQ ID NO: 81 - protein, 409 aa, Lactococcus lactis
MKSWKKVALGGASVLALATLAACGSSASSNKSSSSSSSDSIKGTVRVYVDTQQKATYTDVAKGLTSKYPDLKVQIIANASGSANAKTDIAKDPSKYADVFAVPNDQLGDMADKGFISPVATKFADEIKNDNSKITVAGVTYKDKVYAFPKSTEAQVLFYNKSKLSADDVKSWDTMTSKAVFATDFTNAYNFYPVFFSAGTQLFGASGEDVTGTNVASDKGVTAMKWFADQKANKNVMQTSNALNQLQSGKADAIIDGPWDTANIKKILGDNFAVAPYPTITLNGEQKQLEAFQGIKGFAVNSATKDQAASQTVAQYLTTKAAQLKLFNSQGDVPTNLDAQKDDAVKSSDATKAVITMAKEGNSVVMPKLPQMATFWNNAAPLINGAYTGSIKATDYQAQLQKFQDSISK

SEQ ID NO: 82 - protein, 460 aa, Lactococcus lactis
MTKKKKRKQTESNVSPEEKSIKLREVFQKGNTVTKLTFFVMGLNQIKNKQWVKGFTFLILEIAFIGWLLFSGLSAFSLLSSLGPNKTLKETTDANGFPVIIQPDHSVLILLWGLIACLVVVLFILLYWFNYRSNKHLYYLEREGKHIPTNREELASLLDEKLYATLMAVPLIGVLAFTVLPTVYMISMAFTNYDRLHATAFSWTGFQAFGNVLTGDLAGTFFPVLGWTLVWAIVATATTFLGGVLLALLIESTGIKFKGFWRTVFVIVFAVPQFVTLLMMAQFLDQQGAFNGILMNLHLISNPINFIGAASDPMVARITVIFVNMWIGIPVSMLVSTAIIQNLPQDQIEAARIDGANSLNIFRSITFPQILFVMTPALIQQFIGNINNFNVIYLLTQGWPMNPNYQGAGSTDLLVTWLYNLVFGQTQRYNAAAVLGILIFIVNASISLVAYRRTNAFKEG

SEQ ID NO: 83 - protein, 288 aa, Lactococcus lactis
MKSYKTQRRISLTLRYILLALLAIVWIFPIIWIVLASLTQNNTGFVSTIIPKTFTFENYIQLFQNKSGSFPFVSWIINTFIVAVISATLSTFITIIMSYTLSRLRFAFRKPFLQIALVLGMFPGFMSMIALYYILKAMNMLNLGGLILVYVGGAGLGFYIAKGFFDTIPRSIDEAATIDGANKWQVFTHITLPLSRPIIVYTALMAFIAPWTDFIFSGIILGNNQAHPETFTIAYGLYSMVHSQKGAATAFFTQFIAGCVIIAIPITILFVIMQKFYVNGITAGADKG

SEQ ID NO: 84 - protein, 568 aa, Lactococcus lactis
MHKLIELIEKGKPFFEKISRNIYLRAIRDGFIAGMPVILFSSIFILIAYVPNAWGFHWSKDIETFLMTPYSYSMGILAFFVGGTTAKALTDSKNRDLPATNQINFLSTMLASMVGFLLMAAEPAKEGGFLTAFMGTKGLLTAFIAAFVTVNVYKVCVKNNVTIRMPEDVPPNISQVFKDLIPFTVSVVLLYGLELLVKGTLGVTVAESIGTLIAPLFSAADGYLGITLIFGAYAFFWFVGIHGPSIVEPAIAAITYANIDVNLHLIQAGQHADKVITSGTQMFIATMGGTGATLIVPFLFMWICKSDRNRAIGRASVVPTFFGVNEPILFGAPIVLNPIFFVPFIFTPIVNVWIFKFFVDTLNMNSFSANLPWVTPGPLGIVLGTNFQVLSFILAGLLVVVDTIIYYPFVKVYDEQILEEERSGKTNDALKEKVAENFNTAKADAVLGKAGVAKEDVAANNNITKETNVLVLCAGGGTSGLLANALNKAAAEYNVPVKAAAGGYGAHREMLPEFDLVILAPQVASNFDDMKAETDKLGIKLVKTEGAQYIKLTRDGKGALAFVQQQFD

SEQ ID NO: 85 - protein, 215 aa, Lactococcus lactis
MTIKFKHAYKSFGKKIIFKDASININRNSIYFIMAPNGSGKTTFFKIITNLQTLDKGKVYNDCSNRKQFSIFDDLSLYKNLTGYQNIQLFTNFKFNKFEIEQHSKKYEMLSKLNQKVSTYSLGEGKKISLLLWELLNPDLVIMDEVTNGLDHNTLKELKSSLLKAKEDSIIILTGHELLFYEEIIDDLYILNNGKLLKELNWKEEGLTKTYEKYF

SEQ ID NO: 86 - protein, 462 aa, Lactococcus lactis
MKEGKMKQRLSYAFGALGHDVYYYSISTFFIAFVTAQMFAGTPHEDAMIALVTSLVVIIRLIEIIFDPIIGSIIDNTHTRWGKFKPWLVVGGIMSSLMIMLMFSDFFGLAKSDNRTLFAIVFIIAFIILDAFYSFKDIAFWSMIPALSEKNSERETLGTFARFGSAIGAQGATILAIPITIFFTKGGHAQGARGFFAFGVIAALVQGISALVTAWGTKEQKSVIRQEGTKTNTLDVFKALLKNDQLMWLSLSYILFAIAYVATTATLILNFTFVIGNASLYSITGIVGFIGSIILVPMFPILAKKFGRRKVLTGAIISMLLGYLLFVLGSSVAMTVAGLIFLTAPYQLVFLSVLMTITDSVEYGQWKNGVRNEAVTLAMRPLLDKIAGAFSNGIYGFVAISAGMTGSKYIAGHTYGVATFKLYSFVVPAILMIIALAVYLFKVKLTEKRHEEIVAELEERLK

SEQ ID NO: 87 - protein, 573 aa, Lactococcus lactis
MIDLVIGKIPLANWVSSATDWITSTFSSGFDVIQKSGTVLMNGITGALTAVPFWLMIAVVTILAILVSGKKFAFPLFAFIGLCLIANQGLWSDLMSTITLVLLSSLLSIIIGVPLGIWMAKSELVAKIVQPILDFMQTMPGFVYLIPAVAFFGIGVVPGVFASVIFALPPTVRMTNLGIRQVSTELVEAADSFGSTARQKLFKLEFPLAKGTIMAGVNQTIMLALSMVVIASMIGAPGLGRGVLAAVQSADIGKGFVSGISLVILAIIIDRFTQKLNVSPLEKQGNPKLKKWKRWIAIVSLLALIVGAFSGMSFGKKSSDKKVDLVYMNWDSEVASINVLTQAMEEHSFDVTTTALDNAVAWQTVANSQADGMVSAWLPNTHKTQWKKYGKSVELLGPNLKGAKVGFVVPSYMNVNSIEDLTNQANKTITGIEPGAGVMAASENTLKSYSNLKDWKLVPSSSGAMTVALGEAIKQHKDIVITGWSPHWIFNKYDLKYLADPKGTMGTSENINTIVRKGLKKENPEAYKVLNNFNWTTKDMESVMLDIQNGKTPEAAAKAWIKDHQKQVDKWFK

SEQ ID NO: 88 - protein, 474 aa, Escherichia coli
MSRLVVVSNRIAPPDEHAASAGGLAVGILGALKAAGGLWFGWSGETGNEDQPLKKVKKGNITWASFNLSEQDLDEYYNQFSNAVLWPAFHYRLDLVQFQRPAWDGYLRVNALLADKLLPLLQDDDIIWIHDYHLLPFAHELRKRGVNNRIGFFLHIPFPTPEIFNALPTYDTLLEQLCDYDLLGFQTENDRLAFLDCLSNLTRVTTRSAKSHTAWGKAFRTEVYPIGIEPKEIAKQAAGPLPPKLAQLKAELKNVQNIFSVERLDYSKGLPERFLAYEALLEKYPQHHGKIRYTQIAPTSRGDVQAYQDIRHQLENEAGRINGKYGQLGWTPLYYLNQHFDRKLLMKIFRYSDVGLVTPLRDGMNLVAKEYVAAQDPANPGVLVLSQFAGAANELTSALIVNPYDRDEVAAALDRALTMSLAERISRHAEMLDVIVKNDINHWQECFISDLKQIVPRSAESQQRDKVATFPKLA

SEQ ID NO: 89 - protein, 266 aa, Escherichia coli
MTEPLTETPELSAKYAWFFDLDGTLAEIKPHPDQVVVPDNILQGLQLLATASDGALALISGRSMVELDALAKPYRFPLAGVHGAERRDINGKTHIVHLPDAIARDISVQLHTVIAQYPGAELEAKGMAFALHYRQAPQHEDALMTLAQRITQIWPQMALQQGKCVVEIKPRGTSKGEAIAAFMQEAPFIGRTPVFLGDDLTDESGFAVVNRLGGMSVKIGTGATQASWRLAGVPDVWSWLEMITTALQQKRENNRSDDYESFSRSI

SEQ ID NO: 90 - DNA, 7145 bp, Lactococcus lactis
tatgttgttgaagaaatgaagaaaaataattataacttcggtggtgaacaatctggtcatatgattttccttgattacaacacgactggagatggccaactttcagctattcaattattaaaagtaatgcgtgaaacgggtaaaactttatctgaattagcaagtgaagttacaatttatcctcaaaaattagttaatgttcgtgtaaaagacaatgcagctaagaaatcagcgatggatgtgccagctattcagaaagttatttcagaaatggaaacttcaatgaatggtaaaggacgtattttagttcgcccttcaggtactgagccccttttacgtgtgatggctgaagctccaactcatgaacaagttgaccatgttgtggatacaattgttgaagttgttgaagaggaaattggtgtgaaataaagaaaagacaaggagaatattcttcttgtcttttttcatatcctaaaactctacctactgtggtagagtttttttatcttttttggcgtctagcaaactctgtaaaacgaaaacggtcaacctgatgtcgtgattcagtatactggaaaagtgtattatcggcaagatagacatgagatttcacggaaacaacatgatggtccttcggatttaaatcaagatatgtaaaatcatcttcacaagcaaaatcaatggtaacttctttttgggcataggcaatatcaagtcccaaagccccttctaaataatcgtaagtagaattttgggcatgtgcgggggtcaaaccatcagcgtatttttctaaaaataaatcccaatccaaaatggaaaatttaccatctacttttcttcttctaagaatactaagggcttggtcgccgatggcgaatccagtagtttctgaaagagctggggtaatttttatactttcaaattttattacttcagtttcactgtgaaaacccattgaagtttgcaattctttatatgaagttaagccggaaatagggaaaaggagccgatcgtgagcgaggacaatgctgccatagccatgtcttctttggatgagccctttttcttctaaaatttttaaagcttgtctgacggttgaacggctactttcataactaatagaaagttcattctcgcttggaagaatatcgttcgttttatagatatcattaaaaatctttttttctaaatcttgcaaaatcacttcatatttcttcatactttatattttatcataaaaataattgttaacgcttgctgaaaacgtttttatgtgatacaatgattttgttaacttgtacgtacaactgtaaaagataggagattcttatgtttggaataggaaaaaagaaagaattgagagatgataaaagcctttatgctccagtttctggggaagttatcaacctttcaacagtcaacgaccccgtattttcaaaaaagataatgggagacgggttcgcggttgagccaaaagaaaataaaatttttgccccagtttctgcaaaagtaactttggttcaaggacatgcaattggttttaaacgtgctgatggcttagatgtacttttacatcttggaattgatacagtagctcttaaaggtcttcattttaaaatcaaggtcaaagttgatgatattgtcaatggtggtgatgagcttggaagcgttgattgggcacagattgaagctgcaggtttagataaaacgacaatggttatctttacaaatacaaaagataaactctctgagttcaatgtcaattatggaccagctacttctggaagtgaacttggtaaggcaagtgttaaataaagaataaaaatttaaggttaatccatttataaggcaaaggtgacgagggctttgtatataaattttgtgactattaaaatcgtcaattattattttatattatgaaatacttcataaataatgaagtaaaaggagaacttatggcaaattattcacaacttgcgacagaaattatcgcaaatgtaggtggcgctgagaatgtcacaaaagttattcactgtatcactcgtcttcgttttaccttgaaagacaaagataaagcagatacggcggcgattgaagccttacctggtgtcgctggagctgtttataactcaaacttgaatcaatatcaagtagttattggacaagctgtagaagatgtttatgacgaggttgttgaacagcttggagattcagttgttgatgaagatgcaacggcgcaagcacttgctgcaacagcaccggctagtggtaaaaaacaaaatccaattgttcatgctttccaagtggttattgggacaattacaggttcgatgattccaattattggtttacttgcggctggtgggatgattaatggattattaagtatctttgttaaaggaaatcgtttaattgaagtgattgaccctgcaagttcaacttacgtcattatctcaactctagcaatgacaccattttatttcttacctgttttagtaggattttcagcagcaaaacaattagcacctaaagatactgttttacaatttattggtgctgctgttggtggtttcatgattaatccagggattactaacttggtaaatgctcatgttggaacaaatgcggccggtaaaaatgttgttgttgaagcagcagctccagtagcaaatttccttggagtcacttttaatacaagttattttggaattccggttgctttgccaagttatgcttatacaattttcccaatcattgtggcggtagcaatcgctaaacctttgaatgcttggttgaaaaaggttttaccacttgccttgcgtccaattttccaaccgatgattactttcttcatcactgcttcaatcattttactcttggtcggtcctgttatttcaacaatttcatctggtttgtcattcgttattgaccatatcttgtcattaaacttagggattgcaagtattatcgtcggtggtttgtatcaatgtttggttatatttggtttgcactggttggttgtaccacttatttcacaagagttggcagcaacaggagcaagctcacttaatatgattgttagcttcacaatgcttgcgcaaggagttggtgccttgactgtcttctttaaatctaaaaaagctgaccttaaaggactttctgctccagctgccatttcggctttttgtggagtaactgaacctgccatgtacggaattaacttgaaatatgttcgcgtcttcatcatgtcttcaattggtgcagcaattggtgctgggattgccggatttggtggcttacaaatgtttggattttcagggtcattgattagttttcctaactttatctctaatccattgacgcatcatgcacctgcgggtaacttaatgctcttctggattgccactgcggtatgtgctgttgccactttcttattagtttggttctttggttacaaggatactgatgtcatgggacaaggagttgaacaaaaaaatgcatttaaggatgctgtaaaataattaacagaattgtttgggagcagagcgaataaattctgcttcctttagtttaataaagggggagaaatgactgataaagattggatgatccaatatgacaaacaagaagtagggaagcgttcttatgggcaagaatctttaatgtcattgggaaatggttatttagggcttcgtggggcgcctttgtgggcaacttgttcggataatcattatccgggactctatgtcgcaggggtctttaatcacacaagtacagaagttgcaggtcatgatgttatcaacgaagatatggtcaattggccaaatccacaattgattaaggtttatattgataatgaattggttgacttcgaggcagcaattgagaaaaattcttcgattgatttcaaaaatggattgcaaattgagagctataatgtcagtttagccaagggaggtttgactttagtgaccacaaaatttgttgatcccatccattttcacgattttgggttcgttggagaaatcatcgctgatttttctggaaaattgcgaatagaaacttttattgatggttcggtattgaatcaaaatgttgaacgctatcgggcttttgacagcaaagaatttgaagtgactcaaattgctgatggacttttggtggcaaaaactagaacgacggacatagaattagcagttgcgactaaaacttatttaaatggtcagccattgaaaaaagtagaatctggaaattctgaaatttttaaagaatccattgaagttgatttactaaaaaaccaagaagttcagtttgaaaaatcgattgttattgctagttcttatgaaaccaaaaaccctgttgaatttgtgctgacagaactggcagcaacttctgtcagtaaaattcaggaaaataatgcaaattattgggagaaagtatggcaggatggcgatattgtcatcgaatctgatcatgcggatttgcaaagaatggtgcgaatgaatattttccatattcgccaagcggcacaacacggtgctaatcagtttttagatgcgtccgtaggttcgcgtggattgactggtgaaggttatcgaggacatattttctgggatgaaatttttgttctaccttattatgcggcgaatgaaccagaaacagcgcgtgatttgcttttgtaccgaatcaatcgattgactgctgcacaggaaaatgcaaaggttgatggagaaataggggcaatgtttccttggcaatccggcttaattggggatgaacaggcacaatttgttcatttgaatacagtaaataatgaatgggaaccagataatagtcgccgtcaaagacatgtcagcttagctattgtttacaatctgtggatttacttacagctgacagatgatgaaagtattttgactgacggtggactggatttgctcgttgaaaccacgaagttttggttaaacaaagcagaattgggaagtgatagccgctatcatatcgctggtgtcatgggtcctgatgaatatcatgaggcttatccagggcaagaaggtggtatttgcgataatgcttatacgaatttgatgctgacttggcagttaaattggctgacagagctgtcagtgaaaggttttgaaattccagcagatttgcttgaagagtcacaaaaggttcgggaaaatctttatttagatattgatgagaatggtgtgattgcccaatatgctaagtattttgagcttaaagaagttgattttgcagcttatgaagcaaaatatggcgatattcatcggattgaccgtttgatgaaggctgagggaatttcgcctgacgaatatcaagtggctaaacaagctgataccttgatgttaatgtacaatttgggtcatgaacatgtgatcaaattggtcaaacaattaggttatgagctacccaaaaattggttgaaagttaatcgtgattattatcttgcacgaactgtccatggttcaacgacatctcgtccagtttttgctgggattgatgtcaaattgggtgattttgatgaagcgcttgactttttaatcactgcgattggaagtgattactatgatattcaaggcggaaccacggccgaaggggttcacattggggtcatgggagaaacacttgaagtgattcaaaatgaatttgccggtttgacactacgcgatggatacttttcaattgctccgcatttaccaaaaagttggaccaaattgaaattcagtcaaattttcaaaggttgtcaagtggaaattttgattgaaaaaggtcaattattactgacagcttcatcagacttgctgattaaagtttatgatgaggaagttcagttaaaagcaggagtacaagctaattttgatttaaaataaatagttttgctcttaataaagttttgatacaaggatttacaattattttttgataaaaaaattactgatagaaatgaaaaaaattctgtcagtaattttggaaagtcattctaaaaaattcattttaaaatgacgagaaagaaggtaaaaagatgtttaaagcagtattgtttgatttagatggcgtaattacagataccgcagagtatcattatagagcatggaaagctttagctgaggaaattggtattaatggtgtcgatcgtcagtttaatgagcaacttaaaggagtttctcgtgaagattctctacagaaaattttagatttggctggcaaaaaagtatcggcagaagaatttaaggcactagctcaaagaaaaaatgataactatgtgactatgattcaagatgtttccccagctgatgtttatcctggaatcttacaattactcaaagatttgcgttctaatcatatcaagattgctttagcttctgcctcaaaaaatgggccatttttattggaacggatggccttgactgaatattttgatgcgattgcagatccagcagaagttgcggcatctaaaccagctccagatatttttattgcggcagctcatgccgttggtgtaaaaccgactgaatcaattggtttagaggattctcaagctggaattcaagcgattaaagattcaggggcgctaccaattggagttgggcgaccagaggatttgggaaatgatatcgtcgttgtagctgacacttctcattatactttggattttcttaaagaagtttggttaaaagaaaaaggatagtttctatttaaaacgaccaaaattttgtcaaaaaaaatgccccagttgagtgggcattttaattttttatttgtctttctaaccaatctgaggcccaagagaaccaacgatgaactgaatcaagacaataagcatcagaaggagctgttgtccgattcgccaaggaaatgccatgtggtcctgcttcaaaaaaatgagcttcaaatgggacttggtgtttggacaggcggtcacaatatttaagactgttatagataggaacacttgcatcatcagcggtatgccagatgaaagtagggggtgtagccgaagtcactttttctgaaatattgtattctgaaatattttcaatttcaaagttaaaatgtgataaatcacttggccaaccaaaagtgaaggaagtaacagggtaacaaagaattacaccctt